(19) United States
(12) Patent Application Publication
(10) Pub. No.: US 2018/0170904 A1
(43) Pub. Date: Jun. 21, 2018

(54) PYRIDIN-3-YL ACETIC ACID DERIVATIVES AS INHIBITORS OF HUMAN IMMUNODEFICIENCY VIRUS REPLICATION

(71) Applicant: ViiV HEALTHCARE UK (NO.5) LIMITED, Brentford, Middlesex (GB)

(72) Inventors: John F. KADOW, Wallingford, CT (US); B. Narasimhulu NAIDU, Wallingford, CT (US); Manoj PATEL, Wallingford, CT (US); Jeffrey Lee ROMINE, Wallingford, CT (US); Denis R. ST. LAURENT, Wallingford, CT (US); Tao WANG, Wallingford, CT (US); Zhongxing ZHANG, Wallingford, CT (US)

(21) Appl. No.: 15/579,993

(22) PCT Filed: Jul. 7, 2016
(86) PCT No.: PCT/IB2016/054090

§ 371 Date: Dec. 6, 2017

Related U.S. Application Data
(60) Provisional application No. 62/190,338, filed on Jul. 9, 2015.

(51) Int. Cl. C07D 401/04; C07D 417/14; C07D 413/14; A61P 31/18

(52) U.S. Cl. CPC C07D 401/04; A61P 31/18; C07D 413/14; C07D 417/14

(57) ABSTRACT

Disclosed are compounds of Formula I, including pharmaceutically acceptable salts, pharmaceutical compositions comprising the compounds, methods for making the compounds and their use in inhibiting HIV integrase and treating those infected with HIV or AIDS.

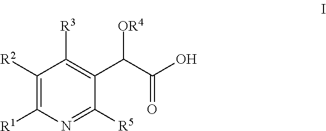

### CROSS REFERENCE TO RELATED INVENTION

This application claims the benefit of U.S. provisional application Ser. No. 62/190,338 filed Jul. 9, 2015.

### FIELD OF THE INVENTION

The invention relates to compounds, compositions, and methods for the treatment of human immunodeficiency virus (HIV) infection. More particularly, the invention provides novel inhibitors of HIV, pharmaceutical compositions containing such compounds, and methods for using these compounds in the treatment of HIV infection. The invention also relates to methods for making the compounds hereinafter described.

### BACKGROUND OF THE INVENTION

Human immunodeficiency virus (HIV) has been identified as the etiological agent responsible for acquired immune deficiency syndrome (AIDS), a fatal disease characterized by destruction of the immune system and the inability to fight off life threatening opportunistic infections. Recent statistics indicate that an estimated 35.3 million people worldwide are infected with the virus (UNAIDS: Report on the Global HIV/AIDS Epidemic, 2013). In addition to the large number of individuals already infected, the virus continues to spread. Estimates from 2013 point to close to 3.4 million new infections in that year alone. In the same year there were approximately 1.6 million deaths associated with HIV and AIDS.

Current therapy for HIV-infected individuals consists of a combination of approved anti-retroviral agents. Over two dozen drugs are currently approved for HIV infection, either as single agents or as fixed dose combinations or single tablet regimens, the latter two containing 2-4 approved agents. These agents belong to a number of different classes, targeting either a viral enzyme or the function of a viral protein during the virus replication cycle. Thus, agents are classified as either nucleotide reverse transcriptase inhibitors (NRTIs), non-nucleotide reverse transcriptase inhibitors (NNRTIs), protease inhibitors (PIs), integrase inhibitors (INIs), or entry inhibitors (one, maraviroc, targets the host CCR5 protein, while the other, enfuvirtide, is a peptide that targets the gp41 region of the viral gp160 protein). In addition, a pharmacokinetic enhancer with no antiviral activity, i.e., cobicistat, available from Gilead Sciences, Inc. under the tradename TYBOST™ (cobicistat) tablets, has recently been approved for use in combinations with certain antiretroviral agents (ARVs) that may benefit from boosting.

In the US, where combination therapy is widely available, the number of HIV-related deaths has dramatically declined (Palella, F. J.; Delany, K. M.; Moorman, A. C.; Loveless, M. O.; Furher, J.; Satten, G. A.; Aschman, D. J.; Holmberg, S. D. N. Engl. J. Med. 1998, 338, 853-860).

Unfortunately, not all patients are responsive and a large number fail this therapy. In fact, initial studies suggest that approximately 30-50% of patients ultimately fail at least one drug in the suppressive combination. Treatment failure in most cases is caused by the emergence of viral resistance. Viral resistance in turn is caused by the replication rate of HIV-1 during the course of infection combined with the relatively high viral mutation rate associated with the viral polymerase and the lack of adherence of HIV-infected individuals in taking their prescribed medications. Clearly, there is a need for new antiviral agents, preferably with activity against viruses already resistant to currently approved drugs. Other important factors include improved safety and a more convenient dosing regimen than many of the currently approved drugs.

Compounds which inhibit HIV replication have been disclosed. See, for example, the following patent applications: WO2007131350, WO2009062285, WO2009062288, WO2009062289, WO2009062308, WO2010130034, WO2010130842, WO2011015641, WO2011076765, WO2012033735, WO2013123148, WO2013134113, WO2014164467, WO2014159959, and WO2015126726.

What is now needed in the art are additional compounds which are novel and useful in the treatment of HIV. Additionally, these compounds may desirably provide advantages for pharmaceutical uses, for example, with regard to one or more of their mechanisms of action, binding, inhibition efficacy, target selectivity, solubility, safety profiles, or bioavailability. Also needed are new formulations and methods of treatment which utilize these compounds.

### SUMMARY OF THE INVENTION

The invention encompasses compounds of Formula I, including pharmaceutically acceptable salts thereof, as well as pharmaceutical compositions, and their use in inhibiting HIV and treating those infected with HIV or AIDS.

By virtue of the present invention, it is now possible to provide compounds that are novel and are useful in the treatment of HIV. Additionally, the compounds may provide advantages for pharmaceutical uses, for example, with regard to one or more of their mechanism of action, binding, inhibition efficacy, target selectivity, solubility, safety profiles, or bioavailability.

The invention also provides pharmaceutical compositions comprising the compounds of the invention, including pharmaceutically acceptable salts thereof, and a pharmaceutically acceptable carrier, excipient, and/or diluent.

In addition, the invention provides methods of treating HIV infection comprising administering a therapeutically effective amount of the compounds of the invention to a patient.

In addition, the invention provides methods for inhibiting HIV integrase.

Also provided in accordance with the invention are methods for making the compounds of the invention.

The present invention is directed to these, as well as other important ends, hereinafter described.

### DESCRIPTION OF THE INVENTION

Unless specified otherwise, these terms have the following meanings.

“Alkyl” means a straight or branched saturated hydrocarbon comprised of 1 to 10 carbons, and preferably 1 to 6 carbons.

“Alkenyl” means a straight or branched alkyl group comprised of 2 to 10 carbons with at least one double bond and optionally substituted with 0-3 halo or alkoxy group.

“Alkynyl” means a straight or branched alkyl group comprised of 2 to 10 carbons, preferably 2 to 6 carbons, containing at least one triple bond and optionally substituted with 0-3 halo or alkoxy group.

“Aryl” mean a carbocyclic group comprised of 1-3 rings that are fused and/or bonded and at least one or a combination of which is aromatic. The non-aromatic carbocyclic portion, where present, will be comprised of C₃ to C₇ alkyl group. Examples of aromatic groups include, but are not limited to indanyl, indenyl, naphthyl, phenyl, tetrahydronaphthyl and cyclopropylphenyl. The aryl group can be attached to the parent structure through any substitutable carbon atom in the group.

“Arylalkyl” is a C₁-C₅ alkyl group attached to 1 to 2 aryl groups and linked to the parent structure through the alkyl moiety. Examples include, but are not limited to, —(CH₂)ₙPh with n=1-5, —CH(CH₃)Ph, —CH(Ph)₂.

“Aryloxy” is an aryl group attached to the parent structure by oxygen.

“Cycloalkyl” means a monocyclic ring system composed of 3 to 7 carbons.

“Halo” includes fluoro, chloro, bromo, and iodo.

“Haloalkyl” and “haloalkoxy” include all halogenated isomers from monohalo to perhalo.

“Heteroaryl” is a subset of heterocyclic group as defined below and is comprised of 1-3 rings where at least one or a combination of which is aromatic and that the aromatic group contains at least one atom chosen from a group of oxygen, nitrogen or sulfur.

“Heterocyclyl or heterocyclic” means a cyclic group of 1-3 rings comprised of carbon and at least one other atom selected independently from oxygen, nitrogen and sulfur. The rings could be bridged, fused and/or bonded, through a direct or spiro attachment, with the option to have one or a combination thereof be aromatic. Examples include, but are not limited to, azaindole, azaindoline, azetidine, benzimidazole, bezodioxolyl, benzoisothiazole, benzothiazole, benzothiadiazole, benzothiophene, benzoxazole, carbazole, chroman, dihalobezodioxolyl, dihydrobenzofuran, dihydrobenzo[1,4]oxazine, 1,3-dihydrobenzo[c]thiophene 2,2-dioxide, 2,3-dihydrobenzo[d]isothiazole 1,1-dioxide, 3,4-dihydro-2H-pyrido[3,2-b][1,4]oxazine, 2,3-dihydro-1H-pyrrolo[3,4-c]pyridine and its regioisomeric variants, 6,7-dihydro-5H-pyrrolo[2,3-b]pyrazine and its regioisomeric variants, furanylphenyl, imidazole, imidazo[1,2-a]pyridine, indazole, indole, indoline, isoquinoline, isoquinolinone, isothiazolidine 1,1-dioxide, morpholine, 2-oxa-5-azabicyclo[2.2.1]heptane, oxadiazole-phenyl, oxazole, phenylaztidine, phenylindazole, phenylpiperidine, phenylpiperizine, phenyloxazole, phenylpyrrolidine, piperidine, pyridine, pyridinylphenyl, pyridinylpyrrolidine, pyrimidine, pyrimidinylphenyl, pyrrazole-phenyl, pyrrolidine, pyrrolidin-2-one, 1H-pyrazolo[4,3-c]pyridine and its regioisomeric variants, pyrrole, 5H-pyrrolo[2,3-b]pyrazine, 7H-pyrrolo[2,3-d]pyrimidine and its regioisomeric variants, quinazoline, quinoline, quinoxaline, tetrahydroisoquinoline, 1,2,3,4-tetrahydro-1,8-naphthyridine, tetrahydroquinoline, 4,5,6,7-tetrahydrothieno[3,2-c]pyridine, 1,2,5-thiadiazolidine 1,1-dioxide, thiophene, thiophenylphenyl, triazole, or triazolone. Unless otherwise specifically set forth, the heterocyclic group can be attached to the parent structure through any suitable atom in the group that results in a stable compound.

It is understood that a subset of the noted heterocyclic examples encompass regioisomers. For instance, “azaindole” refers to any of the following regioisomers: 1H-pyrrolo[2,3-b]pyridine, 1H-pyrrolo[2,3-c]pyridine, 1H-pyrrolo[3,2-c]pyridine, and 1H-pyrrolo[3,2-b]pyridine. In addition the “regioisomer variants” notation as in, for example, “5H-pyrrolo[2,3-b]pyrazine and its regioisomeric variants” would also encompass 7H-pyrrolo[2,3-d]pyrimidine, 7H-pyrrolo[2,3-c]pyridazine, 1H-pyrrolo[2,3-d]pyridazine, 5H-pyrrolo[3,2-c]pyridazine, and 5H-pyrrolo[3,2-d]pyrimidine. Similarly, 6,7-dihydro-5H-pyrrolo[2,3-b]pyrazine and its regioisomeric variants would encompass 6,7-dihydro-5H-pyrrolo[2,3-d]pyrimidine and 6,7-dihydro-5H-pyrrolo[2,3-c]pyridazine. It is also understood that the lack of “regioisomeric variants” notation does not in any way restrict the claim scope to the noted example only.

“Heterocyclylalkyl” is a heterocyclyl moiety attached to the parent structure through C₁-C₅ alkyl group. Examples include, but are not limited to, —(CH₂)ₙ—R^{Z} or —CH(CH₃)—(R^{Z}) where n=1-5 and that R^{Z} is chosen from benzimidazole, imidazole, indazole, isooxazole, phenyl-pyrazole, pyridine, quinoline, thiazole, triazole, triazolone, oxadiazole.

Terms with a hydrocarbon moiety (e.g. alkoxy) include straight and branched isomers for the hydrocarbon portion with the indicated number of carbon atoms.

Bonding and positional bonding relationships are those that are stable as understood by practitioners of organic chemistry.

Parenthetic and multiparenthetic terms are intended to clarify bonding relationships to those skilled in the art. For example, a term such as ((R)alkyl) means an alkyl substituent further substituted with the substituent R.

Substituents which are illustrated by chemical drawing to bond at variable positions on a multiple ring system (for example a bicyclic ring system) are intended to bond to the ring where they are drawn to append. Parenthetic and multiparenthetic terms are intended to clarify bonding relationships to those skilled in the art. For example, a term such as ((R)alkyl) means an alkyl substituent further substituted with the substituent R.

“Combination,” “coadministration,” “concurrent” and similar terms referring to the administration of a compound of Formula I with at least one anti-HIV agent mean that the components are part of a combination antiretroviral therapy or highly active antiretroviral therapy (“HAART”) as understood by practitioners in the field of AIDS and HIV infection.

“Therapeutically effective” means the amount of agent required to provide a benefit to a patient as understood by practitioners in the field of AIDS and HIV infection. In general, the goals of treatment are suppression of viral load, restoration and preservation of immunologic function, improved quality of life, and reduction of HIV-related morbidity and mortality.

“Patient” means a person infected with the HIV virus.

“Treatment,” “therapy,” “regimen,” “HIV infection,” “ARC,” “AIDS” and related terms are used as understood by practitioners in the field of AIDS and HIV infection.

Those terms not specifically set forth herein shall have the meaning which is commonly understood and accepted in the art.

The invention includes all pharmaceutically acceptable salt forms of the compounds. Pharmaceutically acceptable salts are those in which the counter ions do not contribute significantly to the physiological activity or toxicity of the compounds and as such function as pharmacological equivalents. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc.

Some of the compounds of the invention exist in stereoisomeric forms. The invention includes all stereoisomeric forms of the compounds including enantiomers and diastereromers. Methods of making and separating stereoisomers are known in the art. The invention includes all tautomeric forms of the compounds. The invention includes atropisomers and rotational isomers.

The invention is intended to include all isotopes of atoms occurring in the present compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. Such compounds may have a variety of potential uses, for example as standards and reagents in determining biological activity. In the case of stable isotopes, such compounds may have the potential to favorably modify biological, pharmacological, or pharmacokinetic properties.

In an aspect of the invention, there is provided a compound of Formula I:

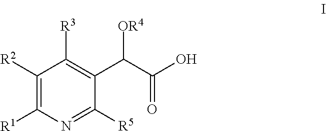

wherein:
R¹ is selected from hydrogen or alkyl;
R² is selected from phenyl, pyridinyl, or pyrimidinyl, and is substituted with 0-1 substituent selected from R⁶, R⁷, R⁸, and R⁹, and also substituted with 0-3 substituents selected from cyano, halo, alkyl, cyanoalkyl, haloalkyl, hydroxyalkyl, alkoxyalkyl, cyanoalkenyl, cycloalkyl, cyanocycloalkyl, hydroxyalkyl, alkoxy, haloalkoxy, formyl, carboxy, and CH₃CONHNHCO—;
R³ is selected from azetidinyl, pyrrolidinyl, piperidinyl, piperazinyl, morpholinyl, homopiperidinyl, homopiperazinyl, or homomorpholinyl, and is substituted with 0-3 substituents selected from cyano, halo, alkyl, haloalkyl, cyanoalkyl, cycloalkyl, alkenyl, alkoxy, haloalkoxy, phenyl, or benzyl;
R⁴ is selected from alkyl or haloalkyl;
R⁵ is alkyl;
R⁶ is selected from CONR¹⁰R¹¹ or (CONR¹⁰R¹¹)alkyl;
R⁷ is selected from Ar², (Ar²)alkyl, (Ar²)hydroxyalkyl , (Ar²)alkenyl, or (Ar²)alkylcarbonyl;
R⁸ is selected from alkylthio, (Ar¹)alkylthio, alkylsulfonyl, (Ar¹)alkylsulfonyl, ((Ar¹)alkyl)(alkoxycarbonylN═)S, ((Ar¹)alkyl)(alkoxycarbonylN═)(O)S, or SONR¹²R¹³;
R⁹ is NR¹⁴R¹⁵;
R¹⁰ is selected from hydrogen, alkyl, cycloalkyl, (Ar¹)alkyl, (Ar¹)haloalkyl, ((Ar¹)CO)alkyl, ((Ar¹)CH₂CO)alkyl;
R¹¹ is selected from hydrogen or alkyl;
or NR¹⁰R¹¹ taken together is selected from azetidinyl, pyrrolidinyl, piperidinyl, piperazinyl, morpholinyl, homopiperidinyl, homopiperazinyl, or homomorpholinyl;
R¹² is selected from hydrogen, alkyl, or cycloalkyl;
R¹³ is selected from hydrogen, alkyl, or cycloalkyl;
or NR¹²R¹³ taken together is selected from azetidinyl, pyrrolidinyl, piperidinyl, piperazinyl, morpholinyl, homopiperidinyl, homopiperazinyl, or homomorpholinyl;
R¹⁴ is selected from hydrogen, alkyl, (Ar¹)alkyl, (Ar¹)hydroxyalkyl, (Ar¹)alkylcarbonyl, or benzyloxycarbonyl;
R¹⁵ is selected from hydrogen, alkyl, hydroxyalkyl, (Ar¹)alkyl, or alkylcarbonyl;
Ar¹ is selected from phenyl or pyridinyl and is substituted with 0-3 substituents selected from halo, alkyl, haloalkyl, alkoxy, haloalkoxy, carboxy, and alkoxycarbonyl; and
Ar² is selected from phenyl or naphthyl and is substituted with 0-3 substituents selected from halo, alkyl, haloalkyl, alkoxy, haloalkoxy, phenyl, and pyridinyl; or Ar² is selected from oxazolyl, thiazolyl, imidazolyl, or tetrazolyl, and is substituted with 0-2 substituents selected from halo, (Ar¹), and (Ar¹)alkyl;
   or a pharmaceutically acceptable salt thereof.

In an aspect of the invention, R² is selected from phenyl, pyridinyl, or pyrimidinyl, and is substituted with 1 R⁶ substituent and also substituted with 0-3 substituents selected from cyano, halo, alkyl, cyanoalkyl, haloalkyl, hydroxyalkyl, alkoxyalkyl, cyanoalkenyl, cycloalkyl, cyanocycloalkyl, hydroxyalkyl, alkoxy, haloalkoxy, formyl, carboxy, and CH₃CONHNHCO—.

In an aspect of the invention, R² is selected from phenyl, pyridinyl, or pyrimidinyl, and is substituted with 1 substituent and also substituted with 0-3 substituents selected from cyano, halo, alkyl, cyanoalkyl, haloalkyl, hydroxyalkyl, alkoxyalkyl, cyanoalkenyl, cycloalkyl, cyanocycloalkyl, hydroxyalkyl, alkoxy, haloalkoxy, formyl, carboxy, and CH₃CONHNHCO—.

In an aspect of the invention, R² is selected from phenyl, pyridinyl, or pyrimidinyl, and is substituted with 1 R⁸ substituent and also substituted with 0-3 substituents selected from cyano, halo, alkyl, cyanoalkyl, haloalkyl, hydroxyalkyl, alkoxyalkyl, cyanoalkenyl, cycloalkyl, cyanocycloalkyl, hydroxyalkyl, alkoxy, haloalkoxy, formyl, carboxy, and CH₃CONHNHCO—.

In an aspect of the invention, R² is selected from phenyl, pyridinyl, or pyrimidinyl, and is substituted with 1 R⁹ substituent and also substituted with 0-3 substituents selected from cyano, halo, alkyl, cyanoalkyl, haloalkyl, hydroxyalkyl, alkoxyalkyl, cyanoalkenyl, cycloalkyl, cyanocycloalkyl, hydroxyalkyl, alkoxy, haloalkoxy, formyl, carboxy, and CH₃CONHNHCO—.

In an aspect of the invention, R³ is piperidinyl, gem-disubstituted in the 4-position with 2 substituents selected from cyano, halo, alkyl, haloalkyl, cycloalkyl, halocycloalkyl, alkenyl, alkoxy, haloalkoxy, CON(R⁶)(R⁷), phenyl, benzyl, or (alkyl)oxadiazolyl.

In an aspect of the invention, R¹⁰ is selected from hydrogen, alkyl, cycloalkyl, (Ar¹)alkyl, (Ar¹)haloalkyl, ((Ar¹)CO)alkyl, ((Ar¹)CH₂CO)alkyl; and R¹¹ is selected from hydrogen or alkyl.

In an aspect of the invention, NR¹⁰R¹¹ taken together is selected from azetidinyl, pyrrolidinyl, piperidinyl, piperazinyl, morpholinyl, homopiperidinyl, homopiperazinyl, or homomorpholinyl.

In an aspect of the invention, R¹² and R¹³ are selected from hydrogen, alkyl, or cycloalkyl.

In an aspect of the invention, NR¹²R¹³ taken together is selected from azetidinyl, pyrrolidinyl, piperidinyl, piperazinyl, morpholinyl, homopiperidinyl, homopiperazinyl, or homomorpholinyl.

In an aspect of the invention, Ar² is selected from phenyl or naphthyl and is substituted with 0-3 substituents selected from halo, alkyl, haloalkyl, alkoxy, haloalkoxy, phenyl, and pyridinyl.

In an aspect of the invention, Ar² is selected from oxazolyl, thiazolyl, imidazolyl, or tetrazolyl, and is substituted with 0-2 substituents selected from halo, (Ar¹), and (Ar¹)alkyl.

For a particular compound of Formula I, the scope of any instance of a variable substituent, including R¹, R², R³, R⁴, R⁵, R⁶, R^{7,} R^{8,} R^{9,} R¹⁰, R¹¹, R^{12,} R^{13,} R^{14,} R¹⁵ Ar¹ and Ar² can be used independently with the scope of any other instance of a variable substituent. As such, the invention includes combinations of the different aspects.

In an aspect of the invention, there is provided a composition useful for treating HIV infection comprising a therapeutic amount of a compound of Formula I and a pharmaceutically acceptable carrier. In an aspect of the invention, the composition further comprises a therapeutically effective amount at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors, and a pharmaceutically acceptable carrier. In an aspect of the invention, the other agent is dolutegravir.

In an aspect of the invention, there is provided a method for treating HIV infection comprising administering a therapeutically effective amount of a compound of Formula I, or a pharmaceutically acceptable salt thereof, to a patient in need thereof. In an aspect of the invention, the method further comprises administering a therapeutically effective amount of at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors. In an aspect of the invention, the other agent is dolutegravir. In an aspect of the invention, the other agent is administered to the patient prior to, simultaneously with, or subsequently to the compound of Formula I.

Preferred compounds in accordance with the present invention include the following:
(S)-2-(tert-Butoxy)-2-(4-(4,4-dimethylpiperidin-1-yl)-5-(4-((4-fluoro-3-methylbenzyl)carbamoyl)phenyl)-2,6-dimethylpyridin-3-yl)acetic acid;
(S)-2-(tert-Butoxy)-2-(4-(4,4-dimethylpiperidin-1-yl)-5-(4-((4-fluorophenethyl)carbamoyl)phenyl)-2,6-dimethylpyridin-3-yl)acetic acid;
(S)-2-(tert-Butoxy)-2-(4-(4,4-dimethylpiperidin-1-yl)-5-(4-(5-(4-fluorobenzyl)thiazol-2-yl)phenyl)-2,6-dimethylpyridin-3-yl)acetic acid;
(S)-2-(tert-Butoxy)-2-(4-(4,4-dimethylpiperidin-1-yl)-5-(4-(5-(4-fluorobenzyl)oxazol-2-yl)phenyl)-2,6-dimethylpyridin-3-yl)acetic acid;
(S)-2-(tert-Butoxy)-2-(4-(4,4-dimethylpiperidin-1-yl)-5-(4-(5-(4-fluorophenyl)oxazol-2-yl)phenyl)-2,6-dimethylpyridin-3-yl)acetic acid;
(S)-2-(tert-Butoxy)-2-(4-(4,4-dimethylpiperidin-1-yl)-5-(4-(5-(4-fluorophenyl)thiazol-2-yl)phenyl)-2,6-dimethylpyridin-3-yl)acetic acid;
(S)-2-(tert-Butoxy)-2-(4-(4,4-dimethylpiperidin-1-yl)-2,6-dimethyl-5-phenylpyridin-3-yl)acetic acid;
(S)-2-(5-([1,1′-Biphenyl]-4-yl)-4-(4,4-dimethylpiperidin-1-yl)-2,6-dimethylpyridin-3-yl)-2-(tert-butoxy)acetic acid;
(S)-2-(tert-Butoxy)-2-(4-(4,4-dimethylpiperidin-1-yl)-2,6-dimethyl-5-(4′-propoxy-[1,1′-biphenyl]-4-yl)pyridin-3-yl)acetic acid;
(S)-2-(tert-Butoxy)-2-(4-(4,4-dimethylpiperidin-1-yl)-5-(4′-isopropoxy-[1,1′-biphenyl]-4-yl)-2,6-dimethylpyridin-3-yl)acetic acid;
(S)-2-(tert-Butoxy)-2-(5-(4′-chloro-[1,1′-biphenyl]-4-yl)-4-(4,4-dimethylpiperidin-1-yl)-2,6-dimethylpyridin-3-yl)acetic acid;
(S)-2-(tert-Butoxy)-2-(4-(4,4-dimethylpiperidin-1-yl)-5-(4′-ethyl-[1,1′-biphenyl]-4-yl)-2,6-dimethylpyridin-3-yl)acetic acid;
(S)-2-(tert-Butoxy)-2-(4-(4,4-dimethylpiperidin-1-yl)-2,6-dimethyl-5-(4-(pyridin-2-yl)phenyl)pyridin-3-yl)acetic;
(S)-2-(tert-Butoxy)-2-(4-(4,4-dimethylpiperidin-1-yl)-5-(2-fluoro-[1,1′-biphenyl]-4-yl)-2,6-dimethylpyridin-3-yl)acetic acid;
(S)-2-(5-(4-Benzylphenyl)-4-(4,4-dimethylpiperidin-1-yl)-2,6-dimethylpyridin-3-yl)-2-(tert-butoxy)acetic acid;
(S)-2-(tert-Butoxy)-2-(4-(4,4-dimethylpiperidin-1-yl)-5-(4′-methoxy-[1,1′-biphenyl]-4-yl)-2,6-dimethylpyridin-3-yl)acetic acid;
(S)-2-(tert-Butoxy)-2-(4-(4,4-dimethylpiperidin-1-yl)-2,6-dimethyl-5-(3′-methyl-[1,1′-biphenyl]-4-yl)pyridin-3-yl)acetic acid;
(S)-2-(tert-Butoxy)-2-(4-(4,4-dimethylpiperidin-1-yl)-2,6-dimethyl-5-(4-(naphthalen-1-yl)phenyl)pyridin-3-yl)acetic acid;
(S)-2-(tert-Butoxy)-2-(4-(4,4-dimethylpiperidin-1-yl)-5-(3-fluoro-4-methylphenyl)-2,6-dimethylpyridin-3-yl)acetic acid;
(S)-2-(tert-Butoxy)-2-(4-(4,4-dimethylpiperidin-1-yl)-5-(4-isobutylphenyl)-2,6-dimethylpyridin-3-yl)acetic acid;
(S)-2-(tert-Butoxy)-2-(4-(4,4-dimethylpiperidin-1-yl)-5-(3′-methoxy-[1,1′-biphenyl]-4-yl)-2,6-dimethylpyridin-3-yl)acetic acid;
(S)-2-(tert-Butoxy)-2-(4-(4,4-dimethylpiperidin-1-yl)-2,6-dimethyl-5-(4-pentylphenyl)pyridin-3-yl)acetic acid;
(S)-2-(tert-Butoxy)-2-(4-(4,4-dimethylpiperidin-1-yl)-5-(4-(isopentylthio)phenyl)-2,6-dimethylpyridin-3-yl)acetic acid;
(S)-2-(5-(4-(Benzylcarbamoyl)phenyl)-4-(4,4-dimethylpiperidin-1-yl)-2,6-dimethylpyridin-3-yl)-2-(tert-butoxy)acetic acid;
(S)-2-(tert-Butoxy)-2-(5-(4-carbamoylphenyl)-4-(4,4-dimethylpiperidin-1-yl)-2,6-dimethylpyridin-3-yl)acetic acid;
(S)-2-(5-(4-(2-Amino-2-oxoethyl)phenyl)-4-(4,4-dimethylpiperidin-1-yl)-2,6-dimethylpyridin-3-yl)-2-(tert-butoxy)acetic acid;
(S)-2-(tert-Butoxy)-2-(5-(4-(1-cyanocyclopropyl)phenyl)-4-(4,4-dimethylpiperidin-1-yl)-2,6-dimethylpyridin-3-yl)acetic acid
(S)-2-(tert-Butoxy)-2-(4-(4,4-dimethylpiperidin-1-yl)-2,6-dimethyl-5-(pyrimidin-5-yl)pyridin-3-yl)acetic acid;
(S)-2-(tert-Butoxy)-2-(4-(4,4-dimethylpiperidin-1-yl)-2,6-dimethyl-5-(4-(piperidin-1-yl)phenyl)pyridin-3-yl)acetic acid;
(S)-2-(tert-Butoxy)-2-(4-(4,4-dimethylpiperidin-1-yl)-5-(4-(isopropylsulfonyl)phenyl)-2,6-dimethylpyridin-3-yl)acetic acid;
(S)-2-(tert-Butoxy)-2-(4-(4,4-dimethylpiperidin-1-yl)-5-(4-(3-hydroxypropyl)phenyl)-2,6-dimethylpyridin-3-yl)acetic acid;
(S)-2-(tert-Butoxy)-2-(4-(4,4-dimethylpiperidin-1-yl)-2,6-dimethyl-5-(4-(N-methylsulfamoyl)phenyl)pyridin-3-yl)acetic acid;
(S)-2-(tert-Butoxy)-2-(4-(4,4-dimethylpiperidin-1-yl)-2,6-dimethyl-5-(3,4,5-trifluorophenyl)pyridin-3-yl)acetic acid;
(S)-2-(tert-Butoxy)-2-(5-(3,5-difluoro-4-(hydroxymethyl)phenyl)-4-(4,4-dimethylpiperidin-1-yl)-2,6-dimethylpyridin-3-yl)acetic acid;
(S)-2-(tert-Butoxy)-2-(5-(4-(N-cyclopropylsulfamoyl)phenyl)-4-(4,4-dimethylpiperidin-1-yl)-2,6-dimethylpyridin-3-yl)acetic acid;
(S)-2-(tert-Butoxy)-2-(5-(4-(2-cyanopropan-2-yl)phenyl)-4-(4,4-dimethylpiperidin-1-yl)-2,6-dimethylpyridin-3-yl)acetic acid;
(S)-2-(5-(4-(2-Acetylhydrazinecarbonyl)phenyl)-4-(4,4-dimethylpiperidin-1-yl)-2,6-dimethylpyridin-3-yl)-2-(tert-butoxy)acetic acid;
(S)-2-(tert-Butoxy)-2-(4-(4,4-dimethylpiperidin-1-yl)-5-(4-(2-hydroxypropan-2-yl)phenyl)-2,6-dimethylpyridin-3-yl)acetic acid;
(S)-2-(tert-Butoxy)-2-(4-(4,4-dimethylpiperidin-1-yl)-5-(4-fluoro-3-methylphenyl)-2,6-dimethylpyridin-3-yl)acetic acid;
(S)-2-(tert-Butoxy)-2-(4-(4,4-dimethylpiperidin-1-yl)-5-(3-isopropylphenyl)-2,6-dimethylpyridin-3-yl)acetic acid;
(S)-2-(tert-Butoxy)-2-(5-(3,4-difluorophenyl)-4-(4,4-dimethylpiperidin-1-yl)-2,6-dimethylpyridin-3-yl)acetic acid;
(S)-2-(tert-Butoxy)-2-(4-(4,4-dimethylpiperidin-1-yl)-2,6-dimethyl-5-(2-(methylthio)pyrimidin-5-yl)pyridin-3-yl)acetic acid;
(S)-2-(tert-Butoxy)-2-(5-(4-(cyclopropylcarbamoyl)phenyl)-4-(4,4-dimethylpiperidin-1-yl)-2,6-dimethylpyridin-3-yl)acetic acid;
(S)-2-(tert-Butoxy)-2-(5-(3,4-dimethylphenyl)-4-(4,4-dimethylpiperidin-1-yl)-2,6-dimethylpyridin-3-yl)acetic acid;
(S)-2-(benzylcarbamoyl)-4-(5-(tert-butoxy(carboxy)methyl)-4-(4,4-dimethylpiperidin-1-yl)-2,6-dimethylpyridin-3-yl)benzoic acid;
(S)-2-(5-(4-(1H-Tetrazol-5-yl)phenyl)-4-(4,4-dimethylpiperidin-1-yl)-2,6-dimethylpyridin-3-yl)-2-(tert-butoxy)acetic acid;
(S)-2-(5-(3-(1H-Tetrazol-5-yl)phenyl)-4-(4,4-dimethylpiperidin-1-yl)-2,6-dimethylpyridin-3-yl)-2-(tert-butoxy)acetic acid′
(S)-2-(tert-Butoxy)-2-(5-(4-(cyanomethyl)phenyl)-4-(4,4-dimethylpiperidin-1-yl)-2,6-dimethylpyridin-3-yl)acetic acid;
(S,E)-2-(tert-Butoxy)-2-(5-(4-(2-cyanovinyl)phenyl)-4-(4,4-dimethylpiperidin-1-yl)-2,6-dimethylpyridin-3-yl)acetic acid;
(S)-2-(tert-Butoxy)-2-(4-(4,4-dimethylpiperidin-1-yl)-5-(4-(3-(4-fluorophenyl)propanoyl)phenyl)-2,6-dimethylpyridin-3-yl)acetic acid;
(2S)-2-(tert-Butoxy)-2-(4-(4,4-dimethylpiperidin-1-yl)-5-(4-(3-(4-fluorophenyl)-1-hydroxypropyl)phenyl)-2,6-dimethylpyridin-3-yl)acetic acid;
(S)-2-(tert-Butoxy)-2-(4-(4,4-dimethylpiperidin-1-yl)-5-(4-(3-(4-fluorophenyl)propyl)phenyl)-2,6-dimethylpyridin-3-yl)acetic acid;
(S)-2-(tert-Butoxy)-2-(4-((3,5-dimethylpiperidin-1-yl)-5-(4-(4-fluorophenethoxy)phenyl)-2,6-dimethylpyridin-3-yl)acetic acid;
(S)-2-(5-(4-(Benzylcarbamoyl)phenyl)-2,6-dimethyl-4-(4-(trifluoromethyl)piperidin-1-yl)pyridin-3-yl)-2-(tert-butoxy)acetic acid;
(2S)-2-(5-(4-(Benzylcarbamoyl)phenyl)-4-(3-cyclopropylpyrrolidin-1-yl)-2,6-dimethylpyridin-3-yl)-2-(tert-butoxy)acetic acid;
(S)-2-(5-(4-(Benzylcarbamoyl)phenyl)-4-(4-ethyl-4-methylpiperidin-1-yl)-2,6-dimethylpyridin-3-yl)-2-(tert-butoxy)acetic acid;
(S)-2-(5-(4-(Benzylcarbamoyl)phenyl)-4-(3,3-dimethylazetidin-1-yl)-2,6-dimethylpyridin-3-yl)-2-(tert-butoxy)acetic acid;
(S)-2-(5-(4-(Benzylcarbamoyl)phenyl)-2,6-dimethyl-4-(4-methylpiperidin-1-yl)pyridin-3-yl)-2-(tert-butoxy)acetic acid;
(S)-2-(5-(4-(Benzylcarbamoyl)phenyl)-4-(3-isopropylazetidin-1-yl)-2,6-dimethylpyridin-3-yl)-2-(tert-butoxy)acetic acid;
(S)-2-(5-(4-(Benzylcarbamoyl)phenyl)-4-(4-methoxy-4-methylpiperidin-1-yl)-2,6-dimethylpyridin-3-yl)-2-(tert-butoxy)acetic acid;
(S)-2-(5-(4-(Benzylcarbamoyl)phenyl)-4-(4-cyano-4-methylpiperidin-1-yl)-2,6-dimethylpyridin-3-yl)-2-(tert-butoxy)acetic acid;
(S)-2-(5-(4-(Benzylcarbamoyl)phenyl)-2,6-dimethyl-4-(4-phenylpiperidin-1-yl)pyridin-3-yl)-2-(tert-butoxy)acetic acid;
(S)-2-(5-(4-(Benzylcarbamoyl)phenyl)-2,6-dimethyl-4-(4-(propan-2-ylidene)piperidin-1-yl)pyridin-3-yl)-2-(tert-butoxy)acetic acid;
(S)-2-(5-(4-(Benzylcarbamoyl)phenyl)-4-(4-benzylpiperidin-1-yl)-2,6-dimethylpyridin-3-yl)-2-(tert-butoxy)acetic acid;
(S)-2-(5-(4-(Benzylcarbamoyl)phenyl)-4-(4-fluoro-4-methylpiperidin-1-yl)-2,6-dimethylpyridin-3-yl)-2-(tert-butoxy)acetic acid;
(S)-2-(tert-Butoxy)-2-(4-(4,4-dimethylpiperidin-1-yl)-2,6-dimethyl-5-(4-((pyridin-2-ylmethyl)carbamoyl)phenyl)pyridin-3-yl)acetic acid;
(S)-2-(tert-Butoxy)-2-(4-(4,4-dimethyl piperidin-1-yl)-5-(4-((2-ethoxy-4,6-difluorobenzyl)carbamoyl)phenyl)-2,6-dimethylpyridin-3-yl)acetic acid;
(S)-4-(5-(tert-Butoxy(carboxy) methyl)-4-(4,4-dimethylpiperidin-1-yl)-2,6-dimethylpyridin-3-yl)benzoic acid;
(S)-2-(tert-Butoxy)-2-(4-(4,4-dimethylpiperidin-1-yl)-2,6-dimethyl-5-(4-((2,4,6-trifluorobenzyl)carbamoyl)phenyl)pyridin-3-yl)acetic acid;
(S)-2-(5-(4-Aminophenyl)-4-(4,4-dimethylpiperidin-1-yl)-2,6-dimethylpyridin-3-yl)-2-(tert-butoxy)acetic acid;
(S)-2-(tert-Butoxy)-2-(4-(4,4-dimethylpiperidin-1-yl)-5-(4-((ethoxycarbonyl)amino)phenyl)-2,6-dimethylpyridin-3-yl)acetic acid;
(S)-2-(tert-Butoxy)-2-(4-(4,4-dimethylpiperidin-1-yl)-5-(4-(2-(4-fluorophenyl)acetamido)phenyl)-2,6-dimethylpyridin-3-yl)acetic acid;
(S)-2-(tert-Butoxy)-2-(4-(4,4-dimethylpiperidin-1-yl)-5-(4-((4-fluorophenethyl)amino)phenyl)-2,6-dimethylpyridin-3-yl)acetic acid;
(2S)-2-(tert-Butoxy)-2-(4-(4,4-dimethylpiperidin-1-yl)-5-(4-((4-fluorophenethyl)(2-(4-fluorophenyl)-1-hydroxyethyl)amino)phenyl)-2,6-dimethylpyridin-3-yl)acetic acid;
(S)-2-(tert-Butoxy)-2-(4-(4,4-dimethylpiperidin-1-yl)-5-(4-(N-(4-fluorophenethyl)acetamido)phenyl)-2,6-dimethylpyridin-3-yl)acetic acid;
(S)-2-(tert-Butoxy)-2-(4-(4,4-dimethylpiperidin-1-yl)-5-(4-((4-fluorophenethyl)(methyl)amino)phenyl)-2,6-dimethylpyridin-3-yl)acetic acid;
(S)-2-(tert-Butoxy)-2-(4-(4,4-dimethylpiperidin-1-yl)-5-(4-((4-fluorophenethyl)(2-hydroxyethyl)amino)phenyl)-2,6-dimethylpyridin-3-yl)acetic acid;
(S)-2-(tert-Butoxy)-2-(4-(4,4-dimethylpiperidin-1-yl)-5-(4-((4-fluorophenethyl)thio)phenyl)-2,6-dimethylpyridin-3-yl)acetic acid;
(S)-2-(tert-Butoxy)-2-(4-(4,4-dimethylpiperidin-1-yl)-5-(4-((4-fluorophenethyl)sulfonyl)phenyl)-2,6-dimethylpyridin-3-yl)acetic acid;
(2S)-2-(tert-Butoxy)-2-(5-(4-((E)-N-(tert-butoxycarbonyl)-S-(4-fluorophenethyl)sulfinimidoyl)phenyl)-4-(4,4-dimethylpiperidin-1-yl)-2,6-dimethylpyridin-3-yl)acetic acid;
(2S)-2-(tert-Butoxy)-2-(5-(4-(N-(tert-butoxycarbonyl)-2-(4-fluorophenyl)ethylsulfonimidoyl)phenyl)-4-(4,4-dimethylpiperidin-1-yl)-2,6-dimethylpyridin-3-yl)acetic acid;
(S)-2-(tert-Butoxy)-2-(4-(4,4-dimethylpiperidin-1-yl)-5-(4-(hydroxymethyl)phenyl)-2,6-dimethylpyridin-3-yl)acetic acid;
(S)-2-(tert-Butoxy)-2-(4-(4,4-dimethylpiperidin-1-yl)-5-(4-formylphenyl)-2,6-dimethylpyridin-3-yl)acetic acid;
(S)-2-(tert-Butoxy)-2-(4-(4,4-dimethylpiperidin-1-yl)-5-(4-(4-fluorostyryl)phenyl)-2,6-dimethylpyridin-3-yl)acetic acid;
(S)-2-(tert-Butoxy)-2-(4-(4,4-dimethylpiperidin-1-yl)-5-(4-(4-fluorophenethyl)-phenyl)-2,6-dimethylpyridin-3-yl)acetic acid;
(S)-2-(tert-Butoxy)-2-(4-(4,4-dimethylpiperidin-1-yl)-5-(4-(1-(4-fluorobenzyl)-1H-tetrazol-5-yl)phenyl)-2,6-dimethylpyridin-3-yl)acetic acid and/or (S)-2-(tert-Butoxy)-2-(4-(4,4-dimethylpiperidin-1-yl)-5-(4-(2-(4-fluorobenzyl)-2H-tetrazol-5-yl)phenyl)-2,6-dimethylpyridin-3-yl)acetic acid;
(S)-2-(tert-Butoxy)-2-(4-(4,4-dimethylpiperidin-1-yl)-5-(3-(1-(4-fluorobenzyl)-1H-tetrazol-5-yl)phenyl)-2,6-dimethylpyridin-3-yl)acetic acid and/or (S)-2-(tert-Butoxy)-2-(4-(4,4-dimethylpiperidin-1-yl)-5-(3-(2-(4-fluorobenzyl)-2H-tetrazol-5-yl)phenyl)-2,6-dimethylpyridin-3-yl)acetic acid; and
(2S)-2-(tert-Butoxy)-2-(4-(4,4-dimethylpiperidin-1-yl)-2,6-dimethyl-5-(4-((2,2,2-trifluoro-1-phenylethyl)carbamoyl)phenyl)pyridin-3-yl)acetic acid; and
   pharmaceutically acceptable salts thereof.

The compounds of the invention herein described may typically be administered as pharmaceutical compositions. These compositions are comprised of a therapeutically effective amount of a compound of Formula I or its pharmaceutically acceptable salt, and a pharmaceutically acceptable carrier and may contain conventional excipients and/or diluents. A therapeutically effective amount is that which is needed to provide a meaningful patient benefit. Pharmaceutically acceptable carriers are those conventionally known carriers having acceptable safety profiles. Compositions encompass all common solid and liquid forms, including capsules, tablets, lozenges, and powders, as well as liquid suspensions, syrups, elixirs, and solutions. Compositions are made using available formulation techniques, and excipients (such as binding and wetting agents) and vehicles (such as water and alcohols) which are generally used for compositions. See, for example, Remington's Pharmaceutical Sciences, 17th edition, Mack Publishing Company, Easton, Pa. (1985).

Solid compositions which are normally formulated in dosage units and compositions providing from about 1 to 1000 milligram (“mg”) of the active ingredient per dose are typical. Some examples of dosages are 1 mg, 10 mg, 100 mg, 250 mg, 500 mg, and 1000 mg. Generally, other antiretroviral agents will be present in a unit range similar to agents of that class used clinically. Typically, this is about 0.25-1000 mg/unit.

Liquid compositions are usually in dosage unit ranges. Generally, the liquid composition will be in a unit dosage range of about 1-100 milligram per milliliter (“mg/mL”). Some examples of dosages are 1 mg/mL, 10 mg/mL, 25 mg/mL, 50 mg/mL, and 100 mg/mL. Generally, other antiretroviral agents will be present in a unit range similar to agents of that class used clinically. Typically, this is about 1-100 mg/mL.

The invention encompasses all conventional modes of administration; oral and parenteral methods are preferred. Generally, the dosing regimen will be similar to other antiretroviral agents used clinically. Typically, the daily dose will be about 1-100 milligram per kilogram (“mg/kg”) body weight daily. Generally, more compound is required orally and less parenterally. The specific dosing regimen, however, will be determined by a physician using sound medical judgment.

The compounds of this invention desirably have activity against HIV. Accordingly, another aspect of the invention is a method for treating HIV infection in a human patient comprising administering a therapeutically effective amount of a compound of Formula I, or a pharmaceutically acceptable salt thereof, with a pharmaceutically acceptable carrier, excipient and/or diluent.

The invention also encompasses methods where the compound is given in combination therapy. That is, the compound can be used in conjunction with, but separately from, other agents useful in treating AIDS and HIV infection. The compound can also be used in combination therapy wherein the compound and one or more of the other agents are physically together in a fixed-dose combination (FDC). Some of these agents include HIV attachment inhibitors, CCR5 inhibitors, CXCR4 inhibitors, HIV cell fusion inhibitors, HIV integrase inhibitors, HIV nucleoside reverse transcriptase inhibitors, HIV non-nucleoside reverse transcriptase inhibitors, HIV protease inhibitors, budding and maturation inhibitors, HIV capsid inhibitors, anti-infectives, and immunomodulators, such as, for example, PD-1 inhibitors, PD-L1 inhinitors, antibodies, and the like. In these combination methods, the compound of Formula I will generally be given in a daily dose of about 1-100 mg/kg body weight daily in conjunction with other agents. The other agents generally will be given in the amounts used therapeutically. The specific dosing regimen, however, will be determined by a physician using sound medical judgment.

Examples of nucleoside HIV reverse transcriptase inhibitors include abacavir, didanosine, emtricitabine, lamivudine, stavudine, tenofovir, zalcitabine, and zidovudine.

Examples of non-nucleoside HIV reverse transcriptase inhibitors include delavirdine, efavirenz, etrivirine, nevirapine, and rilpivirine.

Examples of HIV protease inhibitors include amprenavir, atazanavir, darunavir, fosamprenavir, indinavir, lopinavir, nelfinavir, ritonavir, saquinavir and, tipranavir.

An example of an HIV fusion inhibitor is enfuvirtide or T-1249.

An example of an HIV entry inhibitor is maraviroc.

Examples of HIV integrase inhibitors include dolutegravir, elvitegravir, or raltegravir.

An example of an HIV attachment inhibitor is fostemsavir.

An example of an HIV maturation inhibitor is BMS-955176, having the following structure:

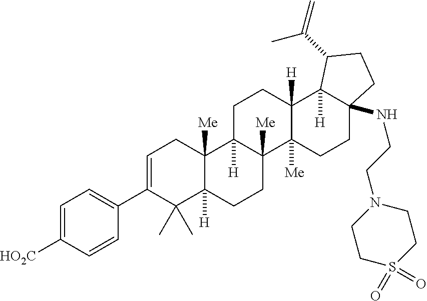

Thus, as set forth above, contemplated herein are combinations of the compounds of Formula I, together with one or more agents useful in the treatment of AIDS. For example, the compounds of the invention may be effectively administered, whether at periods of pre-exposure and/or post-exposure, in combination with effective amounts of the AIDS antivirals, immunomodulators, anti-infectives, or vaccines, such as those in the following non-limiting table:

| | | |
|---|---|---|
| Drug Name | Manufacturer | Indication |

ANTIVIRALS

| | | |
|---|---|---|
| Rilpivirine | Tibotec | HIV infection, AIDS, ARC |
| | | (non-nucleoside reverse |
| | | transcriptase inhibitor) |
| COMPLERA ® | Gilead | HIV infection, AIDS, ARC; |
| | | combination with emtricitabine, |
| | | rilpivirine, and tenofovir |
| | | disoproxil fumarate |
| 097 | Hoechst/Bayer | HIV infection, AIDS, ARC |
| | | (non-nucleoside reverse |
| | | transcriptase (RT) inhibitor) |
| Amprenavir | Glaxo Wellcome | HIV infection, AIDS, ARC |
| 141 W94 | | (protease inhibitor) |
| GW 141 | | |
| Abacavir (1592U89) | Glaxo Wellcome | HIV infection, AIDS, ARC |
| GW 1592 | | (RT inhibitor) |
| Acemannan | Carrington Labs | ARC |
| | (Irving, TX) | |
| Acyclovir | Burroughs Wellcome | HIV infection, AIDS, ARC |
| AD-439 | Tanox Biosystems | HIV infection, AIDS, ARC |
| AD-519 | Tanox Biosystems | HIV infection, AIDS, ARC |
| Adefovir dipivoxil | Gilead Sciences | HIV infection ARC, PGL |
| AL-721 | Ethigen | HIV positive, AIDS |
| | (Los Angeles, CA) | |
| Alpha Interferon | Glaxo Wellcome | Kaposi's sarcoma, |
| | | HIV in combination w/Retrovir |
| Ansamycin | Adria Laboratories | ARC |
| LM 427 | (Dublin, OH) | |
| | Erbamont | |
| | (Stamford, CT) | |
| Antibody which | Advanced Biotherapy | AIDS, ARC |
| Neutralizes pH | Concepts | |
| Labile alpha aberrant | (Rockville, MD) | |
| Interferon | | |
| AR177 | Aronex Pharm | HIV infection, AIDS, ARC |
| Beta-fluoro-ddA | Nat'l Cancer Institute | AIDS-associated diseases |
| CI-1012 | Warner-Lambert | HIV-1 infection |
| Cidofovir | Gilead Science | CMV retinitis, herpes, |
| | | papillomavirus |
| Curdlan sulfate | AJI Pharma USA | HIV infection |
| Cytomegalovirus | MedImmune | CMV retinitis |
| Immune globin | | |
| Cytovene | Syntex | Sight threatening |
| Ganciclovir | | CMV peripheral CMV retinitis |
| Darunavir | Tibotec- J & J | HIV infection, AIDS, ARC |
| | | (protease inhibitor) |
| Delaviridine | Pharmacia-Upjohn | HIV infection, AIDS, ARC |
| | | (RT inhibitor) |
| Dextran Sulfate | Ueno Fine Chem. | AIDS, ARC, HIV positive |
| | Ind. Ltd. (Osaka, | asymptomatic |
| | Japan) | |
| ddC | Hoffman-La Roche | HIV infection, AIDS, ARC |
| Dideoxycytidine | | |
| ddI | Bristol-Myers Squibb | HIV infection, AIDS, ARC; |
| Dideoxyinosine | | combination with AZT/d4T |
| DMP-450 | AVID | HIV infection, AIDS, ARC |
| | (Camden, NJ) | (protease inhibitor) |
| Efavirenz | Bristol Myers Squibb | HIV infection, AIDS, ARC |
| (DMP 266, SUSTIVA ®) | | (non-nucleoside RT |
| (−)6-Chloro-4-(S)- | | inhibitor) |
| cyclopropylethynyl- | | |
| 4(S)-trifluoro- | | |
| methyl-1,4-dihydro- | | |
| 2H-3,1-benzoxazin- | | |
| 2-one, STOCRINE | | |
| EL10 | Elan Corp, PLC | HIV infection |
| | (Gainesville, GA) | |
| Etravirine | Tibotec/J & J | HIV infection, AIDS, ARC |
| | | (non-nucleoside reverse |
| | | transcriptase inhibitor) |
| Famciclovir | Smith Kline | herpes zoster, herpes |
| | | simplex |
| GS 840 | Gilead | HIV infection, AIDS, ARC |
| | | (reverse transcriptase |
| | | inhibitor) |
| HBY097 | Hoechst Marion | HIV infection, AIDS, ARC |
| | Roussel | (non-nucleoside reverse |
| | | transcriptase inhibitor) |
| Hypericin | VIMRx Pharm. | HIV infection, AIDS, ARC |
| Recombinant Human | Triton Biosciences | AIDS, Kaposi's sarcoma, |
| Interferon Beta | (Almeda, CA) | ARC |
| Interferon alfa-n3 | Interferon Sciences | ARC, AIDS |
| Indinavir | Merck | HIV infection, AIDS, ARC, |
| | | asymptomatic HIV positive, |
| | | also in combination with |
| | | AZT/ddI/ddC |
| ISIS 2922 | ISIS Pharmaceuticals | CMV retinitis |
| KNI-272 | Nat'l Cancer Institute | HIV-assoc. diseases |
| Lamivudine, 3TC | Glaxo Wellcome | HIV infection, AIDS, ARC |
| | | (reverse transcriptase |
| | | inhibitor); also with AZT |
| Lobucavir | Bristol-Myers Squibb | CMV infection |
| Nelfinavir | Agouron | HIV infection, AIDS, ARC |
| | Pharmaceuticals | (protease inhibitor) |
| Nevirapine | Boeheringer | HIV infection, AIDS, ARC |
| | Ingleheim | (RT inhibitor) |
| Novapren | Novaferon Labs, Inc. | HIV inhibitor |
| | (Akron, OH) | |
| Peptide T | Peninsula Labs | AIDS |
| Octapeptide | (Belmont, CA) | |
| Sequence | | |
| Trisodium | Astra Pharm. | CMV retinitis, HIV |
| Phosphonoformate | Products, Inc. | infection, other CMV |
| | | infections |
| PNU-140690 | Pharmacia Upjohn | HIV infection, AIDS, ARC |
| | | (protease inhibitor) |
| Probucol | Vyrex | HIV infection, AIDS |
| RBC-CD4 | Sheffield Med. | HIV infection, AIDS, ARC |
| | Tech (Houston, TX) | |
| Ritonavir | Abbott | HIV infection, AIDS, ARC |
| | | (protease inhibitor) |
| Saquinavir | Hoffmann- | HIV infection, AIDS, ARC |
| | LaRoche | (protease inhibitor) |
| Stavudine; d4T | Bristol-Myers Squibb | HIV infection, AIDS, ARC |
| Didehydrodeoxy- | | |
| Thymidine | | |
| Tipranavir | Boehringer Ingelheim | HIV infection, AIDS, ARC |
| | | (protease inhibitor) |
| Valaciclovir | Glaxo Wellcome | Genital HSV & CMV |
| | | Infections |
| Virazole | Viratek/ICN | asymptomatic HIV positive, |
| Ribavirin | (Costa Mesa, CA) | LAS, ARC |
| VX-478 | Vertex | HIV infection, AIDS, ARC |
| Zalcitabine | Hoffmann-LaRoche | HIV infection, AIDS, ARC, |
| | | with AZT |
| Zidovudine; AZT | Glaxo Wellcome | HIV infection, AIDS, ARC, |
| | | Kaposi's sarcoma, in |
| | | combination with other |
| | | therapies |
| Tenofovir disoproxil, | Gilead | HIV infection, AIDS, |
| fumarate salt (VIREAD ®) | | (reverse transcriptase |
| | | inhibitor) |
| EMTRIVA ® | Gilead | HIV infection, AIDS, |
| (Emtricitabine) (FTC) | | (reverse transcriptase |
| | | inhibitor) |
| COMBIVIR ® | GSK | HIV infection, AIDS, |
| | | (reverse transcriptase |
| | | inhibitor) |
| Abacavir succinate | GSK | HIV infection, AIDS, |
| (or ZIAGEN ®) | | (reverse transcriptase |
| | | inhibitor) |
| REYATAZ ® | Bristol-Myers Squibb | HIV infection AIDs, |
| (or atazanavir) | | protease inhibitor |
| FUZEON ® | Roche/Trimeris | HIV infection AIDs, |
| (Enfuvirtide or T-20) | | viral Fusion inhibitor |
| LEXIVA ® | GSK/Vertex | HIV infection AIDs, |
| (or Fosamprenavir calcium) | | viral protease inhibitor |
| SELZENTRY ™ | Pfizer | HIV infection AIDs, |
| Maraviroc; (UK 427857) | | (CCR5 antagonist, in |
| | | development) |
| TRIZIVIR ® | GSK | HIV infection AIDs, |
| | | (three drug combination) |
| Sch-417690 (vicriviroc) | Schering-Plough | HIV infection AIDs, |
| | | (CCR5 antagonist, in |
| | | development) |
| TAK-652 | Takeda | HIV infection AIDs, |
| | | (CCR5 antagonist, in |
| | | development) |
| GSK 873140 | GSK/ONO | HIV infection AIDs, |
| (ONO-4128) | | (CCR5 antagonist, |
| | | in development) |
| Integrase Inhibitor | Merck | HIV infection AIDs |
| MK-0518 | | |
| Raltegravir | | |
| TRUVADA ® | Gilead | Combination of Tenofovir |
| | | disoproxil fumarate salt |
| | | (VIREAD ®) and EMTRIVA ® |
| | | (Emtricitabine) |
| Integrase Inhibitor | Gilead/Japan Tobacco | HIV Infection AIDs |
| GS917/JTK-303 | | in development |
| Elvitegravir | | |
| Triple drug combination | Gilead/Bristol-Myers Squibb | Combination of Tenofovir |
| ATRIPLA ® | | disoproxil fumarate salt |
| | | (VIREAD ®), EMTRIVA ® |
| | | (Emtricitabine), and |
| | | SUSTIVA ® (Efavirenz) |
| FESTINAVIR ® | Oncolys BioPharma | HIV infection AIDs |
| | | in development |
| CMX-157 | Chimerix | HIV infection AIDs |
| Lipid conjugate of | | |
| nucleotide tenofovir | | |
| GSK1349572 | GSK | HIV infection AIDs |
| Integrase inhibitor | | |
| TIVICAY ® | | |
| dolutegravir | | |

IMMUNOMODULATORS

| | | |
|---|---|---|
| AS-101 | Wyeth-Ayerst | AIDS |
| Bropirimine | Pharmacia Upjohn | Advanced AIDS |
| Acemannan | Carrington Labs, Inc. | AIDS, ARC |
| | (Irving, TX) | |
| CL246,738 | Wyeth | AIDS, Kaposi's |
| | Lederle Labs | sarcoma |
| FP-21399 | Fuki ImmunoPharm | Blocks HIV fusion |
| | | with CD4+ cells |
| Gamma Interferon | Genentech | ARC, in combination |
| | | w/TNF (tumor |
| | | necrosis factor) |
| Granulocyte | Genetics Institute | AIDS |
| Macrophage Colony | Sandoz | |
| Stimulating Factor | | |
| Granulocyte | Hoechst-Roussel | AIDS |
| Macrophage Colony | Immunex | |
| Stimulating Factor | | |
| Granulocyte | Schering-Plough | AIDS, combination |
| Macrophage Colony | | w/AZT |
| Stimulating Factor | | |
| HIV Core Particle | Rorer | Seropositive HIV |
| Immunostimulant | | |
| IL-2 | Cetus | AIDS, in combination |
| Interleukin-2 | | w/AZT |
| IL-2 | Hoffman-LaRoche | AIDS, ARC, HIV, in |
| Interleukin-2 | Immunex | combination w/AZT |
| IL-2 | Chiron | AIDS, increase in |
| Interleukin-2 | | CD4 cell counts |
| (aldeslukin) | | |
| Immune Globulin | Cutter Biological | Pediatric AIDS, in |
| Intravenous | (Berkeley, CA) | combination w/AZT |
| (human) | | |
| IMREG-1 | Imreg | AIDS, Kaposi's |
| | (New Orleans, LA) | sarcoma, ARC, PGL |
| IMREG-2 | Imreg | AIDS, Kaposi's |
| | (New Orleans, LA) | sarcoma, ARC, PGL |
| Imuthiol Diethyl | Merieux Institute | AIDS, ARC |
| Dithio Carbamate | | |
| Alpha-2 | Schering Plough | Kaposi's sarcoma |
| Interferon | | w/AZT, AIDS |
| Methionine- | TNI Pharmaceutical | AIDS, ARC |
| Enkephalin | (Chicago, IL) | |
| MTP-PE | Ciba-Geigy Corp. | Kaposi's sarcoma |
| Muramyl-Tripeptide | | |
| Granulocyte | Amgen | AIDS, in combination |
| Colony Stimulating | | w/AZT |
| Factor | | |
| Remune | Immune Response | Immunotherapeutic |
| | Corp. | |
| rCD4 | Genentech | AIDS, ARC |
| Recombinant | | |
| Soluble Human CD4 | | |
| rCD4-IgG | | AIDS, ARC |
| hybrids | | |
| Recombinant | Biogen | AIDS, ARC |
| Soluble Human CD4 | | |
| Interferon | Hoffman-La Roche | Kaposi's sarcoma |
| Alfa 2a | | AIDS, ARC, |
| | | in combination w/AZT |
| SK&F106528 | Smith Kline | HIV infection |
| Soluble T4 | | |
| Thymopentin | Immunobiology | HIV infection |
| | Research Institute | |
| | (Annandale, NJ) | |
| Tumor Necrosis | Genentech | ARC, in combination |
| Factor; TNF | | w/gamma Interferon |

ANTI-INFECTIVES

| | | |
|---|---|---|
| Clindamycin with | Pharmacia Upjohn | PCP |
| Primaquine | | |
| Fluconazole | Pfizer | Cryptococcal |
| | | meningitis, |
| | | candidiasis |
| Pastille | Squibb Corp. | Prevention of |
| Nystatin Pastille | | oral candidiasis |
| Ornidyl | Merrell Dow | PCP |
| Eflornithine | | |
| Pentamidine | LyphoMed | PCP treatment |
| Isethionate (IM & IV) | (Rosemont, IL) | |
| Trimethoprim | | Antibacterial |
| Trimethoprim/sulfa | | Antibacterial |
| Piritrexim | Burroughs Wellcome | PCP treatment |
| Pentamidine | Fisons Corporation | PCP prophylaxis |
| Isethionate for | | |
| Inhalation | | |
| Spiramycin | Rhone-Poulenc | Cryptosporidial |
| | diarrhea | |
| Intraconazole- | Janssen-Pharm. | Histoplasmosis; |
| R51211 | | cryptococcal |
| | | meningitis |
| Trimetrexate | Warner-Lambert | PCP |
| Daunorubicin | NeXstar, Sequus | Kaposi's sarcoma |
| Recombinant Human | Ortho Pharm. Corp. | Severe anemia |
| Erythropoietin | | assoc. with AZT |
| | | therapy |
| Recombinant Human | Serono | AIDS-related |
| Growth Hormone | | wasting, cachexia |
| Megestrol Acetate | Bristol-Myers Squibb | Treatment of |
| | | anorexia assoc. |
| | | W/AIDS |
| Testosterone | Alza, Smith Kline | AIDS-related wasting |
| Total Enteral | Norwich Eaton | Diarrhea and |
| Nutrition | Pharmaceuticals | malabsorption |
| | | related to AIDS |

### Methods of Synthesis

The compounds of this invention can be made by various methods known in the art including those of the following schemes and in the specific embodiments section. The structure numbering and variable numbering shown in the synthetic schemes are distinct from, and should not be confused with, the structure or variable numbering in the claims or the rest of the specification. The variables in the schemes are meant only to illustrate how to make some of the compounds of this invention. The disclosure is not limited to the foregoing illustrative examples and the examples should be considered in all respects as illustrative and not restrictive, reference being made to the appended claims, rather than to the foregoing examples, and all changes which come within the meaning and range of equivalency of the claims are therefore intended to be embraced.

Abbreviations used in the schemes and examples generally follow conventions used in the art. Chemical abbreviations used in the specification and examples are defined as follows: “KHMDS” for potasium bis(trimethylsilyl)amide; “DMF” for N,N-dimethylformamide; “HATU” for O-(t-Azabenzotriazol-1-yl)-N,N,N′,N′-tetramethyluronium hexafluorophosphate, “MeOH” for methanol; “Ar” for aryl; “TFA” for trifluoroacetic acid, “DMSO” for dimethylsulfoxide; “h” for hours; “rt” for room temperature or retention time (context will dictate); “min” for minutes; “EtOAc” for ethyl acetate; “THF” for tetrahydrofuran; “Et₂O” for diethyl ether; “DMAP” for 4-dimethylaminopyridine; “DCE” for 1,2-dichloroethane; “ACN” for acetonitrile; “DME” for 1,2-dimethoxyethane; “HOBt” for 1-hydroxybenzotriazole hydrate; and “DIEA” for diisopropylethylamine.

Certain other abbreviations as used herein, are defined as follows: “1×” for once, “2×” for twice, “3×” for thrice, “° C.” for degrees Celsius, “eq” for equivalent or equivalents, “g” for gram or grams, “mg” for milligram or milligrams, “L” for liter or liters, “mL” for milliliter or milliliters, “μL” for microliter or microliters, “N” for normal, “M” for molar, “mmol” for millimole or millimoles, “atm” for atmosphere, “psi” for pounds per square inch, “conc.” for concentrate, “sat” or “sat'd ” for saturated, “MW” for molecular weight, “mp” for melting point, “ee” for enantiomeric excess, “MS” or “Mass Spec” for mass spectrometry, “ESI” for electrospray ionization mass spectroscopy, “HR” for high resolution, “HRMS” for high resolution mass spectrometry , “LCMS” for liquid chromatography mass spectrometry, “HPLC” for high pressure liquid chromatography, “RP HPLC” for reverse phase HPLC, “TLC” or “tlc” for thin layer chromatography, “NMR” for nuclear magnetic resonance spectroscopy, “¹H” for proton, “δ” for delta, “s” for singlet, “d” for doublet, “t” for triplet, “q” for quartet, “m” for multiplet, “br” for broad, “Hz” for hertz, and “α”, “β”, “R”, “S”, “E”, and “Z” are stereochemical designations familiar to one skilled in the art.

Some compounds can be synthesized from an appropriately substituted heterocycle I-1 according to Scheme I, Compounds I-1 and I-6 are commercially available or synthesized by reactions well known in the art. Treatment of compound I-1 with bromine provided the dibromo intermediates I-2 which was converted to the chloropyridine I-3 by reacting with POCl₃. Intermediate I-3 conveniently transformed to ketoester I-5 using conditions well-known to those skilled in the art, including reacting I-3 with Grignard reagent in the presence of catalytic copper(I) bromide dimethylsulfide complex followed by alkyl 2-chloro-2-oxoacetate. Coupling of amines I-5 with intermediate I-6 in the presence of an organic base such as Hunig's base provided intermediate I-7. Chiral Lewis acid such as I-8 mediated reduction of ketoester I-7 with catecholborane furnished chiral alcohol I-9. Tertiary butylation of alcohol I-9 by well-known conditions, including but not limited to tertiary-butyl acetate and perchloric acid, gave intermediate I-10. Intermediates I-10 are conveniently transformed to intermediates I-11 using conditions well-known in the art, including but not limited to the Suzuki coupling between intermediates I-10 and R⁶B(OR)₂. The boronate or boronic acid coupling reagents, well-known in the art, are commercially available or are prepared by reactions well-known to those skilled in the art. Hydrolysis of intermediate I-11 by using conditions well-known to those skilled in the art furnished carboxylic acid I-12.

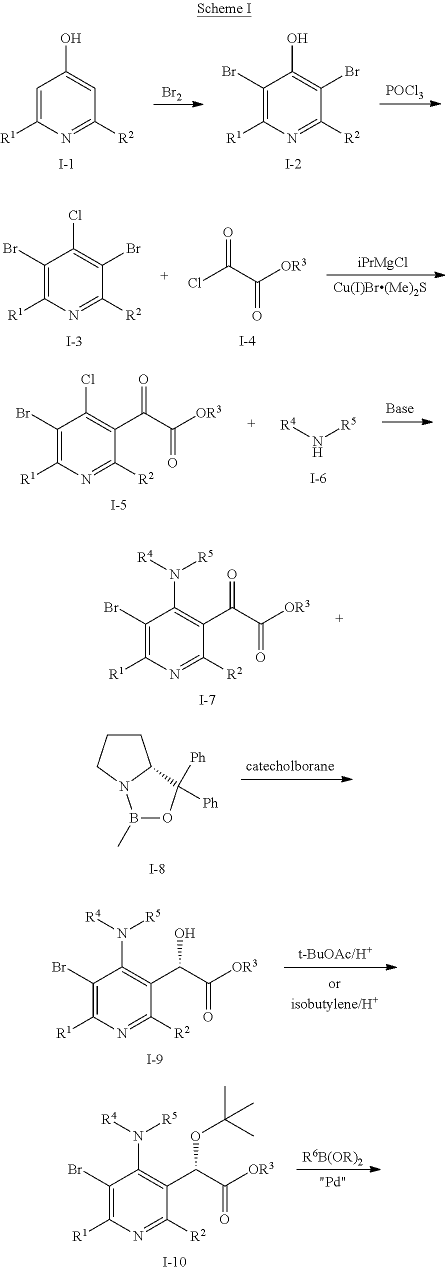

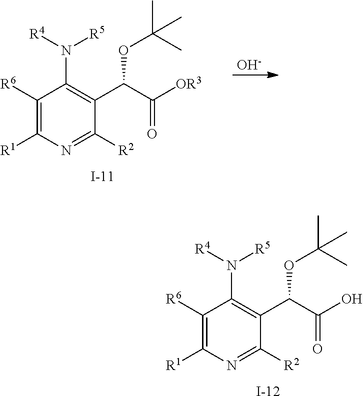

Intermediates I-10 are conveniently transformed to intermediates II-2 using conditions well-known in the art, including but not limited to the Suzuki coupling between intermediates I-10 and II-1. Cleavage of protecting group in II-2 provided carboxylic acid II-3. Coupling of II-3 with appropriate amine II-4 provided II-5 by using conditions well known to those skilled in the art, including but not limited to HATU mediated amide formation reaction. Hydrolysis of intermediate II-5 by using conditions well-known in the literature furnished carboxylic acid II-5.

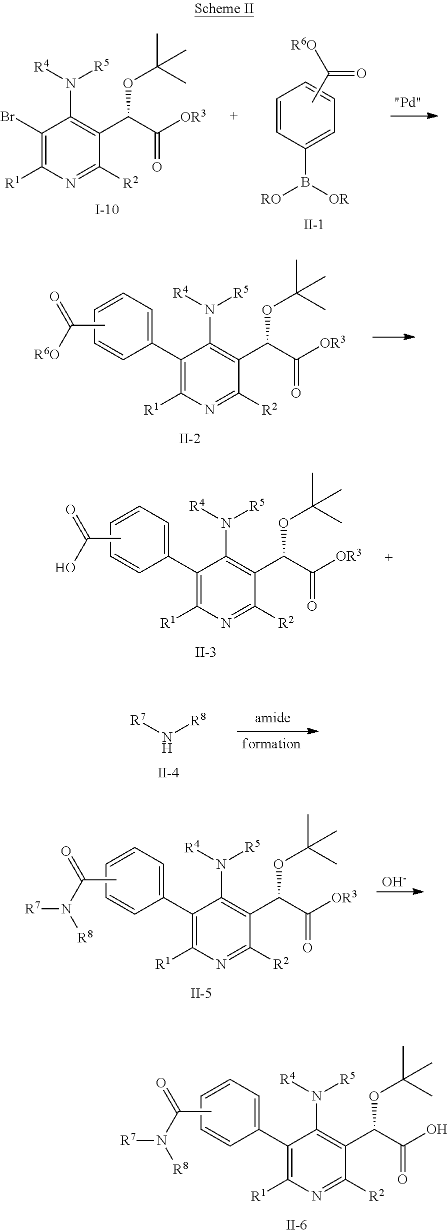

The carboxylic acid IV-3 was coupled with aminomethyl ketone III-1 using conditions well know to those skilled in the art, including but not limited to HATU/DIEA to provide amide III-2. The amide intermediate III-2 were converted to either oxazole or thiazole III-3 reacting with either Birgess or Lawesson's reagent. Hydrolysis of intermediate III-3 by using conditions well-known in the literature furnished carboxylic acid III-4.

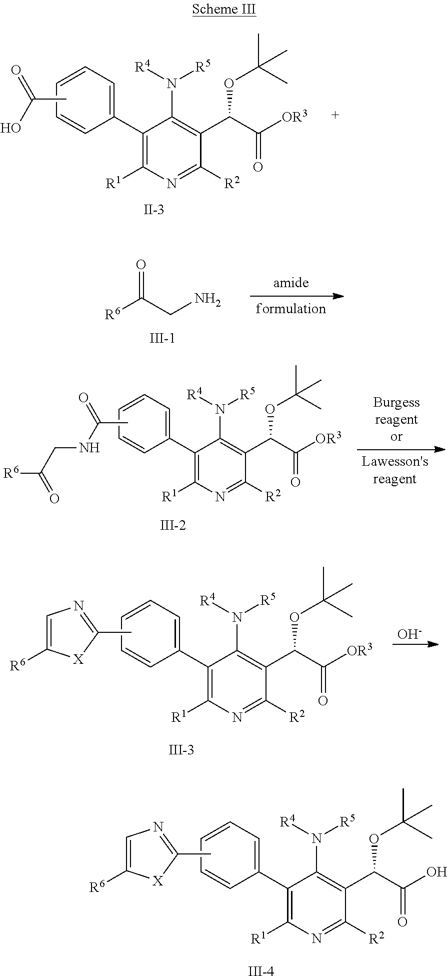

The compounds described herein were purified by the methods well known to those skilled in art by normal phase column chromatography on silica gel column using appropriate solvent system described. Preparative HPLC purifications mentioned in this experimentation section were carried out gradient elution either on Sunfire Prep C18 ODB column (5 μm; 19 or 30×100 mm) or Waters Xbridge column (5 μM; 19 or 30×100 mm) using the following mobile phases: Mobile phase A: 9:1 H₂O/acetonitrile with 10 mM NH₄OAc and mobile phase B:A: 9:1 acetonitrile/H₂O with: 10 mM NH₄OAc; or mobile phase A: 9:1 H₂O/acetonitrile with 0.1% TFA and mobile phase B:A: 9:1 acetonitrile/H₂O with: 0.1% TFA; or mobile phase A: water with 20 mM NH₄OAc and mobile phase B: 95:5 MeOH/H₂O with 20 mM NH₄OAc.

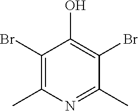

3,5-Dibromo-2,6-dimethylpyridin-4-ol: A 3-neck R.B-flask equipped with mechanical stirrer, addition funnel and condenser is charged with 2,6-dimethylpyridin-4-ol (100 g, 812 mmol), CH₂Cl₂ (1000 mL) and MeOH (120 mL). To the resulting light brown or tan solution was added tert-BuNH₂ (176 ml, 1665 mmol), cooled in water bath maintained between 5-10° C. (ice-water) and added drop wise Br₂ (84 ml, 1624 mmol) over 70 min. After the addition was complete, cold bath was removed and stirred for 1.5 h at rt. Then, the light orange slurry was filtered and the filter cake was washed with ether (250 mL) and dried to afford 3,5-dibromo-2,6-dimethylpyridin-4-ol, hydrobromide (280.75 g, 776 mmol, 96% yield) as white solid which was used in the next step without further purification. ¹H NMR (500 MHz, DMSO-d₆) δ 12.08 (br. s., 1H), 2.41 (s, 6H). LCMS (M+H)=281.9.

Alternative procedure: Bromine (72.8 mL, 1.4 mol) was added via addition funnel over 60 min to a mechanically stirred cold (ice-water bath) solution of 2,6-dimethylpyridin-4-ol (87 g, 706 mmol) and 4-methylmorpholine (156 mL, 1.4 mol) in dichloromethane (1 L) and methanol (100 mL) and then stirred for 2 h at rt. Additional bromine (˜15 mL) was added based on monitoring by LCMS. The product was filtered, washed with ether, and dried under vacuum to give 3,5-dibromo-2,6-dimethylpyridin-4-ol (176.8 g, 88%).

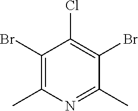

3,5-Dibromo-4-chloro-2,6-dimethyl-pyridine: Triethylamine (28.8 mL, 206 mmol) was added to a nitrogen purged solution of 3,5-dibromo-2,6-dimethylpyridin-4-ol (58 g, 206 mmol) and phosphorous oxychloride (57.7 mL, 619 mmol) in chloroform (450 mL) and stirred for 1 h at rt, then 3 h at 80° C. The reaction was removed from heating and immediately concentrated under house vaccum; then under high vacuum. The appearance was a cream colored solid, which was azeotroped with toluene (2×100 mL); treated with ice (200 g) for 10 min and carefully neutralized with NaHCO₃ (powder), and 1N NaOH solution, and extracted with DCM (2×400 mL). The combined organic layers were dried (MgSO₄), concentrated, and a beige solid was obtained that was washed with hexanes and dried under high vacuum to give 3,5-dibromo-4-chloro-2,6-dimethyl-pyridine 52.74 g (85.1%). Concentration of the hexanes gave 3.5 g of less pure product. ¹H NMR (500 MHz, CDCl₃) δ 2.59 (s, 6H). LCMS (M+H)=300.0.

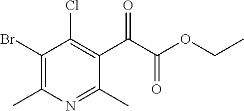 [SMILES: CCOC(=O)C(=O)c1c(C)nc(C)c(Br)c1Cl]

Ethyl 2-(5-bromo-4-chloro-2,6-dimethylpyridin-3-yl)-2-oxoacetate: To a stirred mixture of 3,5-dibromo-4-chloro-2,6-dimethylpyridine (14.94 g, 49.9 mmol) and Cu(I)Br Me₂S (0.513 g, 2.495 mmol) in THF (50 mL) was added drop wise 2M iPrMgCl/THF (26.2 ml, 52.4 mmol) at −30° C. over 5 min. Then, the resulting slurry was warmed to −10° C. over 30 min and stirred for 30 min. The homogeneous brown reaction mixture was rapidly transferred via cannula to a solution of ethyl 2-chloro-2-oxoacetate (6.14 ml, 54.9 mmol, degassed for 5 min by bubbling N2 through the solution) in THF (50 mL) maintained at −30° C. The resulting reaction mixture was stirred (1.5 h) while warming to 0° C. Then, taken up in to Et₂O (200 mL), washed with 1:1 sat Na₂CO₃/1M NH₄Cl (3×50 mL), dried (MgSO₄), filtered and concentrated to give brown viscous oil. Flash chromatography using 2.5, 5 and 7.5% EtOAc/Hex afforded ethyl 2-(5-bromo-4-chloro-2,6-dimethylpyridin-3-yl)-2-oxoacetate (14.37 g, 44.8 mmol, 90% yield) as white solid. ¹H NMR (400 MHz, CDCl₃) δ 4.42 (q, J=7.0 Hz, 2H), 2.76 (s, 3H), 2.46 (s, 3H), 1.41 (t, J=7.2 Hz, 3H). LCMS (M+H)=322.1.

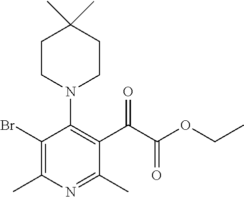 [SMILES: CCOC(=O)C(=O)c1c(C)nc(C)c(Br)c1N1CCC(C)(C)CC1]

Ethyl 2-(5-bromo-4-(4,4-dimethylpiperidin-1-yl)-2,6-dimethylpyridin-3-yl)-2-oxoacetate: To a solution of 4,4-dimethylpiperidine (1.245 g, 11.00 mmol) and DIEA (3.49 ml, 20.00 mmol) in anhydrous CH₃CN (40 mL) was added ethyl 2-(5-bromo-4-chloro-2,6-dimethylpyridin-3-yl)-2-oxoacetate (3.21 g, 10 mmol) at rt. The resulting mixture was placed in a pre-heated oil bath (80° C.). After 22 h, the reaction mixture was concentrated and the residue was purified by flash chromatography using 1-lit each 2.5, 5, 7.5 and 10% EtOAc/Hex to afford ethyl 2-(5-bromo-4-(4,4-dimethylpiperidin-1-yl)-2,6-dimethylpyridin-3-yl)-2-oxoacetate (2.846 g, 7.16 mmol, 71.6% yield) as yellow solid. ¹H NMR (500 MHz, CDCl₃) δ 4.37 (q, J=7.1 Hz, 2H), 3.67-2.75 (br.s., 4H), 2.71 (s, 3H), 2.44 (s, 3H), 1.42 (t, J=7.1 Hz, 3H), 1.38 (t, J=5.6 Hz, 4H), 1.00 (s, 6H). LCMS (M+H)=399.4.

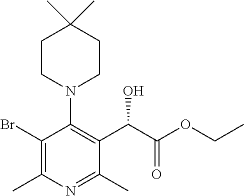 [SMILES: CCOC(=O)[C@@H](O)c1c(C)nc(C)c(Br)c1N1CCC(C)(C)CC1]

(S)-Ethyl 2-(5-bromo-4-chloro-2,6-dimethylpyridin-3-yl)-2-hydroxyacetate: To stirred yellow solution of ethyl 2-(5-bromo-4-(4,4-dimethylpiperidin-1-yl)-2,6-dimethylpyridin-3-yl)-2-oxoacetate (2.25 g, 5.66 mmol) and (R)-1-methyl-3,3-diphenylhexahydropyrrolo[1,2-c][1,3,2]oxazaborole (0.314 g, 1.133 mmol) in toluene (30 mL) at −35° C. was added drop wise 50% catecholborane (1.819 ml, 8.49 mmol) over 10 min. The reaction mixture was slowly warmed to −15° C. over 1 h and then left for 2 h at −15° C. Then, diluted with EtOAc (100 mL), washed with sat Na₂CO₃ (4×25 mL) by vigorously stirring and separating aqueous layers. The organic layer dried (MgSO₄), filtered, concentrated and purified by flash chromatography using 10, 20 and 25% EtOAc/Hex to afford desired (S)-ethyl 2-(5-bromo-4-(4,4-dimethylpiperidin-1-yl)-2,6-dimethylpyridin-3-yl)-2-hydroxyacetate (2.2596 g, 5.66 mmol, 100% yield) contaminated with about 10% of (S)-ethyl 2-(5-bromo-4-chloro-2,6-dimethylpyridin-3-yl)-2-hydroxyacetate. Used in the next step without further purification. ¹H NMR (500 MHz, CDCl₃) δ 5.71 (d, J=7.3 Hz, 1H), 5.54 (d, J=7.4 Hz, 1H), 4.29 (dq, J=10.8, 7.1 Hz, 1H), 4.16 (dq, J=10.8, 7.1 Hz, 1H), 3.94-3.83 (m, 2H), 2.71 (d, J=11.9 Hz, 1H), 2.67 (s, 3H), 2.59 (s, 3H), 2.54 (d, J=12.0 Hz, 1H), 1.71 (td, J=12.7, 4.7 Hz, 1H), 1.62 (td, J=13.0, 4.7 Hz, 1H), 1.42 (dd, J=13.1, 2.2 Hz, 1H), 1.37 (dd, J=12.9, 2.4 Hz, 1H), 1.25 (t, J=7.1 Hz, 3H), 1.09 (s, 3H), 1.04 (s, 3H). LCMS (M+H)=401.3.

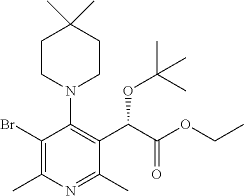 [SMILES: CCOC(=O)[C@@H](OC(C)(C)C)c1c(C)nc(C)c(Br)c1N1CCC(C)(C)CC1]

(S)-Ethyl 2-(5-bromo-4-(4,4-dimethylpiperidin-1-yl)-2,6-dimethylpyridin-3-yl)-2-(tert-butoxy)acetate: A stirred ice-cold yellow mixture of (S)-ethyl 2-(5-bromo-4-(4,4-dimethylpiperidin-1-yl)-2,6-dimethylpyridin-3-yl)-2-hydroxyacetate (2.45 g, 6.14 mmol) and 70% HClO₄ (1.054 ml, 12.27 mmol) in CH₂Cl₂ (100 mL) was saturated with isobutylene gas by bubbling through the reaction mixture (10 min). After 2 h, cold bath was removed and the turbid reaction mixture stirred for 22 h at rt. LCMS at this point showed 4:1 product to sm. So, saturated with isobutylene (5 min) at rt and stirred for additional 24 h. Then, neutralized with sat. Na₂CO₃ (30 mL), organic layer separated and aqueous layer extracted with CH₂Cl₂ (25 mL). The combined organic layers dried (MgSO₄), filtered, concentrated and purified by flash chromatography using 5, 10, 15, 20 and 40% EtOAc/hex to afford (S)-ethyl 2-(5-bromo-4-(4,4-dimethylpiperidin-1-yl)-2,6-dimethylpyridin-3-yl)-2-(tert-butoxy)acetate (2.3074 g, 5.07 mmol, 83% yield) as yellow oil: ¹H NMR (500 MHz, CDCl₃) δ 6.19 (br. s., 1H), 4.17-4.24 (m, 1H), 4.08-4.14 (m, 1H), 4.04 (dt, J=2.5, 12.1 Hz, 1H), 3.51 (dt, J=2.5, 12.1 Hz, 1H), 2.85-2.91 (m, 1H), 2.64 (s, 3H), 2.57-2.62 (m, 1H), 2.55 (s, 3H), 1.55-1.66 (m, 2H), 1.41-1.46 (m, 1H), 1.32-1.37 (m, 1H), 1.21 (s, 9H), 1.20 (t, J=7.2 Hz, 2H), 1.08 (s, 3H), 1.03 (s, 3H). LCMS (M+H)=457.4. And (S)-ethyl 2-(5-bromo-4-(4,4-dimethylpiperidin-1-yl)-2,6-dimethylpyridin-3-yl)-2-hydroxyacetate (0.3 g, 0.751 mmol, 12.24% yield) as pale yellow paste: LCMS (M+H)=401.3.

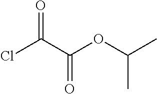

Isopropyl 2-chloro-2-oxoacetate: The propan-2-ol (38.2 mL, 499 mmol) was added drop wise over 15 min to a cold (0° C.), nitrogen purged solution of oxalyl dichloride (101 g, 799 mmol) and the reaction was stirred at room temperature for 2.5 h. Then a reflux condenser was fitted and a slight vacuum was applied for about 1 h until HCl gas was removed (the HCl was trapped in by a sat'd solution of NaHCO₃). The reflux condenser was removed and the flask was fitted with a short path distillation head. Excess reagent was removed by distillation under house vacuum (oil bath heated to 65° C.), and then the temperature was raised to between 85-95° C. and the product was distilled (NOTE: The 1^{st} fraction of ˜5 mL was discarded) to provide isopropyl 2-chloro-2-oxoacetate 52.62 g (70%).

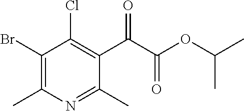 [SMILES: Cc1nc(C)c(C(=O)C(=O)OC(C)C)c(Cl)c1Br]

Isopropyl 2-(5-bromo-4-chloro-2,6-dimethylpyridin-3-yl)-2-oxoacetate: A solution of 2M isopropyl magnesium chloride (84 mL, 168 mmol) was added drop wise over 20 min to a cold (−70° C.), nitrogen purged solution of 3,5-dibromo-4-chloro-2,6-dimethylpyridine (48 g, 160 mmol) and copper(I)bromide-dimethyl sulfide complex (1.65 g, 8.02 mmol) in THF (240 mL), which was then allowed to warm to −10° C. over 60 min. The reaction mixture was transferred via cannula into a 1 L RB-flask containing isopropyl 2-chloro-2-oxoacetate (26.6 g, 176 mmol) in THF (160 mL) maintained at −60° C., and the reaction stirred an additional 2.5 h while being allowed to warm to −10° C. The reaction was quenched upon diluted with a mixture of 10% NH₄Cl solution (80 mL) in ether (320 mL). The organic layer was washed with 160 mL of sat'd NaHCO₃/10% NH₄Cl solution (1:1), brine, and dried (Na₂SO₄). The crude product was charged (DCM solution) to a 330 g ISCO silica gel cartridge and gradient eluted (5-20% EtOAc/hexanes) using an Isolera chromatography station gave isopropyl 2-(5-bromo-4-chloro-2,6-dimethylpyridin-3-yl)-2-oxoacetate 40.38 g (76%). ¹H NMR (500 MHz, CDCl₃) δ 5.28-5.21 (m, 1H), 2.77 (s, 3H), 2.47 (s, 3H), 1.40 (d, J=6.3 Hz, 6H). LCMS (M+H)=336.04.

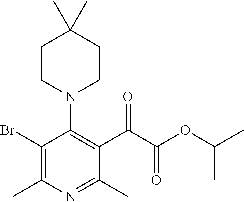 [SMILES: Cc1nc(C)c(C(=O)C(=O)OC(C)C)c(N2CCC(C)(C)CC2)c1Br]

Isopropyl 2-(5-bromo-4-(4,4-dimethylpiperidin-1-yl)-2,6-dimethylpyridin-3-yl)-2-oxoacetate: To a stirred solution of isopropyl 2-(5-bromo-4-chloro-2,6-dimethylpyridin-3-yl)-2-oxoacetate (7.2 g, 21.52 mmol) and DIEA (4.13 mL, 23.67 mmol) in anhydrous acetonitrile (15 mL) was added 4,4-dimethylpiperidine (2.68 g, 23.67 mmol) in acetonitrile (15 mL). The resulting solution was placed in a pre-heated oil bath at 75° C. After heating (75-78° C.) for 24 h and the temperature was raised to 85° C. for 24 h. Another portion of DIEA (3.5 mL, 20.04 mmol) and 4,4-dimethylpiperidine (0.27g, 2.4 mmol) in acetonitrile (3 mL) was added and hearted at 85° C. for a day. The reaction mixture was diluted with ether (100 mL), washed with water (100 mL), brine (50 mL), dried (MgSO₄), filtered, concentrated and purified by ISCO 120 g cartridge (EtOAc/hex: 0 to 20%) to afford isopropyl 2-(5-bromo-4-(4,4-dimethylpiperidin-1-yl)-2,6-dimethylpyridin-3-yl)-2-oxoacetate (6.8 g, 16.53 mmol, 77% yield. ¹H NMR (500 MHz, CDCl₃) δ 5.25-5.11 (m, 1H), 3.17 (br. s., 4H), 2.71 (s, 3H), 2.41 (s, 3H), 1.42-1.37 (m, 10H), 1.00 (s, 6H).). LCMS (M+H)=413.3.

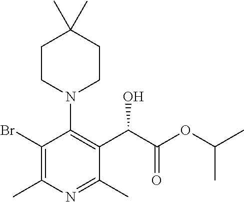 [SMILES: Cc1nc(C)c([C@H](O)C(=O)OC(C)C)c(N2CCC(C)(C)CC2)c1Br]

(S)-Isopropyl 2-(5-bromo-4-(4,4-dimethylpiperidin-1-yl)-2,6-dimethylpyridin-3-yl)-2-hydroxyacetate: To a yellow solution of isopropyl 2-(5-bromo-4-(4,4-dimethylpiperidin-1-yl)-2,6-dimethylpyridin-3-yl)-2-oxoacetate (7.7 g, 18.72 mmol) and (R)-1-methyl-3,3-diphenylhexahydropyrrolo[1,2-c][1,3,2]oxazaborole (7.5 mL, 7.50 mmol) in anhydrous toluene (100 mL) was added drop wise 50% catecholborane/toluene (6 mL, 28.0 mmol) over 5 min at −50° C. Then, the reaction mixture was slowly warmed to −30° C. over 1 h and left in refrigerator (−20° C.) for 3 days. Then, the reaction mixture was diluted with EtOAc (100 mL) and 20 mL of 1M Na₂CO₃, and vigorously stirred for 30 min. Aqueous layer separated and organic layer washed with sat'd Na₂CO₃ (2×25 mL) by vigorously stirring for 15 each time, then dried (MgSO₄), filtered and concentrated to give crude product as light purple paste which was purified by flash chromatography using 0 to 40% EtOAc/hex to afford (S)-isopropyl 2-(5-bromo-4-(4,4-dimethylpiperidin-1-yl)-2,6-dimethylpyridin-3-yl)-2-hydroxyacetate (6.7 g, 15.72 mmol, 84% yield) as colorless thick paste. ¹H NMR (500 MHz, CDCl₃) δ 5.85 (d, J=5.7 Hz, 1H), 5.59 (d, J=7.4 Hz, 1H), 5.08 (dt, J=12.5, 6.3 Hz, 1H), 3.98-3.88 (m, 1H), 3.88-3.78 (m, 1H), 2.76-2.68 (m, 1H), 2.67 (s, 3H), 2.64-2.58 (m, 1H), 2.57 (s, 3H), 1.73 (td, J=12.8, 4.8 Hz, 1H), 1.65-1.59 (m, 1H), 1.47-1.35 (m, 2H), 1.27 (d, J=6.3 Hz, 3H), 1.17 (d, J=6.1 Hz, 3H), 1.09 (s, 3H), 1.04 (s, 3H). LCMS (M+H)=414.6.

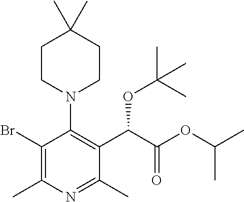 [SMILES: Cc1nc(C)c([C@H](OC(C)(C)C)C(=O)OC(C)C)c(N2CCC(C)(C)CC2)c1Br]

(S)-Isopropyl 2-(5-bromo-4-(4,4-dimethylpiperidin-1-yl)-2,6-dimethylpyridin-3-yl)-2-(tert-butoxy)acetate: A stirred ice-cold yellow mixture of (S)-isopropyl 2-(5-bromo-4-(4,4-dimethylpiperidin-1-yl)-2,6-dimethylpyridin-3-yl)-2-hydroxyacetate (6.7 g, 16.21 mmol) and 70% HClO₄ (2.2 mL, 25.6 mmol) in dichloromethane (400 mL) was saturated with isobutylene gas by bubbling through the reaction mixture (10 min). The reaction mixture was cloudy sealed in a seal tube, stirred for 24 h at rt. The reaction mixture was recooled in a −10° C. bath, bubbled additional isobutylene (˜15 min). The reaction mixture became a clear solution at this point. The tube was sealed and stirred at rt for 16 h. LCMs at this point showed incomplete reaction. So, the reaction mixture was cooled down to −30° C. and bubbled isobutene (˜15 min). After 24 h, reaction mixture was neutralized with sat. Na₂CO₃ (20 mL), organic layer separated and aqueous layer was extracted with CH₂Cl₂ (25 mL). The combined organic layers were dried (MgSO₄), filtered, concentrated and purified on a ISCO 120 g column (EtOAc/hex: 0 to 40%) to afford (S)-isopropyl 2-(5-bromo-4-(4,4-dimethylpiperidin-1-yl)-2,6-dimethylpyridin-3-yl)-2-(tert-butoxy)acetate (5.43 g, 9.83 mmol, 60.7% yield) as a viscous oil. ¹H NMR (500 MHz, CDCl₃) δ 6.26 (br. s., 1H), 5.09-4.97 (m, 1H), 4.06 (br. s., 1H), 3.51 (br. s., 1H), 2.90 (br. s., 1H), 2.65 (s, 3H), 2.56 (s, 3H), 1.72-1.54 (m, 3H), 1.47 (br. s., 1H), 1.37 (br. s., 1H), 1.23-1.20 (m, 12H), 1.15 (d, J=6.1 Hz, 3H), 1.09 (br. s., 3H), 1.04 (br. s., 3H). LCMS (M+H)=471.3.

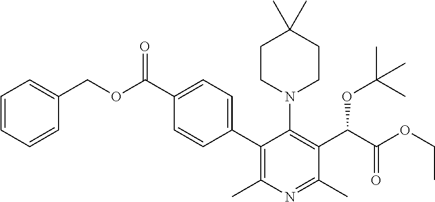 [SMILES: CCOC(=O)[C@@H](OC(C)(C)C)c1c(C)nc(C)c(-c2ccc(C(=O)OCc3ccccc3)cc2)c1N1CCC(C)(C)CC1]

(S)-Benzyl 4-(5-(1-(tert-butoxy)-2-ethoxy-2-oxoethyl)-4-(4,4-dimethylpiperidin-1-yl)-2,6-dimethylpyridin-3-yl)benzoate: A mixture of (S)-ethyl 2-(5-bromo-4-(4,4-dimethylpiperidin-1-yl)-2,6-dimethylpyridin-3-yl)-2-(tert-butoxy)acetate (1.41 g, 3.10 mmol), (4-((benzyloxy)carbonyl)phenyl)boronic acid (1.189 g, 4.64 mmol) and 2M Na₂CO₃ (3.87 ml, 7.74 mmol) in DMF (40 mL) was degassed for 10 min by bubbling N2 through the reaction mixture. Then, Pd(Ph₃P)₄ (0.250 g, 0.217 mmol) was added, degassed for 5 min and heated at 110° C. for 3 h. The reaction mixture was cooled, diluted with ether (100 mL), washed with water (4×25 mL), brine (25 mL), dried (MgSO₄), filtered, concentrated and purified by flash chrmotagraphy using 10,20 and 30% EtOAc/Hex to afford (S)-benzyl 4-(5-(1-(tert-butoxy)-2-ethoxy-2-oxoethyl)-4-(4,4-dimethylpiperidin-1-yl)-2,6-dimethylpyridin-3-yl)benzoate (0.8927 g, 1.491 mmol, 48.2% yield) as white foam. ¹H NMR (500 MHz, CDCl₃) δ 8.16-8.23 (m, 2H), 7.49-7.53 (m, 2H), 7.37-7.47 (m, 4H), 7.26 (d, J=7.72 Hz, 1H), 5.95 (br. s., 1H), 5.44 (s, 2H), 4.17-4.33 (m, 2H), 2.60-2.85 (br.s., 3H), 2.16-2.40 (br.s, 3H), 1.29 (t, J=7.2 Hz, 4H), 1.21 (s, 9H), 0.6-0.93 (br.s., 6H). 4H of piperiodine were not resolved. LCMS (M+H)=587.6.

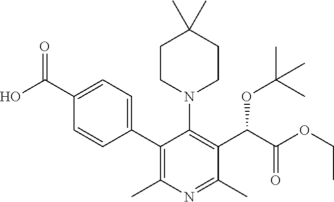 [SMILES: CCOC(=O)[C@@H](OC(C)(C)C)c1c(C)nc(C)c(-c2ccc(C(=O)O)cc2)c1N1CCC(C)(C)CC1]

(S)-4-(5-(1-(tert-Butoxy)-2-ethoxy-2-oxoethyl)-4-(4,4-dimethylpiperidin-1-yl)-2,6-dimethylpyridin-3-yl)benzoic acid: A mixture of (S)-benzyl 4-(5-(1-(tert-butoxy)-2-ethoxy-2-oxoethyl)-4-(4,4-dimethylpiperidin-1-yl)-2,6-dimethylpyridin-3-yl)benzoate (0.891 g, 1.519 mmol) and 10% Pd/C (0.162 g, 0.152 mmol) in EtOAc (50 mL) was evacuated and released to H₂ three times and left under balloon H₂ atmoshpere for 18 h. Then, filtered through a plug of celite and concentrated to give (S)-4-(5-(1-(tert-butoxy)-2-ethoxy-2-oxoethyl)-4-(4,4-dimethylpiperidin-1-yl)-2,6-dimethylpyridin-3-yl)benzoic acid (0.70 g, 1.409 mmol, 93% yield) as white solid. ¹H NMR (500 MHz, CDCl₃) δ 8.22-8.27 (m, 2H), 7.41 (dd, J=1.3, 8.0 Hz, 1H), 7.26 (dd, J=1.1, 8.0 Hz, 1H), 5.93 (br. s., 1H), 3.81-5.07 (m, 6H), 2.77 (br. s., 3H), 2.35 (br. s., 3H), 1.26-1.36 (m, 4H), 1.30 (t, J=7.1 Hz, 3H), 1.21 (s, 9H), 0.79 (br. s., 6H). LCMS (M+H)=497.5.

### EXAMPLE 1

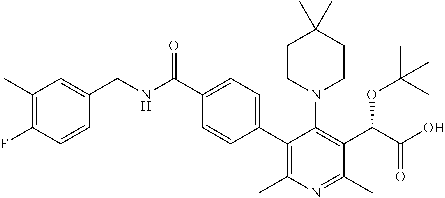 [SMILES: Cc1cc(CNC(=O)c2ccc(-c3c(C)nc(C)c([C@H](OC(C)(C)C)C(=O)O)c3N3CCC(C)(C)CC3)cc2)ccc1F]

(S)-2-(tert-Butoxy)-2-(4-(4,4-dimethylpiperidin-1-yl)-5-(4-((4-fluoro-3-methylbenzyl)carbamoyl)phenyl)-2,6-dimethylpyridin-3-yl)acetic acid: To a stirred solution of (S)-4-(5-(1-(tert-butoxy)-2-ethoxy-2-oxoethyl)-4-(4,4-dimethylpiperidin-1-yl)-2,6-dimethylpyridin-3-yl)benzoic acid (0.025 g, 0.050 mmol), (4-fluoro-3-methylphenyl)methanamine (0.014 g, 0.101 mmol) and DIEA (0.035 ml, 0.201 mmol) in DMF (2 mL) was added at once HATU (0.038 g, 0.101 mmol) at rt. After 4 h, the reaction mixture was taken up in ether (25 mL), washed with water (2×5 mL), brine (5 mL), dried (MgSO₄), filtered and concentrated to give (S)-ethyl 2-(tert-butoxy)-2-(4-(4,4-dimethylpiperidin-1-yl)-5-(4-((4-fluoro-3-methylbenzyl)carbamoyl)phenyl)-2,6-dimethylpyridin-3-yl)acetate as yellow paste which was used in the next step without purification. LCMS (M+H)=618.5.

A solution of above crude ester and 1M NaOH (0.503 ml, 0.503 mmol) in MeOH (1 mL) was heated at reflux for 4 h. Then, cooled and purified by prep-HPLC to afford (S)-2-(tert-butoxy)-2-(4-(4,4-dimethylpiperidin-1-yl)-5-(4-((4-fluoro-3-methylbenzyl)carbamoyl)phenyl)-2,6-dimethylpyridin-3-yl)acetic acid (0.0203 g, 0.033 mmol, 65.0% yield) as. ¹H NMR (400 MHz, CDCl₃) δ 8.03 (d, J=8.3 Hz, 2H), 7.36 (d, J=7.28 Hz, 1H), 7.13-7.26 (m, 3H), 6.98 (t, J=8.9 Hz, 1H), 6.93-6.98 (br. s., 1H), 5.73 (br. s., 1H), 4.63 (d, J=4.8 Hz, 2H), 2.83 (s, 3H), 2.64 (br. s., 4H), 2.38 (br. s., 3H), 2.28 (s, 3H), 1.26-1.38 (m, 4H), 1.22 (s, 9H), 0.80 (br. s., 6H). LCMS (M+H) 590.5.

### EXAMPLE 2

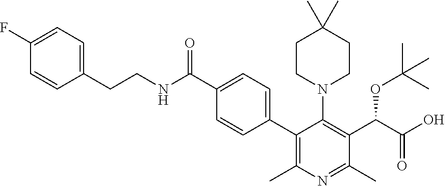 [SMILES: Cc1nc(C)c([C@H](OC(C)(C)C)C(=O)O)c(N2CCC(C)(C)CC2)c1-c1ccc(C(=O)NCCc2ccc(F)cc2)cc1]

(S)-2-(tert-Butoxy)-2-(4-(4,4-dimethylpiperidin-1-yl)-5-(4-((4-fluorophenethyl)carbamoyl)phenyl)-2,6-dimethylpyridin-3-yl)acetic acid: To a stirred solution of (S)-4-(5-(1-(tert-butoxy)-2-ethoxy-2-oxoethyl)-4-(4,4-dimethylpiperidin-1-yl)-2,6-dimethylpyridin-3-yl)benzoic acid (0.024 g, 0.048 mmol), 2-(4-fluorophenyl)ethanamine (0.013 g, 0.097 mmol) and DIEA (0.034 ml, 0.193 mmol) in DMF (2 mL) was added at once HATU (0.037 g, 0.097 mmol) at rt. After stirring overnight (15 h), the reaction mixture was taken up in ether (25 mL), washed with water (2×5 mL), brine (5 mL), dried (MgSO₄), filtered and concentrated to give (S)-ethyl 2-(tert-butoxy)-2-(4-(4,4-dimethylpiperidin-1-yl)-5-(4-((4-fluorophenethyl)carbamoyl)phenyl)-2,6-dimethylpyridin-3-yl)acetate as yellow paste which was used in the next step without purification. LCMS (M+H)=618.6.

A solution of above crude ester and 1M NaOH (0.483 ml, 0.483 mmol) in MeOH (1 mL) was heated at reflux for 18 h. Then cooled and purified by prep-HPLC to afford (S)-2-(tert-butoxy)-2-(4-(4,4-dimethylpiperidin-1-yl)-5-(4-((4-fluorophenethyl)carbamoyl)phenyl)-2,6-dimethylpyridin-3-yl)acetic acid (0.0186 g, 0.030 mmol, 62.7% yield) as. ¹H NMR (400 MHz, CDCl₃) δ 7.95 (t, J=6.8 Hz, 2H), 7.34 (d, J=7.8 Hz, 1H), 7.25 (dd, J=5.52, 8.5 Hz, 2H), 7.14 (d, J=7.5 Hz, 1H), 7.02 (t, J=8.7 Hz, 2H), 6.71 (br. s., 1H), 5.73 (br. s., 1H), 3.71-3.79 (m, 2H), 2.99 (t, J=7.0 Hz, 2H), 2.84 (s, 3H), 2.68 (br. s., 4H), 2.39 (s, 3H), 1.26-1.38 (m, 4H), 1.22 (s, 9H), 0.80 (br. s., 6H). LCMS (M+H)=590.4.

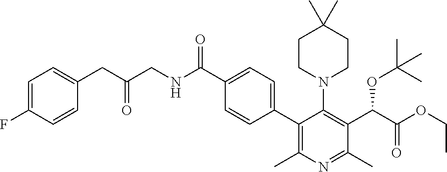 [SMILES: CCOC(=O)[C@@H](OC(C)(C)C)c1c(C)nc(C)c(-c2ccc(C(=O)NCC(=O)Cc3ccc(F)cc3)cc2)c1N1CCC(C)(C)CC1]

(S)-Ethyl 2-(tert-butoxy)-2-(4-(4,4-dimethylpiperidin-1-yl)-5-(4-((3-(4-fluorophenyl)-2-oxopropyl)carbamoyl)phenyl)-2,6-dimethylpyridin-3-yl)acetate: To a stirred solution of (S)-4-(5-(1-(tert-butoxy)-2-ethoxy-2-oxoethyl)-4-(4,4-dimethylpiperidin-1-yl)-2,6-dimethylpyridin-3-yl)benzoic acid (0.099 g, 0.2 mmol), 1-amino-3-(4-fluorophenyl)propan-2-one HCl (0.061 g, 0.300 mmol) and HATU (0.114 g, 0.300 mmol) in DMF (5 mL) was added DIEA (0.105 ml, 0.600 mmol) at once at rt. After 2 h, diluted with ether (50 mL), washed with water (3×5 mL), brine (5 mL), dried (MgSO₄), filtered, concentrated and purified by flash chromatography using 1:1 and 2:1 EtOAc/Hex to afford (S)-ethyl 2-(tert-butoxy)-2-(4-(4,4-dimethylpiperidin-1-yl)-5-(4-((3-(4-fluorophenyl)-2-oxopropyl)carbamoyl)phenyl)-2,6-dimethylpyridin-3-yl)acetate (0.105 g, 0.163 mmol, 81% yield) as white solid. LCMS (M+H)=646.5.

### EXAMPLE 3

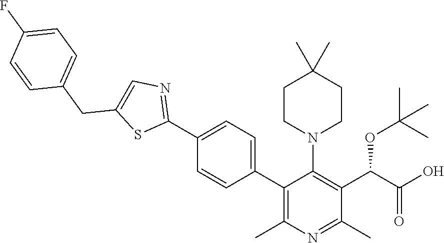 [SMILES: Cc1nc(C)c([C@H](OC(C)(C)C)C(=O)O)c(N2CCC(C)(C)CC2)c1-c1ccc(-c2ncc(Cc3ccc(F)cc3)s2)cc1]

(S)-2-(tert-Butoxy)-2-(4-(4,4-dimethylpiperidin-1-yl)-5-(4-(5-(4-fluorobenzyl)thiazol-2-yl)phenyl)-2,6-dimethylpyridin-3-yl)acetic acid: A mixture of (S)-ethyl 2-(tert-butoxy)-2-(4-(4,4-dimethylpiperidin-1-yl)-5-(4-((3-(4-fluorophenyl)-2-oxopropyl)carbamoyl)phenyl)-2,6-dimethylpyridin-3-yl)acetate (0.056 g, 0.087 mmol) and Lawesson's reagent (0.042 g, 0.104 mmol) in anhydrous toluene (3 mL) was heated at 75° C. for 24 h. Then, cooled and removed solvent to give (S)-ethyl 2-(tert-butoxy)-2-(4-(4,4-dimethylpiperidin-1-yl)-5-(4-(5-(4-fluorobenzyl)thiazol-2-yl)phenyl)-2,6-dimethylpyridin-3-yl)acetate yellow paste which was used in the next step without purification. LCMS (M+H)=644.5.

A solution of above crude product and 1M LiOH (0.867 ml, 0.867 mmol) in EtOH (2 mL) was heated at reflux for 24 h. Then, cooled and purified by prep-HPLC to afford (S)-2-(tert-butoxy)-2-(4-(4,4-dimethylpiperidin-1-yl)-5-(4-(5-(4-fluorobenzyl)thiazol-2-yl)phenyl)-2,6-dimethylpyridin-3-yl)acetic acid (0.0227 g, 0.037 mmol, 42.5% yield) as solid. ¹H NMR (500 MHz, CDCl₃) δ 7.98-8.04 (m, 2H), 7.64 (t, J=0.9 Hz, 1H), 7.32-7.36 (m, 1H), 7.26-7.29 (m, 2H), 7.20 (d, J=8.0 Hz, 1H), 7.03-7.09 (m, 2H), 5.90 (br. s., 1H), 4.21 (s, 2H), 2.75 (br. s., 3H), 2.34 (br. s., 3H), 1.27-1.36 (m, 4H), 1.25 (s, 9H), 0.78 (br. s., 6H). 4H of piperidine were not resolved. LCMS (M+H)=616.45.

### EXAMPLE 4

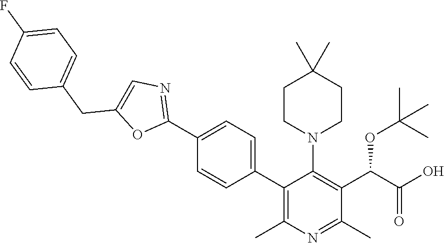 [SMILES: Cc1nc(C)c([C@H](OC(C)(C)C)C(=O)O)c(N2CCC(C)(C)CC2)c1-c1ccc(-c2ncc(Cc3ccc(F)cc3)o2)cc1]

(S)-2-(tert-Butoxy)-2-(4-(4,4-dimethylpiperidin-1-yl)-5-(4-(5-(4-fluorobenzypoxazol-2-yl)phenyl)-2,6-dimethylpyridin-3-yl)acetic acid: A solution of (S)-ethyl 2-(tert-butoxy)-2-(4-(4,4-dimethylpiperidin-1-yl)-5-(4-((3-(4-fluorophenyl)-2-oxopropyl)carbamoyl)phenyl)-2,6-dimethylpyridin-3-yl)acetate (0.048 g, 0.074 mmol) and Burgess reagent (0.027 g, 0.111 mmol) THF (1.5 mL) was sealed in a microwave vial and placed in an oil bath pre-heated to 115° C. After 2 h, the reaction was mixture cooled, concentrated and the crude (S)-ethyl 2-(tert-butoxy)-2-(4-(4,4-dimethylpiperidin-1-yl)-5-(4-(5-(4-fluorobenzyl)oxazol-2-yl)phenyl)-2,6-dimethylpyridin-3-yl)acetate was used in the next step without purification. LCMS (M+H)=628.5.

A solution of above crude ester and 2M LiOH (0.372 ml, 0.743 mmol) in 1:1 MeOH/EtOH (3 mL) was stirred at reflux for 5 h. Then, cooled and purified by prep-HPLC to afford (S)-2-(tert-butoxy)-2-(4-(4,4-dimethylpiperidin-1-yl)-5-(4-(5-(4-fluorobenzyl)oxazol-2-yl)phenyl)-2,6-dimethylpyridin-3-yl)acetic acid (0.0307 g, 0.051 mmol, 68.9% yield) as solid. ¹H NMR (500 MHz, CDCl₃) δ 8.09-8.11 (m, 2H), 7.35-7.39 (m, 1H), 7.29-7.32 (m, 2H), 7.25 (d, J=7.3 Hz, 1H), 7.04-7.10 (m, 2H), 6.92 (s, 1H), 5.89 (br. s., 1H), 4.09 (s, 2H), 2.70 (br. s., 3H), 2.25 (br. s., 3H), 1.26-1.37 (m, 4H), 1.24 (s, 9H), 0.76 (br. s., 6H). 4H of piperidine were not resolved. LCMS (M+H)=600.5.

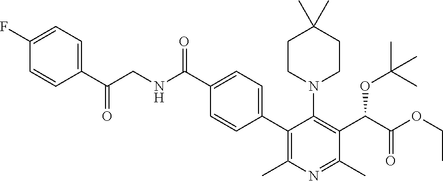 [SMILES: CCOC(=O)[C@@H](OC(C)(C)C)c1c(C)nc(C)c(-c2ccc(C(=O)NCC(=O)c3ccc(F)cc3)cc2)c1N1CCC(C)(C)CC1]

(S)-Ethyl 2-(tert-butoxy)-2-(4-(4,4-dimethylpiperidin-1-yl)-5-(4-((2-(4-fluorophenyl)-2-oxoethyl)carbamoyl)phenyl)-2,6-dimethylpyridin-3-yl)acetate: To a stirred solution of (S)-4-(5-(1-(tert-butoxy)-2-ethoxy-2-oxoethyl)-4-(4,4-dimethylpiperidin-1-yl)-2,6-dimethylpyridin-3-yl)benzoic acid (0.099 g, 0.2 mmol), 2-amino-1-(4-fluorophenyl)ethanone HCl (0.057 g, 0.300 mmol) and HATU (0.114 g, 0.300 mmol) in DMF (5 mL) was added at once DIEA (0.105 ml, 0.600 mmol) at rt. After 2 h, the reaction mixture was diluted with ether (50 mL), washed with water (3×5 mL), brine (5 mL), dried (MgSO₄), filtered, concentrated and purified by flash chromatography using 1:1 and 3:1 EtOAc/Hex to afford (S)-ethyl 2-(tert-butoxy)-2-(4-(4,4-dimethylpiperidin-1-yl)-5-(4-((2-(4-fluorophenyl)-2-oxoethyl)carbamoyl)phenyl)-2,6-dimethylpyridin-3-yl)acetate (0.121 g, 0.192 mmol, 96% yield) as pale yellow solid. ¹H NMR (500 MHz, CDCl₃) δ 8.10-8.16 (m, 2H), 7.97-8.03 (m, 2H), 7.44 (dd, J=1.3, 7.7 Hz, 1H), 7.38 (t, J=4.1 Hz, 1H), 7.30-7.33 (m, 1H), 7.23-7.27 (m, 2H), 6.03 (br. s., 1H), 5.01 (dd, J=2.4, 4.3 Hz, 2H), 4.17-4.32 (m, 2H), 3.13-3.34 (m, 1H), 2.78-3.02 (m, 1H), 2.65 (s, 3H), 2.25-2.44 (m, 1H), 2.22 (br. s., 3H), 1.93-2.11 (m, 1H), 1.50-1.78 (m, 4H), 1.28 (t, J=7.2 Hz, 3H), 1.22 (s, 9H), 0.65 (br. s., 6H). LCMS (M+H)=632.5.

### EXAMPLE 5

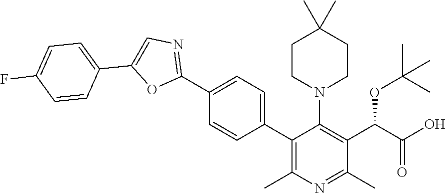 [SMILES: Cc1nc(C)c([C@H](OC(C)(C)C)C(=O)O)c(N2CCC(C)(C)CC2)c1-c1ccc(-c2ncc(-c3ccc(F)cc3)o2)cc1]

(S)-2-(tert-Butoxy)-2-(4-(4,4-dimethylpiperidin-1-yl)-5-(4-(5-(4-fluorophenyl)oxazol-2-yl)phenyl)-2,6-dimethylpyridin-3-yl)acetic acid: A solution of (S)-ethyl 2-(tert-butoxy)-2-(4-(4,4-dimethylpiperidin-1-yl)-5-(4-((2-(4-fluorophenyl)-2-oxoethyl)carbamoyl)phenyl)-2,6-dimethylpyridin-3-yl)acetate (0.051 g, 0.081 mmol) and Burgess reagent (0.038 g, 0.161 mmol) in THF (1.5 mL) was placed in pre-heated oil bath at 115° C. After 2 h, the reaction mixture was cooled, concentrated and the crude (S)-ethyl 2-(tert-butoxy)-2-(4-(4,4-dimethylpiperidin-1-yl)-5-(4-(5-(4-fluorophenyl)oxazol-2-yl)phenyl)-2,6-dimethylpyridin-3-yl)acetate was used in the next step without purification. LCMS (M+H)=614.5.

To a solution of above crude (S)-ethyl 2-(tert-butoxy)-2-(4-(4,4-dimethylpiperidin-1-yl)-2,6-dimethylpyridin-3-yl)acetate in 9:1 EtOH/H₂O (2 mL) was added LiOH (0.019 g, 0.807 mmol) and heated at reflux for 4 h. Then, cooled and purified by prep-HPLC, the fractions containing the compound were combined, concentrated to give white solid which was dissolved in water/CH₃CN and treated with 0.1 mL of 1M HCl and lyophilyzed to afford (S)-2-(tert-butoxy)-2-(4-(4,4-dimethylpiperidin-1-yl)-5-(4-(5-(4-fluorophenyl)oxazol-2-yl)phenyl)-2,6-dimethylpyridin-3-yl)acetic acid HCl (0.042 g, 0.068 mmol, 84% yield) as white solid. ¹H NMR (500 MHz, CDCl₃) δ 8.30 (d, J=8.0 Hz, 2H), 7.74-7.79 (m, 2H), 7.49 (s, 1H), 7.43 (d, J=7.4 Hz, 1H), 7.18-7.25 (m, 3H), 5.73 (s, 1H), 2.99 (s, 3H), 2.63 (s, 3H), 1.38 (br. s., 4H), 1.27 (s, 9H), 0.85 (br. s., 6H). 4H of piperidine are missing. LCMS (M+H)=586.5.

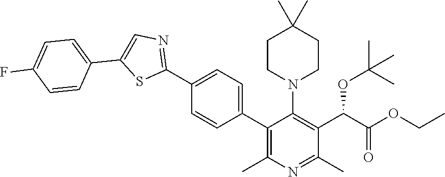 [SMILES: CCOC(=O)[C@@H](OC(C)(C)C)c1c(C)nc(C)c(-c2ccc(-c3ncc(-c4ccc(F)cc4)s3)cc2)c1N1CCC(C)(C)CC1]

(S)-Ethyl 2-(tert-butoxy)-2-(4-(4,4-dimethylpiperidin-1-yl)-5-(4-(5-(4-fluorophenyl)thiazol-2-yl)phenyl)-2,6-dimethylpyridin-3-yl)acetate: A mixture of (S)-ethyl 2-(tert-butoxy)-2-(4-(4,4-dimethylpiperidin-1-yl)-5-(4-((2-(4-fluorophenyl)-2-oxoethyl)carbamoyl)phenyl)-2,6-dimethylpyridin-3-yl)acetate (0.07 g, 0.111 mmol) and Lawesson's reagent (0.067 g, 0.166 mmol) in anhydrous toluene (4 mL) was heated at 80° C. for 6.5 h and concentrated to give residue which was purified by prep-HPLC to afford (S)-ethyl 2-(tert-butoxy)-2-(4-(4,4-dimethylpiperidin-1-yl)-5-(4-(5-(4-fluorophenyl)thiazol-2-yl)phenyl)-2,6-dimethylpyridin-3-yl)acetate (0.0593 g, 0.094 mmol, 85% yield) as off-white solid. ¹H NMR (500 MHz, CDCl₃) δ 8.09 (d, J=8.5 Hz, 2H), 8.02 (s, 1H), 7.60-7.65 (m, 2H), 7.39-7.43 (m, 1H), 7.24-7.28 (m, 1H), 7.14-7.20 (m, 2H), 5.96 (br. s., 1H), 4.18-4.32 (m, 2H), 2.72 (br. s., 3H), 2.34 (br. s., 3H), 1.37-1.78 (m, 4H), 1.30 (t, J=7.1 Hz, 3H), 1.22 (s, 9H), 0.79 (br. s., 6H). 4H of piperidine were not resolved. LCMS (M+H)=630.5.

### EXAMPLE 6

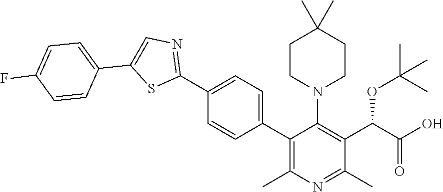 [SMILES: Cc1nc(C)c([C@H](OC(C)(C)C)C(=O)O)c(N2CCC(C)(C)CC2)c1-c1ccc(-c2ncc(-c3ccc(F)cc3)s2)cc1]

(S)-2-(tert-Butoxy)-2-(4-(4,4-dimethylpiperidin-1-yl)-5-(4-(5-(4-fluorophenyl)thiazol-2-yl)phenyl)-2,6-dimethylpyridin-3-yl)acetic acid: A solution of (S)-ethyl 2-(tert-butoxy)-2-(4-(4,4-dimethylpiperidin-1-yl)-5-(4-(5-(4-fluorophenyl)thiazol-2-yl)phenyl)-2,6-dimethylpyridin-3-yl)acetate (0.0448 g, 0.071 mmol) and LiOH (0.017 g, 0.711 mmol) in 9:1 EtOH/H₂O (2 mL) was refluxed for 3 h. Then, cooled and purified by prep-HPLC to afford (S)-2-(tert-butoxy)-2-(4-(4,4-dimethylpiperidin-1-yl)-5-(4-(5-(4-fluorophenyl)thiazol-2-yl)phenyl)-2,6-dimethylpyridin-3-yl)acetic acid (0.0382 g, 0.063 mmol, 89% yield) as white solid. ¹H NMR (500 MHz, CDCl₃) δ 8.08-8.14 (m, 2H), 8.02 (s, 1H), 7.60-7.65 (m, 2H), 7.40 (dd, J=1.4, 8.4 Hz, 1H), 7.26 (d, J=7.9 Hz, 1H), 7.14-7.21 (m, 2H), 5.90 (br. s., 1H), 2.77 (br. s., 3H), 2.38 (br. s., 3H), 1.28-1.40 (m, 4H), 1.26 (s, 9H), 0.80 (br. s., 6H). 4H of piperidine are missing. LCMS (M+H)=602.5.

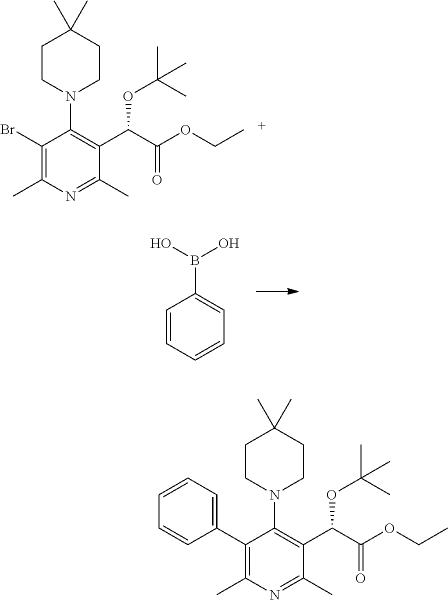 [SMILES: CCOC(=O)[C@@H](OC(C)(C)C)c1c(C)nc(C)c(Br)c1N1CCC(C)(C)CC1 ; OB(O)c1ccccc1 ; CCOC(=O)[C@@H](OC(C)(C)C)c1c(C)nc(C)c(-c2ccccc2)c1N1CCC(C)(C)CC1]

(S)-Ethyl 2-(tert-butoxy)-2-(4-(4,4-dimethylpiperidin-1-yl)-2,6-dimethyl-5-phenylpyridin-3-yl)acetate: A mixture of (S)-ethyl 2-(5-bromo-4-(4,4-dimethylpiperidin-1-yl)-2,6-dimethylpyridin-3-yl)-2-(tert-butoxy)acetate (0.031 g, 0.068 mmol), phenylboronic acid (0.012 g, 0.102 mmol) and 2M Na₂CO₃ (0.085 ml, 0.170 mmol) in DMF (2 mL) was degassed for 10 min. Then, Pd(Ph₃P)₄ (7.87 mg, 6.81 μmol) added, degassed for 5 min and placed in a oil bath pre-heated to 100° C. After 2 h at 110° C., cooled and purified by prep-HPLC to afford (S)-ethyl 2-(tert-butoxy)-2-(4-(4,4-dimethylpiperidin-1-yl)-2,6-dimethyl-5-phenylpyridin-3-yl)acetate (0.0185 g, 0.041 mmol, 60.0% yield) as pale yellow paste. ¹H NMR (500 MHz, CDCl₃) δ 7.37-7.48 (m, 3H), 7.26-7.30 (m, 1H), 7.18 (dd, J=1.3, 7.5 Hz, 1H), 6.09 (s, 1H), 4.13-4.31 (m, 2H), 3.19 (br. s., 1H), 2.88 (br. s., 1H), 2.62 (s, 3H), 2.23-2.33 (m, 1H), 2.21 (s, 3H), 1.89-2.04 (m, 1H), 1.27 (t, J=7.1 Hz, 3H), 1.21 (s, 9H), 0.90 (br.s., 3H), 0.62 (br. s., 3H). 4H of piperidine were not resolved. LCMS (M+H)=453.5.

### EXAMPLE 7

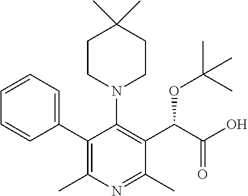 [SMILES: Cc1nc(C)c([C@H](OC(C)(C)C)C(=O)O)c(N2CCC(C)(C)CC2)c1-c1ccccc1]

(S)-2-(tert-Butoxy)-2-(4-(4,4-dimethylpiperidin-1-yl)-2,6-dimethyl-5-phenylpyridin-3-yl)acetic acid: A mixture of (S)-ethyl 2-(tert-butoxy)-2-(4-(4,4-dimethylpiperidin-1-yl)-2,6-dimethyl-5-phenylpyridin-3-yl)acetate (0.0185 g, 0.041 mmol) and LiOH (9.79 mg, 0.409 mmol) in 9:1 EtOH/H₂O (2 mL) was refluxed for 3 h. Then, cooled and purified by prep-HPLC to afford (S)-2-(tert-butoxy)-2-(4-(4,4-dimethylpiperidin-1-yl)-2,6-dimethyl-5-phenylpyridin-3-yl)acetic acid (0.0143 g, 0.034 mmol, 82% yield) as white solid. ¹H NMR (500 MHz, CDCl₃) δ 7.39-7.51 (m, 3H), 7.28 (d, J=7.3 Hz, 1H), 7.16 (d, J=7.3 Hz, 1H), 5.91 (br. s., 1H), 2.71 (s, 3H), 2.27 (s, 3H), 1.27-1.36 (m, 4H), 1.25 (s, 9H), 0.76 (br. s., 6H). 4H of piperidine are missing. LCMS (M+H)=425.5.

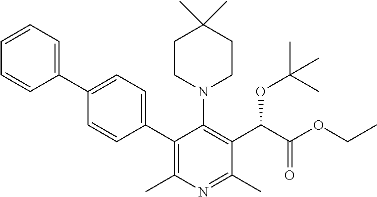 [SMILES: CCOC(=O)[C@@H](OC(C)(C)C)c1c(C)nc(C)c(-c2ccc(-c3ccccc3)cc2)c1N1CCC(C)(C)CC1]

(S)-Ethyl 2-(5-([1,1′-biphenyl]-4-yl)-4-(4,4-dimethylpiperidin-1-yl)-2,6-dimethylpyridin-3-yl)-2-(tert-butoxy)acetate: A mixture of (S)-ethyl 2-(5-bromo-4-(4,4-dimethylpiperidin-1-yl)-2,6-dimethylpyridin-3-yl)-2-(tert-butoxy)acetate (0.0372 g, 0.082 mmol), [1,1′-biphenyl]-4-ylboronic acid (0.024 g, 0.123 mmol) and 2M Na₂CO₃ (0.102 ml, 0.204 mmol) in DMF (2 mL) was degassed for 10 min. Then, Pd(Ph₃P)₄ (9.44 mg, 8.17 μmol) added, degassed for 5 min and placed in a pre-heated oil bath at 100° C. After 2 h at 110° C., cooled and purified by prep-HPLC to afford (S)-ethyl 2-(5-([1,1′-biphenyl]-4-yl)-4-(4,4-dimethylpiperidin-1-yl)-2,6-dimethylpyridin-3-yl)-2-(tert-butoxy)acetate (0.0273 g, 0.052 mmol, 63.2% yield) as white solid. ¹H NMR (500 MHz, CDCl₃) δ 7.66-7.73 (m, 4H), 7.48-7.52 (m, 2H), 7.38-7.43 (m, 1H), 7.36 (dd, J=1.6, 7.9 Hz, 1H), 7.26 (dd, 7.8 Hz, 1H), 6.10 (s, 1H), 4.15-4.32 (m, 2H), 3.24 (d, J=11.8 Hz, 1H), 2.93 (t, J=11.9 Hz, 1H), 2.63 (s, 3H), 2.32 (d, J=11.8 Hz, 1H), 2.27 (s, 3H), 2.09 (t, J=11.6 Hz, 1H), 1.57 (dt, J=4.1, 12.6 Hz, 1H), 1.33-1.42 (m, 1H), 1.28 (t, J=7.1 Hz, 4H), 1.22 (s, 9H), 1.19-1.21 (m, 1H), 1.06 (d, J=12.3 Hz, 1H), 0.90 (s, 3H), 0.63 (s, 3H). LCMS (M+H)=529.4.

### EXAMPLE 8

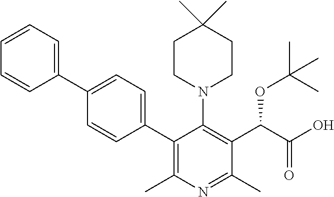 [SMILES: Cc1nc(C)c([C@H](OC(C)(C)C)C(=O)O)c(N2CCC(C)(C)CC2)c1-c1ccc(-c2ccccc2)cc1]

(S)-2-(5-([1,1′-Biphenyl]-4-yl)-4-(4,4-dimethylpiperidin-1-yl)-2,6-dimethylpyridin-3-yl)-2-(tert-butoxy)acetic acid: A mixture of (S)-ethyl 2-(5-([1,1′-biphenyl]-4-yl)-4-(4,4-dimethylpiperidin-1-yl)-2,6-dimethylpyridin-3-yl)-2-(tert-butoxy)acetate (0.0245 g, 0.046 mmol) and LiOH (0.011 g, 0.463 mmol) in 9:1 EtOH/H₂O (2 mL) was refluxed for 3.5 h. Then, cooled and purified by prep-HPLC to afford (S)-2-(5-([1,1′-biphenyl]-4-yl)-4-(4,4-dimethylpiperidin-1-yl)-2,6-dimethylpyridin-3-yl)-2-(tert-butoxy)acetic acid (0.0212 g, 0.042 mmol, 91% yield) as white solid. ¹H NMR (500 MHz, CDCl₃) δ 7.67-7.74 (m, 4H), 7.48-7.53 (m, 2H), 7.38-7.43 (m, 1H), 7.35 (dd, J=1.5, 7.8 Hz, 1H), 7.25 (dd, 7.9 Hz, 1H), 6.01 (br. s., 1H), 3.48-3.74 (m, 1H), 2.90-4.07 (m, 1H), 2.68 (s, 3H), 2.30-2.44 (m, 1H), 2.29 (s, 3H), 1.97-2.23 (m, 2H), 1.50-1.67 (m, 1H), 1.29-1.42 (m, 2H), 1.27 (s, 9H), 0.87 (br.s., 3H), 0.67 (br. s., 3H). LCMS (M+H)=501.4.

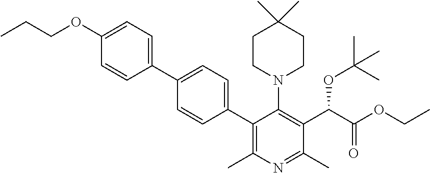 [SMILES: CCCOc1ccc(-c2ccc(-c3c(C)nc(C)c([C@H](OC(C)(C)C)C(=O)OCC)c3N3CCC(C)(C)CC3)cc2)cc1]

(S)-Ethyl 2-(tert-butoxy)-2-(4-(4,4-dimethylpiperidin-1-yl)-2,6-dimethyl-5-(4′-propoxy-[1,1′-biphenyl]-4-yl)pyridin-3-yl)acetate: A mixture of (S)-ethyl 2-(5-bromo-4-(4,4-dimethylpiperidin-1-yl)-2,6-dimethylpyridin-3-yl)-2-(tert-butoxy)acetate (0.0466 g, 0.102 mmol), (4′-propoxy-[1,1′-biphenyl]-4-yl)boronic acid (0.039 g, 0.153 mmol) and 2M Na₂CO₃ (0.128 ml, 0.256 mmol) in DIVIF (2 mL) was degassed for 10 min. Then, Pd(Ph₃P)₄ (0.012 g, 10.23 μmol) was added, degassed for 5 min and placed in a pre-heated oil bath at 110 C. After 2 h, cooled and purified by prep-HPLC to afford (S)-ethyl 2-(tert-butoxy)-2-(4-(4,4-dimethylpiperidin-1-yl)-2,6-dimethyl-5-(4′-propoxy-[1,1′-biphenyl]-4-yl)pyridin-3-yl)acetate (0.0354 g, 0.060 mmol, 59.0% yield) as white solid. ¹H NMR (500 MHz, CDCl₃) δ 7.64-7.67 (m, 2H), 7.60-7.63 (m, 2H), 7.32 (dd, J=1.5, 7.8 Hz, 1H), 7.22 (dd, J=1.7, 7.8 Hz, 1H), 7.01-7.05 (m, 2H), 6.10 (s, 1H), 4.28 (qd, 10.8 Hz, 1H), 4.19 (qd, J=7.1, 10.8 Hz, 1H), 4.01 (t, J=6.6 Hz, 2H), 3.23 (d, J=11.7 Hz, 1H), 2.93 (t, J=12.1 Hz, 1H), 2.63 (s, 3H), 2.31 (d, J=9.5 Hz, 1H), 2.26 (s, 3H), 2.09 (t, J=12.4 Hz, 1H), 1.88 (sxt, J=7.1 Hz, 2H), 1.51-1.58 (m, 1H), 1.33-1.41 (m, 1H), 1.27 (t, J=7.2 Hz, 3H), 1.22 (s, 9H), 1.18-1.21 (m, 1H), 1.09 (t, J=7.5 Hz, 4H), 1.05-1.08 (m, 1H), 0.89 (s, 3H), 0.62 (s, 3H). LCMS (M+H)=587.5.

### EXAMPLE 9

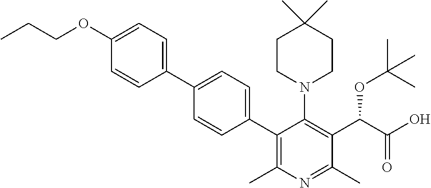 [SMILES: CCCOc1ccc(-c2ccc(-c3c(C)nc(C)c([C@H](OC(C)(C)C)C(=O)O)c3N3CCC(C)(C)CC3)cc2)cc1]

(S)-2-(tert-Butoxy)-2-(4-(4,4-dimethylpiperidin-1-yl)-2,6-dimethyl-5-(4′-propoxy-[1,1′-biphenyl]-4-yl)pyridin-3-yl)acetic acid: A mixture of (S)-ethyl 2-(tert-butoxy)-2-(4-(4,4-dimethylpiperidin-1-yl)-2,6-dimethyl-5-(4′-propoxy-[1,1′-biphenyl]-4-yl)pyridin-3-yl)acetate (0.0336 g, 0.057 mmol) and LiOH (0.014 g, 0.573 mmol) in 9:1 EtOH/H₂O (2 mL) was refluxed for 3 h. Then, cooled and purified by prep-HPLC to afford (S)-2-(tert-butoxy)-2-(4-(4,4-dimethylpiperidin-1-yl)-2,6-dimethyl-5-(4′-propoxy-[1,1′-biphenyl]-4-yl)pyridin-3-yl)acetic acid (0.0312 g, 0.056 mmol, 98% yield) as white solid. ¹H NMR (500 MHz, CDCl₃) δ 7.59-7.68 (m, 4H), 7.31 (dd, J=1.6, 7.9 Hz, 1H), 7.21 (dd, J=1.3, 7.8 Hz, 1H), 7.00-7.06 (m, 2H), 5.96 (br. s., 1H), 4.01 (t, J=6.6 Hz, 2H), 3.50-3.79 (m, 1H), 2.79-3.08 (m, 1H), 2.67 (s, 3H), 2.28 (s, 3H), 1.96-2.21 (m, 1H), 1.82-1.91 (m, 2H), 1.49-1.67 (m, 1H), 1.27-1.40 (m, 2H), 1.25 (s, 9H), 1.09 (t, J=7.4 Hz, 3H), 0.84 (br.s., 3H), 0.67 (br.s., 3H). 2H of piperidine are missing. LCMS (M+H)=559.4.

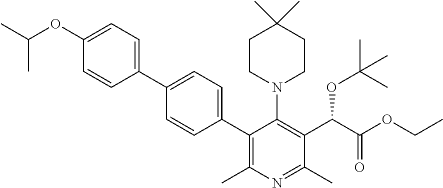 [SMILES: CCOC(=O)[C@@H](OC(C)(C)C)c1c(C)nc(C)c(-c2ccc(-c3ccc(OC(C)C)cc3)cc2)c1N1CCC(C)(C)CC1]

(S)-Ethyl 2-(tert-butoxy)-2-(4-(4,4-dimethylpiperidin-1-yl)-5-(4′-isopropoxy-[1,1′-biphenyl]-4-yl)-2,6-dimethylpyridin-3-yl)acetate: A mixture of (S)-ethyl 2-(5-bromo-4-(4,4-dimethylpiperidin-1-yl)-2,6-dimethylpyridin-3-yl)-2-(tert-butoxy)acetate (0.047 g, 0.103 mmol), (4′-isopropoxy-[1,1′-biphenyl]-4-yl)boronic acid (0.040 g, 0.155 mmol) and 2M Na₂CO₃ (0.129 ml, 0.258 mmol) in DMF (2 mL) was degassed for 10 min. Then, Pd(Ph₃P)₄ (0.012 g, 10.32 μmol) was added, degassed for 5 min and placed in a pre-heated oil bath at 110° C. After 2 h, cooled and purified by prep-HPLC to afford (S)-ethyl 2-(tert-butoxy)-2-(4-(4,4-dimethylpiperidin-1-yl)-5-(4′-isopropoxy-[1,1′-biphenyl]-4-yl)-2,6-dimethylpyridin-3-yl)acetate (0.0365 g, 0.062 mmol, 60.3% yield) as white solid. ¹H NMR (500 MHz, CDCl₃) δ 7.59-7.68 (m, 4H), 7.32 (dd, J=1.6, 7.9 Hz, 1H), 7.22 (dd, J=1.7, 7.8 Hz, 1H), 7.00-7.04 (m, 2H), 6.10 (s, 1H), 4.64 (td, J=6.1, 12.1 Hz, 1H), 4.24-4.32 (m, 1H), 4.19 (qd, J=7.1, 10.7 Hz, 1H), 3.23 (d, J=11.5 Hz, 1H), 2.93 (t, J=11.8 Hz, 1H), 2.63 (s, 3H), 2.31 (d, J=9.9 Hz, 1H), 2.26 (s, 3H), 2.09 (t, J=12.4 Hz, 1H), 1.52-1.58 (m, 1H), 1.41 (d, J=6.2 Hz, 6H), 1.33-1.38 (m, 1H), 1.27 (t, J=7.1 Hz, 3H), 1.22 (s, 9H), 1.17-1.21 (m, 1H), 1.06 (d, J=12.9 Hz, 1H), 0.89 (s, 3H), 0.62 (s, 3H). LCMS (M+H)=587.5.

### EXAMPLE 10

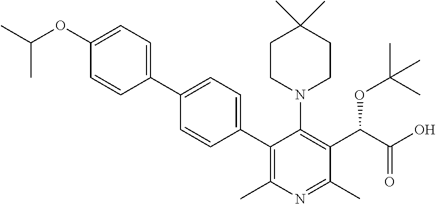 [SMILES: Cc1nc(C)c([C@H](OC(C)(C)C)C(=O)O)c(N2CCC(C)(C)CC2)c1-c1ccc(-c2ccc(OC(C)C)cc2)cc1]

(S)-2-(tert-Butoxy)-2-(4-(4,4-dimethylpiperidin-1-yl)-5-(4′-isopropoxy-[1,1′-biphenyl]-4-yl)-2,6-dimethylpyridin-3-yl)acetic acid: A mixture of (S)-ethyl 2-(tert-butoxy)-2-(4-(4,4-dimethylpiperidin-1-yl)-5-(4′-isopropoxy-[1,1′-biphenyl]-4-yl)-2,6-dimethylpyridin-3-yl)acetate (0.0345 g, 0.059 mmol) and LiOH (0.014 g, 0.588 mmol) in 9:1 EtOH/H₂O (2 mL) was refluxed for 3 h. Then, cooled and purified by prep-HPLC to afford (S)-2-(tert-butoxy)-2-(4-(4,4-dimethylpiperidin-1-yl)-5-(4′-isopropoxy-[1,1′-biphenyl]-4-yl)-2,6-dimethylpyridin-3-yl)acetic acid (0.032 g, 0.057 mmol, 97% yield) as off-white solid. ¹H NMR (500 MHz, CDCl₃) δ 7.64-7.68 (m, 2H), 7.57-7.63 (m, 2H), 7.31 (dd, J=1.6, 7.9 Hz, 1H), 7.21 (dd, J=1.5, 7.8 Hz, 1H), 6.99-7.04 (m, 2H), 5.97 (br. s., 1H), 4.64 (spt, J=6.0 Hz, 1H), 3.65 (br. s., 1H), 2.96 (br. s., 1H), 2.68 (s, 3H), 2.28 (s, 3H), 2.00-2.20 (m, 1H), 1.47-1.66 (m, 1H), 1.41 (d, J=6.0 Hz, 6H), 1.27-1.39 (m, 3H), 1.26 (s, 9H), 0.85 (br.s., 3H), 0.66 (br.s., 3H). One piperidine proton was not resolved. LCMS (M+H)=559.4.

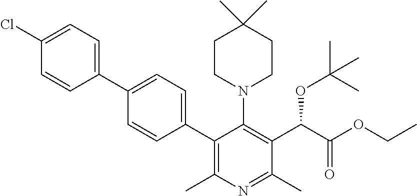 [SMILES: CCOC(=O)[C@@H](OC(C)(C)C)c1c(C)nc(C)c(-c2ccc(-c3ccc(Cl)cc3)cc2)c1N1CCC(C)(C)CC1]

(S)-Ethyl 2-(tert-butoxy)-2-(5-(4′-chloro-[1,1′-biphenyl]-4-yl)-4-(4,4-dimethylpiperidin-1-yl)-2,6-dimethylpyridin-3-yl)acetate: A mixture of (S)-ethyl 2-(5-bromo-4-(4,4-dimethylpiperidin-1-yl)-2,6-dimethylpyridin-3-yl)-2-(tert-butoxy)acetate (0.0427 g, 0.094 mmol), (4′-chloro-[1,1′-biphenyl]-4-yl)boronic acid (0.033 g, 0.141 mmol) and 2M Na₂CO₃ (0.117 ml, 0.234 mmol) in DMF (2 mL) was degassed for 10 min. Then, Pd(Ph₃P)₄ (10.83 mg, 9.38 μmol) added, degassed for 5 min and placed in a pre-heated oil bath at 110° C. After 2 h, cooled and purified by prep-HPLC to afford (S)-ethyl 2-(tert-butoxy)-2-(5-(4′-chloro-[1,1′-biphenyl]-4-yl)-4-(4,4-dimethylpiperidin-1-yl)-2,6-dimethylpyridin-3-yl)acetate (0.0295 g, 0.052 mmol, 55.9% yield) as white solid. ¹H NMR (500 MHz, CDCl₃) δ 7.60-7.68 (m, 4H), 7.45-7.49 (m, 2H), 7.37 (dd, J=1.5, 7.8 Hz, 1H), 7.26 (dd, J=1.6, 7.9 Hz, 1H), 6.08 (s, 1H), 4.28 (qd, J=7.1, 10.7 Hz, 1H), 4.19 (qd, J=7.1, 10.8 Hz, 1H), 3.24 (d, J=12.3 Hz, 1H), 2.91 (t, J=12.5 Hz, 1H), 2.63 (s, 3H), 2.31 (d, J=11.0 Hz, 1H), 2.25 (s, 3H), 2.06 (t, J=11.9 Hz, 1H), 1.54-1.57 (m, 1H), 1.34-1.38 (m, 1H), 1.28 (t, J=7.1 Hz, 3H), 1.22 (s, 9H), 1.18 (br. s., 1H), 1.03-1.10 (m, 1H), 0.90 (s, 3H), 0.62 (s, 3H). LCMS (M+H)=563.4.

### EXAMPLE 11

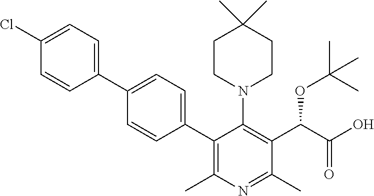 [SMILES: Cc1nc(C)c([C@H](OC(C)(C)C)C(=O)O)c(N2CCC(C)(C)CC2)c1-c1ccc(-c2ccc(Cl)cc2)cc1]

(S)-2-(tert-Butoxy)-2-(5-(4′-chloro-[1,1′-biphenyl]-4-yl)-4-(4,4-dimethylpiperidin-1-yl)-2,6-dimethylpyridin-3-yl)acetic acid: A mixture of (S)-ethyl 2-(tert-butoxy)-2-(5-(4′-chloro-[1,1′-biphenyl]-4-yl)-4-(4,4-dimethylpiperidin-1-yl)-2,6-dimethylpyridin-3-yl)acetate (0.028 g, 0.050 mmol) and LiOH (0.012 g, 0.497 mmol) in 9:1 EtOH/H₂O (2 mL) was refluxed for 3 h. Then, cooled and purified by prep-HPLC to afford (S)-2-(tert-butoxy)-2-(5-(4′-chloro-[1,1′-biphenyl]-4-yl)-4-(4,4-dimethylpiperidin-1-yl)-2,6-dimethylpyridin-3-yl)acetic acid (0.026 g, 0.049 mmol, 98% yield) as white solid. ¹H NMR (500 MHz, CDCl₃) δ 7.64-7.69 (m, 2H), 7.59-7.63 (m, 2H), 7.45-7.49 (m, 2H), 7.36 (dd, J=1.5, 8.0 Hz, 1H), 7.26 (dd, J=1.4, 7.9 Hz, 1H), 6.00 (br. s., 1H), 3.62 (br. s., 1H), 2.95 (br. s., 1H), 2.65 (s, 3H), 2.26 (s, 3H), 1.93-2.17 (m, 1H), 1.27-1.36 (m, 2H), 1.26 (s, 9H), 1.02-1.21 (m, 2H), 0.86 (br.s., 3H, 0.64 (br. s., 3H). One proton of piperidine was not resolved. LCMS (M+H)=535.3.

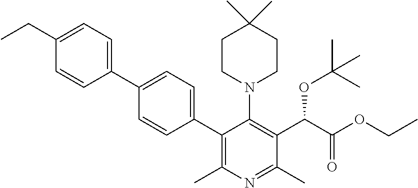 [SMILES: CCOC(=O)[C@@H](OC(C)(C)C)c1c(C)nc(C)c(-c2ccc(-c3ccc(CC)cc3)cc2)c1N1CCC(C)(C)CC1]

(S)-Ethyl 2-(tert-butoxy)-2-(4-(4,4-dimethylpiperidin-1-yl)-5-(4′-ethyl-[1,1′-biphenyl]-4-yl)-2,6-dimethylpyridin-3-yl)acetate: A mixture of (S)-ethyl 2-(5-bromo-4-(4,4-dimethylpiperidin-1-yl)-2,6-dimethylpyridin-3-yl)-2-(tert-butoxy)acetate (0.0381 g, 0.084 mmol), (4′-ethyl-[1,1′-biphenyl]-4-yl)boronic acid (0.028 g, 0.125 mmol) and 2M Na₂CO₃ (0.105 ml, 0.209 mmol) in DIVIF (2 mL) was degassed for 10 min. Then, Pd(Ph₃P)₄ (9.67 mg, 8.37 μmol) added, degassed for 5 min and placed in a pre-heated oil bat at 110° C. After 2 h, cooled and purified by prep-HPLC to afford (S)-ethyl 2-(tert-butoxy)-2-(4-(4,4-dimethylpiperidin-1-yl)-5-(4′-ethyl-[1,1′-biphenyl]-4-yl)-2,6-dimethylpyridin-3-yl)acetate (0.0276 g, 0.050 mmol, 59.3% yield) as white solid. ¹H NMR (500 MHz, CDCl₃) δ 7.69 (ddd, J=1.8, 7.9, 13.8 Hz, 2H), 7.60-7.64 (m, 2H), 7.32-7.36 (m, 3H), 7.24 (dd, J=1.7, 7.8 Hz, 1H), 6.09 (br. s., 1H), 4.24-4.32 (m, 1H), 4.19 (qd, J=7.1, 10.7 Hz, 1H), 3.22 (br. s., 1H), 2.93 (br. s., 1H), 2.75 (q, J=7.6 Hz, 2H), 2.64 (s, 3H), 2.29-2.36 (m, 1H), 2.27 (s, 3H), 2.03-2.13 (m, 1H), 1.50-1.58 (m, 1H), 1.35-1.41 (m, 1H), 1.32 (t, J=7.6 Hz, 3H), 1.28 (t, J=7.3 Hz, 3H), 1.22 (s, 9H), 1.01-1.11 (m, 1H), 0.89 (br.s, 3H), 0.62 (br. s., 3H). One proton of piperidine was not resolved. LCMS (M+H)=557.4.

### EXAMPLE 12

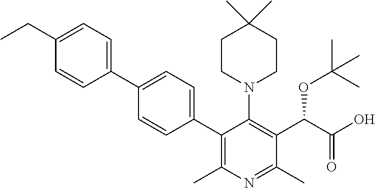 [SMILES: CCc1ccc(-c2ccc(-c3c(C)nc(C)c([C@H](OC(C)(C)C)C(=O)O)c3N3CCC(C)(C)CC3)cc2)cc1]

(S)-2-(tert-Butoxy)-2-(4-(4,4-dimethylpiperidin-1-yl)-5-(4′-ethyl-[1,1′-biphenyl]-4-yl)-2,6-dimethylpyridin-3-yl)acetic acid: A mixture of (S)-ethyl 2-(tert-butoxy)-2-(4-(4,4-dimethylpiperidin-1-yl)-5-(4′-ethyl-[1,1′-biphenyl]-4-yl)-2,6-dimethylpyridin-3-yl)acetate (0.025 g, 0.045 mmol) and LiOH (10.75 mg, 0.449 mmol) in 9:1 EtOH/H₂O (2 mL) was refluxed for 3 h. Then, cooled and purified by pre-HPLC to afford (S)-2-(tert-butoxy)-2-(4-(4,4-dimethylpiperidin-1-yl)-5-(4′-ethyl-[1,1′-biphenyl]-4-yl)-2,6-dimethylpyridin-3-yl)acetic acid (0.0233 g, 0.044 mmol, 98% yield) as pale yellow solid. ¹H NMR (500 MHz, CDCl₃) δ 7.69 (ddd, J=1.8, 7.9, 14.2 Hz, 2H), 7.60-7.64 (m, 2H), 7.31-7.36 (m, 3H), 7.24 (dd, J=1.6, 7.9 Hz, 1H), 5.99 (br. s., 1H), 3.63 (br. s., 1H), 2.82-3.12 (m, 1H), 2.74 (q, J=7.6 Hz, 2H), 2.66 (s, 3H), 2.28 (s, 3H), 1.95-2.21 (m, 2H), 1.32 (t, J=7.6 Hz, 3H), 1.28-1.33 (m, 2H), 1.26 (s, 9H), 1.02-1.20 (m, 2H), 0.87 (br.s., 3H), 0.64 (br. s., 3H). LCMS (M+H)=529.5.

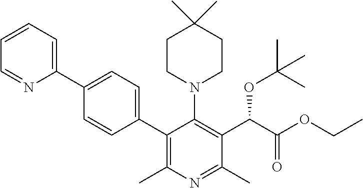 [SMILES: CCOC(=O)[C@@H](OC(C)(C)C)c1c(C)nc(C)c(-c2ccc(-c3ccccn3)cc2)c1N1CCC(C)(C)CC1]

(S)-Ethyl 2-(tert-butoxy)-2-(4-(4,4-dimethylpiperidin-1-yl)-2,6-dimethyl-5-(4-(pyridin-2-yl)phenyl)pyridin-3-yl)acetate: A mixture of (S)-ethyl 2-(5-bromo-4-(4,4-dimethylpiperidin-1-yl)-2,6-dimethylpyridin-3-yl)-2-(tert-butoxy)acetate (0.042 g, 0.092 mmol), (4-(pyridin-2-yl)phenyl)boronic acid (0.028 g, 0.138 mmol) and 2M Na₂CO₃ (0.115 ml, 0.231 mmol) in DMF (2 mL) degassed for 10 min. Then, Pd(Ph₃P)₄ (10.66 mg, 9.22 μmol) was added, degassed for 5 min and placed in a pre-heated oil bath at 110° C. After 2 h, cooled and purified by pre-HPLC to afford (S)-ethyl 2-(tert-butoxy)-2-(4-(4,4-dimethylpiperidin-1-yl)-2,6-dimethyl-5-(4-(pyridin-2-yl)phenyl)pyridin-3-yl)acetate (0.0244 g, 0.046 mmol, 49.9% yield) as white solid. ¹H NMR (500 MHz, CDCl₃) δ 8.74-8.77 (m, 1H), 8.11 (ddd, J=1.7, 7.9, 16.7 Hz, 2H), 7.79-7.86 (m, 2H), 7.42 (dd, J=1.6, 7.9 Hz, 1H), 7.29-7.32 (m, 2H), 6.09 (s, 1H), 4.28 (qd, J=7.1, 10.7 Hz, 1H), 4.20 (qd, J=7.1, 10.8 Hz, 1H), 3.25 (d, J=11.5 Hz, 1H), 2.93 (t, J=12.1 Hz, 1H), 2.63 (s, 3H), 2.34 (d, J=11.7 Hz, 1H), 2.23 (s, 3H), 2.14 (t, J=11.3 Hz, 1H), 1.52-1.58 (m, 1H), 1.34-1.42 (m, 1H), 1.28 (t, J=7.3 Hz, 3H), 1.22 (s, 9H), 1.18 (br. s., 1H), 1.04-1.10 (m, 1H), 0.89 (br. s., 3H), 0.62 (s, 3H). LCMS (M+H)=530.3.

### EXAMPLE 13

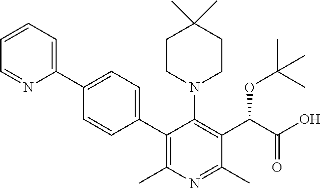 [SMILES: Cc1nc(C)c([C@H](OC(C)(C)C)C(=O)O)c(N2CCC(C)(C)CC2)c1-c1ccc(-c2ccccn2)cc1]

(S)-2-(tert-Butoxy)-2-(4-(4,4-dimethylpiperidin-1-yl)-2,6-dimethyl-5-(4-(pyridin-2-yl)phenyl)pyridin-3-yl)acetic: A mixture of (S)-ethyl 2-(tert-butoxy)-2-(4-(4,4-dimethylpiperidin-1-yl)-2,6-dimethyl-5-(4-(pyridin-2-yl)phenyl)pyridin-3-yl)acetate (0.023 g, 0.043 mmol) and LiOH (10.40 mg, 0.434 mmol) in 9:1 EtOH/H₂O (2 mL) was refluxed for 3 h. Then, cooled and purified by prep-HPLC to afford (S)-2-(tert-butoxy)-2-(4-(4,4-dimethylpiperidin-1-yl)-2,6-dimethyl-5-(4-(pyridin-2-yl)phenyl)pyridin-3-yl)acetic acid (0.0199 g, 0.040 mmol, 91% yield) as white solid. ¹H NMR (500 MHz, CDCl₃) δ 8.76 (td, J=1.4, 4.7 Hz, 1H), 8.15 (dd, J=1.9, 8.0 Hz, 1H), 8.09 (dd, J=1.8, 8.0 Hz, 1H), 7.79-7.85 (m, 2H), 7.42 (dd, J=1.7, 8.0 Hz, 1H), 7.29-7.32 (m, 2H), 5.99 (br. s., 1H), 3.66 (br. s., 1H), 2.95 (br. s., 1H), 2.66 (s, 3H), 2.27-2.43 (m, 1H), 2.25 (s, 3H), 2.04-2.20 (m, 1H), 1.27-1.38 (m, 2H), 1.26 (s, 9H), 1.04-1.22 (m, 2H), 0.86 (br.s., 3H), 0.65 (br. s., 3H). LCMS (M+H)=502.4.

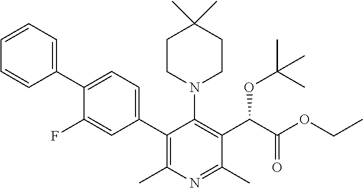 [SMILES: CCOC(=O)[C@@H](OC(C)(C)C)c1c(C)nc(C)c(-c2ccc(-c3ccccc3)c(F)c2)c1N1CCC(C)(C)CC1]

(S)-Ethyl 2-(tert-butoxy)-2-(4-(4,4-dimethylpiperidin-1-yl)-5-(2-fluoro-[1,1′-biphenyl]-4-yl)-2,6-dimethylpyridin-3-yl)acetate: A mixture of (S)-ethyl 2-(5-bromo-4-(4,4-dimethylpiperidin-1-yl)-2,6-dimethylpyridin-3-yl)-2-(tert-butoxy)acetate (0.0399 g, 0.088 mmol), (2-fluoro-[1,1′-biphenyl]-4-yl)boronic acid (0.028 g, 0.131 mmol) and 2M Na₂CO₃ (0.110 ml, 0.219 mmol) in DMF (2 mL) was degassed for 10 min. Then, Pd(Ph₃P)₄ (10.12 mg, 8.76 μmol) was added, degassed for 5 min and placed in a pre-heated oil bath at 110° C. After 2 h, cooled and purified by pre-HPLC to afford (S)-ethyl 2-(tert-butoxy)-2-(4-(4,4-dimethylpiperidin-1-yl)-5-(2-fluoro-[1,1′-biphenyl]-4-yl)-2,6-dimethylpyridin-3-yl)acetate (0.0315 g, 0.058 mmol, 65.8% yield) as white solid. ¹H NMR (500 MHz, CCl₃) δ 7.63-7.67 (m, 2H), 7.49-7.59 (m, 3H), 7.40-7.46 (m, 1H), 7.10-7.18 (m, 1H), 7.00-7.07 (m, 1H), 6.04 (br. s., 1H), 4.25-4.32 (m, 1H), 4.16-4.24 (m, 1H), 3.18-3.32 (m, 1H), 2.86-2.99 (m, 1H), 2.66 (br. s., 3H), 2.34-2.42 (m, 1H), 2.32 (br. s., 3H), 2.00-2.24 (m, 1H), 1.49-1.58 (m, 1H), 1.31-1.44 (m, 1H), 1.28 (dt, J=0.6, 7.1 Hz, 3H), 1.22 (s, 9H), 1.04-1.17 (m, 2H), 0.90 (br.s, 3H), 0.70 (br. s., 3H). LCMS (M+H)=547.4.

### EXAMPLE 14

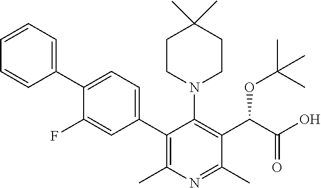 [SMILES: Cc1nc(C)c([C@H](OC(C)(C)C)C(=O)O)c(N2CCC(C)(C)CC2)c1-c1ccc(-c2ccccc2)c(F)c1]

(S)-2-(tert-Butoxy)-2-(4-(4,4-dimethylpiperidin-1-yl)-5-(2-fluoro-[1,1′-biphenyl]-4-yl)-2,6-dimethylpyridin-3-yl)acetic acid: A mixture of (S)-ethyl 2-(tert-butoxy)-2-(4-(4,4-dimethylpiperidin-1-yl)-5-(2-fluoro-[1,1′-biphenyl]-4-yl)-2,6-dimethylpyridin-3-yl)acetate (0.0293 g, 0.054 mmol) and LiOH (0.013 g, 0.536 mmol) in 9:1 EtOH/H₂O (2 mL0 was refluxed for 3 h. Then, cooled and purified by prep-HPLC to afford (S)-2-(tert-butoxy)-2-(4-(4,4-dimethylpiperidin-1-yl)-5-(2-fluoro-[1,1′-biphenyl]-4-yl)-2,6-dimethylpyridin-3-yl)acetic acid (0.0258 g, 0.050 mmol, 93% yield) as white solid. ¹H NMR (500 MHz, CDCl₃) δ 7.64 (d, J=7.3 Hz, 2H), 7.49-7.60 (m, 3H), 7.41-7.46 (m, 1H), 7.10-7.17 (m, 1H), 7.01-7.08 (m, 1H), 6.05 (br. s., 1H), 3.59 (br. s., 1H), 2.96 (br. s., 1H), 2.64 (s, 1.4H), 2.63 (s, 1.6H), 2.32-2.40 (m, 1H), 2.30 (s, 1.6H), 2.29 (s, 1.4H), 2.08-2.20 (m, 1H), 1.29-1.40 (m, 2H), 1.26 (s, 9H), 1.03-1.19 (m, 2H), 0.90 (br.s., 3H), 0.69 (br. s., 3H). LCMS (M+H)=519.4.

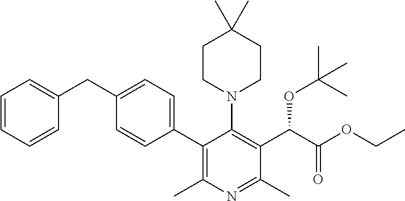 [SMILES: CCOC(=O)[C@@H](OC(C)(C)C)c1c(C)nc(C)c(-c2ccc(Cc3ccccc3)cc2)c1N1CCC(C)(C)CC1]

(S)-Ethyl 2-(5-(4-benzylphenyl)-4-(4,4-dimethylpiperidin-1-yl)-2,6-dimethylpyridin-3-yl)-2-(tert-butoxy)acetate: A mixture of (S)-ethyl 2-(5-bromo-4-(4,4-dimethylpiperidin-1-yl)-2,6-dimethylpyridin-3-yl)-2-(tert-butoxy)acetate (0.0392 g, 0.086 mmol), (4-benzylphenyl)boronic acid (0.027 g, 0.129 mmol) and 2M Na₂CO₃ (0.108 ml, 0.215 mmol) in DMF (2 mL) was degassed for 10 min. Then, Pd(Ph₃P)₄ (9.95 mg, 8.61 μmol) added, degassed for 5 min and placed in a pre-heated oil bath at 110° C. After 2 h, cooled and purified by prep-HPLC to afford (S)-ethyl 2-(5-(4-benzylphenyl)-4-(4,4-dimethylpiperidin-1-yl)-2,6-dimethylpyridin-3-yl)-2-(tert-butoxy)acetate (0.0256 g, 0.047 mmol, 54.8% yield) as colorless paste. ¹H NMR (500 MHz, CDCl₃) δ 7.24-7.33 (m, 4H), 7.19-7.24 (m, 4H), 7.09-7.12 (m, 1H), 6.09 (s, 1H), 4.13-4.30 (m, 2H), 4.08 (s, 2H), 3.17 (d, J=12.1 Hz, 1H), 2.86 (t, J=11.7 Hz, 1H), 2.61 (s, 3H), 2.26 (d, J=11.7 Hz, 1H), 2.22 (s, 3H), 2.00 (t, J=11.4 Hz, 1H), 1.54 (dt, J=4.2, 12.6 Hz, 1H), 1.32-1.40 (m, 1H), 1.26 (t, J=7.1 Hz, 3H), 1.21 (s, 9H), 1.17-1.20 (m, 1H), 1.05 (d, J=11.4 Hz, 1H), 0.90 (s, 3H), 0.61 (s, 3H). LCMS (M+H)=543.5.

### EXAMPLE 15

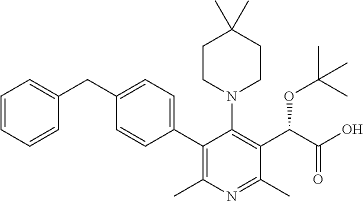 [SMILES: Cc1nc(C)c([C@H](OC(C)(C)C)C(=O)O)c(N2CCC(C)(C)CC2)c1-c1ccc(Cc2ccccc2)cc1]

(S)-2-(5-(4-Benzylphenyl)-4-(4,4-dimethylpiperidin-1-yl)-2,6-dimethylpyridin-3-yl)-2-(tert-butoxy)acetic acid: A mixture of (S)-ethyl 2-(5-(4-benzylphenyl)-4-(4,4-dimethylpiperidin-1-yl)-2,6-dimethylpyridin-3-yl)-2-(tert-butoxy)acetate (0.0254 g, 0.047 mmol) and LiOH (0.011 g, 0.468 mmol) in 9:1 EtOH/H₂O (2 mL) was refluxed for 3 h. Then, cooled and purified by prep-HPLC to afford (S)-2-(5-(4-benzylphenyl)-4-(4,4-dimethylpiperidin-1-yl)-2,6-dimethylpyridin-3-yl)-2-(tert-butoxy)acetic acid (0.0217 g, 0.042 mmol, 90% yield) as solid. ¹H NMR (500 MHz, CDCl₃) δ 7.27-7.33 (m, 7H), 7.18-7.25 (m, 4H), 7.09 (d, J=7.7 Hz, 1H), 5.97 (br. s., 1H), 4.08 (s, 2H), 3.36-3.73 (m, 1H), 2.75-3.05 (m, 1H), 2.65 (s, 3H), 2.26-2.40 (m, 1H), 2.23 (s, 3H), 1.93-2.13 (m, 1H), 1.19-1.40 (m, 4H), 1.25 (s, 9H), 0.87 (br.s., 3H), 0.63 (br. s., 3H). LCMS (M+H)=515.4.

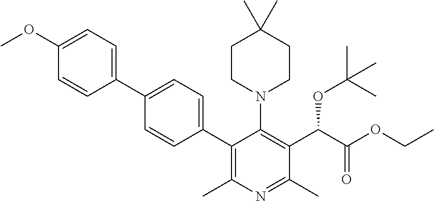 [SMILES: CCOC(=O)[C@@H](OC(C)(C)C)c1c(C)nc(C)c(-c2ccc(-c3ccc(OC)cc3)cc2)c1N1CCC(C)(C)CC1]

(S)-Ethyl 2-(tert-butoxy)-2-(4-(4,4-dimethylpiperidin-1-yl)-5-(4′-methoxy-[1,1′-biphenyl]-4-yl)-2,6-dimethylpyridin-3-yl)acetate: A mixture of (S)-ethyl 2-(5-bromo-4-(4,4-dimethylpiperidin-1-yl)-2,6-dimethylpyridin-3-yl)-2-(tert-butoxy)acetate (0.02 g, 0.044 mmol), (4′-methoxy-[1,1′-biphenyl]-4-yl)boronic acid (0.020 g, 0.088 mmol) and 2M Na2CO3 (0.055 ml, 0.110 mmol) in DMF (1.5 mL) was degassed for 3 min. Then, Pd(Ph₃P)₄ (5.07 mg, 4.39 μmol) was degassed for 1 min and placed in a pre-heated oil bath at 90° C. After 5 h, cooled and purified by prep-HPLC to afford (S)-ethyl 2-(tert-butoxy)-2-(4-(4,4-dimethylpiperidin-1-yl)-5-(4′-methoxy-[1,1′-biphenyl]-4-yl)-2,6-dimethylpyridin-3-yl)acetate (0.0074 g, 0.013 mmol, 30.2% yield) as colorless paste. LCMS (M+H)=559.4.

### EXAMPLE 16

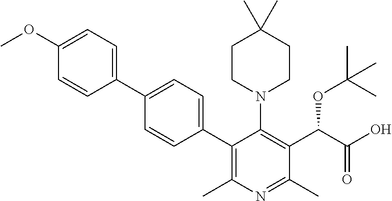 [SMILES: COc1ccc(-c2ccc(-c3c(C)nc(C)c([C@H](OC(C)(C)C)C(=O)O)c3N3CCC(C)(C)CC3)cc2)cc1]

(S)-2-(tert-Butoxy)-2-(4-(4,4-dimethylpiperidin-1-yl)-5-(4′-methoxy-[1,1′-biphenyl]-4-yl)-2,6-dimethylpyridin-3-yl)acetic acid: A solution of (S)-ethyl 2-(tert-butoxy)-2-(4-(4,4-dimethylpiperidin-1-yl)-5-(4′-methoxy-[1,1′-biphenyl]-4-yl)-2,6-dimethylpyridin-3-yl)acetate (0.0074 g, 0.013 mmol) and 1M NaOH (0.132 ml, 0.132 mmol) in EtOH (1.5 mL) was heated at 90° C. for 6 h. Then, cooled and purified by prep-HPLC to afford (S)-2-(tert-butoxy)-2-(4-(4,4-dimethylpiperidin-1-yl)-5-(4′-methoxy-[1,1′-biphenyl]-4-yl)-2,6-dimethylpyridin-3-yl)acetic acid (0.0065 g, 0.012 mmol, 92% yield) as solid. ¹H NMR (500 MHz, DMSO-d₆) δ 7.74 (dd, J=9.9, 8.1 Hz, 2H), 7.70 (d, J=8.8 Hz, 2H), 7.39 (d, J=8.8 Hz, 1H), 7.21 (d, J=8.1 Hz, 1H), 7.06 (d, J=8.8 Hz, 2H), 5.85 (s, 1H), 3.31 (br. s., 1H), 2.89-2.80 (m, 1H), 2.46 (s, 3H), 2.23 (d, J=12.1 Hz, 1H), 2.12 (s, 3H), 1.97 (t, J=11.9 Hz, 1H), 1.55-1.45 (m, 1H), 1.28 (d, J=15.4 Hz, 1H), 1.19 (d, J=12.8 Hz, 1H), 1.14 (s, 9H), 1.01 (d, J=11.4 Hz, 1H), 0.84 (s, 3H), 0.57 (s, 3H). LCMS (M+H)=531.4.

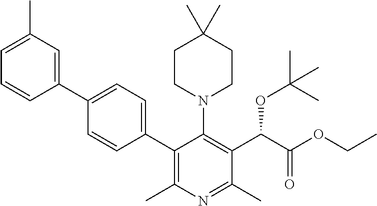 [SMILES: CCOC(=O)[C@@H](OC(C)(C)C)c1c(C)nc(C)c(-c2ccc(-c3cccc(C)c3)cc2)c1N1CCC(C)(C)CC1]

(S)-Ethyl 2-(tert-butoxy)-2-(4-(4,4-dimethylpiperidin-1-yl)-2,6-dimethyl-5-(3′-methyl-[1,1′-biphenyl]-4-yl)pyridin-3-yl)acetate: A mixture of (S)-ethyl 2-(5-bromo-4-(4,4-dimethylpiperidin-1-yl)-2,6-dimethylpyridin-3-yl)-2-(tert-butoxy)acetate (0.02 g, 0.044 mmol), (3′-methyl-[1,1′-biphenyl]-4-yl)boronic acid (0.019 g, 0.088 mmol) and 2M Na2CO3 (0.055 ml, 0.110 mmol) in DMF (1 mL) was degassed for 3 min. Then, Pd(Ph₃P)₄ (5.07 mg, 4.39 μmol) was degassed for 1 min and placed in a pre-heated oil bath at 90° C. After 9 h, cooled and purified by prep-HPLC to afford (S)-ethyl 2-(tert-butoxy)-2-(4-(4,4-dimethylpiperidin-1-yl)-2,6-dimethyl-5-(3′-methyl-[1,1′-biphenyl]-4-yl)pyridin-3-yl)acetate (0.0128 g, 0.024 mmol, 53.7% yield) as brown solid. LCMS(M+H)=543.4.

### EXAMPLE 17

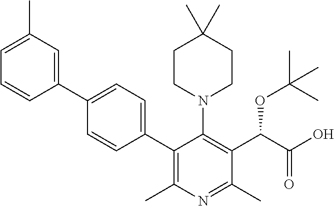 [SMILES: Cc1cccc(-c2ccc(-c3c(C)nc(C)c([C@H](OC(C)(C)C)C(=O)O)c3N3CCC(C)(C)CC3)cc2)c1]

(S)-2-(tert-Butoxy)-2-(4-(4,4-dimethylpiperidin-1-yl)-2,6-dimethyl-5-(3′-methyl-[1,1′-biphenyl]-4-yl)pyridin-3-yl)acetic acid: A solution of (S)-ethyl 2-(tert-butoxy)-2-(4-(4,4-dimethylpiperidin-1-yl)-2,6-dimethyl-5-(3′-methyl-[1,1′-biphenyl]-4-yl)pyridin-3-yl)acetate (0.0128 g, 0.024 mmol) and 1M NaOH (0.236 ml, 0.236 mmol) in EtOH (1 mL) was refluxed for 6 h. Then, cooled and purified by prep-HPLC to afford (S)-2-(tert-butoxy)-2-(4-(4,4-dimethylpiperidin-1-yl)-2,6-dimethyl-5-(3′-methyl-[1,1′-biphenyl]-4-yl)pyridin-3-yl)acetic acid (0.0078 g, 0.015 mmol, 64.3% yield) as solid. ¹H NMR (500 MHz, DMSO-d₆) δ 7.78 (dd, J=9.9, 8.1 Hz, 2H), 7.56 (s, 1H), 7.53 (d, J=7.7 Hz, 1H), 7.42 (d, J=7.7 Hz, 1H), 7.38 (t, J=7.7 Hz, 1H), 7.22 (dd, J=13.6, 7.7 Hz, 2H), 5.86 (s, 1H), 3.31 (d, J=9.9 Hz, 1H), 2.90-2.82 (m, 1H), 2.46 (s, 3H), 2.40 (s, 3H), 2.23 (d, J=8.4 Hz, 1H), 2.11 (s, 3H), 2.01-1.93 (m, 1H), 1.54-1.45 (m, 1H), 1.28 (d, J=16.1 Hz, 1H), 1.19 (d, J=12.5 Hz, 1H), 1.14 (s, 9H), 1.01 (d, J=10.3 Hz, 1H), 0.84 (br. s., 3H), 0.57 (s, 3H). LCMS(M+H)=515.15.

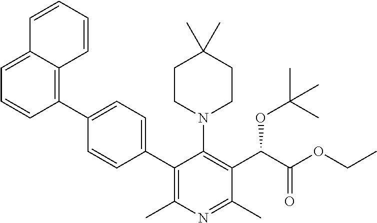 [SMILES: CCOC(=O)[C@@H](OC(C)(C)C)c1c(C)nc(C)c(-c2ccc(-c3cccc4ccccc34)cc2)c1N1CCC(C)(C)CC1]

(S)-Ethyl 2-(tert-butoxy)-2-(4-(4,4-dimethylpiperidin-1-yl)-2,6-dimethyl-5-(4-(naphthalen-1-yl)phenyl)pyridin-3-yl)acetate: A mixture of (S)-ethyl 2-(5-bromo-4-(4,4-dimethylpiperidin-1-yl)-2,6-dimethylpyridin-3-yl)-2-(tert-butoxy)acetate (0.02 g, 0.044 mmol), (4-(naphthalen-1-yl)phenyl)boronic acid (0.022 g, 0.088 mmol) and 2M Na2CO3 (0.055 ml, 0.110 mmol) in DMF (1 mL) was degassed for 3 min. Then, Pd(Ph₃P)₄ (5.07 mg, 4.39 μmol) was degassed for 1 min and placed in a pre-heated oil bath at 90° C. After 9 h, cooled and purified by prep-HPLC to afford (S)-ethyl 2-(tert-butoxy)-2-(4-(4,4-dimethylpiperidin-1-yl)-2,6-dimethyl-5-(4-(naphthalen-1-yl)phenyl)pyridin-3-yl)acetate (0.0128 g, 0.022 mmol, 50.4% yield) as brown paste. LCMS(M+H)=579.4.

### EXAMPLE 18

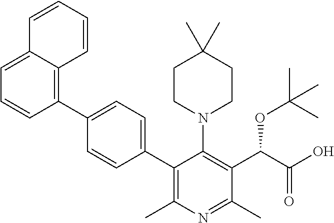 [SMILES: Cc1nc(C)c([C@H](OC(C)(C)C)C(=O)O)c(N2CCC(C)(C)CC2)c1-c1ccc(-c2cccc3ccccc23)cc1]

(S)-2-(tert-Butoxy)-2-(4-(4,4-dimethylpiperidin-1-yl)-2,6-dimethyl-5-(4-(naphthalen-1-yl)phenyl)pyridin-3-yl)acetic acid: A solution of (S)-ethyl 2-(tert-butoxy)-2-(4-(4,4-dimethylpiperidin-1-yl)-2,6-dimethyl-5-(4-(naphthalen-1-yl)phenyl)pyridin-3-yl)acetate (0.0128 g, 0.022 mmol) and 1M NaOH (0.221 ml, 0.221 mmol) in EtOH (1 mL) was refluxed for 6 h. Then, cooled and purified by prep-HPLC to afford (S)-2-(tert-butoxy)-2-(4-(4,4-dimethylpiperidin-1-yl)-2,6-dimethyl-5-(4-(naphthalen-1-yl)phenyl)pyridin-3-yl)acetic acid (0.0099 g, 0.018 mmol, 81% yield) as solid. ¹H NMR (500 MHz, DMSO-d₆) δ 8.03 (d, J=8.1 Hz, 1H), 7.99 (d, J=8.1 Hz, 1H), 7.82 (d, J=8.4 Hz, 1H), 7.63 (t, J=7.7 Hz, 1H), 7.59-7.53 (m, 3H), 7.52-7.45 (m, 3H), 7.32 (d, J=7.7 Hz, 1H), 5.90 (s, 1H), 3.32 (d, J=11.4 Hz, 1H), 2.49 (s, 3H), 2.31 (d, J=10.6 Hz, 1H), 2.21 (s, 3H), 2.08-2.00 (m, 1H), 1.57-1.49 (m, 1H), 1.36 (t, J=10.8 Hz, 1H), 1.23 (d, J=11.4 Hz, 1H), 1.16 (s, 9H), 1.09 (d, J=12.1 Hz, 1H), 0.89 (s, 3H), 0.71 (s, 3H). LCMS(M+H)=551.20.

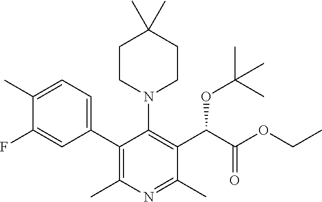 [SMILES: CCOC(=O)[C@@H](OC(C)(C)C)c1c(C)nc(C)c(-c2ccc(C)c(F)c2)c1N1CCC(C)(C)CC1]

(S)-Ethyl 2-(tert-butoxy)-2-(4-(4,4-dimethylpiperidin-1-yl)-5-(3-fluoro-4-methylphenyl)-2,6-dimethylpyridin-3-yl)acetate: A mixture of (S)-ethyl 2-(5-bromo-4-(4,4-dimethylpiperidin-1-yl)-2,6-dimethylpyridin-3-yl)-2-(tert-butoxy)acetate (0.02 g, 0.044 mmol), (3-fluoro-4-methylphenyl)boronic acid (0.014 g, 0.088 mmol) and 2M Na2CO3 (0.055 ml, 0.110 mmol) in DMF (1 mL) was degassed for 3 min. Then, Pd(Ph₃P)₄ (5.07 mg, 4.39 μmol) was degassed for 1 min and placed in a pre-heated oil bath at 90° C. After 9 h, cooled and purified by prep-HPLC to afford (S)-ethyl 2-(tert-butoxy)-2-(4-(4,4-dimethylpiperidin-1-yl)-5-(3-fluoro-4-methylphenyl)-2,6-dimethylpyridin-3-yl)acetate (0.0105 g, 0.022 mmol, 49.3% yield) as brown paste. LCMS (M+H)=485.3.

### EXAMPLE 19

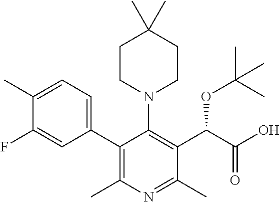 [SMILES: Cc1ccc(-c2c(C)nc(C)c([C@H](OC(C)(C)C)C(=O)O)c2N2CCC(C)(C)CC2)cc1F]

(S)-2-(tert-Butoxy)-2-(4-(4,4-dimethylpiperidin-1-yl)-5-(3-fluoro-4-methylphenyl)-2,6-dimethylpyridin-3-yl)acetic acid: A solution of (S)-ethyl 2-(tert-butoxy)-2-(4-(4,4-dimethylpiperidin-1-yl)-5-(3-fluoro-4-methylphenyl)-2,6-dimethylpyridin-3-yl)acetate (0.01015 g, 0.021 mmol) and 1M NaOH (0.209 ml, 0.209 mmol) in EtOH (1 mL) was refluxed for 6 h. Then, cooled and purified by prep-HPLC to afford (S)-2-(tert-butoxy)-2-(4-(4,4-dimethylpiperidin-1-yl)-5-(3-fluoro-4-methylphenyl)-2,6-dimethylpyridin-3-yl)acetic acid (0.0085 g, 0.019 mmol, 89% yield) as solid and as ˜1:1 mixture of atropisomers. ¹H NMR (500 MHz, DMSO-d₆) δ 7.38 (dt, J=12.7, 8.2 Hz, 1H), 7.19 (d, J=10.6 Hz, 0.5H), 7.07 (d, J=7.7 Hz, 0.5H), 6.95 (d, J=10.3 Hz, 0.5H), 6.89 (d, J=7.7 Hz, 0.5H), 5.83 (br. s., 1H), 3.29 (d, J=10.6 Hz, 1H), 2.80 (t, J=12.5 Hz, 1H), 2.44 (s, 3H), 2.31 (s, 3H), 2.27-2.18 (m, 1H), 2.07 (s, 3H), 1.97-1.88 (m, 1H), 1.54-1.46 (m, 1H), 1.35-1.26 (m, 1H), 1.22-1.16 (m, 1H), 1.13 (s, 9H), 1.09-1.00 (m, 1H), 0.86 (s, 3H), 0.61 (s, 1.5H), 0.62 (s, 1.5H). LCMS(M+H)=457.25.

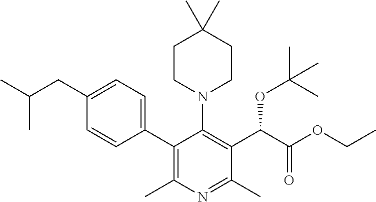 [SMILES: CCOC(=O)[C@@H](OC(C)(C)C)c1c(C)nc(C)c(-c2ccc(CC(C)C)cc2)c1N1CCC(C)(C)CC1]

(S)-Ethyl 2-(tert-butoxy)-2-(4-(4,4-dimethylpiperidin-1-yl)-5-(4-isobutylphenyl)-2,6-dimethylpyridin-3-yl)acetate: A mixture of (S)-ethyl 2-(5-bromo-4-(4,4-dimethylpiperidin-1-yl)-2,6-dimethylpyridin-3-yl)-2-(tert-butoxy)acetate (0.02 g, 0.044 mmol), (4-isobutylphenyl)boronic acid (0.016 g, 0.088 mmol) and 2M Na2CO3 (0.055 ml, 0.110 mmol) in DMF (1 mL) was degassed for 3 min. Then, Pd(Ph₃P)₄ (5.07 mg, 4.39 μmol) was degassed for 1 min and placed in a pre-heated oil bath at 90° C. After 9 h, cooled and purified by prep-HPLC to afford (S)-ethyl 2-(tert-butoxy)-2-(4-(4,4-dimethylpiperidin-1-yl)-5-(4-isobutylphenyl)-2,6-dimethylpyridin-3-yl)acetate (0.0132 g, 0.026 mmol, 59.1% yield) as brown paste. LCMS (M+H)=509.4.

### EXAMPLE 20

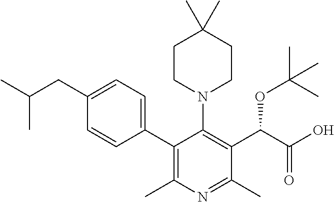 [SMILES: Cc1nc(C)c([C@H](OC(C)(C)C)C(=O)O)c(N2CCC(C)(C)CC2)c1-c1ccc(CC(C)C)cc1]

(S)-2-(tert-Butoxy)-2-(4-(4,4-dimethylpiperidin-1-yl)-5-(4-isobutylphenyl)-2,6-dimethylpyridin-3-yl)acetic acid: A solution of (S)-ethyl 2-(tert-butoxy)-2-(4-(4,4-dimethylpiperidin-1-yl)-5-(4-isobutylphenyl)-2,6-dimethylpyridin-3-yl)acetate (0.0132 g, 0.026 mmol) and 1M NaOH (0.259 ml, 0.259 mmol) in EtOH (1 mL) was refluxed for 6 h. Then, cooled and purified by prep-HPLC to afford (S)-2-(tert-butoxy)-2-(4-(4,4-dimethylpiperidin-1-yl)-5-(4-isobutylphenyl)-2,6-dimethylpyridin-3-yl)acetic acid (0.0108 g, 0.022 mmol, 87% yield) as solid. ¹H NMR (500 MHz, DMSO-d₆) δ 7.27-7.20 (m, 3H), 7.05 (d, J=7.7 Hz, 1H), 5.84 (s, 1H), 3.27 (d, J=10.3 Hz, 1H), 2.80 (t, J=12.7 Hz, 1H), 2.54-2.51 (m, 2H), 2.44 (s, 3H), 2.17 (d, J=11.0 Hz, 1H), 2.07 (s, 3H), 1.90-1.82 (m, 2H), 1.51-1.43 (m, 1H), 1.29 (t, J=10.8 Hz, 1H), 1.17 (d, J=12.5 Hz, 1H), 1.13 (s, 9H), 0.96 (d, J=11.0 Hz, 1H), 0.88 (d, J=6.6 Hz, 6H), 0.84 (s, 3H), 0.56 (s, 3H). LCMS(M+H)=481.25.

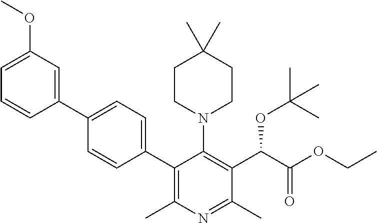 [SMILES: CCOC(=O)[C@@H](OC(C)(C)C)c1c(C)nc(C)c(-c2ccc(-c3cccc(OC)c3)cc2)c1N1CCC(C)(C)CC1]

(S)-Ethyl 2-(tert-butoxy)-2-(4-(4,4-dimethylpiperidin-1-yl)-5-(3′-methoxy-[1,1′-biphenyl]-4-yl)-2,6-dimethylpyridin-3-yl)acetate: A mixture of (S)-ethyl 2-(5-bromo-4-(4,4-dimethylpiperidin-1-yl)-2,6-dimethylpyridin-3-yl)-2-(tert-butoxy)acetate (0.02 g, 0.044 mmol), (3′-methoxy-[1,1′-biphenyl]-4-yl)boronic acid (0.020 g, 0.088 mmol) and 2M Na2CO3 (0.055 ml, 0.110 mmol) in DMF (1 mL) was degassed for 3 min. Then, Pd(Ph₃P)₄ (5.07 mg, 4.39 μmol) was degassed for 1 min and placed in a pre-heated oil bath at 90° C. After 9 h, cooled and purified by prep-HPLC to afford (S)-ethyl 2-(tert-butoxy)-2-(4-(4,4-dimethylpiperidin-1-yl)-5-(3′-methoxy-[1,1′-biphenyl]-4-yl)-2,6-dimethylpyridin-3-yl)acetate (0.0128 g, 0.023 mmol, 52.2% yield) as brown paste. LCMS(M+H)=559.4.

### EXAMPLE 21

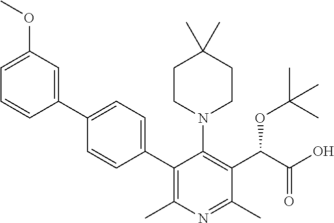 [SMILES: COc1cccc(-c2ccc(-c3c(C)nc(C)c([C@H](OC(C)(C)C)C(=O)O)c3N3CCC(C)(C)CC3)cc2)c1]

(S)-2-(tert-Butoxy)-2-(4-(4,4-dimethylpiperidin-1-yl)-5-(3′-methoxy-[1,1′-biphenyl]-4-yl)-2,6-dimethylpyridin-3-yl)acetic acid: A solution of (S)-ethyl 2-(tert-butoxy)-2-(4-(4,4-dimethylpiperidin-1-yl)-5-(3′-methoxy-[1,1′-biphenyl]-4-yl)-2,6-dimethylpyridin-3-yl)acetate (0.0128 g, 0.023 mmol) and 1M NaOH (0.229 ml, 0.229 mmol) in EtOH (1 mL) was refluxed for 6 h. Then, cooled and purified by prep-HPLC to afford (S)-2-(tert-butoxy)-2-(4-(4,4-dimethylpiperidin-1-yl)-5-(3′-methoxy-[1,1′-biphenyl]-4-yl)-2,6-dimethylpyridin-3-yl)acetic acid (0.0091 g, 0.017 mmol, 74.9% yield) as solid. ¹H NMR (500 MHz, DMSO-d₆) δ 7.81 (dd, J=11.0, 8.1 Hz, 2H), 7.46-7.39 (m, 2H), 7.32 (d, J=8.1 Hz, 1H), 7.28-7.23 (m, 2H), 6.99-6.93 (m, 1H), 5.88 (s, 1H), 3.85 (s, 3H), 3.28 (d, J=10.6 Hz, 1H), 2.88-2.82 (m, 1H), 2.47 (s, 3H), 2.23 (d, J=12.5 Hz, 1H), 2.12 (s, 3H), 1.96 (t, J=11.2 Hz, 1H), 1.54-1.46 (m, 1H), 1.34-1.26 (m, 1H), 1.20 (d, J=12.8 Hz, 1H), 1.15 (s, 9H), 1.01 (d, J=12.1 Hz, 1H), 0.84 (s, 3H), 0.57 (s, 3H). LCMS(M+H)=531.2.

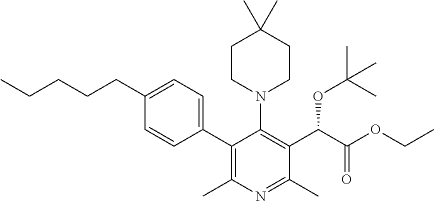 [SMILES: CCCCCc1ccc(-c2c(C)nc(C)c([C@H](OC(C)(C)C)C(=O)OCC)c2N2CCC(C)(C)CC2)cc1]

(S)-Ethyl 2-(tert-butoxy)-2-(4-(4,4-dimethylpiperidin-1-yl)-2,6-dimethyl-5-(4-pentylphenyl)pyridin-3-yl)acetate: A mixture of (S)-ethyl 2-(5-bromo-4-(4,4-dimethylpiperidin-1-yl)-2,6-dimethylpyridin-3-yl)-2-(tert-butoxy)acetate (0.02 g, 0.044 mmol), (4-pentylphenyl)boronic acid (0.017 g, 0.088 mmol) and 2M Na₂CO₃ (0.055 ml, 0.110 mmol) was degassed for 2 min. To this was added Pd(Ph₃P)₄ (5.07 mg, 4.39 degassed for 1 min and placed in pre-heated oil bath at 90° C. After 8 h, cooled and purified by prep-HPLC to afford (S)-ethyl 2-(tert-butoxy)-2-(4-(4,4-dimethylpiperidin-1-yl)-2,6-dimethyl-5-(4-pentylphenyl)pyridin-3-yl)acetate (0.0127 g, 0.024 mmol, 55.3% yield) as brown paste. LCMS (M+H)=523.5.

### EXAMPLE 22

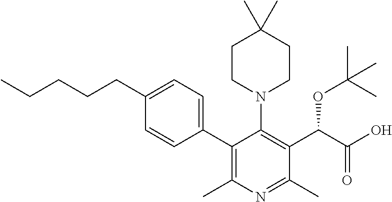 [SMILES: CCCCCc1ccc(-c2c(C)nc(C)c([C@H](OC(C)(C)C)C(=O)O)c2N2CCC(C)(C)CC2)cc1]

(S)-2-(tert-Butoxy)-2-(4-(4,4-dimethylpiperidin-1-yl)-2,6-dimethyl-5-(4-pentylphenyl)pyridin-3-yl)acetic acid: A solution of (S)-ethyl 2-(tert-butoxy)-2-(4-(4,4-dimethylpiperidin-1-yl)-2,6-dimethyl-5-(4-pentylphenyl)pyridin-3-yl)acetate (0.0127 g, 0.024 mmol) and 1M NaOH (0.243 ml, 0.243 mmol) in EtOH (1 mL) was heated at refluxed for 6 h. Then, cooled and purified by prep-HPLC to afford (S)-2-(tert-butoxy)-2-(4-(4,4-dimethylpiperidin-1-yl)-2,6-dimethyl-5-(4-pentylphenyl)pyridin-3-yl)acetic acid (0.01 g, 0.020 mmol, 83% yield) as solid. ¹H NMR (500 MHz, DMSO-d₆) δ 7.31-7.25 (m, 2H), 7.24-7.20 (m, 1H), 7.05 (d, J=7.7 Hz, 1H), 5.89 (s, 1H), 3.19 (d, J=16.1 Hz, 1H), 2.81 (t, J=11.4 Hz, 1H), 2.65 (t, J=7.2 Hz, 2H), 2.45 (s, 3H), 2.17 (d, J=12.1 Hz, 1H), 2.07 (s, 3H), 1.87 (t, J=12.3 Hz, 1H), 1.61 (quin, J=7.2 Hz, 2H), 1.52-1.44 (m, 1H), 1.33-1.23 (m, 4H), 1.18 (d, J=13.9 Hz, 1H), 1.13 (s, 9H), 0.97 (d, J=12.1 Hz, 1H), 0.85 (t, J=7.0 Hz, 3H), 0.84 (s, 3H), 0.56 (s, 3H). LCMS (M+H)=495.5.

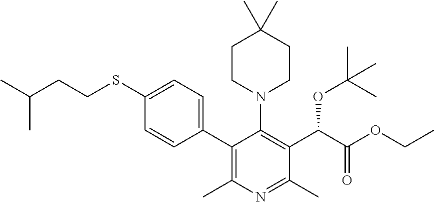 [SMILES: CCOC(=O)[C@@H](OC(C)(C)C)c1c(C)nc(C)c(-c2ccc(SCCC(C)C)cc2)c1N1CCC(C)(C)CC1]

(S)-Ethyl 2-(tert-butoxy)-2-(4-(4,4-dimethylpiperidin-1-yl)-5-(4-(isopentylthio)phenyl)-2,6-dimethylpyridin-3-yl)acetate: A mixture of (S)-ethyl 2-(5-bromo-4-(4,4-dimethylpiperidin-1-yl)-2,6-dimethylpyridin-3-yl)-2-(tert-butoxy)acetate (0.02 g, 0.044 mmol), (4-(isopentylthio)phenyl)boronic acid (0.020 g, 0.088 mmol) and 2M Na₂CO₃ (0.055 ml, 0.110 mmol) was degassed for 2 min. To this was added Pd(Ph₃P)₄ (5.07 mg, 4.39 μmol), degassed for 1 min and placed in pre-heated oil bath at 90° C. After 8 h, cooled and purified by prep-HPLC to afford (S)-ethyl 2-(tert-butoxy)-2-(4-(4,4-dimethylpiperidin-1-yl)-5-(4-(isopentylthio)phenyl)-2,6-dimethylpyridin-3-yl)acetate (0.0144 g, 0.026 mmol, 59.1% yield) as brown paste. LCMS (M+H)=555.5.

### EXAMPLE 23

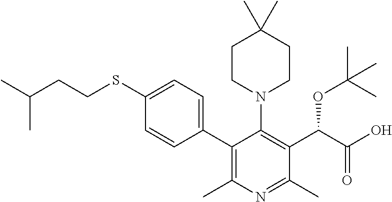 [SMILES: Cc1nc(C)c([C@H](OC(C)(C)C)C(=O)O)c(N2CCC(C)(C)CC2)c1-c1ccc(SCCC(C)C)cc1]

(S)-2-(tert-Butoxy)-2-(4-(4,4-dimethylpiperidin-1-yl)-5-(4-(isopentylthio)phenyl)-2,6-dimethylpyridin-3-yl)acetic acid: A solution of (S)-ethyl 2-(tert-butoxy)-2-(4-(4,4-dimethylpiperidin-1-yl)-5-(4-(isopentylthio)phenyl)-2,6-dimethylpyridin-3-yl)acetate (0.0144 g, 0.026 mmol) and1M NaOH (0.260 ml, 0.260 mmol) in EtOH (1 mL) was heated at refluxed for 6 h. Then, cooled and purified by prep-HPLC to afford (S)-2-(tert-butoxy)-2-(4-(4,4-dimethylpiperidin-1-yl)-5-(4-(isopentylthio)phenyl)-2,6-dimethylpyridin-3-yl)acetic acid (0.0092 g, 0.017 mmol, 67.3% yield) as solid. ¹H NMR (500 MHz, DMSO-d₆) δ 7.44 (d, J=7.7 Hz, 1H), 7.42 (d, J=8.1 Hz, 1H), 7.28 (d, J=8.1 Hz, 1H), 7.11 (d, J=8.1 Hz, 1H), 5.87 (s, 1H), 3.23 (d, J=10.3 Hz, 1H), 3.05-2.94 (m, 2H), 2.81 (t, J=11.9 Hz, 1H), 2.45 (s, 3H), 2.21 (d, J=10.3 Hz, 1H), 2.07 (s, 3H), 1.97-1.90 (m, 1H), 1.74-1.67 (m, 1H), 1.50-1.44 (m, 3H), 1.34-1.27 (m, 1H), 1.19 (d, J=13.2 Hz, 1H), 1.14 (s, 9H), 1.02 (d, J=10.3 Hz, 1H), 0.89 (d, J=2.9 Hz, 3H), 0.88 (d, J=2.9 Hz, 3H), 0.85 (s, 3H), 0.60 (s, 3H). LCMS (M+H)=527.5.

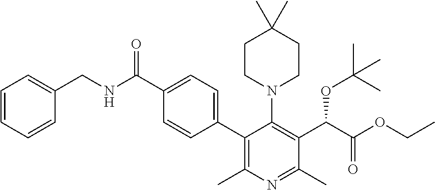 [SMILES: CCOC(=O)[C@@H](OC(C)(C)C)c1c(C)nc(C)c(-c2ccc(C(=O)NCc3ccccc3)cc2)c1N1CCC(C)(C)CC1]

(S)-Ethyl 2-(5-(4-(benzylcarbamoyl)phenyl)-4-(4,4-dimethylpiperidin-1-yl)-2,6-dimethylpyridin-3-yl)-2-(tert-butoxy)acetate: A mixture of (S)-ethyl 2-(5-bromo-4-(4,4-dimethylpiperidin-1-yl)-2,6-dimethylpyridin-3-yl)-2-(tert-butoxy)acetate (0.02 g, 0.044 mmol), (4-(benzylcarbamoyl)phenyl)boronic acid (0.022 g, 0.088 mmol) and 2M Na₂CO₃ (0.055 ml, 0.110 mmol) was degassed for 2 min. To this was added Pd(Ph₃P)₄ (5.07 mg, 4.39 μmol), degassed for 1 min and placed in pre-heated oil bath at 90° C. After 8 h, cooled and purified by prep-HPLC to afford (S)-ethyl 2-(5-(4-(benzylcarbamoyl)phenyl)-4-(4,4-dimethylpiperidin-1-yl)-2,6-dimethylpyridin-3-yl)-2-(tert-butoxy)acetate (0.0136 g, 0.023 mmol, 52.9% yield) as white solid. LCMS (M+H)=586.5.

### EXAMPLE 24

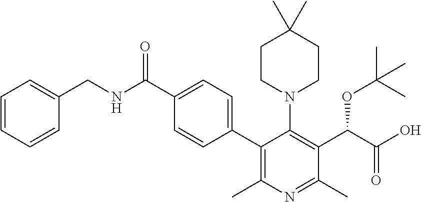 [SMILES: Cc1nc(C)c([C@H](OC(C)(C)C)C(=O)O)c(N2CCC(C)(C)CC2)c1-c1ccc(C(=O)NCc2ccccc2)cc1]

(S)-2-(5-(4-(Benzylcarbamoyl)phenyl)-4-(4,4-dimethylpiperidin-1-yl)-2,6-dimethylpyridin-3-yl)-2-(tert-butoxy)acetic acid: A solution of (S)-ethyl 2-(5-(4-(benzylcarbamoyl)phenyl)-4-(4,4-dimethylpiperidin-1-yl)-2,6-dimethylpyridin-3-yl)-2-(tert-butoxy)acetate (0.0136 g, 0.023 mmol) and 1M NaOH (0.232 ml, 0.232 mmol) in EtOH (1 mL) was heated at refluxed for 6 h. Then, cooled and purified by prep-HPLC to afford (S)-2-(5-(4-(benzylcarbamoyl)phenyl)-4-(4,4-dimethylpiperidin-1-yl)-2,6-dimethylpyridin-3-yl)-2-(tert-butoxy)acetic acid (0.0116 g, 0.021 mmol, 90% yield) as solid. ¹H NMR (500 MHz, DMSO-d₆) δ 9.17 (t, J=5.7 Hz, 1H), 8.02 (d, J=8.1 Hz, 1H), 7.99 (d, J=7.7 Hz, 1H), 7.47 (d, J=7.7 Hz, 1H), 7.37-7.31 (m, 4H), 7.26 (d, J=6.6 Hz, 2H), 5.81 (s, 1H), 4.51 (d, J=5.9 Hz, 2H), 3.36 (d, J=10.3 Hz, 1H), 2.84-2.76 (m, 1H), 2.46 (s, 3H), 2.22 (d, J=11.0 Hz, 1H), 2.05 (s, 3H), 1.86 (t, J=11.7 Hz, 1H), 1.52-147 (m, 1H), 1.31-1.26 (m, 1H), 1.18 (d, J=12.1 Hz, 1H), 1.13 (s, 9H), 1.01 (d, J=12.8 Hz, 1H), 0.84 (br. s., 3H), 0.57 (br. s., 3H). LCMS (M+H)=558.5.

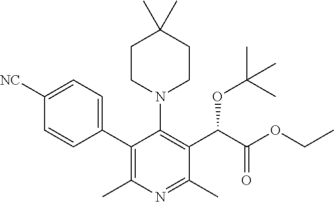 [SMILES: CCOC(=O)[C@@H](OC(C)(C)C)c1c(C)nc(C)c(-c2ccc(C#N)cc2)c1N1CCC(C)(C)CC1]

(S)-Ethyl 2-(tert-butoxy)-2-(5-(4-cyanophenyl)-4-(4,4-dimethylpiperidin-1-yl)-2,6-dimethylpyridin-3-yl)acetate: A mixture of (S)-ethyl 2-(5-bromo-4-(4,4-dimethylpiperidin-1-yl)-2,6-dimethylpyridin-3-yl)-2-(tert-butoxy)acetate (0.02 g, 0.044 mmol), (4-cyanophenyl)boronic acid (0.013 g, 0.088 mmol) and 2M Na2CO3 (0.055 ml, 0.110 mmol) was degassed for 2 min. To this was added Pd(Ph3P)4 (5.07 mg, 4.39 μmol), degassed for 1 min and placed in pre-heated oil bath at 90° C. After 8 h, cooled and purified by prep-HPLC to afford (S)-ethyl 2-(tert-butoxy)-2-(5-(4-cyanophenyl)-4-(4,4-dimethylpiperidin-1-yl)-2,6-dimethylpyridin-3-yl)acetate (0.012 g, 0.025 mmol, 57.2% yield) as light brown solid. LCMS (M+H)=478.4.

### EXAMPLE 25

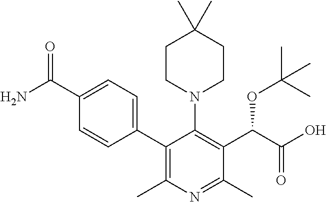 [SMILES: Cc1nc(C)c([C@H](OC(C)(C)C)C(=O)O)c(N2CCC(C)(C)CC2)c1-c1ccc(C(N)=O)cc1]

(S)-2-(tert-Butoxy)-2-(5-(4-carbamoylphenyl)-4-(4,4-dimethylpiperidin-1-yl)-2,6-dimethylpyridin-3-yl)acetic acid: A solution of (S)-ethyl 2-(tert-butoxy)-2-(5-(4-cyanophenyl)-4-(4,4-dimethylpiperidin-1-yl)-2,6-dimethylpyridin-3-yl)acetate (0.012 g, 0.025 mmol) and 1M NaOH (0.126 ml, 0.126 mmol) in MeOH (1 mL) was heated at 75° C. for 6 h. LCMS at this point showed no desired product. So, added addition 1M NaOH (0.126 ml, 0.126 mmol) and heated at 75° C. for additional 8 h. Then, cooled and purified by prep-HPLC to afford (S)-2-(tert-butoxy)-2-(5-(4-carbamoylphenyl)-4-(4,4-dimethylpiperidin-1-yl)-2,6-dimethylpyridin-3-yl)acetic acid (0.0084 g, 0.018 mmol, 71.5% yield) as solid. ¹H NMR (500 MHz, DMSO-d₆) δ 8.07 (s, 1H), 7.99 (d, J=8.1 Hz, 1H), 7.98-7.93 (d, J=8.1 Hz, 2H), 7.44 (d, J=8.4 Hz, 1H), 7.43 (s, 1H), 7.23 (d, J=7.7 Hz, 1H), 5.78 (s, 1H), 2.82-2.75 (m, 1H), 2.45 (s, 3H), 2.20 (d, J=10.3 Hz, 1H), 2.05 (s, 3H), 1.86-1.81 (m, 1H), 1.53-1.47 (m, 1H), 1.32-1.26 (m, 1H), 1.18 (d, J=12.5 Hz, 1H), 1.13 (s, 9H), 1.00 (d, J=11.4 Hz, 1H), 0.84 (s, 3H), 0.57 (s, 3H). LCMS (M+H)=468.4.

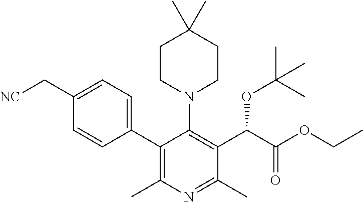 [SMILES: CCOC(=O)[C@@H](OC(C)(C)C)c1c(C)nc(C)c(-c2ccc(CC#N)cc2)c1N1CCC(C)(C)CC1]

(S)-Ethyl 2-(tert-butoxy)-2-(5-(4-(cyanomethyl)phenyl)-4-(4,4-dimethylpiperidin-1-yl)-2,6-dimethylpyridin-3-yl)acetate: A mixture of (S)-ethyl 2-(5-bromo-4-(4,4-dimethylpiperidin-1-yl)-2,6-dimethylpyridin-3-yl)-2-(tert-butoxy)acetate (0.02 g, 0.044 mmol), (4-(cyanomethyl)phenyl)boronic acid (0.014 g, 0.088 mmol) and 2M Na₂CO₃ (0.055 ml, 0.110 mmol) was degassed for 2 min. To this was added Pd(Ph₃P)₄ (5.07 mg, 4.39 μmol), degassed for 1 min and placed in pre-heated oil bath at 90° C. After 8 h, cooled and purified by prep-HPLC to afford (S)-ethyl 2-(tert-butoxy)-2-(5-(4-(cyanomethyl)phenyl)-4-(4,4-dimethylpiperidin-1-yl)-2,6-dimethylpyridin-3-yl)acetate (0.0098 g, 0.020 mmol, 45.4% yield) as brown paste. LCMS (M+H)=492.5.

### EXAMPLE 26

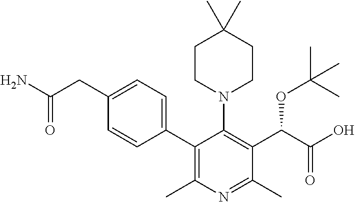 [SMILES: Cc1nc(C)c([C@H](OC(C)(C)C)C(=O)O)c(N2CCC(C)(C)CC2)c1-c1ccc(CC(N)=O)cc1]

(S)-2-(5-(4-(2-Amino-2-oxoethyl)phenyl)-4-(4,4-dimethylpiperidin-1-yl)-2,6-dimethylpyridin-3-yl)-2-(tert-butoxy)acetic acid: A solution of (S)-ethyl 2-(tert-butoxy)-2-(5-(4-(cyanomethyl)phenyl)-4-(4,4-dimethylpiperidin-1-yl)-2,6-dimethylpyridin-3-yl)acetate (0.0098 g, 0.020 mmol) and 1M NaOH (0.100 ml, 0.100 mmol) in MeOH (1 mL) was heated at 75° C. for 6 h. LCMS at this point showed no desired product. So, added additional 1M NaOH (0.100 ml, 0.100 mmol) and heated at 75° C. for 6 h. Then, cooled and purified by prep-HPLC to afford (S)-2-(5-(4-(2-amino-2-oxoethyl)phenyl)-4-(4,4-dimethylpiperidin-1-yl)-2,6-dimethylpyridin-3-yl)-2-(tert-butoxy)acetic acid (0.0021 g, 4.36 μmol, 21.88% yield) as solid. ¹H NMR (500 MHz, DMSO-d₆) δ 7.46 (br. s., 1H), 7.37 (dd, J=12.3, 7.9 Hz, 2H), 7.25 (d, J=7.7 Hz, 1H), 7.07 (d, J=7.7 Hz, 1H), 6.91 (br. s., 1H), 5.80 (s, 1H), 3.34 (d, J=10.6 Hz, 1H), 2.79 (t, J=12.8 Hz, 1H), 2.44 (s, 3H), 2.16 (d, J=9.9 Hz, 1H), 2.06 (s, 3H), 1.91 (s, 2H), 1.90-1.83 (m, 1H), 1.54-1.42 (m, 1H), 1.26 (d, J=18.3 Hz, 1H), 1.16 (d, J=13.6 Hz, 1H), 1.12 (s, 9H), 0.99 (d, J=9.5 Hz, 1H), 0.84 (br. s., 3H), 0.57 (br. s., 3H). LCMS (M+H)=482.4.

The following examples were prepared according to the general procedure outlined here.

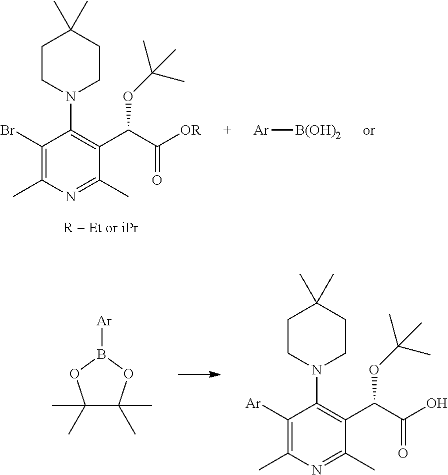

General Procedure: To a mixture of (S)-ethyl or (S)-isopropyl 2-(5-bromo-4-(4,4-dimethylpiperidin-1-yl)-2,6-dimethylpyridin-3-yl)-2-(tert-butoxy)acetate (1 eq.), aryl boronic acid or ester (1-5 eq.) and Cs₂CO₃ (2-10 eq.) in 1,4-dioxane and water (volume ratio 20:1 to 1:1) was added Pd(PPh₃)₄ (0.01-1 eq.). The mixture was flushed with nitrogen and then heated at 50-150° C. for 1 to 48 hours. The mixture was diluted with water and then extracted with EtOAc. The organic layers were combined, washed with brine and concentrated to give a crude product, which was diluted with MeOH/H₂O or THF/H₂O (20:1 to 1:1), before NaOH (0.1-5 eq.) was added. The mixture was heated at 50-150° C. for 1 to 48 hours. All the solvents were removed under vacuum and the residue was purified by preparative HPLC to give the desired product.

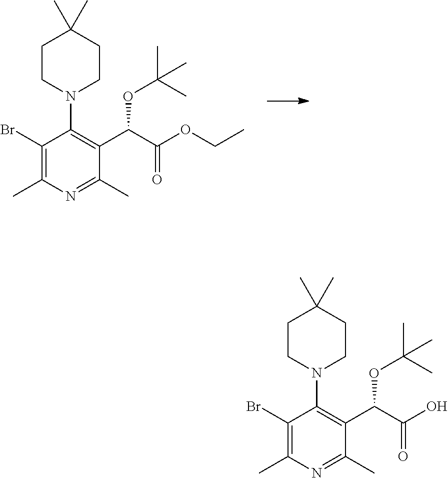 [SMILES: CCOC(=O)[C@@H](OC(C)(C)C)c1c(C)nc(C)c(Br)c1N1CCC(C)(C)CC1 ; Cc1nc(C)c([C@H](OC(C)(C)C)C(=O)O)c(N2CCC(C)(C)CC2)c1Br]

(S)-2-(5-Bromo-4-(4,4-dimethylpiperidin-1-yl)-2,6-dimethylpyridin-3-yl)-2-(tert-butoxy)acetic acid: NaOH (1 mL, 1N) was added into a solution of (S)-ethyl 2-(5-bromo-4-(4,4-dimethylpiperidin-1-yl)-2,6-dimethylpyridin-3-yl)-2-(tert-butoxy)acetate (50 mg) in THF (5 mL) and water (1 mL). The reaction mixture was heated at 80° C. for 3 hours. The mixture was neutralized with 1N HCl (10 mL) and extracted with EtOAc (3×10 mL). The organic layer was concentrated under vaccum to give (S)-2-(5-bromo-4-(4,4-dimethylpiperidin-1-yl)-2,6-dimethylpyridin-3-yl)-2-(tert-butoxy)acetic acid which was used as was. LCMS (M+H): 427.0.

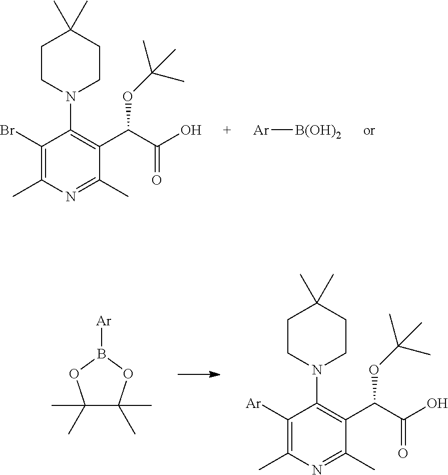

General Procedure for the preparation of examples XX-YY: To a mixture of (S)-2-(5-bromo-4-(4,4-dimethylpiperidin-1-yl)-2,6-dimethylpyridin-3-yl)-2-(tert-butoxy)acetic acid (1-5 eq., aryl boronic acid or ester (1-5 eq.) and Cs₂CO₃ (2-3 eq.) in 1,4-dioxane and water (volume ratio 20:1 to 1:1) was added Pd(PPh₃)₄ (0.01-1 eq.). The mixture was flushed with nitrogen and then heated at 50-100° C. for 1 to 8 hours. The mixture was diluted with water and then extracted with EtOAc. The organic layers were combined, washed with brine, concentrated and the residue was purified by preparative HPLC to give the desired product.

| Name | 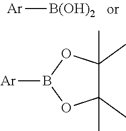 | Structure | LCMS (M + H) |
|---|---|---|---|
| (S)-2-(tert-Butoxy)-2- (5-(4-(cyanomethyl)- phenyl)-4-(4,4- dimethylpiperidin-1-yl)- 2,6-dimethylpyridin-3- yl)acetic acid | 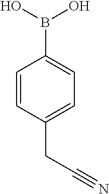 [SMILES: N#CCc1ccc(B(O)O)cc1] | 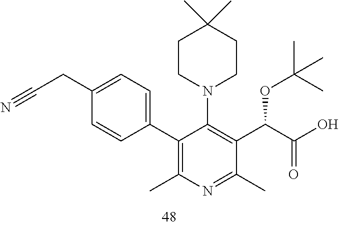 [SMILES: Cc1nc(C)c([C@H](OC(C)(C)C)C(=O)O)c(N2CCC(C)(C)CC2)c1-c1ccc(CC#N)cc1] | 464.3 |
| (S,E)-2-(tert-Butoxy)- 2-(5-(4-(2- cyanovinyl)phenyl)- 4-(4,4-dimethylpiperidin- 1-yl)-2,6-dimethyl- pyridin-3-yl)acetic acid | 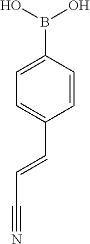 [SMILES: N#C/C=C/c1ccc(B(O)O)cc1] | 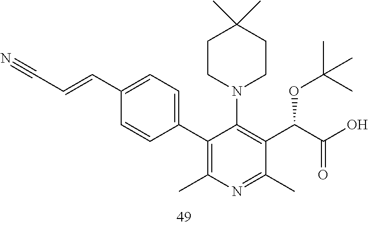 [SMILES: Cc1nc(C)c([C@H](OC(C)(C)C)C(=O)O)c(N2CCC(C)(C)CC2)c1-c1ccc(/C=C/C#N)cc1] | 476.5 |

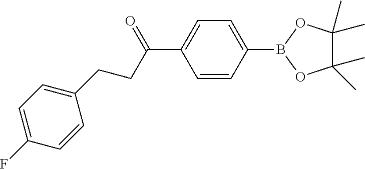 [SMILES: CC1(C)OB(c2ccc(C(=O)CCc3ccc(F)cc3)cc2)OC1(C)C]

3-(4-Fluorophenyl)-1-(4-(4,4,5,5-tetramethyl-1,3,2-dioxaborolan-2-yl)phenyl)pro-pan-1-one: A mixture of 1-(4-bromophenyl)-3-(4-fluorophenyl)propan-1-one (300 mg, 0.977 mmol), bis(pinacolateo)diboron (372 mg, 1.465 mmol) and KOAc (288 mg, 2.93 mmol) in 1,4-dioxane (10 mL) was sparged with N₂ for 15 min. Then, 1,1-bis(diphenylphosphino)ferrocene]dichloropalladium(II) CH₂Cl₂ complex (39.9 mg, 0.049 mmol) was added, sparged for additional 5 min and heated at 90° C. for 16 h. Then, cooled, diluted with ethyl acetate (50 mL), washed with water (2×25 mL), brine (25 mL), dried (Na₂SO₄), filtered and concentrated to give brown paste which was purified by flash chromatography (5-40% EtOAc/hexane) to afford 3-(4-fluorophenyl)-1-(4-(4,4,5,5-tetramethyl-1,3,2-dioxaborolan-2-yl)phenyl)propan-1-one (250 mg, 0.706 mmol, 72.3% yield) as off-white solid. ¹H NMR (500 MHz, CDCl₃) δ 8.01-7.82 (m, 4H), 7.23 (dd, J=8.3, 5.6 Hz, 2H), 7.00 (t, J=8.7 Hz, 2H), 3.31 (t, J=7.6 Hz, 2H), 3.07 (t, J=7.6 Hz, 2H), 1.38 (s, 12H). LCMS (M+H)=355.4.

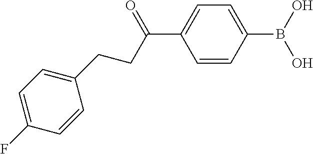 [SMILES: O=C(CCc1ccc(F)cc1)c1ccc(B(O)O)cc1]

(4-(3-(4-Fluorophenyl)propanoyl)phenyl)boronic acid: To a solution of 3-(4-fluorophenyl)-1-(4-(4,4,5,5-tetramethyl-1,3,2-dioxaborolan-2-yl)phenyl)propan-1-one (150 mg, 0.423 mmol) in acetone (4 mL)/water (2.000 mL) was added NaIO₄ (453 mg, 2.117 mmol) and NH₄OAc (163 mg, 2.117 mmol) and the resulting mixture was stirred at room temp for 16 h. Then, 1N HCl (1 mL) was added and the mixture was stirred for 1 h. The mixture was then diluted with EtOAc (50 mL) and washed with brine (10 mL), dried (Na₂SO₄), filtered and concentrated to afford (4-(3-(4-fluorophenyl)propanoyl)phenyl)boronic acid (90 mg, 0.331 mmol, 78% yield) as white solid. ¹H NMR (500 MHz, DMSO-d₆) δ 8.05-7.86 (m, 4H), 7.33 (t, J=6.7 Hz, 2H), 7.10 (t, J=8.7 Hz, 2H), 3.38 (t, J=7.4 Hz, 2H), 2.98-2.86 (m, 2H). LCMS (M+H)=273.3.

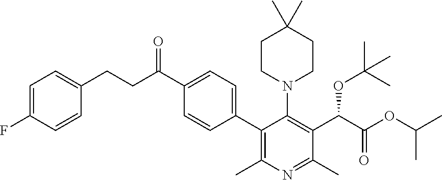 [SMILES: Cc1nc(C)c([C@H](OC(C)(C)C)C(=O)OC(C)C)c(N2CCC(C)(C)CC2)c1-c1ccc(C(=O)CCc2ccc(F)cc2)cc1]

(S)-Isopropyl 2-(tert-butoxy)-2-(4-(4,4-dimethylpiperidin-1-yl)-5-(4-(3-(4-fluorophenyl)propanoyl)phenyl)-2,6-dimethylpyridin-3-yl)acetate: A mixture of (S)-isopropyl 2-(5-bromo-4-(4,4-dimethylpiperidin-1-yl)-2,6-dimethylpyridin-3-yl)-2-(tert-butoxy)acetate (100 mg, 0.213 mmol), (4-(3-(4-fluorophenyl)propanoyl)-phenyl)boronic acid (116 mg, 0.426 mmol) and 2M Na₂CO₃ (0.266 mL, 0.533 mmol) in 1,4-dioxane (4 mL) was degassed for 10 min. Then, Pd(Ph₃P)₄ (12.31 mg, 10.65 μmol) was added, degassed for 5 min and placed in a pre-heated oil bath at 90° C. After 16 h, cooled, diluted with ether (50 mL), washed with water (10 mL), brine (25 mL), dried (Na₂SO₄), filtered, concentrated and purified by flash chromatography using (5-40% EtOAc/Hex to afford (S)-isopropyl 2-(tert-butoxy)-2-(4-(4,4-dimethylpiperidin-1-yl)-5-(4-(3-(4-fluorophenyl)propanoyl)phenyl)-2,6-dimethylpyridin-3-yl)acetate (80 mg, 0.130 mmol, 60.9% yield) as white foam. ¹H NMR (500 MHz, CDCl₃) δ 8.09-8.01 (m, 2H), 7.42 (dd, J=8.0, 1.3 Hz, 1H), 7.26 (dd, J=8.4, 5.4 Hz, 3H), 7.02 (t, J=8.7 Hz, 2H), 6.02 (br. s., 1H), 5.10 (dq, J=12.3, 6.1 Hz, 1H), 3.41-3.34 (m, 2H), 3.29-3.17 (m, 1H), 3.15-3.08 (m, 2H), 2.62 (s, 3H), 2.54-2.39 (m, 2H), 2.29 (d, J=11.0 Hz, 1H), 2.17 (s, 3H), 1.94 (t, J=11.6 Hz, 1H), 1.57-1.49 (m, 2H), 1.37 (t, J=10.8 Hz, 1H), 1.28-1.23 (m, 6H), 1.20 (s, 9H), 0.90 (s, 3H), 0.60 (s, 3H). LCMS (M+H)=617.8.

### EXAMPLE 50

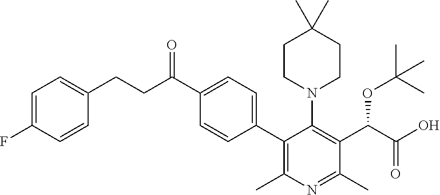 [SMILES: Cc1nc(C)c([C@H](OC(C)(C)C)C(=O)O)c(N2CCC(C)(C)CC2)c1-c1ccc(C(=O)CCc2ccc(F)cc2)cc1]

(S)-2-(tert-Butoxy)-2-(4-(4,4-dimethylpiperidin-1-yl)-5-(4-(3-(4-fluorophenyl)propanoyl)phenyl)-2,6-dimethylpyridin-3-yl)acetic acid: A solution of (S)-isopropyl 2-(tert-butoxy)-2-(4-(4,4-dimethylpiperidin-1-yl)-5-(4-(3-(4-fluorophenyl)propanoyl)phenyl)-2,6-dimethylpyridin-3-yl)acetate (10 mg, 0.016 mmol) and 1N NaOH (0.081 mL, 0.081 mmol) in ethanol (1 mL) was heated at 85° C. for 16 h. Mixture was then cooled and purified by prep HPLC to afford (S)-2-(tert-butoxy)-2-(4-(4,4-dimethylpiperidin-1-yl)-5-(4-(3-(4-fluorophenyl)propanoyl)-phenyl)-2,6-dimethylpyridin-3-yl)acetic acid (3.2 mg, 5.57 μmol, 34.3% yield). ¹H NMR (500 MHz, DMSO-d₆) δ 8.10 (d, J=8.1 Hz, 1H), 8.05 (d, J=8.4 Hz, 1H), 7.52 (d, J=8.1 Hz, 1H), 7.41-7.24 (m, 3H), 7.10 (t, J=8.8 Hz, 2H), 5.82 (br. s., 1H), 3.48-3.35 (m, 2H), 3.37-3.32 (br. s., 1H), 2.97 (t, J=7.5 Hz, 2H), 2.80 (t, J=11.4 Hz, 1H), 2.46 (s, 3H), 2.21 (d, J=8.4 Hz, 1H), 2.05 (s, 3H), 1.87-1.76 (m, 1H), 1.55-1.42 (m, 1H), 1.27 (d, J=12.1 Hz, 1H), 1.18 (d, J=12.5 Hz, 1H), 1.13 (s, 9H), 0.99 (d, J=13.6 Hz, 1H), 0.84 (br. s., 3H), 0.55 (br. s., 3H). LCMS (M+H)=575.3.

### EXAMPLE 51

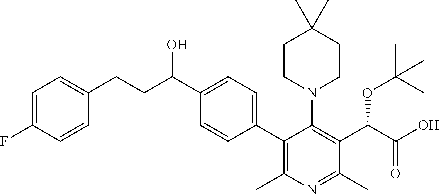 [SMILES: Cc1nc(C)c([C@H](OC(C)(C)C)C(=O)O)c(N2CCC(C)(C)CC2)c1-c1ccc(C(O)CCc2ccc(F)cc2)cc1]

(2S)-2-(tert-Butoxy)-2-(4-(4,4-dimethylpiperidin-1-yl)-5-(4-(3-(4-fluorophenyl)-1-hydroxypropyl)phenyl)-2,6-dimethylpyridin-3-yl)acetic acid: To a solution of (S)-isopropyl 2-(tert-butoxy)-2-(4-(4,4-dimethylpiperidin-1-yl)-5-(4-(3-(4-fluorophenyl)propanoyl)phenyl)-2,6-dimethylpyridin-3-yl)acetate (60 mg, 0.097 mmol) in Methanol (2 mL) was added NaBH₄ (3.68 mg, 0.097 mmol) and the resulting mixture was stirred at room temp for 1 h. Mixture was then concentrated and re-dissolved in EtOH (2 mL) and treated with 1N NaOH (0.973 mL, 0.973 mmol) at 85° C. for 16 h. Mixture was then cooled and purified by prep HPLC to afford (2S)-2-(tert-butoxy)-2-(4-(4,4-dimethylpiperidin-1-yl)-5-(4-(3-(4-fluorophenyl)-1-hydroxypropyl)phenyl)-2,6-dimethylpyridin-3-yl)acetic acid (13 mg, 0.021 mmol, 22.01% yield) as inseparable mixture of diastereomers. ¹H NMR (500 MHz, METHANOL-d₄) δ 7.60 (d, J=7.7 Hz, 1H), 7.56-7.48 (m, 1H), 7.42 (dd, J=12.2, 8.0 Hz, 1H), 7.24-7.14 (m, 3H), 7.01 (t, J=8.7 Hz, 2H), 5.57 (s, 1H), 4.76 (q, J=5.9 Hz, 1H), 2.82-2.58 (m, 6H), 2.29 (s, 3H), 2.17-2.01 (m, 2H), 1.44-1.37 (m, 1H), 1.35 (br. s., 3H), 1.21 (s, 9H), 0.79 (br. s., 6H). 4H of piperidine were not resolved. LCMS (M+H)=577.7.

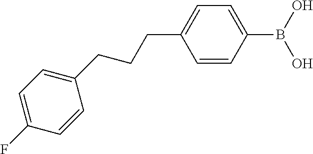 [SMILES: OB(O)c1ccc(CCCc2ccc(F)cc2)cc1]

(4-(3-(4-Fluorophenyl)propyl)phenyl)boronic acid: To a solution of (4-(3-(4-fluorophenyl)propanoyl)phenyl)boronic acid (100 mg, 0.368 mmol) in TFA (1 mL) was added Et₃SiH (0.235 mL, 1.470 mmol) and the resulting mixture was stirred at room temp for 3 h. Mixture was then concentrated and purifid by Biotage (50-100% EtOAc/hexane) to afford (4-(3-(4-fluorophenyl)propyl)phenyl)boronic acid (40 mg, 0.155 mmol, 42.2% yield) as white solid. ¹H NMR (500 MHz, DMSO-d₆) δ 7.79 (d, J=7.7 Hz, 2H), 7.29-7.19 (m, 4H), 7.10 (t, J=8.8 Hz, 2H), 2.61 (t, J=7.3 Hz, 4H), 1.88 (quin, J=7.6 Hz, 2H).

### EXAMPLE 52

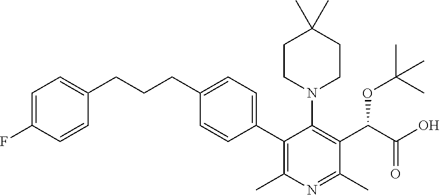 [SMILES: Cc1nc(C)c([C@H](OC(C)(C)C)C(=O)O)c(N2CCC(C)(C)CC2)c1-c1ccc(CCCc2ccc(F)cc2)cc1]

(S)-2-(tert-Butoxy)-2-(4-(4,4-dimethylpiperidin-1-yl)-5-(4-(3-(4-fluorophenyl)propyl)phenyl)-2,6-dimethylpyridin-3-yl)acetic acid: A mixture of (S)-isopropyl 2-(5-bromo-4-(4,4-dimethylpiperidin-1-yl)-2,6-dimethylpyridin-3-yl)-2-(tert-butoxy)acetate (40 mg, 0.085 mmol), (4-(3-(4-fluorophenyl)propyl)phenyl)-boronic acid (33.0 mg, 0.128 mmol) and 2M Na₂CO₃ (0.107 mL, 0.213 mmol) in 1,4-dioxane (2 mL) was degassed for 10 min. Then, Pd(Ph₃P)₄ (9.85 mg, 8.52 μmol) was added, degassed for 5 min and placed in a pre-heated oil bath at 90° C. After 3 h, cooled, filtered and purified by pre HPLC to afford desired ester; (M+H)=603.8. Ester was then treated with 1N NaOH (0.852 mL, 0.852 mmol) in EtOH (2 mL) at 80° C. for 5 h. Mixture was then cooled and purifid by prep HPLC to afford (S)-2-(tert-butoxy)-2-(4-(4,4-dimethylpiperidin-1-yl)-5-(4-(3-(4-fluorophenyl)-propyl)phenyl)-2,6-dimethylpyridin-3-yl)acetic acid (4.8 mg, 8.56 μmol, 10.05% yield) as white foam. ¹H NMR (500 MHz, DMSO-d₆) δ 7.36-7.26 (m, 2H), 7.26-7.17 (m, 3H), 7.15-7.00 (m, 3H), 5.83 (br. s., 1H), 3.29 (d, J=9.9 Hz, 2H), 2.86-2.76 (m, 1H), 2.68 (t, J=7.2 Hz, 2H), 2.57 (t, J=7.3 Hz, 2H), 2.44 (s, 3H), 2.18 (d, J=10.3 Hz, 1H), 1.95-1.81 (m, 5H), 1.47 (br. s., 1H), 1.26 (d, J=15.8 Hz, 1H), 1.17 (br. s., 1H), 1.13 (s, 9H), 0.96 (d, J=12.1 Hz, 1H), 0.81 (br. s., 3H), 0.53 (br. s., 3H). LCMS (M+H)=561.4.

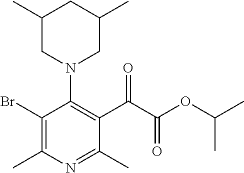 [SMILES: Cc1nc(C)c(C(=O)C(=O)OC(C)C)c(N2CC(C)CC(C)C2)c1Br]

Isopropyl 2-(5-bromo-4-(3,5-dimethylpiperidin-1-yl)-2,6-dimethylpyridin-3-yl)-2-oxoacetate: To a solution of 3,5-dimethylpiperidine (1.1 g, 9.86 mmol) and DIEA (3.1 mL, 18 mmol) in anhydrous CH₃CN (40 mL) was added isopropyl 2-(5-bromo-4-chloro-2,6-dimethylpyridin-3-yl)-2-oxoacetate (3.0 g, 8.87 mmol) at rt. The resulting mixture was placed in a pre-heated oil bath (80° C.) and stirred for 24 h; cooled, concentrated, and charged (DCM) to a 120 g ISCO silica gel cartridge and gradient eluted (5-15% EtOAc/hexanes) using an Isolera chromatography station to give isopropyl 2-(5-bromo-4-(3,5-dimethylpiperidin-1-yl)-2,6-dimethylpyridin-3-yl)-2-oxoacetate 756 mg (20%) as a mixture of diastereomers. A sample of the product subjected to prep HPLC on Waters-Atlantis column (30×100 mm S5) running 15 min gradient from 10-100% B (MeOH/water/TFA), yielding two bands. The major isomer. ¹H NMR (500 MHz, CDCl3) δ 5.21-5.14 (m, 1H), 2.87 (br. s, 4H), 2.70 (s, 3H), 2.41 (s, 3H), 1.77-1.71 (m, 3H), 1.29 (d, J=6.2 Hz, 6H), 0.85 (d, J=6.6 Hz, 6H), 0.68 (q, J=11.9 Hz, 1H). UPLC (M+H)=413.15.

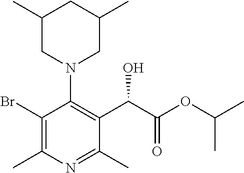 [SMILES: Cc1nc(C)c([C@H](O)C(=O)OC(C)C)c(N2CC(C)CC(C)C2)c1Br]

(S)-Isopropyl 2-(5-bromo-4-((3,5-dimethylpiperidin-1-yl)-2,6-dimethylpyridin-3-yl)-2-hydroxyacetate: The 1.7 mL of benzo[d][1,3,2]dioxaborole (426 mg, 3.56 mmol) was added to a nitrogen purged solution of isopropyl 2-(5-bromo-4-(3,5-dimethylpiperidin-1-yl)-2,6-dimethylpyridin-3-yl)-2-oxoacetate (975 mg, 2.37 mmol) and 0.7 mL of (R)-1-methyl-3,3-diphenylhexahydropyrrolo[1,2-c][1,3,2]oxazaborole (197 mg, 0.7 mmol) in toluene (28 mL) at −60° C. and allowed to warm to −15° C. before being placed in the freezer overnight. The reaction was quenched with 1M Na₂CO₃, diluted with EtOAc, and stirred for 30 min. The organic layer was washed with sat'd Na₂CO₃ solution, brine and dried (MgSO₄). The crude product was charged (DCM) to a 80 g ISCO silica gel cartridge and gradient elution (0-30% EtOAc/hexanes) using an Isolera chromatography station gave (S)-isopropyl 2-(5-bromo-4-((3,5-dimethylpiperidin-1-yl)-2,6-dimethylpyridin-3-yl)-2-hydroxyacetate 720 mg (58%) as a mixture of diastereomers. ¹H NMR (500 MHz, CDCl3) δ 5.36 (s, 1H), 5.10-5.05 (m, 1H), 3.22-3.17 (m, 2H), 2.86-2.84 (m, 1H), 2.71-2.70 (m, 1H), 2.66 (s, 3H), 2.07 (s, 3H), 1.93 (br. s, 1H), 1.83-1.81 (m, 2H), 1.30-1.17 (m, 6H), 0.90-0.88 (m, 6H), 0.74 (q, J=12.5 Hz, 1H). UPLC (M+H)=415.3.

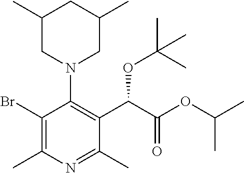 [SMILES: Cc1nc(C)c([C@H](OC(C)(C)C)C(=O)OC(C)C)c(N2CC(C)CC(C)C2)c1Br]

(S)-Isopropyl 2-(5-bromo-4-((3,5-dimethylpiperidin-1-yl)-2,6-dimethylpyridin-3-yl)-2-(tert-butoxy)acetate: The isobutylene gas was bubbled into a nitrogen purged, cooled (0° C.) solution (S)-isopropyl 2-(5-bromo-4-((3,5-dimethylpiperidin-1-yl)-2,6-dimethylpyridin-3-yl)-2-hydroxyacetate (710 mg, 1.7 mmol) and 0.17 mL of 70% HClO₄ in DCM (25 mL) for 20 min. The reaction mixture was allowed to warm to rt and stirred for 24 h in a pressure sealed vessel. The reaction was then diluted with DCM, washed with 1M Na₂CO₃ solution, and dried over MgSO₄. The crude product was charged (DCM) to a 40 g ISCO silica gel cartridge and gradient elution (0-12% EtOAc/hexanes) using an Isolera chromatography station gave (S)-isopropyl 2-(5-bromo-4-((3,5-dimethylpiperidin-1-yl)-2,6-dimethylpyridin-3-yl)-2-(tert-butoxy)acetate 373 mg (46%) as a mixture of diastereomers. ¹H NMR (500 MHz, CDCl3) δ 6.22 (s, 1H), 5.07-5.02 (m, 1H), 3.38 (t, J=10.7 Hz, 1H), 3.01-2.99 (m, 1H), 2.86 (t, J=10.9 Hz, 1H), 2.76 (br. s, 1H), 2.64 (s, 3H), 2.55 (s, 3H), 1.86-1.84 (m, 3H), 1.22 (d, J=6.3 Hz, 3H), 1.20 (s, 9H), 1.14 (d, J=6.3 Hz, 3H), 0.91 (d, J=6.3 Hz, 3H), 0.88 (d, J=6.6 Hz, 3H), 0.81-0.74 (m, 1H). UPLC (M+H)=471.4.

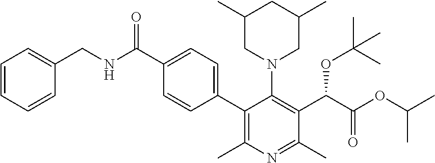 [SMILES: Cc1nc(C)c([C@H](OC(C)(C)C)C(=O)OC(C)C)c(N2CC(C)CC(C)C2)c1-c1ccc(C(=O)NCc2ccccc2)cc1]

(S)-Isopropyl 2-(tert-butoxy)-2-(4-(3,5-dimethylpiperidin-1-yl)-2,6-dimethyl-5-(4-(2-phenylacetamido)phenyl)pyridin-3-yl)acetate: The Tetrakis (45.3 mg, 0.021 mmol) was added to a argon purged and degassed solution of (S)-isopropyl 2-(5-bromo-4-((3,5-dimethylpiperidin-1-yl)-2,6-dimethylpyridin-3-yl)-2-(tert-butoxy)acetate (92 mg, 0.196 mmol), (4-(benzylcarbamoyl)phenyl)boronic acid (55 mg, 0.216 mmol), and potassium phosphate tribasic (312 mg, 1.5 mmol) in dioxane (3 mL) and water (0.6 mL) and stirred in a screw-capped pressure vessel for 4 h at 90° C. The reaction was allowed to cool, diluted with EtOAc, and the organic layer was washed with brine and dried (MgSO4). The crude product was charged (DCM) to a 24 g ISCO silica gel cartridge and gradient elution (5-75% EtOAc/hexanes) using an Isolera chromatography station gave (S)-isopropyl 2-(tert-butoxy)-2-(4-(3,5-dimethylpiperidin-1-yl)-2,6-dimethyl-5-(4-(2-phenylacetamido)phenyl)pyridin-3-yl)acetate 45 mg (38%). ¹H NMR (500 MHz, DMSO) δ 9.18-9.16 (m, 1H), 7.99-7.98 (m, 2H), 7.47 (d, J=6.2 Hz, 1H), 7.36-7.33 (m, 4H), 7.27-7.25 (m, 1H), 7.17 (d, J=6.9 Hz, 1H), 5.91 (s, 1H), 4.98-4.95 (m, 1H), 4.52 (d, J=5.9 Hz, 2H), 3.40-3.35 (m, 3H), 3.28-3.25 (m, 1H), 2.45 (s, 3H), 2.04 (s, 3H), 1.76-1.65 (m, 2H), 1.54 (br. s, 1H), 1.21 (d, J=5.9 Hz, 3H), 1.16 (d, J=5.9 Hz, 3H), 1.12 (s, 9H), 0.74 (d, J=5.5 Hz, 3H), 0.52 (d, J=6.2 Hz, 3H), 0.30-0.25 (m, 1H). UPLC (M+H)=600.7.

### EXAMPLE 53

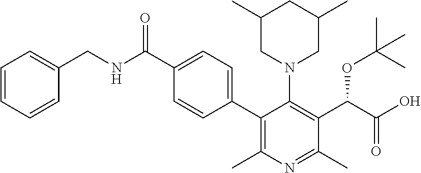 [SMILES: Cc1nc(C)c([C@H](OC(C)(C)C)C(=O)O)c(N2CC(C)CC(C)C2)c1-c1ccc(C(=O)NCc2ccccc2)cc1]

(S)-2-(tert-Butoxy)-2-(4-((3,5-dimethylpiperidin-1-yl)-5-(4-(4-fluorophenethoxy)phenyl)-2,6-dimethylpyridin-3-yl)acetic acid: The 0.1 mL of 1M sodium hydroxide (4 mg, 0.1 mmol) was added to a solution of (S)-isopropyl 2-(tert-butoxy)-2-(4-(3,5-dimethylpiperidin-1-yl)-2,6-dimethyl-5-(4-(2-phenylacetamido)phenyl)pyridin-3-yl)acetate (30 mg, 0.05 mmol) in ethanol (1.0 mL) and stirred for 24 h at 90° C. The reaction mixture was neutralized with 1N HCl soln, extracted with EtOAc, and the organic layer was washed with brine, and dried (MgSO4). The crude material was purified by prep HPLC (XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 25-95% B over 15 minutes, then a 5-minute hold at 100% B; Flow: 20 mL/min. There was obtained (S)-2-(tert-butoxy)-2-(4-((3,5-dimethylpiperidin-1-yl)-5-(4-(4-fluorophenethoxy)phenyl)-2,6-dimethylpyridin-3-yl)acetic acid 19 mg (67%) as a mixture of diastereomers. ¹H NMR (500 MHz, DMSO) δ 9.18-9.16 (m, 1H), 7.97-7.94 (m, 2H), 7.4 (d, J=8.1 Hz, 1H), 7.35-7.32 (m, 4H), 7.26 (br. s, 1H), 7.15 (d, J=7.7 Hz, 1H), 5.72 (s, 1H), 4.51 (d, J=5.5 Hz, 2H), 3.59 (br. s, 4H), 2.45 (s, 3H), 2.02 (s, 3H), 1.77 (br. s, 1H), 1.64 (br. s, 1H), 1.53 (br. s,1H), 1.11 (s, 9H), 0.71 (d, J=4.8 Hz, 3H), 0.50 (d, J=5.5 Hz, 3H), 0.26 (q, J=11.3 Hz, 1H). UPLC (M+H)=558.7.

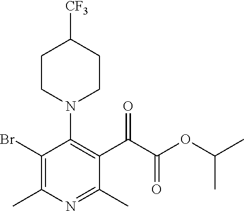 [SMILES: Cc1nc(C)c(C(=O)C(=O)OC(C)C)c(N2CCC(C(F)(F)F)CC2)c1Br]

Isopropyl 2-(5-bromo-2,6-dimethyl-4-(4-(trifluoromethyl)piperidin-1-yl)pyridin-3-yl)-2-oxoacetate: To a solution of 4-(trifluoromethyl)piperidine HCl (1.7 g, 8.97 mmol) and DIEA (3.13 mL, 17.9 mmol) in anhydrous CH₃CN (15 mL) was added isopropyl 2-(5-bromo-4-chloro-2,6-dimethylpyridin-3-yl)-2-oxoacetate (3.0 g, 8.97 mmol) at rt. The resulting mixture was placed in a pre-heated oil bath (80° C.) and stirred for 24 h; cooled, diluted with ether, washed with water, brine, and dried (MgSO₄). The crude product was charged (DCM) to a 80 g ISCO silica gel cartridge and gradient eluted (5-20% EtOAc/hexanes) using an Isolera chromatography station to give isopropyl 2-(5-bromo-2,6-dimethyl-4-(4-(trifluoromethyl)piperidin-1-yl)pyridin-3-yl)-2-oxoacetate 2.9 g (71%). ¹H NMR (500 MHz, CDCl₃) δ 5.25-5.20 (m, 1H), 3.54-3.51 (m, 2H), 3.15 (dt, J=12.5, 2.0 Hz, 2H), 2.87 (s, 3H), 2.57 (s, 3H), 2.30-2.23 (m, 1H), 2.02-1.99 (m, 2H), 1.79 (dq, J=12.4, 4.1 Hz, 2H) 1.43 (d, J=6.2 Hz, 6H). UPLC (M+H)=453.2.

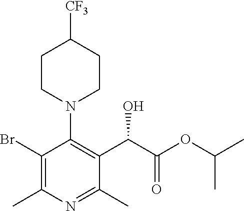 [SMILES: Cc1nc(C)c([C@H](O)C(=O)OC(C)C)c(N2CCC(C(F)(F)F)CC2)c1Br]

Isopropyl 2-(5-bromo-2,6-dimethyl-4-(4-(trifluoromethyl)piperidin-1-yl)pyridin-3-yl)-2-hydroxyacetate: The 1.4 mL of benzo[d][1,3,2]dioxaborole (797 mg, 6.65 mmol) was added to a nitrogen purged solution of isopropyl 2-(5-bromo-2,6-dimethyl-4-(4-(trifluoromethyl)piperidin-1-yl)pyridin-3-yl)-2-oxoacetate (2 g, 4.43 mmol) and 0.89 mL of (R)-1-methyl-3,3-diphenylhexahydropyrrolo[1,2-c][1,3,2]oxazaborole (246 mg, 0.89 mmol) in toluene (40 mL) at −60° C. and allowed to warm to −15° C. before being placed in the freezer overnight. The reaction was quenched with 1M Na₂CO₃, diluted with EtOAc, and stirred for 30 min. The organic layer was washed with sat'd Na₂CO₃ solution, brine, and dried (MgSO₄). The crude product was charged (DCM) to a 40 g ISCO silica gel cartridge and gradient elution (0-30% EtOAc/hexanes) using an Isolera chromatography station gave isopropyl 2-(5-bromo-2,6-dimethyl-4-(4-(trifluoromethyl)piperidin-1-yl)pyridin-3-yl)-2-hydroxyacetate 2.04 g (100%) as a mixture of diastereomers. ¹H NMR (500 MHz, DMSO-d₆) δ 5.97/5.82 (d, J=4.4 Hz, 1H), 4.97-4.92 (m, 1H), 3.65 (t, J=11.4 Hz, 1H), 3.20-3.17 (m, 1H), 2.99 (d, J=9.5 Hz, 1H), 2.85 (t, J=12.1 Hz, 1H), 2.53 (s, 3H), 2.40 (s, 3H), 2.31 (br. s, 1H), 1.83-1.77 (m, 2H), 1.69-1.55 (m, 2H), 1.15 (d, J=6.2 Hz, 3H), 1.07 (d, J=6.2 Hz, 3H). UPLC (M+H)=453.4.

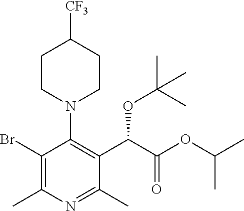 [SMILES: Cc1nc(C)c([C@H](OC(C)(C)C)C(=O)OC(C)C)c(N2CCC(C(F)(F)F)CC2)c1Br]

(S)-Isopropyl 2-(5-bromo-2,6-dimethyl-4-(4-(trifluoromethyl)piperidin-1-yl)pyridin-3-yl)-2-(tert-butoxy)acetate: The isobutylene gas was bubbled into a nitrogen purged, cooled (0° C.) solution of 2-(5-bromo-2,6-dimethyl-4-(4-(trifluoromethyl)piperidin-1-yl)pyridin-3-yl)-2-hydroxyacetate (1.9 g, 4.19 mmol) and 0.4 mL of 70% HClO₄ in DCM (20 mL) for 20 min. The reaction mixture was allowed to warm to rt and stirred for 18 h in a pressure sealed vessel, after which it was recooled, and an additional 0.4 mL of 70% HClO4 was added, and the reaction stirred for 24 h at rt. The reaction was then diluted with DCM, washed with 1M Na₂CO₃ solution, and dried over MgSO₄. The crude product was charged (DCM) to a 80 g ISCO silica gel cartridge and gradient elution (5-12% EtOAc/hexanes) using an Isolera chromatography station gave (S)-isopropyl 2-(5-bromo-2,6-dimethyl-4-(4-(trifluoromethyl)piperidin-1-yl)pyridin-3-yl)-2-(tert-butoxy)acetate 1.9 g (90%) as a mixture of diastereomers. ¹H NMR (500 MHz, DMSO) δ 6.11 (br. s, 1H), 4.93-4.91 (m, 1H), 3.84 (br. s, 1H), 3.209 (br. s, 1H), 3.08 (br. s, 1H), 2.89 (br. s, 1H), 2.53 (s, 3H), 2.44 (br. s, 3H), 1.96 (br. s, 1H), 1.86 (br. s, 2H), 1.64-1.53 (m, 2H), 1.16 (d, J=6.3 Hz, 3H), 1.14 (s, 9H), 1.07 (d, J=6.3 Hz, 3H). UPLC (M+H)=511.4.

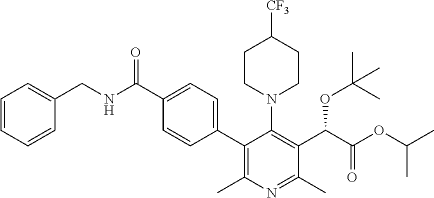 [SMILES: Cc1nc(C)c([C@H](OC(C)(C)C)C(=O)OC(C)C)c(N2CCC(C(F)(F)F)CC2)c1-c1ccc(C(=O)NCc2ccccc2)cc1]

(S)-Isopropyl 2-(5-(4-(benzylcarbamoyl)phenyl)-2,6-dimethyl-4-(4-(trifluoromethyl)piperidin-1-yl)pyridin-3-yl)-2-(tert-butoxy)acetate: Tetrakis (34 mg, 0.029 mmol) was added to an argon-degassed solution of (S)-isopropyl 2-(5-bromo-2,6-dimethyl-4-(4-(trifluoromethyl)piperidin-1-yl)pyridin-3-yl)-2-(tert-butoxy)acetate (150 mg, 0.294 mmol), (4-(benzylcarbamoyl)phenyl)boronic acid (83 mg, 0.324 mmol), and potassium phosphate tribasic (469 mg, 2.208 mmol) in dioxane (2 ml) and water (0.4 ml) stirred for 16 h at 80° C. and stirred in a screw-capped pressure vessel. The reaction was allowed to cool, diluted with EtOAc, and the organic layer was washed with brine and dried (MgSO4). The crude product was charged (DCM) to a 40 g ISCO silica gel cartridge and gradient elution (0-70% EtOAc/hexanes) using an Isolera chromatography station gave (S)-isopropyl 2-(5-(4-(benzylcarbamoyl)phenyl)-2,6-dimethyl-4-(4-(trifluoromethyl)piperidin-1-yl)pyridin-3-yl)-2-(tert-butoxy)acetate 165 mg (87%) as a mixture of diastereomers. ¹H NMR (500 MHz, DMSO) δ 9.16-9.15 (m, 1H), 8.00 (t, J=7.0 Hz, 2H), 7.49 (d, J=7.7 Hz, 1H), 7.38-7.33 (m, 4H), 7.27-7.25 (m, 1H), 7.22 (d, J=7.7 Hz, 1H), 5.92 (br. s, 1H), 4.97 (br. s, 1H), 4.52 (d, J=5.9 Hz, 2H), 3.43-3.41 (m, 2H), 2.63 (br. s 2H), 2.48 (s, 3H), 2.11 (br. s, 1H), 2.04 (s, 3H), 1.75-1.71 (m, 2H), 1.60 (br. s, 1H), 1.39-1.32 (m, 1H), 1.20 (d, J=6.2 Hz, 3H), 1.14 (br. s, 12H). UPLC (M+H)=640.4.

### EXAMPLE 54

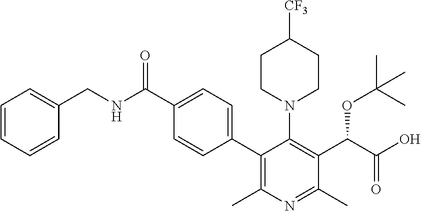 [SMILES: Cc1nc(C)c([C@H](OC(C)(C)C)C(=O)O)c(N2CCC(C(F)(F)F)CC2)c1-c1ccc(C(=O)NCc2ccccc2)cc1]

(S)-2-(5-(4-(Benzylcarbamoyl)phenyl)-2,6-dimethyl-4-(4-(trifluoromethyl)piperidin-1-yl)pyridin-3-yl)-2-(tert-butoxy)acetic acid: The 0.23 mL of 1M sodium hydroxide (9.3 mg, 0.23 mmol) was added to a solution of (S)-isopropyl 2-(5-(4-(benzylcarbamoyl)phenyl)-2,6-dimethyl-4-(4-(trifluoromethyl)piperidin-1-yl)pyridin-3-yl)-2-(tert-butoxy)acetate (50 mg, 0.078 mmol) in ethanol (1 mL) and stirred for 24 h at 90° C. An additional 0.23 mL sodium hydroxide was added and the reaction was continued for 24 h. The reaction mixture was neutralized with 1N HCl soln, extracted with EtOAc, and the organic layer was washed with brine, and dried (MgSO4). The crude material was purified by prep HPLC (XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 25-65% B over 15 minutes, then a 5-minute hold at 100% B; Flow: 20 mL/min. There was obtained (S)-2-(5-(4-(benzylcarbamoyl)phenyl)-2,6-dimethyl-4-(4-(trifluoromethyl)piperidin-1-yl)pyridin-3-yl)-2-(tert-butoxy)acetic acid 16 mg (35%) as a mixture of diastereomers. ¹H NMR (500 MHz, DMSO) δ 9.21-9.19 (m, 1H), 8.02-7.96 (m, 2H), 7.48 (d, J=7.6 Hz, 1H), 7.39-7.34 (m, 4H) 7.28-7.25 (m, 1H), 7.16 (d, J=7.6 Hz, 1H), 5.55 (br. s, 1H), 4.52 (d J=5.8 Hz, 2H), 4.01-3.91 (m, 1H), 2.88-2.83 (m, 1H), 2.60-2.56 (m, 2H), 2.49 (s, 3H), 2.04 (s, 3H), 1.67-1.63 (m, 3H), 1.56-1.54 (m, 1H), 1.44-1.38 (m, 1H), 1.11 (s, 9H). UPLC (M+H)=598.5.

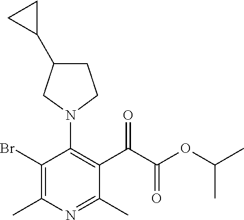 [SMILES: Cc1nc(C)c(C(=O)C(=O)OC(C)C)c(N2CCC(C3CC3)C2)c1Br]

Isopropyl 2-(5-bromo-4-(3-cyclopropylpyrrolidin-1-yl)-2,6-dimethylpyridin-3-yl)-2-oxoacetate: To a solution of 3-cyclopropylpyrrolidine (250 mg, 2.25 mmol) and DIEA (1.178 mL, 6.74 mmol) in anhydrous CH₃CN (15 mL) was added isopropyl 2-(5-bromo-4-chloro-2,6-dimethylpyridin-3-yl)-2-oxoacetate (752 mg, 2.25 mmol) at rt. The resulting mixture was placed in a pre-heated oil bath (80° C.) and stirred for 18 h before being cooled, concentrated, and charged (DCM) to a 40 g ISCO silica gel cartridge and gradient eluted (5-35% EtOAc/hexanes) using an Isolera chromatography station to give isopropyl 2-(5-bromo-4-(3-cyclopropylpyrrolidin-1-yl)-2,6-dimethylpyridin-3-yl)-2-oxoacetate 745 mg (81%). ¹H NMR (500 MHz, DMSO-d₆) δ 5.05-5.00 (m, 1H), 3.25-3.19 (m, 2H), 3.15-3.11 (m, 1H), 2.94-2.90 (m, 1H), 2.62 (s, 3H), 2.36 (s, 3H), 2.06 (br. s., 1H), 1.73-1.69 (m, 2H), 1.29-1.27 (m, 6H), 0.72-0.69 (m, 1H), 0.45-0.40 (m, 2H), 0.16-0.10 (m, 2H). UPLC (M+H)=411.1.

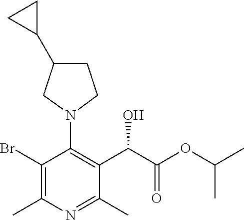 [SMILES: Cc1nc(C)c([C@H](O)C(=O)OC(C)C)c(N2CCC(C3CC3)C2)c1Br]

(2S)-Isopropyl 2-(5-bromo-4-(3-cyclopropylpyrrolidin-1-yl)-2,6-dimethylpyridin-3-yl)-2-hydroxyacetate: The benzo[d][1,3,2]dioxaborole (0.66 mL, 2.68 mmol; 50% soln in toluene) was added to a nitrogen purged solution of isopropyl 2-(5-bromo-4-(3-cyclopropylpyrrolidin-1-yl)-2,6-dimethylpyridin-3-yl)-2-oxoacetate (730 mg, 1.78 mmol) and 0.6 mL of 1M (R)-1-methyl-3,3-diphenylhexahydropyrrolo[1,2-c][1,3,2]oxazaborole (148 mg, 0.54 mmol) in toluene (18 mL) cooled to −50° C. The reaction was allowed to slowly warm to −15° C. and placed in the freezer for 18 h before being quenched with 1M Na₂CO₃ (5 mL) and stirred for 20 min. The organic layer was washed with brine and dried (MgSO₄). The crude product was charged (DCM) to a 40 g ISCO silica gel cartridge and gradient elution (5-50% EtOAc/hexanes) using an Isolera chromatography station gave (2S)-isopropyl 2-(5-bromo-4-(3-cyclopropylpyrrolidin-1-yl)-2,6-dimethylpyridin-3-yl)-2-hydroxyacetate 540 mg (74%) as a mixture of diastereomers; major isomer. ¹H NMR (500 MHz, DMSO-d₆) δ 5.52 (s, 1H), 4.96-4.89 (m, 1H), 3.40-3.35 (m, 2H), 3.17-3.09 (m, 1H), 2.94-2.90 (m, 1H), 2.53 (s, 3H), 2.40 (s, 3H), 2.03 (br. s., 1H), 1.82-1.74 (m, 2H), 1.17-1.08 (m, 6H), 0.76-0.75 (m, 1H), 0.43-0.41 (m, 2H), 0.15-0.14 (m, 2H). UPLC (M+H)=413.2.

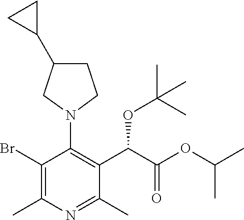 [SMILES: Cc1nc(C)c([C@H](OC(C)(C)C)C(=O)OC(C)C)c(N2CCC(C3CC3)C2)c1Br]

(2S)-Isopropyl 2-(5-bromo-4-(3-cyclopropylpyrrolidin-1-yl)-2,6-dimethylpyridin-3-yl)-2-(tert-butoxy)acetate: The isobutylene gas was bubbled into a nitrogen purged, cooled (0° C.) solution of (2S)-isopropyl 2-(5-bromo-4-(3-cyclopropylpyrrolidin-1-yl)-2,6-dimethylpyridin-3-yl)-2-hydroxyacetate (525 mg, 1.28 mmol) and 0.15 mL of 70% HClO₄ in DCM (10 mL) for 20 min. The reaction mixture was allowed to warm to rt and stirred for 18 h in a pressure sealed vessel, diluted with DCM, washed with 1M Na₂CO₃ solution, and dried over MgSO₄. The crude product was charged (DCM) to a 40 g ISCO silica gel cartridge and gradient elution (5-35% EtOAc/hexanes) using an Isolera chromatography station gave (2S)-isopropyl 2-(5-bromo-4-(3-cyclopropylpyrrolidin-1-yl)-2,6-dimethylpyridin-3-yl)-2-(tert-butoxy)acetate 264 mg (44%) as a mixture of diastereomers. ¹H NMR (500 MHz, DMSO-d₆) δ 5.92/5.87 (s, 1H), 4.91-4.85 (m, 1H), 3.47/3.27 (br. s, 1H), 3.17-3.10 (m, 2H), 2.94-2.91 (m, 1H), 2.52 (s, 3H), 2.41 (s, 3H), 2.16/2.07 (br. s., 1H), 1.86-1.77 (m, 2H), 1.15-1.12 (m, 12H), 1.06-1.03 (m, 3H), 0.92-0.87/0.80-0.78 (m, 1H), 0.47-0.37 (m, 2H), 0.19-0.10 (m, 2H). UPLC (M+H)=469.3.

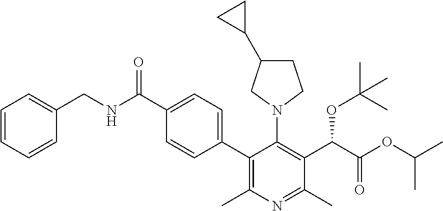 [SMILES: Cc1nc(C)c([C@H](OC(C)(C)C)C(=O)OC(C)C)c(N2CCC(C3CC3)C2)c1-c1ccc(C(=O)NCc2ccccc2)cc1]

(2S)-Isopropyl 2-(5-(4-(benzylcarbamoyl)phenyl)-4-(3-cyclopropylpyrrolidin-1-yl)-2,6-dimethylpyridin-3-yl)-2-(tert-butoxy)acetate: The tetrakis (61.8 mg, 0.053 mmol) was added to a nitrogen purged and degassed solution of (2S)-isopropyl 2-(5-bromo-4-(3-cyclopropylpyrrolidin-1-yl)-2,6-dimethylpyridin-3-yl)-2-(tert-butoxy)acetate (125 mg, 0.27 mmol), (4-(benzylcarbamoyl)phenyl)boronic acid (75 mg, 0.29 mmol), and potassium phosphate tribasic (397 mg, 1.9 mmol) in 1,4-dioxane (3.5 mL) and water (0.9 mL). The reaction mixture was stirred in a screw-capped pressure vessel for 4 h at 90° C., cooled, diluted with EtOAc, and the organic layer was washed with brine and dried (Na2CO3). The crude product was charged (DCM) to a 24 g ISCO silica gel cartridge and gradient elution (5-65% EtOAc/hexanes) using an Isolera chromatography station gave (2S)-isopropyl 2-(5-(4-(benzylcarbamoyl)phenyl)-4-(3-cyclopropylpyrrolidin-1-yl)-2,6-dimethylpyridin-3-yl)-2-(tert-butoxy)acetate 53 mg (33%) as a mixture of diasteromers: ¹H NMR (500 MHz, DMSO) δ 9.15-9.13 (m, 1H), 8.01-7.96 (m, 2H), 7.48-7.46 (m, 1H), 7.35-7.33 (m, 4H), 7.27-7.19 (m, 2H), 5.75/5.72 (s, 1H), 4.98-4.93 (m, 1H), 4.52-4.50 (m, 2H), 3.13-3.09/3.00-2.96 (m, 1H), 2.88-2.72 (m, 2H), 2.67-2.62 (m, 1H), 2.44/2.43 (s, 3H), 2.08/2.06 (s, 3H), 1.70-1.62 (m, 1H), 1.43-1.38/1.43-1.30 (m, 1H), 1.27-1.23/1.07-1.02 (m, 1H), 1.21-1.19 (m, 3H), 1.15 (d, J=6.2 Hz, 3H), 1.12/1.11 (s, 9H), 0.51-0.47 (m, 1H), 0.33-0.25 (m, 2H), −0.04-−0.17 (m, 2H). UPLC (M+H)=598.5.

### EXAMPLE 55

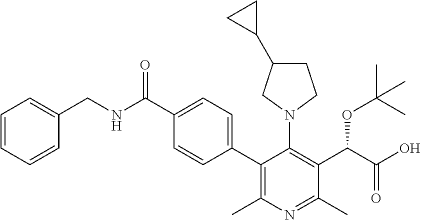 [SMILES: Cc1nc(C)c([C@H](OC(C)(C)C)C(=O)O)c(N2CCC(C3CC3)C2)c1-c1ccc(C(=O)NCc2ccccc2)cc1]

(2S)-2-(5-(4-(Benzylcarbamoyl)phenyl)-4-(3-cyclopropylpyrrolidin-1-yl)-2,6-dimethylpyridin-3-yl)-2-(tert-butoxy)acetic acid: The 0.35 mL of 1M sodium hydroxide (14.13 mg, 0.35 mmol) was added to a solution (2S)-isopropyl 2-(5-(4-(benzylcarbamoyl)phenyl)-4-(3-cyclopropylpyrrolidin-1-yl)-2,6-dimethylpyridin-3-yl)-2-(tert-butoxy)acetate (52.8 mg, 0.088 mmol) in ethanol (2 mL) and stirred for 18 h at 90° C. The reaction mixture was neutralized with 1N HCl soln, extracted with EtOAc, and the organic layer was washed with brine, and dried (MgSO4). The crude material was purified by prep HPLC (XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 25-65% B over 15 minutes, then a 5-minute hold at 100% B; Flow: 20 mL/min. There was obtained (2S)-2-(5-(4-(benzylcarbamoyl)phenyl)-4-(3-cyclopropylpyrrolidin-1-yl)-2,6-dimethylpyridin-3-yl)-2-(tert-butoxy)acetic acid 38.8 mg (79%) as a mixture of diastereomers. ¹H NMR (500 MHz, DMSO) δ 9.16-9.14 (m, 1H), 8.01-7.96 (m, 2H), 7.47-7.46 (m, 1H), 7.35-7.34 (m, 4H), 7.25-7.19 (m, 2H), 5.60/5.58 (s, 1H), 4.52-4.50 (m, 2H), 3.18-3.15 (m, 1H), 3.06 (br. s, 1H) 2.84-2.81 (m, 1H), 2.64-2.60 (m, 1H), 2.46/2.45 (s, 3H), 2.07/2.05 (s, 3H), 1.71-1.65 (m, 1H), 1.41-1.23/1.05-1.01 (m, 2H), 1.11/1.10 (s, 9H), 0.51-0.43 (m, 1H), 0.31-0.24 (m, 2H), -0.70-0.17 (m, 2H). UPLC (M+H)=556.5.

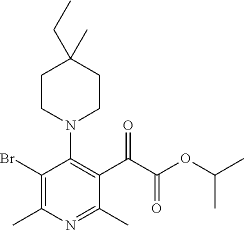 [SMILES: CCC1(C)CCN(c2c(Br)c(C)nc(C)c2C(=O)C(=O)OC(C)C)CC1]

Isopropyl 2-(5-bromo-4-(4-ethyl-4-methylpiperidin-1-yl)-2,6-dimethylpyridin-3-yl)-2-oxoacetate: To a solution of 4-ethyl-4-methylpiperidine (0.760 g, 5.98 mmol) and DIEA (2.1 mL, 11.9 mmol) in anhydrous CH₃CN (15 mL) was added isopropyl 2-(5-bromo-4-chloro-2,6-dimethylpyridin-3-yl)-2-oxoacetate (2.0 g, 5.98 mmol) at rt. The resulting mixture was placed in a pre-heated oil bath (80° C.) and stirred for 24 h; cooled, diluted with ether, washed with water, brine, and dried (MgSO₄). The crude product was charged (DCM) to a 80 g ISCO silica gel cartridge and gradient eluted (5-15% EtOAc/hexanes) using an Isolera chromatography station to give isopropyl 2-(5-bromo-4-(4-ethyl-4-methylpiperidin-1-yl)-2,6-dimethylpyridin-3-yl)-2-oxoacetate 2.51 g (99%). ¹H NMR (500 MHz, CDCl₃) δ 5.07-5.02 (m, 1H), 3.32 (br. s, 2H), 3.18 (br.s, 2H), 2.61 (s, 3H), 2.29 (s, 3H), 1.29-1.28 (m 12H), 0.89 (s, 3H), 0.80 (t, J=7.3 Hz, 3H). UPLC (M+H)=427.3.

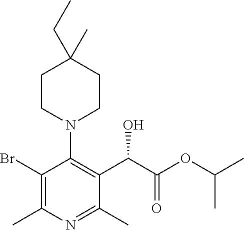 [SMILES: CCC1(C)CCN(c2c(Br)c(C)nc(C)c2[C@H](O)C(=O)OC(C)C)CC1]

Isopropyl (S)-isopropyl 2-(5-bromo-4-(4-ethyl-4-methylpiperidin-1-yl)-2,6-dimethylpyridin-3-yl)-2-hydroxyacet: The 2.2 mL of benzo[d][1,3,2]dioxaborole (1.23 g, 10.4 mmol) was added to a nitrogen purged solution of isopropyl 2-(5-bromo-4-(4-ethyl-4-methylpiperidin-1-yl)-2,6-dimethylpyridin-3-yl)-2-oxoacetate (2.2 g, 5.17 mmol) and 2.1 mL of (R)-1-methyl-3,3-diphenylhexahydropyrrolo[1,2-c][1,3,2]oxazaborole (570 mg, 2.1 mmol) in toluene (50 mL) at −60° C. and allowed to warm to −15° C. before being placed in the freezer overnight. The reaction was quenched with 1M Na₂CO₃, diluted with EtOAc, and stirred for 30 min. The organic layer was washed with sat'd Na₂CO₃ solution, brine and dried (MgSO₄). The crude product was charged (DCM) to a 80 g ISCO silica gel cartridge and gradient elution (0-50% EtOAc/hexanes) using an Isolera chromatography station gave isopropyl (S)-isopropyl 2-(5-bromo-4-(4-ethyl-4-methylpiperidin-1-yl)-2,6-dimethylpyridin-3-yl)-2-hydroxyacetate 2.2 g (100%) as a mixture of diastereomers. ¹H NMR (500 MHz, DMSO-d₆) δ 5.93 (m, 1H), 4.96-4.92 (m, 1H), 3.72 (t, J=11.7 Hz, 1H), 3.63-3.59 (m, 1H), 3.52 (t, J=10.6 Hz, 1H), 3.44 (br. s, 1H), 2.52 (s, 3H), 2.37 (s, 3H), 1.46-1.45 (m, 4H), 1.33-1.24 (m, 2H), 1.14 (d, J=6.2 Hz, 3H), 1.07 (d, J=6.2 Hz, 3H), 0.95/0.88 (s, 3H), 0.83 (t, J=7.0 Hz, 3H). UPLC (M+H)=429.3.

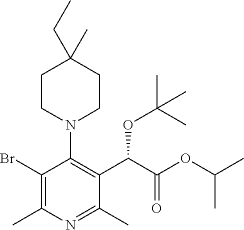 [SMILES: CCC1(C)CCN(c2c(Br)c(C)nc(C)c2[C@H](OC(C)(C)C)C(=O)OC(C)C)CC1]

(S)-Isopropyl 2-(5-bromo-4-(4-ethyl-4-methylpiperidin-1-yl)-2,6-dimethylpyridin-3-yl)-2-(tert-butoxy)acetate: The isobutylene gas was bubbled into a nitrogen purged, cooled (0° C.) solution of (S)-isopropyl 2-(5-bromo-4-(4-ethyl-4-methylpiperidin-1-yl)-2,6-dimethylpyridin-3-yl)-2-hydroxyacetate (1.9 g, 4.19 mmol) and 0.6 mL of 70% HClO₄ in DCM (15 mL) for 20 min. The reaction mixture was allowed to warm to rt and stirred for 18 h in a pressure sealed vessel, after which it was recooled, and an additional 0.4 mL of 70% HClO4 was added at 0° C., and the reaction was stirred for 24 h at rt. The reaction was diluted with DCM, washed with 1M Na₂CO₃ solution, and dried over MgSO₄. The crude product was charged (DCM) to a 80 g ISCO silica gel cartridge and gradient elution (5-12% EtOAc/hexanes) using an Isolera chromatography station gave (S)-isopropyl 2-(5-bromo-4-(4-ethyl-4-methylpiperidin-1-yl)-2,6-dimethylpyridin-3-yl)-2-(tert-butoxy)acetate 1.67 g (64%) as a mixture of diastereomers. ¹H NMR (500 MHz, DMSO-d₆) δ (br. s, 1H), 4.94-4.89 (m, 1H), 3.98 (br. s, 1H), 3.45-3.39 (m, 2H), 2.82 (m, 1H), 2.53 (s, 3H), 2.42 (s, 3H), 1.53-1.43 (m, 4H), 1.32-1.26 (m, 2H), 1.14 (br. s, 12H), 1.07 (d, J=6.2 Hz, 3H), 0.99/0.91 (s, 3H), 0.8 (t, J=7.3 Hz, 3H). UPLC (M+H)=485.4.

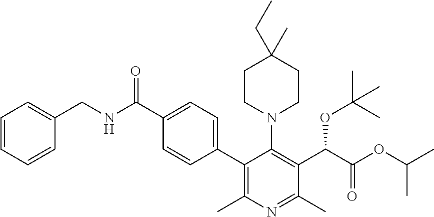 [SMILES: CCC1(C)CCN(c2c(-c3ccc(C(=O)NCc4ccccc4)cc3)c(C)nc(C)c2[C@H](OC(C)(C)C)C(=O)OC(C)C)CC1]

(S)-Isopropyl 2-(5-(4-(benzylcarbamoyl)phenyl)-4-(4-ethyl-4-methylpiperidin-1-yl)-2,6-dimethylpyridin-3-yl)-2-(tert-butoxy)acetate: The tetrakis (71 mg, 0.062 mmol) was added to a nitrogen purged and degassed solution of (S)-isopropyl 2-(5-bromo-2,6-dimethyl-4-(4-phenylpiperidin-1-yl)pyridin-3-yl)-2-(tert-butoxy)acetate (150 mg, 0.31 mmol), (4-(benzylcarbamoyl)phenyl)boronic acid (87 mg, 0.34 mmol), and potassium phosphate tribasic (494 mg, 2.3 mmol) in dioxane (2 mL) and water (1.25 mL) and stirred in a screw-capped pressure vessel for 16 h at 80° C. The reaction was allowed to cool, diluted with EtOAc, and the organic layer was washed with brine and dried (MgSO4). The crude product was charged (DCM) to a 40 g ISCO silica gel cartridge and gradient elution (25-75% EtOAc/hexanes) using an Isolera chromatography station gave (S)-isopropyl 2-(5-(4-(benzylcarbamoyl)phenyl)-4-(4-ethyl-4-methylpiperidin-1-yl)-2,6-dimethylpyridin-3-yl)-2-(tert-butoxy)acetate 160 mg (84%) as a mixture of diastereomers. ¹H NMR (500 MHz, DMSO) δ 9.19-9.17 (m,1H), 8.02 (d, J=8.1 Hz, 1H), 7.98 (d, J=7.7 Hz, 1H), 7.45-7.43 (m, 1H), 7.33-7.32 (m,4H), 7.25-7.24 (m, 2H), 5.96 (br. s, 1H), 4.96-4.92 (m, 1H), 4.51 (d, J=5.5 Hz, 2H), 3.64-3.62 (m, 2H), 3.14-3.07 (m, 1H), 2.85-2.81 (m, 1H), 2.44 (s, 3H), 2.24-2.17 (m, 1H), 2.04 (s, 3H), 1.90-1.85 (m, 1H), 1.45-1.22 (m, 2H), 1.16 (d, J=6.2 Hz, 3H), 1.13-1.11 (m, 12H), 1.01-0.94 (m, 2H) 0.73-0.70 (m, 3H), 0.50 (s, 3H). UPLC (M+H)=614.6.

### EXAMPLE 56

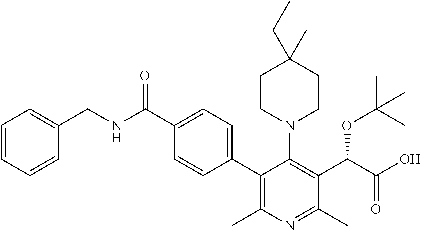 [SMILES: CCC1(C)CCN(c2c(-c3ccc(C(=O)NCc4ccccc4)cc3)c(C)nc(C)c2[C@H](OC(C)(C)C)C(=O)O)CC1]

(S)-2-(5-(4-(Benzylcarbamoyl)phenyl)-4-(4-ethyl-4-methylpiperidin-1-yl)-2,6-dimethylpyridin-3-yl)-2-(tert-butoxy)acetic acid: The 1.96 mL of 1M sodium hydroxide (79 mg, 1.96 mmol) was added to a solution of (S)-isopropyl 2-(5-(4-(benzylcarbamoyl)phenyl)-4-(4-ethyl-4-methylpiperidin-1-yl)-2,6-dimethylpyridin-3-yl)-2-(tert-butoxy)acetate (134 mg, 0.22 mmol) in ethanol (2 mL) and stirred for 24 h at 85° C. The reaction mixture was neutralized with 1N HCl soln, extracted with EtOAc, and the organic layer was washed with brine, and dried (MgSO4). The crude material was purified by prep HPLC (XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 35-75% B over 15 minutes, then a 5-minute hold at 100% B; Flow: 20 mL/min. There was obtained (S)-2-(5-(4-(benzylcarbamoyl)phenyl)-4-(4-ethyl-4-methylpiperidin-1-yl)-2,6-dimethylpyridin-3-yl)-2-(tert-butoxy)acetic acid 67.5 mg (54%) as a mixture of diastereomers. ¹H NMR (500 MHz, DMSO) δ 9.18-9.15 (m, 1H), 8.02 (d, J=8.1 Hz, 1H), 7.99 (d, J=8.1 Hz, 1H), 7.48-7.47 (m, 1H), 7.35-7.34 (m, 4H), 7.27-7.26 (m, 2H), 5.83 (s, 1H), 4.51 (d, J=5.1 Hz, 2H), 3.35-3.25 (m, 3H), 2.85-2.80 (m,1H), 2.45 (s, 3H), 2.23-2.18 (m, 1H), 2.05 (s, 3H), 1.89-1.82 (m, 1H), 1.50-1.41 (m, 1H), 1.31-1.24 (m, 1H),1.13 (br. s, 9H), 1.01-0.96 (m, 2H), 0.75-0.73 (m, 3H), 0.52 (br. s, 3H). UPLC (M+H)=614.6.

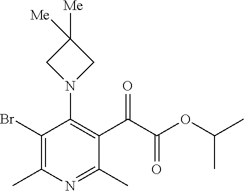 [SMILES: Cc1nc(C)c(C(=O)C(=O)OC(C)C)c(N2CC(C)(C)C2)c1Br]

Isopropyl 2-(5-bromo-4-(3,3-dimethylazetidin-1-yl)-2,6-dimethylpyridin-3-yl)-2-oxoacetate: To a solution of 3,3-dimethylazetidine, HCl (1.0 g, 8.22 mmol) and DIEA (4.3 mL, 24.7 mmol) in anhydrous CH₃CN (40 mL) was added isopropyl 2-(5-bromo-4-chloro-2,6-dimethylpyridin-3-yl)-2-oxoacetate (2.75 g, 8.2 mmol) at rt. The resulting mixture was placed in a pre-heated oil bath (80° C.) and stirred for 24 h; concentrated, and charged (DCM) to a 80 g ISCO silica gel cartridge and gradient eluted (5-35% EtOAc/hexanes) using an Isolera chromatography station to give isopropyl 2-(5-bromo-4-(3,3-dimethylazetidin-1-yl)-2,6-dimethylpyridin-3-yl)-2-oxoacetate 1.6 g (50.8%). ¹H NMR (500 MHz, DMSO-d₆) δ 5.15-5.11 (m, 1H), 3.79 (s, 4H), 2.48 (s, 3H), 2.19 (s, 3H), 1.31 (d, J=6.2 Hz, 6H), 1.18 (s, 6H). UPLC (M+H)=385.2.

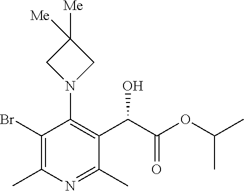 [SMILES: Cc1nc(C)c([C@H](O)C(=O)OC(C)C)c(N2CC(C)(C)C2)c1Br]

(S)-Isopropyl 2-(5-bromo-4-(3,3-dimethylazetidin-1-yl)-2,6-dimethylpyridin-3-yl)-2-hydroxyacetate: The 1.5 mL of benzo[d][1,3,2]dioxaborole (751 mg, 6.26 mmol) was added to a nitrogen purged solution of isopropyl 2-(5-bromo-4-(3,3-dimethylazetidin-1-yl)-2,6-dimethylpyridin-3-yl)-2-oxoacetate (1.6 g, 4.17 mmol) and 1.25 mL of (R)-1-methyl-3,3-diphenylhexahydropyrrolo[1,2-c][1,3,2]oxazaborole (347 mg, 1.25 mmol) in toluene (40 mL) at −60° C. and allowed to warm to −15° C. before being placed in the freezer overnight. The reaction was quenched with 1M Na₂CO₃ (15 mL), diluted with EtOAc, and stirred for 20 min. The organic layer was washed with brine and dried (Na₂SO₄). The crude product was charged (DCM) to an 80 g ISCO silica gel cartridge and gradient elution (5-35% EtOAc/hexanes) using an Isolera chromatography station gave (S)-isopropyl 2-(5-bromo-4-(3,3-dimethylazetidin-1-yl)-2,6-dimethylpyridin-3-yl)-2-hydroxyacetate 987 mg (61%). ¹H NMR (500 MHz, DMSO-d₆) δ 5.16 (s, 1H), 4.97-4.92 (m, 1H), 4.13 (d, J=7.7 Hz, 2H), 4.04 (d, J=7.7 Hz, 2H), 2.42 (s, 3H), 2.21 (s, 3H), 1.22 (s, 6H), 1.17 (d, J=6.2 Hz, 3H), 1.12 (d, J=6.2 Hz, 3H). UPLC (M+H)=387.2.

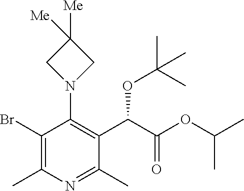 [SMILES: Cc1nc(C)c([C@H](OC(C)(C)C)C(=O)OC(C)C)c(N2CC(C)(C)C2)c1Br]

(S)-Isopropyl 2-(5-bromo-4-(3,3-dimethylazetidin-1-yl)-2,6-dimethylpyridin-3-yl)-2-(tert-butoxy)acetate: The isobutylene gas was bubbled into a nitrogen purged, cooled (0° C.) solution of (S)-isopropyl 2-(5-bromo-4-(3,3-dimethylazetidin-1-yl)-2,6-dimethylpyridin-3-yl)-2-hydroxyacetate (970 mg, 2.5 mmol) and 0.5 mL of 70% HClO₄ in DCM (20 mL) for 20 min. The reaction mixture was allowed to warm to rt and stirred for 18 h in a pressure sealed vessel, diluted with DCM, washed with 1M Na₂CO₃ solution, and dried over MgSO₄. The crude product was charged (DCM) to a 40 g ISCO silica gel cartridge and gradient elution (5-35% EtOAc/hexanes) using an Isolera chromatography station gave (S)-isopropyl 2-(5-bromo-4-(3,3-dimethylazetidin-1-yl)-2,6-dimethylpyridin-3-yl)-2-(tert-butoxy)acetate 578 mg (52%). ¹H NMR (500 MHz, DMSO) δ 5.30 (s, 1H), 5.0-4.95 (m, 1H), 4.12 (d, J=7.2 Hz, 2H), 4.02 (br. s, 2H), 2.44 (s, 3H), 2.25 (br. s, 3H), 1.26 (s, 6H), 1.20 (d, J=6.3 Hz, 3H), 1.17 (d, J=6.3 Hz, 3H), 1.07 (s, 9H). UPLC (M+H)=443.3.

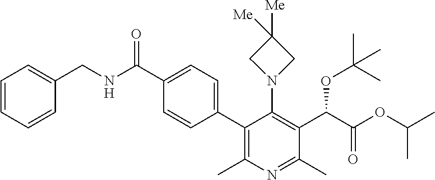 [SMILES: Cc1nc(C)c([C@H](OC(C)(C)C)C(=O)OC(C)C)c(N2CC(C)(C)C2)c1-c1ccc(C(=O)NCc2ccccc2)cc1]

(S)-Isopropyl 2-(5-(4-(benzylcarbamoyl)phenyl)-4-(3,3-dimethylazetidin-1-yl)-2,6-dimethylpyridin-3-yl)-2-(tert-butoxy)acetate: The tetrakis (57.6 mg, 0.05 mmol) was added to a nitrogen purged and degassed solution of (S)-isopropyl 2-(5-bromo-4-(3,3-dimethylazetidin-1-yl)-2,6-dimethylpyridin-3-yl)-2-(tert-butoxy)acetate (110 mg, 0.25 mmol), (4-(benzylcarbamoyl)phenyl)boronic acid (69.9 mg, 0.274 mmol), and sodium carbonate (185 mg, 1.74 mmol) in dioxane (3 mL) and water (0.6 mL) and stirred in a screw-capped pressure vessel for 4 h at 90° C. The reaction was allowed to cool, diluted with EtOAc, and the organic layer was washed with brine and dried (Na2SO4). The crude product was charged (DCM) to a 24 g ISCO silica gel cartridge and gradient elution (5-65% EtOAc/hexanes) using an Isolera chromatography station gave (S)-isopropyl 2-(5-(4-(benzylcarbamoyl)phenyl)-4-(3,3-dimethylazetidin-1-yl)-2,6-dimethylpyridin-3-yl)-2-(tert-butoxy)acetate 33 mg (23%). UPLC (M+H)=572.5.

### EXAMPLE 57

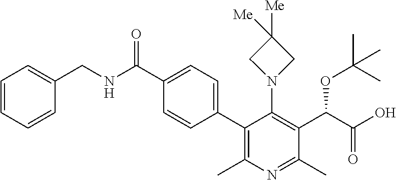 [SMILES: Cc1nc(C)c([C@H](OC(C)(C)C)C(=O)O)c(N2CC(C)(C)C2)c1-c1ccc(C(=O)NCc2ccccc2)cc1]

(S)-2-(5-(4-(Benzylcarbamoyl)phenyl)-4-(3,3-dimethylazetidin-1-yl)-2,6-dimethylpyridin-3-yl)-2-(tert-butoxy)acetic acid: The 0.2 mL of 1M sodium hydroxide (7.84 mg, 0.19 mmol) was added to a solution of (S)-isopropyl 2-(5-(4-(benzylcarbamoyl)phenyl)-4-(3,3-dimethylazetidin-1-yl)-2,6-dimethylpyridin-3-yl)-2-(tert-butoxy)acetate (28 mg, 0.05 mmol) in ethanol (1 mL) and stirred for 18 h at 90° C. The reaction mixture was neutralized with 1N HCl soln, extracted with EtOAc, and the organic layer was washed with brine, and dried (MgSO4). The crude material was purified by prep HPLC (XBridge C18, 19×200 mm, 5-μm particles; Mobile Phase A: 5:95 MeOH:water with 10-mM ammonium acetate; Mobile Phase B: 95:5 MeOH:water with 10-mM ammonium acetate; Gradient: 50-100% B over 15 minutes, then a 5-minute hold at 100% B; Flow: 20 mL/min. There was obtained (S)-2-(5-(4-(benzylcarbamoyl)phenyl)-4-(3,3-dimethylazetidin-1-yl)-2,6-dimethylpyridin-3-yl)-2-(tert-butoxy)acetic acid 19 mg (73%). ¹H NMR (500 MHz, DMSO) δ 9.14-9.11 (m, 1H), 7.96 (d, J=7.7 Hz, 1H), 7.91 (d, J=7.7 Hz, 1H), 7.52 (br. s, 1H), 7.37-7.33 (m, 4H), 7.27-7.25 (m, 1H), 7.05 (br. s, 1H), 5.10 (s, 1H), 4.51 (d, J=5.5 Hz, 2H) 3.38 (br. s, 4H), 2.00 (s, 3H), 1.91 (s, 3H), 1.12 (s, 9H), 1.01 (s, 6H). UPLC (M+H)=530.45.

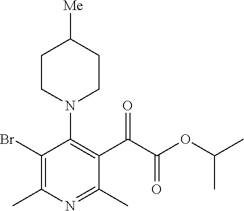 [SMILES: Cc1nc(C)c(C(=O)C(=O)OC(C)C)c(N2CCC(C)CC2)c1Br]

Isopropyl 2-(5-bromo-2,6-dimethyl-4-(4-methylpiperidin-1-yl)pyridin -3-yl)-2-oxoacetate: To a solution of 4-methylpiperidine (593 mg, 5.98 mmol) and DIEA (2.1 mL, 12 mmol) in anhydrous CH₃CN (15 mL) was added isopropyl 2-(5-bromo-4-chloro-2,6-dimethylpyridin-3-yl)-2-oxoacetate (2.0 g, 5.98 mmol) at rt. The resulting mixture was placed in a pre-heated oil bath (80° C.) and stirred for 24 h; cooled, diluted with ether, washed with water, brine, and dried (MgSO₄). The crude product was charged (DCM) to a 80 g ISCO silica gel cartridge and gradient eluted (5-35% EtOAc/hexanes) using an Isolera chromatography station gave isopropyl 2-(5-bromo-2,6-dimethyl-4-(4-methylpiperidin-1-yl)pyridin -3-yl)-2-oxoacetate 1.99 g (84%). ¹H NMR (500 MHz, DMSO-d₆) δ 5.08-5.03 (m, 1H), 3.41-3.39 (m, 2H), 2.83 (br. s, 2H), 2.60 (s, 3H), 2.29 (s, 3H), 1.58 (d, J=12.5 Hz, 2H), 1.44 (br. s, 1H), 1.29 (d, J=6.2 Hz, 6H), 1.09-1.07 (m, 2H), 0.92 (d, J=6.2 Hz, 3H). UPLC (M+H)=399.2.

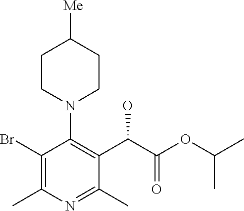 [SMILES: Cc1nc(C)c([C@H](O)C(=O)OC(C)C)c(N2CCC(C)CC2)c1Br]

(S)-Isopropyl 2-(5-bromo-2,6-dimethyl-4-(4-methylpiperidin-1-yl)pyridin-3-yl)-2-hydroxyacetate: The 2.0 mL of benzo[d][1,3,2]dioxaborole (2.3 g, 9.56 mmol) was added to a nitrogen purged solution of isopropyl 2-(5-bromo-2,6-dimethyl-4-(4-methylpiperidin-1-yl)pyridin -3-yl)-2-oxoacetate (1.9 g, 4.78 mmol) and 1.9 mL of (R)-1-methyl-3,3-diphenylhexahydropyrrolo[1,2-c][1,3,2]oxazaborole (530 mg, 1.9 mmol) in toluene (45 mL) at −60° C. and allowed to warm to −15° C. before being placed in the freezer overnight. The reaction was quenched with 1M Na₂CO₃, diluted with EtOAc, and stirred for 30 min. The organic layer was washed with sat'd Na₂CO₃ solution, brine and dried (MgSO₄). The crude product was charged (DCM) to a 80 g ISCO silica gel cartridge and gradient elution (0-30% EtOAc/hexanes) using an Isolera chromatography station gave (S)-isopropyl 2-(5-bromo-2,6-dimethyl-4-(4-methylpiperidin-1-yl)pyridin-3-yl)-2-hydroxyacetate 1.9 g (100%) as a mixture of diastereomers. ¹H NMR (500 MHz, DMSO-d₆) δ 5.94 (s, 1H), 4.94 (dt, J=12.3, 6.3 Hz, 1H), 3.57 (t, J=11.0 Hz, 1H), 3.34 (t, J=11.4 Hz 1H), 2.86 (d, J=11 Hz, 1H), 2.76 (d, J=11.0 Hz, 1H), 2.51 (s, 3H), 2.37 (s, 3H), 1.64-1.56 (m, 2H), 1.43 (br. s, 1H), 1.29-1.25 (m, 2H), 1.14 (d, J=6.2 Hz, 3H), 1.07 (d, J=6.2 Hz, 3H), 0.95 (d, J=6.2 Hz, 3H). UPLC (M+H)=401.2.

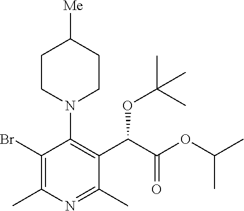 [SMILES: Cc1nc(C)c([C@H](OC(C)(C)C)C(=O)OC(C)C)c(N2CCC(C)CC2)c1Br]

(S)-Isopropyl 2-(5-bromo-2,6-dimethyl-4-(4-methylpiperidin-1-yl)pyridin-3-yl)-2-(tert-butoxy)acetate: The isobutylene gas was bubbled into a nitrogen purged, cooled (0° C.) solution of (S)-isopropyl 2-(5-bromo-2,6-dimethyl-4-(4-methylpiperidin-1-yl)pyridin-3-yl)-2-hydroxyacetate (2.25 g, 5.63 mmol) and 0.53 mL of 70% HClO₄ in DCM (30 mL) for 20 min. The reaction mixture was allowed to warm to rt and stirred for 72 h in a pressure sealed vessel. The reaction was then diluted with DCM, washed with 1M Na₂CO₃ solution, and dried over MgSO₄. The crude product was charged (DCM) to a 80 g ISCO silica gel cartridge and gradient elution (0-12% EtOAc/hexanes) using an Isolera chromatography station gave (S)-isopropyl 2-(5-bromo-2,6-dimethyl-4-(4-methylpiperidin-1-yl)pyridin-3-yl)-2-(tert-butoxy)acetate 1.82 g (71%) as a mixture of diastereomers. ¹H NMR (500 MHz, DMSO-d₆) δ 6.16 (s, 1H), 4.93-4.90 (m, 1H), 3.82-3.77 (m, 1H), 3.24 (t, J=10.6 Hz 1H), 2.98 (d, J=9.9 Hz, 1H), 2.74 (br. s, 1H), 2.51 (s, 3H), 2.41 (s, 3H), 1.73 (br. s, 1H), 1.62 (br. s, 1H), 1.49 (br. s, 1H), 1.28-1.23 (m, 2H), 1.16-1.14 (m, 12H), 1.07 (d, J=6.2 Hz, 3H), 0.97 (d, J=6.2 Hz, 3H). UPLC (M+H)=457.3.

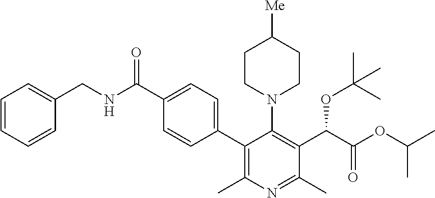 [SMILES: Cc1nc(C)c([C@H](OC(C)(C)C)C(=O)OC(C)C)c(N2CCC(C)CC2)c1-c1ccc(C(=O)NCc2ccccc2)cc1]

(S)-Isopropyl 2-(5-(4-(benzylcarbamoyl)phenyl)-2,6-dimethyl-4-(4-methylpiperidin-1-yl)pyridin-3-yl)-2-(tert-butoxy)acetate: Tetrakis (76 mg, 0.066 mmol) was added to an argon-degassed solution of (S)-isopropyl 2-(5-bromo-2,6-dimethyl-4-(4-methylpiperidin-1-yl)pyridin-3-yl)-2-(tert-butoxy)acetate (150 mg, 0.329 mmol), (4-(benzylcarbamoyl)phenyl)boronic acid (92 mg, 0.362 mmol), potassium phosphate tribasic (524 mg, 2.47 mmol) in dioxane (2 ml) and water (0.5 ml) stirred in a screw-capped pressure vessel for 16 h at 80° C. The reaction was allowed to cool, diluted with EtOAc, and the organic layer was washed with brine and dried (MgSO4). The crude product was charged (DCM) to a 40 g ISCO silica gel cartridge and gradient elution (0-60% EtOAc/hexanes) using an Isolera chromatography station gave (S)-isopropyl 2-(5-(4-(benzylcarbamoyl)phenyl)-2,6-dimethyl-4-(4-methylpiperidin-1-yl)pyridin-3-yl)-2-(tert-butoxy)acetate 163 mg (85%) as a mixture of diastereomers. ¹H NMR (500 MHz, DMSO-d6) δ 9.17-9.15 (m, 1H), 7.98 (t, J=7.3 Hz, 2H), 7.47 (d, J=8.1 Hz, 1H), 7.38-7.34 (m, 4H), 7.28-7.25 (m, 1H), 7.21 (d, J=7.7 Hz, 1H), 5.96 (br. s, 1H), 5.00-4.95 (m, 1H), 4.52 (d, J=5.9 Hz, 2H), 3.35-3.31 (m, 3H), 2.65-2.59 (m, 1H), 2.46 (s, 3H), 2.04 (s, 3H), 1.68 (t, J=11.0 Hz, 1H), 1.52-1.48 (m, 1H), 1.37 (br. s, 1H), 1.20 (d, J=6.2 Hz, 3H), 1.16-1.13 (m, 12H), 1.07-1.01 (m, 2H), 0.84 (d, J=5.1 Hz, 3H). UPLC (M+H)=586.5.

### EXAMPLE 58

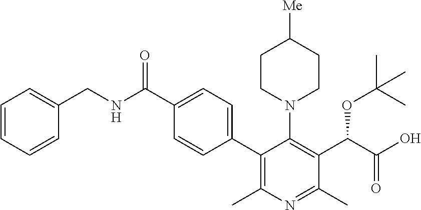 [SMILES: Cc1nc(C)c([C@H](OC(C)(C)C)C(=O)O)c(N2CCC(C)CC2)c1-c1ccc(C(=O)NCc2ccccc2)cc1]

(S)-2-(5-(4-(Benzylcarbamoyl)phenyl)-2,6-dimethyl-4-(4-methylpiperidin-1-yl)pyridin-3-yl)-2-(tert-butoxy)acetic acid: The 0.51 mL of 1M sodium hydroxide (20.5 mg, 0.51 mmol) was added to a solution of (S)-isopropyl 2-(5-(4-(benzylcarbamoyl)phenyl)-2,6-dimethyl-4-(4-methylpiperidin-1-yl)pyridin-3-yl)-2-(tert-butoxy)acetate (50 mg, 0.09 mmol) in ethanol (1 mL) and stirred for 24 h at 90° C. An additional 0.51 mL sodium hydroxide was added and the reaction was continued for 24 h. The reaction mixture was neutralized with 1N HCl soln, extracted with EtOAc, and the organic layer was washed with brine, and dried (MgSO4). The crude material was purified by prep HPLC (XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 25-65% B over 15 minutes, then a 5-minute hold at 100% B; Flow: 20 mL/min. There was obtained (S)-2-(5-(4-(benzylcarbamoyl)phenyl)-2,6-dimethyl-4-(4-methylpiperidin-1-yl)pyridin-3-yl)-2-(tert-butoxy)acetic acid 19.4 mg (42%) as a mixture of diastereomers. ¹H NMR (500 MHz, DMSO) δ 9.17-9.15 (m,1H), 7.97 (t, J=8.4 Hz, 2H), 7.47 (d, J=7.7 Hz, 1H), 7.37-7.33 (m, 4H), 7.27-7.25 (m, 1H), 7.19 (d, J=7.3 Hz, 1H), 5.80 (s, 1H), 4.51 (d, J=5.9 Hz, 2H), 3.48 (br. s, 3H), 2.62-2.57 (m, 1H), 2.45 (s, 3H), 2.03 (s, 3H), 1.65-1.61 (m, 1H), 1.50 (d, J=12.8 Hz, 1H), 1.35-1.33 (m, 1H), 1.25-1.22 (m, 1H), 1.13 (s, 9H), 1.06-1.02 (m, 1H), 0.82 (d, J=3.7 Hz, 3H). UPLC (M+H)=544.5.

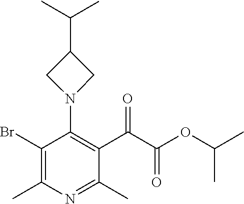 [SMILES: Cc1nc(C)c(C(=O)C(=O)OC(C)C)c(N2CC(C(C)C)C2)c1Br]

Isopropyl 2-(5-bromo-4-(3-isopropylazetidin-1-yl)-2,6-dimethylpyridin-3-yl)-2-oxoacetate: To a solution of 3-isopropylazetidine, HCl (1.0 g, 7.37 mmol) and DIEA (3.8 mL, 22.1 mmol) in anhydrous CH₃CN (35 mL) was added isopropyl 2-(5-bromo-4-chloro-2,6-dimethylpyridin-3-yl)-2-oxoacetate (2.47 g, 7.38 mmol) at rt. The resulting mixture was placed in a pre-heated oil bath (80° C.) and stirred for 24 h; concentrated, and charged (DCM) to a 80 g ISCO silica gel cartridge and gradient eluted (5-35% EtOAc/hexanes) using an Isolera chromatography station to give isopropyl 2-(5-bromo-4-(3-isopropylazetidin-1-yl)-2,6-dimethylpyridin-3-yl)-2-oxoacetate 895 mg (30.5%). ¹H NMR (500 MHz, DMSO-d₆) δ 5.18-5.11 (m, 1H), 4.14 (t, J=8.8 Hz, 2H), 3.73 (t, J=8.1 Hz, 2H), 2.48 (s, 3H), 2.26-2.22 (m, 1H), 2.19 (s, 3H), 1.70-1.63 (m, 1H), 1.30 (d, J=6.2 Hz, 6H), 0.79 (d, J=6.6 Hz, 6H). UPLC (M+H)=399.2.

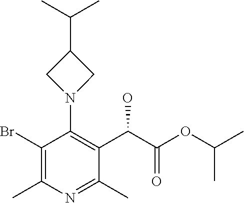 [SMILES: Cc1nc(C)c([C@H](O)C(=O)OC(C)C)c(N2CC(C(C)C)C2)c1Br]

(S)-Isopropyl 2-(5-bromo-4-(3-isopropylazetidin-1-yl)-2,6-dimethylpyridin-3-yl)-2-hydroxyacetate: The 0.7 mL of benzo[d][1,3,2]dioxaborole (396 mg, 3.3 mmol) was added to a nitrogen purged solution of isopropyl 2-(5-bromo-4-(3-isopropylazetidin-1-yl)-2,6-dimethylpyridin-3-yl)-2-oxoacetate (875 mg, 2.2 mmol) and 0.66 mL of (R)-1-methyl-3,3-diphenylhexahydropyrrolo[1,2-c][1,3,2]oxazaborole (183 mg, 0.66 mmol) in toluene (20 mL) at −60° C. and allowed to warm to −15° C. before being placed in the freezer overnight. The reaction was quenched with 1M Na₂CO₃ (5 mL), diluted with EtOAc, and stirred for 20 min. The organic layer was washed with brine and dried (MgSO₄). The crude product was charged (DCM) to a 80 g ISCO silica gel cartridge and gradient elution (5-35% EtOAc/hexanes) using an Isolera chromatography station gave (S)-isopropyl 2-(5-bromo-4-(3-isopropylazetidin-1-yl)-2,6-dimethylpyridin-3-yl)-2-hydroxyacetate 621 mg (71%) as a mixture of diastereomers. ¹H NMR (500 MHz, DMSO-d₆) δ 5.19 (d, J=4.4 Hz, 1H), 4.96-4.92 (m, 1H), 4.50 (t, J=8.1 Hz, 1H), 4.39 (t, J=8.4 Hz, 1H), 4.07 (t, J=7.0 Hz, 1H), 3.97 (t, J=7.3 Hz, 1H), 2.42 (s, 3H), 2.21 (s, 3H), 2.21-2.17 (m, 1H), 1.73-1.67 (m, 1H), 1.17 (d, J=6.2 Hz, 3H), 1.12 (d, J=6.2 Hz, 3H), 0.83 (d, J=6.6 Hz, 6H). UPLC (M+H)=401.3.

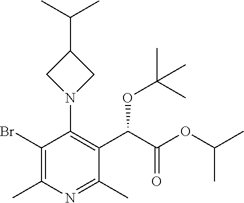 [SMILES: Cc1nc(C)c([C@H](OC(C)(C)C)C(=O)OC(C)C)c(N2CC(C(C)C)C2)c1Br]

(S)-Isopropyl 2-(5-bromo-4-(3-isopropylazetidin-1-yl)-2,6-dimethylpyridin-3-yl)-2-(tert-butoxy)acetate: The isobutylene gas was bubbled into a nitrogen purged, cooled (0° C.) solution of (S)-isopropyl 2-(5-bromo-4-(3-isopropylazetidin-1-yl)-2,6-dimethylpyridin-3-yl)-2-hydroxyacetate (601 mg, 1.5 mmol) and 0.5 mL of 70% HClO₄ in DCM (12 mL) for 20 min. The reaction mixture was allowed to warm to rt and stirred for 18 h in a pressure sealed vessel, diluted with DCM, washed with 1M Na₂CO₃ solution, and dried over MgSO₄. The crude product was charged (DCM) to a 40 g ISCO silica gel cartridge and gradient elution (5-50% EtOAc/hexanes) using an Isolera chromatography station gave (S)-isopropyl 2-(5-bromo-4-(3-isopropylazetidin-1-yl)-2,6-dimethylpyridin-3-yl)-2-(tert-butoxy)acetate 621 mg (90%) as a mixture of diastereomers. ¹H NMR (500 MHz, DMSO-d₆) δ 5.28 (s, 1H), 5.02-4.97 (m, 1H), 4.57 (t, J=8.1 Hz, 1H), 4.40 (t, J=7.7 Hz, 1H), 4.02 (t, J=7.0 Hz, 1H), 3.91 (t, J=7.3 Hz, 1H), 2.44 (s, 3H), 2.33-2.29 (m, 1H), 2.25 (s, 3H), 1.74-1.70 (m, 1H), 1.21 (d, J=6.2 Hz, 3H), 1.17 (d, J=6.2 Hz, 3H), 1.06 (s, 9H), 0.84 (d, J=6.6 Hz, 6H). UPLC (M+H)=457.4.

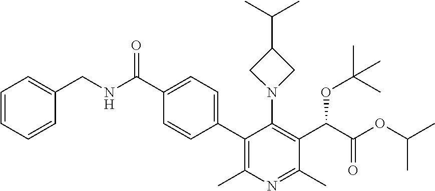 [SMILES: Cc1nc(C)c([C@H](OC(C)(C)C)C(=O)OC(C)C)c(N2CC(C(C)C)C2)c1-c1ccc(C(=O)NCc2ccccc2)cc1]

(S)-Isopropyl 2-(5-(4-(benzylcarbamoyl)phenyl)-4-(3-isopropylazetidin-1-yl)-2,6-dimethylpyridin-3-yl)-2-(tert-butoxy)acetate: The tetrakis (38.1 mg, 0.033 mmol) was added to a nitrogen purged and degassed solution of (S)-isopropyl 2-(5-bromo-4-(3-isopropylazetidin-1-yl)-2,6-dimethylpyridin-3-yl)-2-(tert-butoxy)acetate (150 mg, 0.33 mmol), (4-(benzylcarbamoyl)phenyl)boronic acid (92 mg, 0.36 mmol), and sodium carbonate (209 mg, 1.98 mmol) in dioxane (4.5 mL) and water (0.9 mL) and stirred in a screw-capped pressure vessel for 4 h at 90° C. The reaction was allowed to cool, diluted with EtOAc, and the organic layer was washed with brine and dried (Na2SO4). The crude product was charged (DCM) to a 24 g ISCO silica gel cartridge and gradient elution (5-75% EtOAc/hexanes) using an Isolera chromatography station gave (S)-isopropyl 2-(5-(4-(benzylcarbamoyl)phenyl)-4-(3-isopropylazetidin-1-yl)-2,6-dimethylpyridin-3-yl)-2-(tert-butoxy)acetate 170 mg (88%) as a mixture of diastereomers. ¹H NMR (500 MHz, DMSO) δ 9.12-9.10 (m, 1H), 7.96 (d, J=7.7 Hz, 1H), 7.90 (d, J=8.1 Hz, 1H), 7.56 (br. s, 1H), 7.35-7.32 (m, 4H), 7.27-7.24 (m, 1H), 7.01 (br. s, 1H), 5.20 (s, 1H), 5.04-4.99 (m, 1H), 4.51 (d, J=5.5 Hz, 2H), 3.41-3.25 (series m, 4H), 2.29 (s, 3H), 2.02 (s, 3H), 2.02-2.00 (m 1H), 1.49-1.44 (m, 1H), 1.22-1.20 (m, 6H), 1.12 (s, 9H), 0.68 (d, J=6.6 Hz, 3H), 0.63 (d, J=6.6 Hz, 3H). UPLC (M+H)=586.6.

### EXAMPLE 59

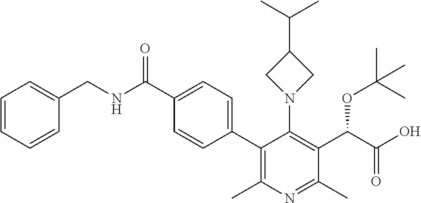 [SMILES: Cc1nc(C)c([C@H](OC(C)(C)C)C(=O)O)c(N2CC(C(C)C)C2)c1-c1ccc(C(=O)NCc2ccccc2)cc1]

(S)-2-(5-(4-(Benzylcarbamoyl)phenyl)-4-(3-isopropylazetidin-1-yl)-2,6-dimethylpyridin-3-yl)-2-(tert-butoxy)acetic acid: The 1.0 mL of 1M sodium hydroxide (41 mg, 1.0 mmol) was added to a solution of (S)-isopropyl 2-(5-(4-(benzylcarbamoyl)phenyl)-4-(3-isopropylazetidin-1-yl)-2,6-dimethylpyridin-3-yl)-2-(tert-butoxy)acetate (150 mg, 0.256 mmol) in ethanol (3 mL) and stirred for 18 h at 90° C. The reaction mixture was neutralized with 1N HCl soln, extracted with EtOAc, and the organic layer was washed with brine, and dried (MgSO4). The crude material was purified by prep HPLC (XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-60% B over 15 minutes, then a 5-minute hold at 100% B; Flow: 20 mL/min. There was obtained (S)-2-(5-(4-(benzylcarbamoyl)phenyl)-4-(3-isopropylazetidin-1-yl)-2,6-dimethylpyridin-3-yl)-2-(tert-butoxy)acetic acid 80 mg (58%) as a mixture of diastereomers. ¹H NMR (500 MHz, DMSO) δ 9.17-9.14 (m, 1H), 7.97 (d, J=8.1 Hz, 1H), 7.93 (d, J=7.7 Hz, 1H), 7.54 (br. s, 1H), 7.35-7.32 (m, 4H), 7.25 (m, 1H), 7.05 (br. s, 1H), 5.06 (s, 1H), 4.51 (d, J=5.9 Hz, 2H) 3.90 (br. s, 1H), 3.69 (br. s, 1H), 3.57 (br. s, 1H), 3.41 (br. s, 1H), 2.38 (s, 3H), 2.05 (s, 3H), 2.04-2.00 (m, 1H), 1.51-1.47 (m, 1H), 1.11 (s, 9H), 0.66-0.63 (m, 6H). UPLC (M+H)=544.5.

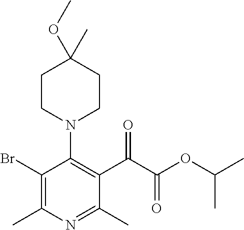 [SMILES: COC1(C)CCN(c2c(Br)c(C)nc(C)c2C(=O)C(=O)OC(C)C)CC1]

Isopropyl 2-(5-bromo-4-(4-methoxy-4-methylpiperidin-1-yl)-2,6-dimethylpyridin-3-yl)-2-oxoacetate: To a solution of 4-methoxy-4-methylpiperidine, HCl (1.0 g, 6.04 mmol) and DIEA (4.2 mL, 24.5 mmol) in anhydrous CH₃CN (30 mL) was added isopropyl 2-(5-bromo-4-chloro-2,6-dimethylpyridin-3-yl)-2-oxoacetate (3.4 g, 6.04 mmol) at rt. The resulting mixture was placed in a pre-heated oil bath (80° C.) and stirred for 24 h; cooled, diluted with ether, washed with water, brine, and dried (MgSO₄). The crude product was charged (DCM) to a 80 g ISCO silica gel cartridge and gradient eluted (5-35% EtOAc/hexanes) using an Isolera chromatography station gave isopropyl 2-(5-bromo-4-(4-methoxy-4-methylpiperidin-1-yl)-2,6-dimethylpyridin-3-yl)-2-oxoacetate 1.57 g (61%). ¹H NMR (500 MHz, DMSO-d₆) δ 5.08-5.03 (m, 1H), 3.44-3.40 (m, 4H), 3.11 (s, 3H), 2.60 (s, 3H), 2.29 (s, 3H), 1.67 (d, J=13.2 Hz, 2H), 1.43 (br. s., 2H), 1.29 (d, J=6.2 Hz, 6H), 1.11 (s, 3H). UPLC (M+H)=429.3.

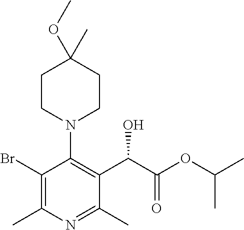 [SMILES: COC1(C)CCN(c2c(Br)c(C)nc(C)c2[C@H](O)C(=O)OC(C)C)CC1]

(S)-Isopropyl 2-(5-bromo-4-(4-methoxy-4-methylpiperidin-1-yl)-2,6-dimethylpyridin-3-yl)-2-hydroxyacetate: The 1.55 mL of benzo[d][1,3,2]dioxaborole (870 mg, 7.25 mmol) was added to a nitrogen purged solution of isopropyl 2-(5-bromo-4-(4-methoxy-4-methylpiperidin-1-yl)-2,6-dimethylpyridin-3-yl)-2-oxoacetate (1.55 g, 3.63 mmol) and 1.45 mL of (R)-1-methyl-3,3-diphenylhexahydropyrrolo[1,2-c][1,3,2]oxazaborole (402 mg, 1.45 mmol) in toluene (30 mL) at −60° C. and allowed to warm to −15° C. before being placed in the freezer overnight. The reaction was quenched with 1M Na₂CO₃, diluted with EtOAc, and stirred for 30 min. The organic layer was washed with sat'd Na₂CO₃ solution, brine, and dried (MgSO₄). The crude product was charged (DCM) to a 80 g ISCO silica gel cartridge and gradient elution (0-30% EtOAc/hexanes) using an Isolera chromatography station gave (S)-isopropyl 2-(5-bromo-4-(4-methoxy-4-methylpiperidin-1-yl)-2,6-dimethylpyridin-3-yl)-2-hydroxyacetate 1.48 g (95%) as a mixture of diastereomers. ¹H NMR (500 MHz, DMSO-d₆) δ 5.89 (br. s, 1H), 4.95-4.92 (m, 1H), 3.78 (t, J=11.0 Hz, 1H), 3.53 (t, J=10.6 Hz, 1H), 3.37-3.35 (m, 1H), 3.25 (br. s, 1H), 3.14 (s, 3H), 2.52 (s, 3H), 2.37 (s, 3H), 1.75-1.69 (m, 2H), 1.64-1.52 (m, 2H), 1.15-1.16 (m, 6H). 1.07 (d, J=6.2 Hz, 3H). UPLC (M+H)=431.3.

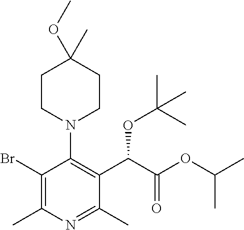 [SMILES: COC1(C)CCN(c2c(Br)c(C)nc(C)c2[C@H](OC(C)(C)C)C(=O)OC(C)C)CC1]

(S)-Isopropyl 2-(5-bromo-4-(4-methoxy-4-methylpiperidin-1-yl)-2,6-dimethylpyridin-3-yl)-2-(tert-butoxy)acetate: The isobutylene gas was bubbled into a nitrogen purged, cooled (0° C.) solution of (S)-isopropyl 2-(5-bromo-4-(4-methoxy-4-methylpiperidin-1-yl)-2,6-dimethylpyridin-3-yl)-2-hydroxyacetate (1.45 g, 3.38 mmol) and 0.32 mL of 70% HClO₄ in DCM (30 mL) for 20 min. The reaction mixture was allowed to warm to rt and stirred for 48 h in a pressure sealed vessel. The reaction was then diluted with DCM, washed with 1M Na₂CO₃ solution, and dried over MgSO₄. The crude product was charged (DCM) to a 80 g ISCO silica gel cartridge and gradient elution (0-30% EtOAc/hexanes) using an Isolera chromatography station gave (S)-isopropyl 2-(5-bromo-4-(4-methoxy-4-methylpiperidin-1-yl)-2,6-dimethylpyridin-3-yl)-2-(tert-butoxy)acetate 1.35 g (82%) as a mixture of diastereomers. ¹H NMR (500 MHz, DMSO-d₆) δ 6.18 (br. s, 1H), 4.93-4.89 (m, 1H), 4.01 (br. s, 1H), 3.46 (t, J=11.0 Hz 1H), 3.37-3.28 (m, 2H), 3.15 (s, 3H), 2.52 (s, 3H), 2.42 (s, 3H), 1.79-1.51 (series m, 4H), 1.16-1.14 (m, 15H), 1.07 (d, J=6.2 Hz, 3H). UPLC (M+H)=487.4.

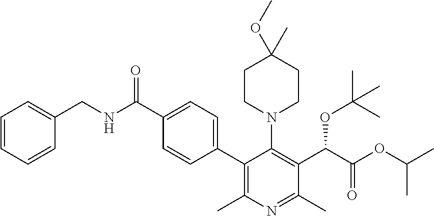 [SMILES: COC1(C)CCN(c2c(-c3ccc(C(=O)NCc4ccccc4)cc3)c(C)nc(C)c2[C@H](OC(C)(C)C)C(=O)OC(C)C)CC1]

(S)-Isopropyl 2-(5-(4-(benzylcarbamoyl)phenyl)-4-(4-methoxy-4-methylpiperidin-1-yl)-2,6-dimethylpyridin-3-yl)-2-(tert-butoxy)acetate: Tetrakis (143 mg, 0.124 mmol) was added to an argon-degassed solution of (S)-isopropyl 2-(5-bromo-4-(4-methoxy-4-methylpiperidin-1-yl)-2,6-dimethylpyridin-3-yl)-2-(tert-butoxy)acetate (300 mg, 0.618 mmol), (4-(benzylcarbamoyl)phenyl)boronic acid (173 mg, 0.68 mmol), potassium phosphate tribasic (984 mg, 4.63 mmol) in dioxane (4 ml) and water (0.8 ml) stirred in a screw-capped pressure vessel for 16 h at 90° C. The reaction was allowed to cool, diluted with EtOAc, and the organic layer was washed with brine and dried (MgSO4). The crude product was charged (DCM) to a 40 g ISCO silica gel cartridge and gradient elution (0-50% EtOAc/hexanes) using an Isolera chromatography station gave (S)-isopropyl 2-(5-(4-(benzylcarbamoyl)phenyl)-4-(4-methoxy-4-methylpiperidin-1-yl)-2,6-dimethylpyridin-3-yl)-2-(tert-butoxy)acetate 316 mg (83%) as a mixture of diastereomers. ¹H NMR (500 MHz, DMSO-d6) δ 9.17-9.16 (m, 1H), 8.02-7.97 (m, 2H), 7.49-7.43 (m, 1H), 7.34 (s, 4H), 7.26-7.21 (m, 2H), 5.97 (br. s, 1H), 4.97-4.94 (m, 1H), 4.52 (d, J=5.1 Hz, 2H), 3.45-3.39 (m, 2H), 3.29-3.27/2.92-2.82 (m, 1H), 3.05/2.79 (s, 3H), 2.76-2.71 (m, 1H), 2.46 (s, 3H), 2.05 (s, 3H), 1.63-1.25 (m, 4H), 1.18 (d, J=6.2 Hz, 3H), 1.14-1.12 (m, 12H), 0.99/0.79 (s, 3H). UPLC (M+H)=616.6.

### EXAMPLE 60

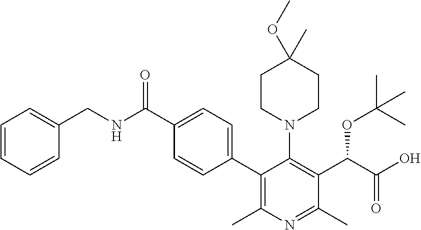 [SMILES: COC1(C)CCN(c2c(-c3ccc(C(=O)NCc4ccccc4)cc3)c(C)nc(C)c2[C@H](OC(C)(C)C)C(=O)O)CC1]

(S)-2-(5-(4-(Benzylcarbamoyl)phenyl)-4-(4-methoxy-4-methylpiperidin-1-yl)-2,6-dimethylpyridin-3-yl)-2-(tert-butoxy)acetic acid: The potassium hydroxide (227 mg, 4.0 mmol) was added to a solution of (S)-isopropyl 2-(5-(4-(benzylcarbamoyl)phenyl)-4-(4-methoxy-4-methylpiperidin-1-yl)-2,6-dimethylpyridin-3-yl)-2-(tert-butoxy)acetate (248 mg, 0.40 mmol) in ethanol (4 mL) and stirred for 6 h at 90° C. The reaction mixture was neutralized with 1N HCl soln, extracted with EtOAc, and the organic layer was washed with brine, and dried (MgSO4). The crude material was purified by prep HPLC (XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 25-65% B over 15 minutes, then a 5-minute hold at 100% B; Flow: 20 mL/min. There was obtained (S)-2-(5-(4-(benzylcarbamoyl)phenyl)-4-(4-methoxy-4-methylpiperidin-1-yl)-2,6-dimethylpyridin-3-yl)-2-(tert-butoxy)acetic acid 48 mg (21%) and recovered starting, both as a mixture of diastereomers. ¹H NMR (500 MHz, DMSO) δ 9.16-9.15 (m, 1H), 8.02 (d, J=8.1 Hz, 1H), 7.98 (d, J=7.0 Hz, 1H), 7.49-7.44 (m, 1H), 7.34 (s, 4H), 7.27-7.21 (m, 2H), 5.82/5.79 (s, 1H), 4.52 (d, J=5.5 Hz, 2H), m, 2H), 3.40 (br. s, 2H), 3.19-3.18 (m, 1H), 3.05/2.79 (s, 3H), 2.91-2.86 (m, 1H), 2.47 (s, 3H), 2.05 (s, 3H), 1.83-1.77/1.68-1.62 (m, 1H), 1.60-1.41 (m, 2H), 1.32-1.22 (m, 1H), 1.14 (br. s, 9H), 0.99/0.79 (s, 3H). UPLC (M+H)=574.6.

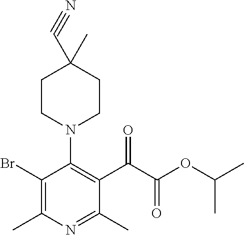 [SMILES: Cc1nc(C)c(C(=O)C(=O)OC(C)C)c(N2CCC(C)(C#N)CC2)c1Br]

Isopropyl 2-(5-bromo-4-(4-cyano-4-methylpiperidin-1-yl)-2,6-dimethylpyridin-3-yl)-2-oxoacetate: To a solution of 4-methylpiperidine-4-carbonitrile, HCl (1.0 g, 6.22 mmol) and DIEA (4.4 mL, 24.9 mmol) in anhydrous CH₃CN (30 mL) was added isopropyl 2-(5-bromo-4-chloro-2,6-dimethylpyridin-3-yl)-2-oxoacetate (2.1 g, 6.22 mmol) at rt. The resulting mixture was placed in a pre-heated oil bath (80° C.) and stirred for 72 h; cooled, diluted with ether, washed with water, brine, and dried (MgSO₄). The crude product was charged (DCM) to a 80 g ISCO silica gel cartridge and gradient eluted (5-35% EtOAc/hexanes) using an Isolera chromatography station to give isopropyl 2-(5-bromo-4-(4-cyano-4-methylpiperidin-1-yl)-2,6-dimethylpyridin-3-yl)-2-oxoacetate 1.0 g (38%). ¹H NMR (500 MHz, DMSO-d₆) δ 5.08-5.03 (m, 1H), 3.54-3.40 (m, 4H), 2.61 (s, 3H), 2.30 (s, 3H), 1.92 (d, J=12.4 Hz, 2H), (2H missing), 1.35 (s, 3H), 1.28 (d, J=5.9 Hz, 6H). UPLC (M+H)=424.3.

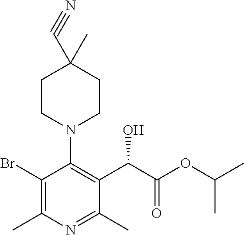 [SMILES: Cc1nc(C)c([C@H](O)C(=O)OC(C)C)c(N2CCC(C)(C#N)CC2)c1Br]

(S)-Isopropyl 2-(5-bromo-4-(4-cyano-4-methylpiperidin-1-yl)-2,6-dimethylpyridin-3-yl)-2-hydroxyacetate: The 1.55 mL of benzo[d][1,3,2]dioxaborole (443 mg, 3.69 mmol) was added to a nitrogen purged solution of isopropyl 2-(5-bromo-4-(4-cyano-4-methylpiperidin-1-yl)-2,6-dimethylpyridin-3-yl)-2-oxoacetate (780 mg, 1.85 mmol) and 0.74 mL of (R)-1-methyl-3,3-diphenylhexahydropyrrolo[1,2-c][1,3,2]oxazaborole (205 mg, 0.74 mmol) in toluene (18 mL) at −60° C. and allowed to warm to −15° C. before being placed in the freezer overnight. The reaction was quenched with 1M Na₂CO₃, diluted with EtOAc, and stirred for 30 min. The organic layer was washed with sat'd Na₂CO₃ solution, brine, and dried (MgSO₄). The crude product was charged (DCM) to a 80 g ISCO silica gel cartridge and gradient elution (0-30% EtOAc/hexanes) using an Isolera chromatography station gave (S)-isopropyl 2-(5-bromo-4-(4-cyano-4-methylpiperidin-1-yl)-2,6-dimethylpyridin-3-yl)-2-hydroxyacetate 674 mg (86%) as a mixture of diastereomers. ¹H NMR (500 MHz, CD₃OD) δ 5.89 (s, 1H), 5.10-5.05 (m, 1H), 4.13-4.05 (m, 1H), 3.78 (dt, J=11.7, 3.7 Hz, 1H), 3.06 (d, J=12.2 Hz, 1H), 2.86 (d, J=12.2 Hz, 1H), 2.63 (s, 3H), 2.51 (s, 3H), 1.94-1.86 (m, 3H), 1.79-1.73 (m, 1H), 1.46 (s, 3H), 1.24 (d, J=6.1 Hz, 3H), 1.15 (d, J=6.2 Hz, 3H). UPLC (M+H)=426.3.

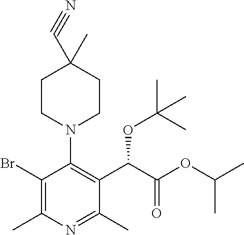 [SMILES: Cc1nc(C)c([C@H](OC(C)(C)C)C(=O)OC(C)C)c(N2CCC(C)(C#N)CC2)c1Br]

(S)-Isopropyl 2-(5-bromo-4-(4-cyano-4-methylpiperidin-1-yl)-2,6-dimethylpyridin-3-yl)-2-(tert-butoxy)acetate: The isobutylene gas was bubbled into a nitrogen purged, cooled (0° C.) solution of (S)-isopropyl 2-(5-bromo-4-(4-cyano-4-methylpiperidin-1-yl)-2,6-dimethylpyridin-3-yl)-2-hydroxyacetate (670 mg, 1.58 mmol) and 0.15 mL of 70% HClO₄ in DCM (15 mL) for 20 min. The reaction mixture was allowed to warm to rt and stirred for 48 h in a pressure sealed vessel. The reaction was then diluted with DCM, washed with 1M Na₂CO₃ solution, and dried over MgSO₄. The crude product was charged (DCM) to a 80 g ISCO silica gel cartridge and gradient elution (0-50% EtOAc/hexanes) using an Isolera chromatography station gave (S)-isopropyl 2-(5-bromo-4-(4-cyano-4-methylpiperidin-1-yl)-2,6-dimethylpyridin-3-yl)-2-(tert-butoxy)acetate 651 mg (86%) as a mixture of diastereomers. ¹H NMR (500 MHz, DMSO-d₆) δ 6.06 (br. s, 1H), 4.93-4.89 (m, 1H), 4.03 (br. s, 1H), 3.43-3.41 (m, 2H), 3.00 (br. s, 1H), 2.53 (s, 3H), 2.43 (s, 3H), 2.05 (br. s, 1H), 1.93 (br. s, 1H), 1.80-1.71 (m, 1H), 1.58 (br. s, 1H), 1.42 (s, 3H), 1.15-1.13 (m, 12H), 1.05 (d, J=6.2 Hz, 3H). UPLC (M+H)=482.4.

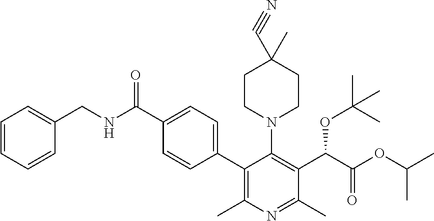 [SMILES: Cc1nc(C)c([C@H](OC(C)(C)C)C(=O)OC(C)C)c(N2CCC(C)(C#N)CC2)c1-c1ccc(C(=O)NCc2ccccc2)cc1]

(S)-Isopropyl 2-(5-(4-(benzylcarbamoyl)phenyl)-4-(4-cyano-4-methylpiperidin-1-yl)-2,6-dimethylpyridin-3-yl)-2-(tert-butoxy)acetate: Tetrakis (144 mg, 0.125 mmol) was added to an argon-degassed solution of (S)-isopropyl 2-(5-bromo-4-(4-cyano-4-methylpiperidin-1-yl)-2,6-dimethylpyridin-3-yl)-2-(tert-butoxy)acetate (300 mg, 0.624 mmol), (4-(benzylcarbamoyl)phenyl)boronic acid (175 mg, 0.687 mmol), potassium phosphate tribasic (994 mg, 4.68 mmol) in dioxane (4 ml) and water (0.8 ml) stirred in a screw-capped pressure vessel for 16 h at 90° C. The reaction was allowed to cool, diluted with EtOAc, and the organic layer was washed with brine and dried (MgSO4). The crude product was charged (DCM) to a 40 g ISCO silica gel cartridge and gradient elution (0-100% EtOAc/hexanes) using an Isolera chromatography station gave (S)-isopropyl 2-(5-(4-(benzylcarbamoyl)phenyl)-4-(4-cyano-4-methylpiperidin-1-yl)-2,6-dimethylpyridin-3-yl)-2-(tert-butoxy)acetate 351 mg (92%) as a mixture of diastereomers. ¹H NMR (500 MHz, DMSO-d6) δ 9.13-9.11 (m, 1H), 8.04-8.00 (m, 2H), 7.48-7.46 (m, 1H), 7.34-7.29 (m, 5H), 7.26-7.23 (m, 1H), 5.90 (br. s, 1H), 4.95 (br. s, 1H), 4.52-4.50 (m, 2H), 3.43-3.38 (m, 3H), 2.87 (br. s, 1H), 2.48 (s, 3H), 2.04 (s, 3H), 1.80 (br. s, 1H), 1.69 (br. s, 1H), 1.56 (br. s, 1H), 1.35-1.28 (m, 1H), 1.19 (d, J=5.9 Hz, 3H), 1.16-1.12 (m, 12H). UPLC (M+H)=611.6.

### EXAMPLE 61

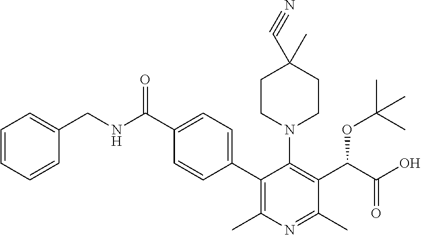 [SMILES: Cc1nc(C)c([C@H](OC(C)(C)C)C(=O)O)c(N2CCC(C)(C#N)CC2)c1-c1ccc(C(=O)NCc2ccccc2)cc1]

(S)-2-(5-(4-(Benzylcarbamoyl)phenyl)-4-(4-cyano-4-methylpiperidin-1-yl)-2,6-dimethylpyridin-3-yl)-2-(tert-butoxy)acetic acid: The potassium hydroxide (272 mg, 4.9 mmol) was added to a solution of (S)-isopropyl 2-(5-(4-(benzylcarbamoyl)phenyl)-4-(4-cyano-4-methylpiperidin-1-yl)-2,6-dimethylpyridin-3-yl)-2-(tert-butoxy)acetate (296 mg, 0.49 mmol) in ethanol (4 mL) and stirred for 6 h at 90° C. The reaction mixture was neutralized with 1N HCl soln, extracted with EtOAc, and the organic layer was washed with brine, and dried (MgSO4). The crude material was purified by prep HPLC (XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-60% B over 15 minutes, then a 5-minute hold at 100% B; Flow: 20 mL/min. There was obtained (S)-2-(5-(4-(benzylcarbamoyl)phenyl)-4-(4-cyano-4-methylpiperidin-1-yl)-2,6-dimethylpyridin-3-yl)-2-(tert-butoxy)acetic acid 183 mg (67%) as a mixture of diastereomers. ¹H NMR (500 MHz, DMSO) δ 9.13-9.11 (m, 1H), 8.03 (d, J=8.1 Hz, 1H), 8.00 (d, J=7.7 Hz, 1H), 7.46 (d, J=8.1 Hz, 1H), 7.33-7.24 (m, 6H), 5.61 (s, 1H), 4.52-4.50 (m, 2H), 3.52 (br. s, 2H), 2.84-2.80 (m, 1H), 2.50 (s, 3H), 2.46-2.43 (m, 1H), 2.04 (s, 3H), 1.96 (t, J=11.7 Hz, 1H), 1.71 (br. s, 1H), 1.61-1.58 (m, 1H), 1.41-1.36 (m, 1H), 1.26 (s, 3H), 1.14 (s, 9H). UPLC (M+H)=569.6.

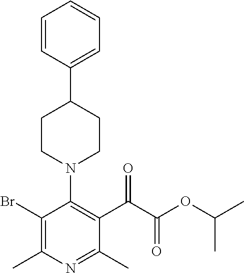 [SMILES: Cc1nc(C)c(C(=O)C(=O)OC(C)C)c(N2CCC(c3ccccc3)CC2)c1Br]

Isopropyl 2-(5-bromo-2,6-dimethyl-4-(4-phenylpiperidin-1-yl)pyridin-3-yl)-2-oxoacetate: To a solution of 4-phenylpiperidine (1.25 g, 7.75 mmol) and DIEA (4.1 mL, 23.3 mmol) in anhydrous CH₃CN (40 mL) was added isopropyl 2-(5-bromo-4-chloro-2,6-dimethylpyridin-3-yl)-2-oxoacetate (2.59 g, 7.75 mmol) at rt. The resulting mixture was placed in a pre-heated oil bath (80° C.) and stirred for 24 h; concentrated, and charged (DCM) to a 80 g ISCO silica gel cartridge and gradient eluted (5-35% EtOAc/hexanes) using an Isolera chromatography station to give isopropyl 2-(5-bromo-2,6-dimethyl-4-(4-phenylpiperidin-1-yl)pyridin-3-yl)-2-oxoacetate 2.63 g (74%). ¹H NMR (500 MHz, DMSO-d₆) δ 7.34-7.31 (m, 2H), 7.25-7.20 (m, 3H), 5.11-5.06 (m, 1H), 3.50 (br. s, 2H), 2.94 (br. s, 2H), 2.68-2.63 (m, 1H), 2.63 (s, 3H), 2.13 (s, 3H), 1.81-1.78 (m, 2H), 1.60 (br. s, 2H), 1.26 (d, J=6.2 Hz, 6H). UPLC (M+H)=461.05.

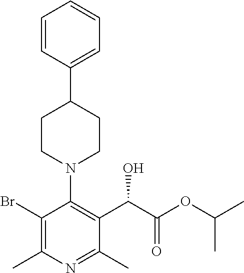 [SMILES: Cc1nc(C)c([C@H](O)C(=O)OC(C)C)c(N2CCC(c3ccccc3)CC2)c1Br]

(S)-Isopropyl 2-(5-bromo-2,6-dimethyl-4-(4-phenylpiperidin-1-yl)pyridin-3-yl)-2-hydroxyacetate: The 2.2 mL of benzo[d][1,3,2]dioxaborole (1.22 g, 10.2 mmol) was added to a nitrogen purged solution of isopropyl 2-(5-bromo-2,6-dimethyl-4-(4-phenylpiperidin-1-yl)pyridin-3-yl)-2-oxoacetate (2.6 g, 5.66 mmol) and 1.7 mL of (R)-1-methyl-3,3-diphenylhexahydropyrrolo[1,2-c][1,3,2]oxazaborole (471 mg, 1.7 mmol) in toluene (50 mL) at −60° C. and allowed to warm to −15° C. before being placed in the freezer overnight. The reaction was quenched with 1M Na₂CO₃ (15 mL), diluted with EtOAc, and stirred for 20 min. The organic layer was washed with brine and dried (MgSO₄). The crude product was charged (DCM) to a 80 g ISCO silica gel cartridge and gradient elution (5-45% EtOAc/hexanes) using an Isolera chromatography station gave (S)-isopropyl 2-(5-bromo-2,6-dimethyl-4-(4-phenylpiperidin-1-yl)pyridin-3-yl)-2-hydroxyacetate 2.0 g (71%) as a mixture of diastereomers. ¹H NMR (500 MHz, DMSO-d₆) δ 7.34-7.29 (m, 4H), 7.22-7.19 (m, 1H), 5.95 (s, 1H), 4.99-4.94 (m, 1H), 3.78-3.75 (m, 1H), 3.51-3.47 (m, 1H), 3.04-3.02 (m, 1H), 2.90-2.87 (m, 1H), 2.66-2.61 (m, 1H), 2.54 (s, 3H), 2.41 (s, 3H), 1.87-1.72 (m, 4H), 1.14 (d, J=6.2 Hz, 3H), 1.07 (d, J=5.9 Hz, 3H). UPLC (M+H)=463.2.

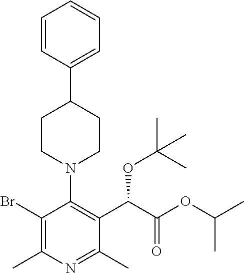 [SMILES: Cc1nc(C)c([C@H](OC(C)(C)C)C(=O)OC(C)C)c(N2CCC(c3ccccc3)CC2)c1Br]

(S)-Isopropyl 2-(5-bromo-2,6-dimethyl-4-(4-phenylpiperidin-1-yl)pyridin-3-yl)-2-(tert-butoxy)acetate: The isobutylene gas was bubbled into a nitrogen purged, cooled (0° C.) solution of (S)-isopropyl 2-(5-bromo-2,6-dimethyl-4-(4-phenylpiperidin-1-yl)pyridin-3-yl)-2-hydroxyacetate (1.95 g, 1.5 mmol) and 0.6 mL of 70% HClO₄ in DCM (25 mL) for 20 min. The reaction mixture was allowed to warm to rt and stirred for 18 h in a pressure sealed vessel, diluted with DCM, washed with 1M Na₂CO₃ solution, and dried over MgSO₄. The crude product was charged (DCM) to a 80 g ISCO silica gel cartridge and gradient elution (5-50% EtOAc/hexanes) using an Isolera chromatography station gave (S)-isopropyl 2-(5-bromo-2,6-dimethyl-4-(4-phenylpiperidin-1-yl)pyridin-3-yl)-2-(tert-butoxy)acetate 970 mg (44.4%) as a mixture of diastereomers. ¹H NMR (500 MHz, DMSO-d₆) δ 7.36-7.33 (m, 2H), 7.29-7.28 (m, 2H), 7.23-7.20 (m, 1H), 6.26 (s, 1H), 4.95-4.90 (m, 1H), 3.98 (br. s, 1H), 3.41 (br. s, 1H), 3.12 (br. s, 1H), 2.98 (br. s, 1H), 2.71 (br. s, 1H), 2.51 (s, 3H), 2.44 (s, 3H), 1.93 (br. s, 1H), 1.82-1.75 (m, 3H), 1.18 (s, 9H), 1.15 (d, J=6.2 Hz, 3H), 1.07 (d, J=5.9 Hz, 3H). UPLC (M+H)=519.2.

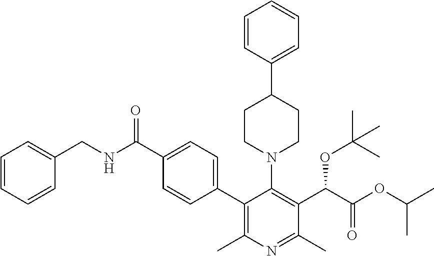 [SMILES: Cc1nc(C)c([C@H](OC(C)(C)C)C(=O)OC(C)C)c(N2CCC(c3ccccc3)CC2)c1-c1ccc(C(=O)NCc2ccccc2)cc1]

Isopropyl 2-(5-(4-(benzylcarbamoyl)phenyl)-2,6-dimethyl-4-(4-phenylpiperidin-1-yl)pyridin-3-yl)-2-(tert-butoxy)acetate: The tetrakis (33.5 mg, 0.029 mmol) was added to a nitrogen purged and degassed solution of (S)-isopropyl 2-(5-bromo-2,6-dimethyl-4-(4-phenylpiperidin-1-yl)pyridin-3-yl)-2-(tert-butoxy)acetate (150 mg, 0.29 mmol), (4-(benzylcarbamoyl)phenyl)boronic acid (81.3 mg, 0.32 mmol), and sodium carbonate (184 mg, 1.74 mmol) in dioxane (4.5 mL) and water (0.9 mL) and stirred in a screw-capped pressure vessel for 4 h at 90° C. The reaction was allowed to cool, diluted with EtOAc, and the organic layer was washed with brine and dried (Na2SO4). The crude product was charged (DCM) to a 24 g ISCO silica gel cartridge and gradient elution (5-65% EtOAc/hexanes) using an Isolera chromatography station gave (S)-isopropyl 2-(5-(4-(benzylcarbamoyl)phenyl)-2,6-dimethyl-4-(4-phenylpiperidin-1-yl)pyridin-3-yl)-2-(tert-butoxy)acetate 80 mg (44%) as a mixture of diastereomers. ¹H NMR (500 MHz, DMSO) δ 9.21-9.18 (m, 1H), 8.05 (d, J=8.1 Hz, 1H), 8.01 (d, J=7.3 Hz, 1H), 7.52 (d, J=7.7 Hz, 1H), 7.39-7.34 (m, 4H), 7.27-7.26 (m, 4H), 7.18-7.16 (m, 3H), 6.06 (s, 1H), 4.99-4.96 (m, 1H), 4.53 (d, J=5.1 Hz, 2H), 3.46 (br. s, 1H), 2.78 (br. s, 1H), 2.64 (br. s, 1H), 2.49 (s, 3H), 2.29 (br. s, 1H), 2.03 (s, 3H), 1.86 (br. s, 1H), 1.78-1.68 (m, 2H), 1.57 (br. s, 2H), 1.19-1.15 (m, 15H). UPLC (M+H)=648.5.

### EXAMPLE 62

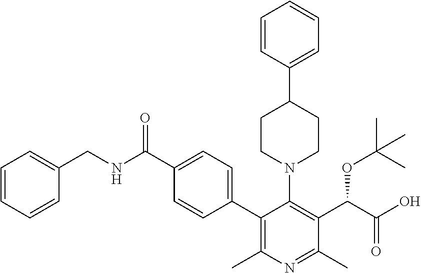 [SMILES: Cc1nc(C)c([C@H](OC(C)(C)C)C(=O)O)c(N2CCC(c3ccccc3)CC2)c1-c1ccc(C(=O)NCc2ccccc2)cc1]

(S)-2-(5-(4-(Benzylcarbamoyl)phenyl)-2,6-dimethyl-4-(4-phenylpiperidin-1-yl)pyridin-3-yl)-2-(tert-butoxy)acetic acid: The potassium hydroxide (65 mg, 1.16 mmol) was added to a solution of (S)-isopropyl 2-(5-(4-(benzylcarbamoyl)phenyl)-2,6-dimethyl-4-(4-phenylpiperidin-1-yl)pyridin-3-yl)-2-(tert-butoxy)acetate (75 mg, 0.11 mmol) in ethanol (3 mL) and stirred for 3 h at 90° C. The reaction mixture was neutralized with 1N HCl soln, extracted with EtOAc, and the organic layer was washed with brine, and dried (MgSO4). The crude material was purified by prep HPLC (XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 35-75% B over 15 minutes, then a 5-minute hold at 100% B; Flow: 20 mL/min. There was obtained (S)-2-(5-(4-(benzylcarbamoyl)phenyl)-2,6-dimethyl-4-(4-phenylpiperidin-1-yl)pyridin-3-yl)-2-(tert-butoxy)acetic acid 65.6 mg (93%) as a mixture of diastereomers. ¹H NMR (500 MHz, DMSO) δ 9.21-9.19 (m, 1H), 8.06 (d, J=7.7 Hz, 1H), 8.02 (d, J=8.1 Hz, 1H), 7.51 (d, J=8.1 Hz, 1H), 7.39-7.34 (m, 4H), 7.27-7.25 (m, 4H), 7.18-7.14 (m, 3H), 5.89 (br. s, 1H), 4.52 (s, 2H), 3.62 (d, J=10.6 Hz, 1H), 2.78-2.74 (m, 1H), 2.61 (d, J=10.6 Hz, 1H), 2.51 (s, 3H), 2.28-2.23 (m, 1H), 2.06 (s, 3H), 1.86-1.81 (m, 2H), 1.67-1.50 (m, 3H), 1.20 (s, 9H). UPLC (M+H)=606.4.

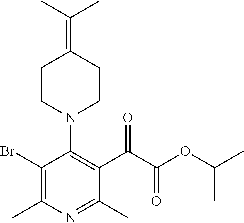 [SMILES: CC(C)=C1CCN(c2c(Br)c(C)nc(C)c2C(=O)C(=O)OC(C)C)CC1]

Isopropyl 2-(5-bromo-2,6-dimethyl-4-(4-(propan-2-ylidene)piperidin-1-yl)pyridin-3-yl)-2-oxoacetate: To a solution of 4-(propan-2-ylidene)piperidine (500 mg, 3.99 mmol) and DIEA (2.1 mL, 11.98 mmol) in anhydrous CH₃CN (30 mL) was added isopropyl 2-(5-bromo-4-chloro-2,6-dimethylpyridin-3-yl)-2-oxoacetate (1.48 g, 3.99 mmol) at rt. The resulting mixture was placed in a pre-heated oil bath (80° C.) and stirred for 24 h; cooled, diluted with ether, washed with water, brine, and dried (MgSO₄). The crude product was charged (DCM) to an 80 g ISCO silica gel cartridge and gradient eluted (0-35% EtOAc/hexanes) using an Isolera chromatography station gave isopropyl 2-(5-bromo-2,6-dimethyl-4-(4-(propan-2-ylidene)piperidin-1-yl)pyridin-3-yl)-2-oxoacetate 1.04 g (61.8%). ¹H NMR (500 MHz, DMSO-d₆) δ 5.09-5.04 (m, 1H), 3.50-3.48 (br. s, 2H), 3.03 (br. s, 2H), 2.60 (s, 3H), 2.30 (s, 3H), 2.23 (br. s, 4), 1.64 (s, 6H), 1.25 (d, J=6.2 Hz, 6H). UPLC (M+H)=425.3.

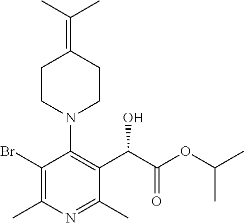 [SMILES: CC(C)=C1CCN(c2c(Br)c(C)nc(C)c2[C@H](O)C(=O)OC(C)C)CC1]

(S)-Isopropyl 2-(5-bromo-2,6-dimethyl-4-(4-(propan-2-ylidene)piperidin-1-yl)pyridin-3-yl)-2-hydroxyacetate: The 1.0 mL of benzo[d][1,3,2]dioxaborole (567 mg, 4.72 mmol) was added to a nitrogen purged solution of isopropyl 2-(5-bromo-2,6-dimethyl-4-(4-(propan-2-ylidene)piperidin-1-yl)pyridin-3-yl)-2-oxoacetate (1.0 g, 2.36 mmol) and 0.95 mL of (R)-1-methyl-3,3-diphenylhexahydropyrrolo[1,2-c][1,3,2]oxazaborole (262 mg, 0.95 mmol) in toluene (20 mL) at −60° C. and allowed to warm to −15° C. before being placed in the freezer overnight. The reaction was quenched with 1M Na₂CO₃, diluted with EtOAc, and stirred for 30 min. The organic layer was washed with sat'd Na₂CO₃ solution, brine and dried (MgSO₄). The crude product was charged (DCM) to a 80 g ISCO silica gel cartridge and gradient elution (0-60% EtOAc/hexanes) using an Isolera chromatography station gave (S)-isopropyl 2-(5-bromo-2,6-dimethyl-4-(4-(propan-2-ylidene)piperidin-1-yl)pyridin-3-yl)-2-hydroxyacetate 1.0 g (100%). ¹H NMR (500 MHz, DMSO-d₆) δ 5.90 (d, J=3.3 Hz, 1H), 4.95 (dt, J=12.3, 6.2 Hz, 1H), 3.43-3.41 (m, 2H), 3.23 (t, J=10.6 Hz, 1H), 2.86 (d, J=10.3 Hz, 1H), 2.56-2.51 (m, 2H), 2.51 (s, 3H), 2.38 (s, 3H), 2.16-2.08 (m, 2H), 1.67 (s, 6H), 1.15 (d, J=6.2 Hz, 3H), 1.08 (d, J=6.2 Hz, 3H). UPLC (M+H)=427.2.

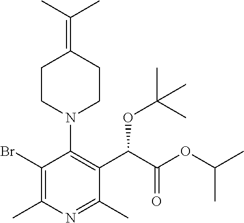 [SMILES: CC(C)=C1CCN(c2c(Br)c(C)nc(C)c2[C@H](OC(C)(C)C)C(=O)OC(C)C)CC1]

(S)-Isopropyl 2-(5-bromo-2,6-dimethyl-4-(4-(propan-2-ylidene)piperidin-1-yl)pyridin-3-yl)-2-(tert-butoxy)acetate: The isobutylene gas was bubbled into a nitrogen purged, cooled (0° C.) solution of (S)-isopropyl 2-(5-bromo-2,6-dimethyl-4-(4-(propan-2-ylidene)piperidin-1-yl)pyridin-3-yl)-2-hydroxyacetate (1.0 g, 2.3 mmol) and 0.30 mL of 70% HClO₄ in DCM (30 mL) for 20 min. The reaction mixture was allowed to warm to rt and stirred for 48 h in a pressure sealed vessel. The reaction was then diluted with DCM, washed with 1M Na₂CO₃ solution, and dried over MgSO₄. The crude product was charged (DCM) to a 80 g ISCO silica gel cartridge and gradient elution (0-50% EtOAc/hexanes) using an Isolera chromatography station gave (S)-isopropyl 2-(5-bromo-2,6-dimethyl-4-(4-(propan-2-ylidene)piperidin-1-yl)pyridin-3-yl)-2-(tert-butoxy)acetate 1.0 g (67%). ¹H NMR (500 MHz, DMSO-d₆) δ 6.24 (br. s, 1H), 4.95-4.90 (m, 1H), 3.68 (br. s, 1H), 3.18-3.12 (m, 1H), 3.06 (br. s, 1H), 2.83 (br. s, 1H), 2.72 (br. s, 1H), 2.59 (br. s, 1H), 2.51 (s, 3H), 2.43 (s, 3H), 2.13-2.01 (m, 2H), 1.68 (s, 6H), 1.17-1.15 (m, 12H), 1.08 (d, J=6.2 Hz, 3H). UPLC (M+H)=483.2.

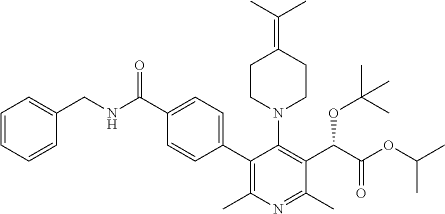 [SMILES: CC(C)=C1CCN(c2c(-c3ccc(C(=O)NCc4ccccc4)cc3)c(C)nc(C)c2[C@H](OC(C)(C)C)C(=O)OC(C)C)CC1]

(S)-Isopropyl 2-(5-(4-(benzylcarbamoyl)phenyl)-2,6-dimethyl-4-(4-(propan-2-ylidene)piperidin-1-yl)pyridin-3-yl)-2-(tert-butoxy)acetate: The palladium tetrakis (122 mg, 0.11 mmol) was added to a argon purged and degassed solution of (S)-isopropyl 2-(5-bromo-2,6-dimethyl-4-(4-(propan-2-ylidene)piperidin-1-yl)pyridin-3-yl)-2-(tert-butoxy)acetate (255 mg, 0.53 mmol), (4-(benzylcarbamoyl)phenyl)boronic acid (149 mg, 0.583 mmol), and potassium phosphate tribasic (843 mg, 3.97 mmol) in dioxane (4 mL) and water (0.8 mL) and stirred in a screw-capped pressure vessel for 16 h at 90° C. The reaction was allowed to cool, diluted with EtOAc, and the organic layer was washed with brine and dried (MgSO4). The crude product was charged (DCM) to a 40 g ISCO silica gel cartridge and gradient elution (0-70% EtOAc/hexanes) using an Isolera chromatography station gave (S)-isopropyl 2-(5-(4-(benzylcarbamoyl)phenyl)-2,6-dimethyl-4-(4-(propan-2-ylidene)piperidin-1-yl)pyridin-3-yl)-2-(tert-butoxy)acetate 276 mg (85%). ¹H NMR (500 MHz, DMSO) δ 9.18-9.15 (m, 1H), 7.98-7.95 (m, 2H), 7.48 (d, J=7.3 Hz, 1H), 7.35-7.33 (m, 4H), 7.27-7.25 (m, 1H), 7.16 (d, J=7.7 Hz, 1H), 6.03 (s, 1H), 5.01-4.96 (m, 1H), 4.51 (d, J=5.9 Hz, 2H), 3.35 (br. s, 2H), 2.55 (br. s, 2H), 2.48 (s, 3H), 2.32 (br. s, 2H), 2.03 (s, 3H), 1.84 (br. s, 2H), 1.53 (br. s, 6H), 1.22 (d, J=6.2 Hz, 3H), 1.17 (d, J=6.2 Hz, 3H), 1.15 (s, 9H). UPLC (M+H)=612.4.

### EXAMPLE 63

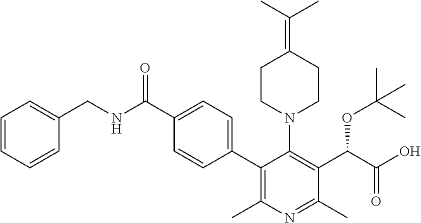 [SMILES: CC(C)=C1CCN(c2c(-c3ccc(C(=O)NCc4ccccc4)cc3)c(C)nc(C)c2[C@H](OC(C)(C)C)C(=O)O)CC1]

(S)-2-(5-(4-(Benzylcarbamoyl)phenyl)-2,6-dimethyl-4-(4-(propan-2-ylidene)piperidin-1-yl)pyridin-3-yl)-2-(tert-butoxy)acetic acid: The potassium hydroxide (73 mg, 1.31 mmol) was added to a solution of (S)-isopropyl 2-(5-(4-(benzylcarbamoyl)phenyl)-2,6-dimethyl-4-(4-(propan-2-ylidene)piperidin-1-yl)pyridin-3-yl)-2-(tert-butoxy)acetate (80 mg, 0.131 mmol) in ethanol (2 mL) and stirred for 6 h at 90° C. The reaction mixture was neutralized with 1N HCl soln, extracted with EtOAc, and the organic layer was washed with brine, and dried (MgSO4). The crude material was purified by prep HPLC (XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 25-65% B over 15 minutes, then a 5-minute hold at 100% B; Flow: 20 mL/min. There was obtained (S)-2-(5-(4-(benzylcarbamoyl)phenyl)-2,6-dimethyl-4-(4-(propan-2-ylidene)piperidin-1-yl)pyridin-3-yl)-2-(tert-butoxy)acetic acid 50.5 mg (65%). ¹H NMR (500 MHz, DMSO) δ 9.18-9.15 (m, 1H), 7.97-7.95 (m, 2H), 7.47 (d, J=7.7 Hz, 1H), 7.35-7.33 (m, 4H), 7.27-7.24 (m, 1H), 7.14 (d, J=8.4 Hz, 1H), 5.86 (s, 1H), 4.51 (t, J=5.9 Hz, 2H), 3.61 (br. s, 1H), 3.39 (br. s, 2H), 2.57-2.55 (m, 1H), 2.48 (s, 3H), 2.31-2.28 (m, 1H), 2.08 (br. s, 1H), 2.02 (s, 3H), 1.86-1.82 (m, 1H), 1.63-1.59 (m, 1H), 1.56 (s, 3H), 1.49 (s, 3H), 1.15 (s, 9H). UPLC (M+H)=570.4.

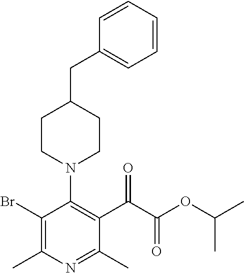 [SMILES: Cc1nc(C)c(C(=O)C(=O)OC(C)C)c(N2CCC(Cc3ccccc3)CC2)c1Br]

Isopropyl 2-(4-(4-benzylpiperidin-1-yl)-5-bromo-2,6-dimethylpyridin-3-yl)-2-oxoacetate: To a solution of 4-benzylpiperidine (0.80 g, 4.56 mmol) and DIEA (2.4 mL, 13.7 mmol) in anhydrous CH₃CN (30 mL) was added isopropyl 2-(5-bromo-4-chloro-2,6-dimethylpyridin-3-yl)-2-oxoacetate (1.7 g, 4.56 mmol) at rt. The resulting mixture was placed in a pre-heated oil bath (80° C.) and stirred for 18 h; cooled, diluted with ether, washed with water, brine, and dried (MgSO₄). The crude product was charged (DCM) to a 80 g ISCO silica gel cartridge and gradient eluted (0-25% EtOAc/hexanes) using an Isolera chromatography station gave isopropyl 2-(4-(4-benzylpiperidin-1-yl)-5-bromo-2,6-dimethylpyridin-3-yl)-2-oxoacetate 1.48 g (68%). ¹H NMR (500 MHz, DMSO-d₆) δ 7.3-7.27 (m, 2H), 7.20-7.15 (m, 3H), 5.09-5.04 (m, 1H), 3.23 (br. s, 2H), 2.84 (br. s, 2H), 2.59 (s, 3H), 2.51-2.49 (m, 2H), 2.28 (s, 3H), 1.61 (br. s, 1), 1.55 (d, J=13.6 Hz, 2H), 1.30 (d, J=6.2 Hz, 6H), 1.14 (br. s, 2H). UPLC (M+H)=475.2.

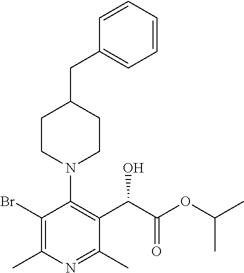 [SMILES: Cc1nc(C)c([C@H](O)C(=O)OC(C)C)c(N2CCC(Cc3ccccc3)CC2)c1Br]

(S)-Isopropyl 2-(4-(4-benzylpiperidin-1-yl)-5-bromo-2,6-dimethylpyridin-3-yl)-2-hydroxyacetate: The 1.3 mL of benzo[d][1,3,2]dioxaborole (709 mg, 5.91 mmol) was added to a nitrogen purged solution of isopropyl 2-(4-(4-benzylpiperidin-1-yl)-5-bromo-2,6-dimethylpyridin-3-yl)-2-oxoacetate (1.4 g, 2.96 mmol) and 1.2 mL of (R)-1-methyl-3,3-diphenylhexahydropyrrolo[1,2-c][1,3,2]oxazaborole (328 mg, 1.18 mmol) in toluene (30 mL) at −60° C. and allowed to warm to −15° C. before being placed in the freezer overnight. The reaction was quenched with 1M Na₂CO₃, diluted with EtOAc, and stirred for 30 min. The organic layer was washed with sat'd Na₂CO₃ solution, brine and dried (MgSO₄). The crude product was charged (DCM) to a 80 g ISCO silica gel cartridge and gradient elution (0-50% EtOAc/hexanes) using an Isolera chromatography station gave (S)-isopropyl 2-(4-(4-benzylpiperidin-1-yl)-5-bromo-2,6-dimethylpyridin-3-yl)-2-hydroxyacetate as a mixture of diastereomers 1.4 g (100%). ¹H NMR (500 MHz, DMSO-d₆) δ 7.30-7.27 (m, 2H), 7.19-7.18 (m, 3H), 5.95/5.93 (s, 1H), 4.97-4.93 (m, 1H), 3.53 (t, J=11.4 Hz, 1H), 3.40-3.38 (m, 1H), 3.31 (t, J=11.7 Hz, 1H), 3.25 (br. s, 1H), 2.87 (d, J=10.3 Hz, 1H), 2.77 (d, J=10.6 Hz, 1H), 2.55/2.54 (s, 3H), 2.38 (s, 3H), 1.60-1.52 (m, 3H), 1.38-1.63 (m, 2H), 1.14 (d, J=5.9 Hz, 3H), 1.05 (d, J=5.9 Hz, 3H). UPLC (M+H)=477.1.

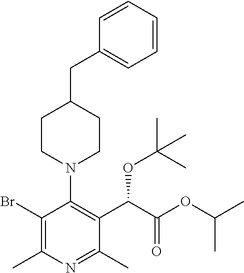 [SMILES: Cc1nc(C)c([C@H](OC(C)(C)C)C(=O)OC(C)C)c(N2CCC(Cc3ccccc3)CC2)c1Br]

(S)-Isopropyl 2-(4-(4-benzylpiperidin-1-yl)-5-bromo-2,6-dimethylpyridin-3-yl)-2-(tert-butoxy)acetate: The isobutylene gas was bubbled into a nitrogen purged, cooled (0° C.) solution of (S)-isopropyl 2-(4-(4-benzylpiperidin-1-yl)-5-bromo-2,6-dimethylpyridin-3-yl)-2-hydroxyacetate (1.35 g, 2.84 mmol) and 0.27 mL of 70% HClO₄ in DCM (25 mL) for 20 min. The reaction mixture was allowed to warm to rt and stirred for 48 h in a pressure sealed vessel. The reaction was then diluted with DCM, washed with 1M Na₂CO₃ solution, and dried over MgSO₄. The crude product was charged (DCM) to a 80 g ISCO silica gel cartridge and gradient elution (0-25% EtOAc/hexanes) using an Isolera chromatography station gave (S)-isopropyl 2-(4-(4-benzylpiperidin-1-yl)-5-bromo-2,6-dimethylpyridin-3-yl)-2-(tert-butoxy)acetate 1.27 g (84%) as a mixture of diastereomers. ¹H NMR (500 MHz, DMSO-d₆) δ 7.30-7.27 (m, 2H), 7.20-7.18 (m, 3H), 6.12 (br. s, 1H), 4.93-4.88 (m, 1H), 3.79-3.74 (m, 1H), 3.20 (t, J=12.5 Hz, 1H), 2.97-2.95 (m, 1H), 2.74 (br. s, 1H), 2.60 (d, J=5.1 Hz, 2H), 2.51 (s, 3H), 2.40 (s, 3H), 1.68 (br. s, 2H), 1.60-1.57 (m, 1H), 1.36-1.29 (m, 2H), 1.18 (d, J=6.2 Hz, 3H), 1.11 (s, 9H), 1.06 (d, J=6.2 Hz, 3H). UPLC (M+H)=533.3.

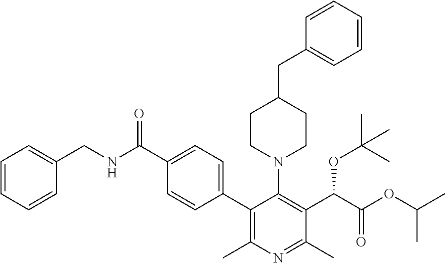 [SMILES: Cc1nc(C)c([C@H](OC(C)(C)C)C(=O)OC(C)C)c(N2CCC(Cc3ccccc3)CC2)c1-c1ccc(C(=O)NCc2ccccc2)cc1]

(S)-Isopropyl 2-(5-(4-(benzylcarbamoyl)phenyl)-4-(4-benzylpiperidin-1-yl)-2,6-dimethylpyridin-3-yl)-2-(tert-butoxy)acetate: The palladium tetrakis (122 mg, 0.11 mmol) was added to a argon purged and degassed solution of (S)-isopropyl 2-(4-(4-benzylpiperidin-1-yl)-5-bromo-2,6-dimethylpyridin-3-yl)-2-(tert-butoxy)acetate (267 mg, 0.502 mmol), (4-(benzylcarbamoyl)phenyl)boronic acid (148 mg, 0.582 mmol), and potassium phosphate tribasic (842 mg, 3.97 mmol) in dioxane (4 mL) and water (0.8 mL) and stirred in a screw-capped pressure vessel for 16 h at 90° C. The reaction was allowed to cool, diluted with EtOAc, and the organic layer was washed with brine and dried (MgSO4). The crude product was charged (DCM) to a 40 g ISCO silica gel cartridge and gradient elution (0-70% EtOAc/hexanes) using an Isolera chromatography station gave (S)-isopropyl 2-(5-(4-(benzylcarbamoyl)phenyl)-4-(4-benzylpiperidin-1-yl)-2,6-dimethylpyridin-3-yl)-2-(tert-butoxy)acetate 282.6 mg (81%) as a mixture of diastereomers. ¹H NMR (500 MHz, DMSO) δ 9.16-9.13 (m, 1H), 7.96 (t, J=8.8 Hz, 2H), 7.45 (d, J=7.3 Hz, 1H), 7.36-7.33 (m, 4H), 7.28-7.21 (m, 3H), 7.18-7.13 (m, 2H), 7.09 (d, J=7.0 Hz, 2H), 5.90 (s, 1H), 4.98-4.93 (m, 1H), 4.51 (d, J=5.9 Hz, 2H), 3.39 (br. s, 2H), 3.30-3.27 (m, 1H), 2.58-2.53 (m, 1H), 2.45 (br. s, 5H), 2.02 (s, 3H), 1.64 (t, J=12.1 Hz, 1H), 1.44-1.42 (m, 1H), 1.36-1.33 (m, 1H), 1.27-1.22 (m, 2H), 1.19 (d, J=6.2 Hz, 3H), 1.15 (d, J=6.2 Hz, 3H), 1.09 (m, 9H). UPLC (M+H)=662.4.

### EXAMPLE 64

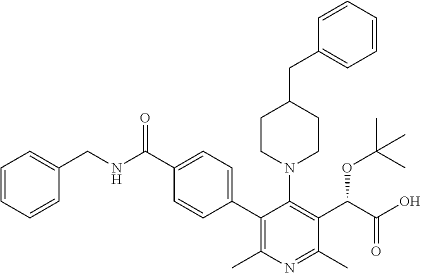 [SMILES: Cc1nc(C)c([C@H](OC(C)(C)C)C(=O)O)c(N2CCC(Cc3ccccc3)CC2)c1-c1ccc(C(=O)NCc2ccccc2)cc1]

(S)-2-(5-(4-(Benzylcarbamoyl)phenyl)-4-(4-benzylpiperidin-1-yl)-2,6-dimethylpyridin-3-yl)-2-(tert-butoxy)acetic acid: The potassium hydroxide (85 mg, 1.51 mmol) was added to a solution of (S)-isopropyl 2-(5-(4-(benzylcarbamoyl)phenyl)-4-(4-benzylpiperidin-1-yl)-2,6-dimethylpyridin-3-yl)-2-(tert-butoxy)acetate (100 mg, 0.151 mmol) in ethanol (4 mL) and stirred for 12 h at 90° C. The reaction mixture was neutralized with 1N HCl soln, extracted with EtOAc, and the organic layer was washed with brine, and dried (MgSO4). The crude material was purified by prep HPLC (XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 40-80% B over 15 minutes, then a 5-minute hold at 100% B; Flow: 20 mL/min. There was obtained (S)-2-(5-(4-(benzylcarbamoyl)phenyl)-4-(4-benzylpiperidin-1-yl)-2,6-dimethylpyridin-3-yl)-2-(tert-butoxy)acetic acid 68.4 mg (73%) as a mixture of diastereomers. ¹H NMR (500 MHz, DMSO) δ 9.16-9.14 (m, 1H), 7.96 (t, J=8.8 Hz, 2H), 7.45 (d, J=7.3 Hz, 1H), 7.37-7.33 (m, 4H), 7.28-7.21 (m, 3H), 7.16-7.13 (m, 2H), 7.09 (d, J=7.3 Hz, 2H), 5.77 (s, 1H), 4.51 (d, J=5.9 Hz, 2H), 3.52-3.50 (m, 2H), 3.44 (br. s, 2H), 2.47-2.42 (m, 5H), 2.02 (s, 3H), 1.60 (t, J=12.1 Hz, 1H), 1.43-1.41 (m, 1H), 1.33-1.18 (m, 3H), 1.10 (m, 9H). UPLC (M+H)=620.4.

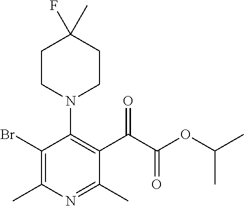 [SMILES: Cc1nc(C)c(C(=O)C(=O)OC(C)C)c(N2CCC(C)(F)CC2)c1Br]

Isopropyl 2-(5-bromo-4-(4-fluoro-4-methylpiperidin-1-yl)-2,6-dimethylpyridin-3-yl)-2-oxoacetate: To a solution 4-fluoro-4-methylpiperidine, HCl (0.50 g, 3.25 mmol) and DIEA (2.3 mL, 13.0 mmol) in anhydrous CH₃CN (30 mL) was added isopropyl 2-(5-bromo-4-chloro-2,6-dimethylpyridin-3-yl)-2-oxoacetate (1.2 g, 3.25 mmol) at rt. The resulting mixture was placed in a pre-heated oil bath (80° C.) and stirred for 24 h; cooled, diluted with ether, washed with water, brine, and dried (MgSO₄). The crude product was charged (DCM) to an 80 g ISCO silica gel cartridge and gradient eluted (0-25% EtOAc/hexanes) using an Isolera chromatography station to give isopropyl 2-(5-bromo-4-(4-fluoro-4-methylpiperidin-1-yl)-2,6-dimethylpyridin-3-yl)-2-oxoacetate 678 mg (50%). ¹H NMR (500 MHz, CDCl₃) δ 5.09-5.05 (m, 1H), 3.34 (br. s, 2H), 2.85 (br. s., 2H), 2.62 (s, 3H), 2.31 (s, 3H), 1.81-1.77 (m 2H), 1.66-1.57 (m, 2H), 1.37/1.33 (s, 3H), 1.28 (d, J=6.2 Hz, 6H). UPLC (M+H)=417.1.

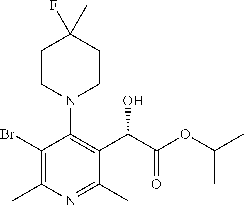 [SMILES: Cc1nc(C)c([C@H](O)C(=O)OC(C)C)c(N2CCC(C)(F)CC2)c1Br]

(S)-Isopropyl 2-(5-bromo-4-(4-fluoro-4-methylpiperidin-1-yl)-2,6-dimethylpyridin-3-yl)-2-hydroxyacetate: The 0.67 mL of benzo[d][1,3,2]dioxaborole (751 mg, 3.13 mmol) was added to a nitrogen purged solution of isopropyl 2-(5-bromo-4-(4-fluoro-4-methylpiperidin-1-yl)-2,6-dimethylpyridin-3-yl)-2-oxoacetate (650 mg, 1.57 mmol) and 0.63 mL of (R)-1-methyl-3,3-diphenylhexahydropyrrolo[1,2-c][1,3,2]oxazaborole (174 mg, 0.63 mmol) in toluene (15 mL) at −60° C. and allowed to warm to −15° C. before being placed in the freezer overnight. The reaction was quenched with 1M Na₂CO₃, diluted with EtOAc, and stirred for 30 min. The organic layer was washed with sat'd Na₂CO₃ solution, brine and dried (MgSO₄). The crude product was charged (DCM) to a 80 g ISCO silica gel cartridge and gradient elution (0-50% EtOAc/hexanes) using an Isolera chromatography station gave (S)-isopropyl 2-(5-bromo-4-(4-fluoro-4-methylpiperidin-1-yl)-2,6-dimethylpyridin-3-yl)-2-hydroxyacetate 620 mg (95%) as a mixture of diastereomers. ¹H NMR (500 MHz, DMSO-d₆) δ 5.88/5.86 (s, 1H), 4.95-4.93 (m, 1H), 3.85-3.81 (m, 1H), 3.56 (t, J=11.4 Hz, 1H), 3.23/3.03 (br. s, 1H), 2.76-2.74/2.65-2.62 (m, 1H), 2.53 (s, 3H), 2.39 (s, 3H), 1.91-1.71 (m, 4H), 1.39/1.35 (s, 3H), 1.14 (d, J=5.9 Hz, 3H), 1.07 (d, J=6.2 Hz, 3H). UPLC (M+H)=419.1.

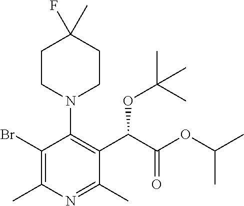 [SMILES: Cc1nc(C)c([C@H](OC(C)(C)C)C(=O)OC(C)C)c(N2CCC(C)(F)CC2)c1Br]

(S)-Isopropyl 2-(5-bromo-4-(4-fluoro-4-methylpiperidin-1-yl)-2,6-dimethylpyridin-3-yl)-2-(tert-butoxy)acetate: The isobutylene gas was bubbled into a nitrogen purged, cooled (0° C.) solution of (S)-isopropyl 2-(5-bromo-4-(4-fluoro-4-methylpiperidin-1-yl)-2,6-dimethylpyridin-3-yl)-2-hydroxyacetate (600 mg, 1.44 mmol) and 0.14 mL of 70% HClO₄ in DCM (15 mL) for 20 min. The reaction mixture was allowed to warm to rt and stirred for 5 days. The reaction was diluted with DCM, washed with 1M Na₂CO₃ solution, and dried over MgSO₄. The crude product was charged (DCM) to a 80 g ISCO silica gel cartridge and gradient elution (0-25% EtOAc/hexanes) using an Isolera chromatography station gave (S)-isopropyl 2-(5-bromo-4-(4-fluoro-4-methylpiperidin-1-yl)-2,6-dimethylpyridin-3-yl)-2-(tert-butoxy)acetate 529 mg (78%) as a mixture of diastereomers. ¹H NMR (500 MHz, DMSO-d₆) δ (br. s, 1H), 4.92-4.89 (m, 1H), 4.04 (br. s, 1H), 3.51-3.48 (m, 1H), 3.37-3.36 (m, 1H), 2.85 (br. s, 1H), 2.53 (s, 3H), 2.44 (br. s, 3H), 1.95-1.67 (m, 4H), 1.42/1.36 (s, 3H), 1.17-1.14 (m, 12H), 1.0 (d, J=6.2 Hz, 3H). UPLC (M+H)=475.2.

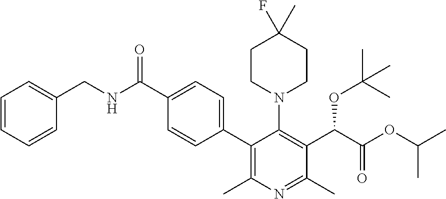 [SMILES: Cc1nc(C)c([C@H](OC(C)(C)C)C(=O)OC(C)C)c(N2CCC(C)(F)CC2)c1-c1ccc(C(=O)NCc2ccccc2)cc1]

(S)-Isopropyl 2-(5-(4-(benzylcarbamoyl)phenyl)-4-(4-fluoro-4-methylpiperidin-1-yl)-2,6-dimethylpyridin-3-yl)-2-(tert-butoxy)acetate: The palladium tetrakis (102 mg, 0.088 mmol) was added to a argon purged and degassed solution of (S)-isopropyl 2-(5-bromo-4-(4-fluoro-4-methylpiperidin-1-yl)-2,6-dimethylpyridin-3-yl)-2-(tert-butoxy)acetate (209 mg, 0.442 mmol), (4-(benzylcarbamoyl)phenyl)boronic acid (124 mg, 0.486 mmol), and potassium phosphate tribasic (702 mg, 3.31 mmol) in dioxane (4 mL) and water (0.8 mL) and stirred in a screw-capped pressure vessel for 16 h at 90° C. The reaction was allowed to cool, diluted with EtOAc, and the organic layer was washed with brine, and dried (MgSO4). The crude product was charged (DCM) to a 40 g ISCO silica gel cartridge and gradient elution (0-75% EtOAc/hexanes) using an Isolera chromatography station gave (S)-isopropyl 2-(5-(4-(benzylcarbamoyl)phenyl)-4-(4-fluoro-4-methylpiperidin-1-yl)-2,6-dimethylpyridin-3-yl)-2-(tert-butoxy)acetate 201 mg (75.3%) as a mixture of diastereomers. ¹H NMR (500 MHz, DMSO) δ 9.18-9.16 (m,1H), 7.99 (t, J=7.7 Hz, 2H), 7.48-7.46 (m, 1H), 7.36-7.34 (m, 4H), 7.26 (br. s, 2H), 5.97 (br. s, 1H), 4.96 (br. s, 1H), 4.52 (d, J=5.5 Hz, 2H), 3.37 (br. s, 1H), 3.18 (br. s, 1H), 2.87 (br. s, 1H), 2.48 (s, 3H), 2.36 (br. s, 1H), 2.03 (s, 3H), 1.84-1.46 (m, 4H), 1.27/1.23 (s, 3H), 1.20 (d, J=5.5 Hz, 3H), 1.16-1.12 (m, 12H). UPLC (M+H)=604.4.

### EXAMPLE 65

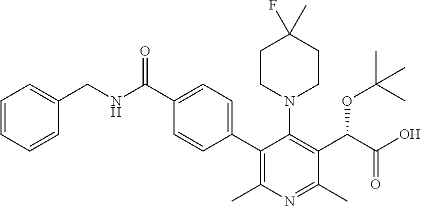 [SMILES: Cc1nc(C)c([C@H](OC(C)(C)C)C(=O)O)c(N2CCC(C)(F)CC2)c1-c1ccc(C(=O)NCc2ccccc2)cc1]

(S)-2-(5-(4-(Benzylcarbamoyl)phenyl)-4-(4-fluoro-4-methylpiperidin-1-yl)-2,6-dimethylpyridin-3-yl)-2-(tert-butoxy)acetic acid: The potassium hydroxide (162 mg, 2.90 mmol) was added to a solution of (S)-isopropyl 2-(5-(4-(benzylcarbamoyl)phenyl)-4-(4-fluoro-4-methylpiperidin-1-yl)-2,6-dimethylpyridin-3-yl)-2-(tert-butoxy)acetate (175 mg, 0.29 mmol) in ethanol (3 mL) and stirred for 4 h at 90° C. The reaction mixture was neutralized with 1N HCl soln, extracted with EtOAc, and the organic layer was washed with brine, and dried (MgSO4). The crude material was purified by prep HPLC (XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-60% B over 15 minutes, then a 5-minute hold at 100% B; Flow: 20 mL/min. There was (S)-2-(5-(4-(benzylcarbamoyl)phenyl)-4-(4-fluoro-4-methylpiperidin-1-yl)-2,6-dimethylpyridin-3-yl)-2-(tert-butoxy)acetic acid 142 mg (87%) as a mixture of diastereomers. ¹H NMR (500 MHz, DMSO) δ 9.18-9.16 (m,1H), 7.99 (t, J=8.4 Hz, 2H), 7.47 (d, J=7.3 Hz, 1H), 7.36-7.33 (m, 4H), 7.27-7.23 (m, 2H), 5.72 (br. s, 1H), 4.52 (t, J=5.5 Hz, 2H), 3.37-3.34 (m, 2H), 2.83 (t, J=12.5 Hz, 1H), 2.50 (s, 3H), 2.32-2.30 (m, 1H), 2.03 (s, 3H), 1.83-1.77 (m, 1H), 1.64-1.49 (m, 3H), 1.26/1.21 (s, 3H), 1.14 (s, 9H). UPLC (M+H)=562.3.

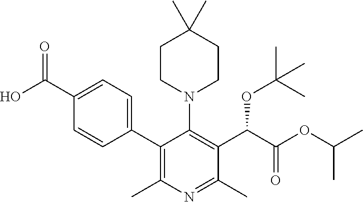 [SMILES: Cc1nc(C)c([C@H](OC(C)(C)C)C(=O)OC(C)C)c(N2CCC(C)(C)CC2)c1-c1ccc(C(=O)O)cc1]

(S)-4-(5-(1-(tert-Butoxy)-2-isopropoxy-2-oxoethyl)-4-(4,4-dimethylpiperidin-1-yl)-2,6-dimethylpyridin-3-yl)benzoic acid: The palladium tetrakis (118 mg, 0.102 mmol) was added to a nitrogen purged and degassed solution of (S)-isopropyl 2-(5-bromo-4-(4,4-dimethylpiperidin-1-yl)-2,6-dimethylpyridin-3-yl)-2-(tert-butoxy)acetate (320 mg, 0.68 mmol), 4-boronobenzoic acid (124 mg, 0.75 mmol), and sodium carbonate (433 mg, 4.09 mmol) in 1,4-dioxane (3 mL) and water (0.6 mL). The reaction mixture was stirred in a screw-capped pressure vessel for 4 h at 90° C., cooled, adjusted with 1M HCl soln to pH=2, and extracted with EtOAc. The organic layer was washed with brine and dried (Na2CO3). The crude product was charged (DCM) to a 24 g ISCO silica gel cartridge and gradient elution (5-30% MeOH/EtOAc) using an Isolera chromatography station a crude product. A second purification was performed on a Prep HPLC on WATERS-Atlantis column 30×100 mm S5 running 25 min gradient (10-100%B; MeOH/water/TFA) and gave (S)-4-(5-(1-(tert-butoxy)-2-isopropoxy-2-oxoethyl)-4-(4,4-dimethylpiperidin-1-yl)-2,6-dimethylpyridin-3-yl)benzoic acid 167 mg (48%): ¹H NMR (500 MHz, DMSO) δ 8.06 (d, J=8.1 Hz, 1H), 8.02 (d, J=8.1 Hz, 1H), 7.50 (d, J=7.7 Hz, 1H), 7.31 (d, J=7.7 Hz, 1H), 5.97 (s, 1H), 4.99-4.94 (m, 1H), 3.35 (br. s, 1H) 3.13 (br. s, 1H), 2.82 (br. s, 1H), 2.46 (s, 3H), 2.23 (br. s, 1H), 2.06 (s, 3H), 1.88-1.82 (m, 1H), 1.49-1.43 (m, 1H), 1.27-1.24 (m, 1H), 1.19 (d, J=6.2 Hz, 3H), 1.15-1.13 (m, 12H), 1.05-1.01 (m, 1H), 0.85 (s, 3H), 0.56 (s, 3H). UPLC (M+H)=511.4.

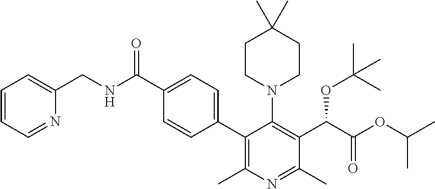 [SMILES: Cc1nc(C)c([C@H](OC(C)(C)C)C(=O)OC(C)C)c(N2CCC(C)(C)CC2)c1-c1ccc(C(=O)NCc2ccccn2)cc1]

PyBOP (238 mg, 0.458 mmol) was added to a nitrogen purged solution of (S)-4-(5-(1-(tert-butoxy)-2-isopropoxy-2-oxoethyl)-4-(4,4-dimethylpiperidin-1-yl)-2,6-dimethylpyridin-3-yl)benzoic acid (167 mg, 0.327 mmol), pyridin-2-ylmethanamine (0.050 mL, 0.491 mmol), and Hunig's base (0.171 mL, 0.981 mmol) in DMF (5 mL) and stirred for 3 h at 24° C. The reaction mixture was diluted with EtOAc and washed with sat'd NaHCO3 soln, water, brine, and dried (Na2SO4).). The crude product was charged (DCM) to a 40 g ISCO silica gel cartridge and gradient elution (5-65% MeOH/EtOAc) using an Isolera chromatography station gave (S)-isopropyl 2-(tert-butoxy)-2-(4-(4,4-dimethylpiperidin-1-yl)-2,6-dimethyl-5-(4-((pyridin-2-ylmethyl)carbamoyl)phenyl)pyridin-3-yl)acetate 150 mg (76%). ¹H NMR (500 MHz, DMSO) δ 9.24-9.21 (m, 1H), 8.53 (d, J=4.4 Hz, 1H), 8.05 (d, J=8.1 Hz, 1H), 8.03 (d, J=8.4 Hz, 1H), 7.78 (t, J=7.7 Hz, 1H), 7.50 (d, J=7.3 Hz, 1H), 7.36 (d, J=7.7 Hz, 1H), 7.31-7.27 (m, 2H), 5.99 (s, 1H), 4.99-4.94 (m, 1H), 4.61 (d, J=5.9 Hz, 2H), 3.35 (br. s, 1H) 3.18-3.13 (m, 1H), 2.84 (t, J=11.7 Hz, 1H), 2.46 (s, 3H), 2.26 (d, J=11.4 Hz, 1H), 2.07 (s, 3H), 1.94-1.89 (m, 1H), 1.50-1.45 (m, 1H), 1.31-1.27 (m, 1H), 1.27-1.24 (m, 1H), 1.20 (d, J=6.2 Hz, 3H), 1.16-1.14 (m, 12H), 1.06-1.03 (m, 1H), 0.86 (s, 3H), 0.59 (s, 3H). UPLC (M+H)=601.4.

### EXAMPLE 66

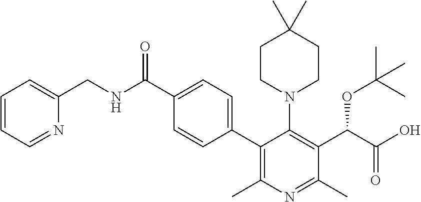 [SMILES: Cc1nc(C)c([C@H](OC(C)(C)C)C(=O)O)c(N2CCC(C)(C)CC2)c1-c1ccc(C(=O)NCc2ccccn2)cc1]

(S)-2-(tert-Butoxy)-2-(4-(4,4-dimethylpiperidin-1-yl)-2,6-dimethyl-5-(4-((pyridin-2-ylmethyl)carbamoyl)phenyl)pyridin-3-yl)acetic acid: The potassium hydroxide (56 mg, 1.00 mmol) was added to a solution of (S)-isopropyl 2-(tert-butoxy)-2-(4-(4,4-dimethylpiperidin-1-yl)-2,6-dimethyl-5-(4-((pyridin-2-ylmethyl)carbamoyl)phenyl) pyridin-3-yl)acetate (60 mg, 0.10 mmol) in ethanol (4 mL) and stirred for 6.5 h at 90° C. The reaction mixture was neutralized with 1N HCl soln, extracted with EtOAc, and the organic layer was washed with brine, and dried (MgSO4). The crude material was purified by prep HPLC (XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 15-55% B over 30 minutes, then a 5-minute hold at 100% B; Flow: 20 mL/min. There was obtained (S)-2-(tert-butoxy)-2-(4-(4,4-dimethylpiperidin-1-yl)-2,6-dimethyl-5-(4-((pyridin-2-ylmethyl)carbamoyl)phenyl)pyridin-3-yl)acetic acid 50 mg (89%). ¹H NMR (500 MHz, DMSO) δ 9.24-9.22 (m, 1H), 8.53 (d, J=4.8 Hz, 1H), 8.05 (d, J=7.7 Hz, 1H), 8.03 (d, J=8.1 Hz, 1H), 7.78 (t, J=7.7 Hz, 1H), 7.49 (d, J=7.7 Hz, 1H), 7.36 (d, J=8.1 Hz, 1H), 7.29-7.27 (m, 2H), 5.81 (s, 1H), 4.61 (d, J=5.9 Hz, 2H), 3.38 (br. s, 2H) 2.81 (t, J=12.0 Hz, 1H), 2.46 (s, 3H), 2.23 (d, J=9.9 Hz, 1H), 2.06 (s, 3H), 1.54-1.48 (m, 1H), 1.32-1.28 (m, 1H), 1.19 (d, J=11.7 Hz, 1H), 1.14 (s, 9H), 1.02 (d, J=9.9 Hz, 1H), 0.85 (s, 3H), 0.58 (s, 3H). UPLC (M+H)=559.5.

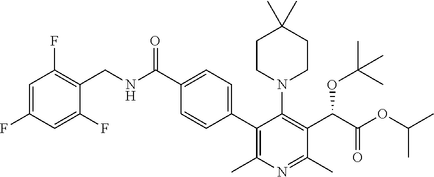 [SMILES: Cc1nc(C)c([C@H](OC(C)(C)C)C(=O)OC(C)C)c(N2CCC(C)(C)CC2)c1-c1ccc(C(=O)NCc2c(F)cc(F)cc2F)cc1]

(S)-Isopropyl 2-(tert-butoxy)-2-(4-(4,4-dimethylpiperidin-1-yl)-2,6-dimethyl-5-(4-((2,4,6-trifluorobenzyl)carbamoyl)phenyl)pyridin-3-yl)acetate: The PyBOP (51.0 mg, 0.098 mmol) was added to a nitrogen purged solution of (S)-4-(5-(1-(tert-butoxy)-2-isopropoxy-2-oxoethyl)-4-(4,4-dimethylpiperidin-1-yl)-2,6-dimethylpyridin-3-yl)benzoic acid (25 mg, 0.049 mmol), (2,4,6-trifluorophenyl)methanamine (15.78 mg, 0.098 mmol), and Hunig's base (0.026 mL, 0.147 mmol) in DMF (1 mL) and stirred for 3 h at 24° C. The reaction mixture was diluted with EtOAc and washed with sat'd NaHCO3 soln, water, brine, and dried (Na2SO4).). The crude product was charged (DCM) to a 12 g ISCO silica gel cartridge and gradient elution (15-100% EtOAc/Hex) using an Isolera chromatography station gave (S)-isopropyl 2-(tert-butoxy)-2-(4-(4,4-dimethylpiperidin-1-yl)-2,6-dimethyl-5-(4-((2,4,6-trifluorobenzyl)carbamoyl)phenyl)pyridin-3-yl)acetate 20 mg (63%). ¹H NMR (500 MHz, DMSO) δ 9.00-8.99 (m, 1H), 7.97 (d, J=8.1 Hz, 1H), 7.93 (d, J=7.7 Hz, 1H), 7.46 (d, J=7.3 Hz, 1H), 7.26 (d, J=8.1 Hz, 1H), 7.21-7.18 (m, 2H), 5.98 (s, 1H), 4.98-4.94 (m, 1H), 4.51 (d, J=4.4 Hz, 2H), 3.32 (br. s, 1H) 3.13 (d, J=12.4 Hz, 1H), 2.81 (t, J=12.1 Hz, 1H), 2.45 (s, 3H), 2.23 (d, J=9.9 Hz, 1H), 2.04 (s, 3H), 1.91-1.86 (m, 1H), 1.50-1.44 (m, 1H), 1.30-1.25 (m, 1H), 1.19 (d, J=6.2 Hz, 3H), 1.15-1.13 (m, 12H), 1.05-1.02 (m, 1H), 0.85 (s, 3H), 0.56 (s, 3H). UPLC (M+H)=654.4.

### EXAMPLE 67

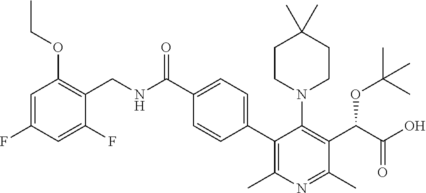 [SMILES: CCOc1cc(F)cc(F)c1CNC(=O)c1ccc(-c2c(C)nc(C)c([C@H](OC(C)(C)C)C(=O)O)c2N2CCC(C)(C)CC2)cc1]

(S)-2-(tert-Butoxy)-2-(4-(4,4-dimethyl piperidin-1-yl)-5-(4-((2-ethoxy-4,6-difluorobenzyl)carbamoyl)phenyl)-2,6-dimethylpyridin-3-yl)acetic acid: The potassium hydroxide (17 mg, 0.31 mmol) was added to a solution of (S)-isopropyl 2-(tert-butoxy)-2-(4-(4,4-dimethylpiperidin-1-yl)-2,6-dimethyl-5-(4-((2,4,6-trifluorobenzyl)carbamoyl)phenyl)pyridin-3-yl)acetate (20 mg, 0.031 mmol) in ethanol (1 mL) and stirred for 18 h at 90° C. The reaction mixture was neutralized with 1N HCl soln, extracted with EtOAc, and the organic layer was washed with brine, and dried (MgSO4). The crude material was purified by prep HPLC (XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 45-85% B over 30 minutes, then a 5-minute hold at 100% B; Flow: 20 mL/min. There was obtained (S)-2-(tert-butoxy)-2-(4-(4,4-dimethyl piperidin-1-yl)-5-(4-((2-ethoxy-4,6-difluorobenzyl)carbamoyl)phenyl)-2,6-dimethylpyridin-3-yl)acetic acid 8.4 mg (44%). ¹H NMR (500 MHz, DMSO) δ 8.58-8.56 (m, 1H), 7.97 (d, J=7.7 Hz, 1H), 7.92 (d, J=7.3 Hz, 1H), 7.43 (d, J=8.1 Hz, 1H), 7.21 (d, J=8.1 Hz, 1H), 6.83-6.78 (m, 2H), 5.73 (s, 1H), 4.47 (d, J=4.4 Hz, 2H), 4.08 (q, J=7.0 Hz, 2H), 3.45-3.43 (m, 2H) 2.77 (t, J=12.1 Hz, 1H), 2.44 (s, 3H), 2.19 (d, J=9.9 Hz, 1H), 2.03 (s, 3H), 1.85-1.81 (m, 1H), 1.52-1.47 (m, 1H), 1.30 (t, J=6.6 Hz, 3H), 1.17 (d, J=11.4, 1H), 1.12 (s, 9H), 0.98 (d, J=11.3 Hz, 1H), 0.84 (s, 3H), 0.56 (s, 3H). UPLC (M+H)=638.4.

### EXAMPLE 68

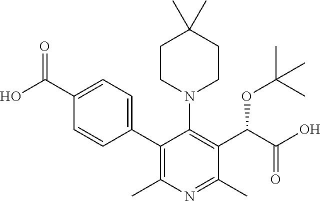 [SMILES: Cc1nc(C)c([C@H](OC(C)(C)C)C(=O)O)c(N2CCC(C)(C)CC2)c1-c1ccc(C(=O)O)cc1]

(S)-4-(5-(tert-Butoxy(carboxy) methyl)-4-(4,4-dimethylpiperidin-1-yl)-2,6-dimethylpyridin-3-yl)benzoic acid: The potassium hydroxide (198 mg, 3.52 mmol) was added to a solution of (S)-4-(5-(1-(tert-butoxy)-2-isopropoxy-2-oxoethyl)-4-(4,4-dimethylpiperidin-1-yl)-2,6-dimethylpyridin-3-yl)benzoic acid (180 mg, 0.35 mmol) in ethanol (4 mL) and stirred for 4 h at 90° C., and 16 h at 70° C. The reaction mixture was diluted with water (15 mL) and washed with ether, and the aqueous layer was acidified (pH=3) with 1N HCl, extracted with EtOAc. NOTE: The aqueous layer was then diluted with brine and reextracted. The combined organic layers were dried (MgSO4). There was obtained (S)-4-(5-(tert-butoxy(carboxy) methyl)-4-(4,4-dimethylpiperidin-1-yl)-2,6-dimethylpyridin-3-yl)benzoic acid 99 mg (60%). The material was used without further purification, however, a portion of the crude material was purified characterization purposes: HPLC (XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 10-50% B over 15 minutes, then a 5-minute hold at 100% B; Flow: 20 mL/min. ¹H NMR (500 MHz, DMSO) δ 8.01 (d, J=7.7 Hz, 1H), 7.97 (d, J=8.1 Hz, 1H), 7.38 (d, J=7.7 Hz, 1H), 7.18 (d, J=7.7 Hz, 1H), 5.75 (s, 1H), 3.46-3.43 (m, 2H) 2.79 (t, J=11.7 Hz, 1H), 2.45 (s, 3H), 2.18 (d, J=11.4 Hz, 1H), 2.05 (s, 3H), 1.52-1.47 (m, 1H), 1.30-1.25 (m, 1H), 1.17 (d, J=10.6 Hz, 1H), 1.12 (s, 9H), 1.00 (d, J=12.8, 1H), 0.84 (s, 3H), 0.57 (s, 3H). UPLC (M+H)=469.3.

### EXAMPLE 69

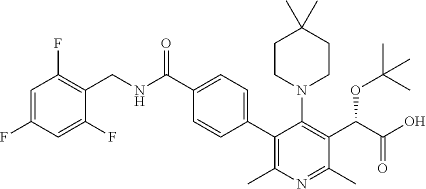 [SMILES: Cc1nc(C)c([C@H](OC(C)(C)C)C(=O)O)c(N2CCC(C)(C)CC2)c1-c1ccc(C(=O)NCc2c(F)cc(F)cc2F)cc1]

(S)-2-(tert-Butoxy)-2-(4-(4,4-dimethylpiperidin-1-yl)-2,6-dimethyl-5-(4-((2,4,6-trifluorobenzyl)carbamoyl)phenyl)pyridin-3-yl)acetic acid: The PyBOP (26.7 mg, 0.051 mmol) was added to a nitrogen purged solution (S)-4-(5-(tert-butoxy(carboxy)methyl)-4-(4,4-dimethylpiperidin-1-yl)-2,6-dimethylpyridin-3-yl)benzoic acid (40 mg, 0.049 mmol), (2,4,6-trifluorophenyl)methanamine (8.3 mg, 0.051 mmol), and Hunig's base (0.075 mL, 0.427 mmol) in DMF (1 mL) and stirred for 1 h at 24° C. An additional 1 eqv of reagents were added and the reaction was continued for 2 h and directly injected onto prep HPLC on WATERS-Atlantis column (30×100 mm S5) running 25 min gradient from 10-100% B (MeOH/water/TFA) (2 injections). The first peak eluting at 17.5 min contained both mono coupled products, and a second peak eluting at 19 min was a bis-coupled product. The first peak was further submitted to prep HPLC (XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 20-80% B over 20 minutes, then a 5-minute hold at 100% B; Flow: 20 mL/min. There was obtained (S)-2-(tert-butoxy)-2-(4-(4,4-dimethylpiperidin-1-yl)-2,6-dimethyl-5-(442,4,6-trifluorobenzyl)carbamoyl)phenyl)pyridin-3-yl)acetic acid as the first eluting peak 5.7 mg (11%). ¹H NMR (500 MHz, DMSO) δ 9.00-8.98 (m, 1H), 7.97 (d, J=8.1 Hz, 1H), 7.93 (d, J=7.7 Hz, 1H), 7.45 (d, J=7.7 Hz, 1H), 7.25 (d, J=8.1 Hz, 1H), 7.21-7.18 (m, 2H), 5.83 (s, 1H), 4.50 (d, J=4.8 Hz, 2H), 3.31-3.29 (m, 1H) 2.79 (t, J=11.7 Hz, 1H), 2.45 (s, 3H), 2.21 (d, J=11.4 Hz, 1H), 2.03 (s, 3H), 1.85 (t, J=10.6 Hz, 1H), 1.51-1.47 (m, 1H), 1.31-1.25 (m, 1H), 1.18 (d, J=9.2 Hz, 1H), 1.13 (s, 9H), 1.00 (d, J=11.0 Hz, 1H), 0.84 (s, 3H), 0.56 (s, 3H). UPLC (M+H)=612.4.

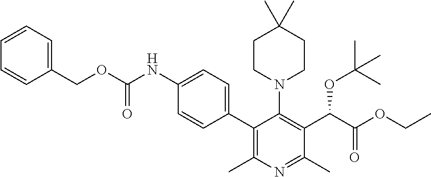 [SMILES: CCOC(=O)[C@@H](OC(C)(C)C)c1c(C)nc(C)c(-c2ccc(NC(=O)OCc3ccccc3)cc2)c1N1CCC(C)(C)CC1]

(S)-Ethyl 2-(5-(4-(((benzyloxy)carbonyl)amino)phenyl)-4-(4,4-dimethylpiperidin-1-yl)-2,6-dimethylpyridin-3-yl)-2-(tert-butoxy)acetate: A mixture of (S)-ethyl 2-(5-bromo-4-(4,4-dimethylpiperidin-1-yl)-2,6-dimethylpyridin-3-yl)-2-(tert-butoxy)acetate (0.47 g, 1.032 mmol), (4-(((benzyloxy)carbonyl)amino)phenyl) boronic acid (0.559 g, 2.064 mmol) and 2M Na2CO3 (1.548 ml, 3.10 mmol) in DMF (10 mL) was degassed for 10 min. Then, Pd(Ph3P)4 (0.060 g, 0.052 mmol) was added, degassed for 5 min and placed in an oil bath pre-heated to 90° C. After 8 h, cooled, diluted with ether (75 mL), washed with water (4×10 mL), brine (10 mL), dried (MgSO₄), filtered, concentrated and purified the yellow residue by flash chromatography using EtOAc/Hex to afford (S)-ethyl 2-(5-(4-(((benzyloxy)carbonyl)amino)phenyl)-4-(4,4-dimethylpiperidin-1-yl)-2,6-dimethylpyridin-3-yl)-2-(tert-butoxy)acetate (0.4394 g, 0.730 mmol, 70.8% yield) as pale yellow solid contaminated with about 10% of des-bromopyridine derivative. ¹H NMR (500 MHz, CDCl₃) δ 7.54 (d, J=7.4 Hz, 1H), 7.47 (d, J=7.9 Hz, 1H), 7.45-7.36 (m, 4H), 7.20 (dd, J=8.2, 1.6 Hz, 1H), 7.12-7.07 (m, 2H), 6.07 (s, 1H), 5.28-5.21 (m, 2H), 4.29-4.14 (m, 2H), 3.23-3.15 (m, 1H), 2.86 (t, J=12.1 Hz, 1H), 2.60 (s, 3H), 2.28 (d, J=10.9 Hz, 1H), 2.19 (s, 3H), 2.11-2.04 (m, 1H), 1.63-1.49 (m, 2H), 1.42-1.32 (m, 2H), 1.25 (t, J=7.1 Hz, 3H), 1.20 (s, 9H), 1.04 (s, 3H), 0.90 (br. s., 3H). LCMS (M+H)=601.4.

### EXAMPLE 70 AND 71

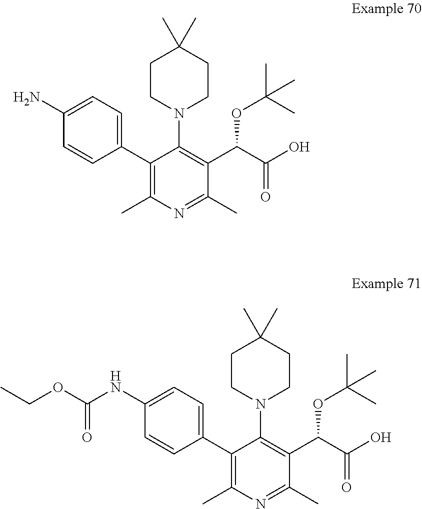 [SMILES: Cc1nc(C)c([C@H](OC(C)(C)C)C(=O)O)c(N2CCC(C)(C)CC2)c1-c1ccc(N)cc1 ; CCOC(=O)Nc1ccc(-c2c(C)nc(C)c([C@H](OC(C)(C)C)C(=O)O)c2N2CCC(C)(C)CC2)cc1]

A mixture of (S)-ethyl 2-(5-(4-(((benzyloxy)carbonyl)amino)phenyl)-4-(4,4-dimethylpiperidin-1-yl)-2,6-dimethylpyridin-3-yl)-2-(tert-butoxy)acetate (0.139 g, 0.208 mmol) and 1M NaOH (0.624 ml, 0.624 mmol) in EtOH (4 mL) was refluxed for 4 h. Then, cooled and purified by prep-HPLC to afford to afford examples 1 and 2.

### EXAMPLE 70

(S)-2-(5-(4-Aminophenyl)-4-(4,4-dimethylpiperidin-1-yl)-2,6-dimethylpyridin-3-yl)-2-(tert-butoxy)acetic acid (0.037 g, 0.084 mmol, 40.5% yield), white solid. ¹H NMR (500 MHz, CDCl₃) δ 7.01 (d, J=7.6 Hz, 1H), 6.92 (d, J=6.9 Hz, 1H), 6.77 (d, J=7.7 Hz, 2H), 5.91 (br. s., 1H), 3.64 (br. s., 2H), 3.07-2.75 (m, 2H), 2.68 (br. s., 3H), 2.25 (s, 3H), 1.57-1.28 (m, 6H), 1.25 (s, 9H), 0.80 (br. s., 6H). LCMS (M+H)=440.4

### EXAMPLE 71

(S)-2-(tert-Butoxy)-2-(4-(4,4-dimethylpiperidin-1-yl)-5-(4-((ethoxycarbonyl)amino)phenyl)-2,6-dimethylpyridin-3-yl)acetic acid (0.0144 g, 0.028 mmol, 13.54% yield), white solid. ¹H NMR (500 MHz, CDCl₃) δ 7.57 (d, J=7.7 Hz, 1H), 7.42 (dd, J=8.4, 1.9 Hz, 1H), 7.21 (dd, J=8.4, 1.9 Hz, 1H), 7.10 (dd, J=8.2, 1.9 Hz, 1H), 6.71 (s, 1H), 5.97 (br. s., 1H), 4.28 (q, J=7.2 Hz, 2H), 3.60 (br. s., 1H), 2.91 (br. s., 1H), 2.66 (s, 3H), 2.41-2.24 (m, 1H), 2.22 (s, 3H), 2.18-1.99 (m, 1H), 1.67-1.49 (m, 2H), 1.36 (t, J=7.2 Hz, 3H), 1.34-1.27 (m, 2H), 1.25 (s, 9H), 0.78 (br.s., 6H). LCMS (M+H)=512.4.

### EXAMPLE 72

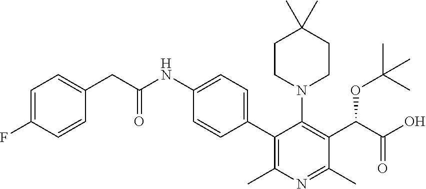 [SMILES: Cc1nc(C)c([C@H](OC(C)(C)C)C(=O)O)c(N2CCC(C)(C)CC2)c1-c1ccc(NC(=O)Cc2ccc(F)cc2)cc1]

(S)-2-(tert-Butoxy)-2-(4-(4,4-dimethylpiperidin-1-yl)-5-(4-(2-(4-fluorophenyl)acetamido)phenyl)-2,6-dimethylpyridin-3-yl)acetic acid: To a stirred solution of (S)-2-(5-(4-aminophenyl)-4-(4,4-dimethylpiperidin-1-yl)-2,6-dimethylpyridin-3-yl)-2-(tert-butoxy)acetic acid (0.025 g, 0.057 mmol) and DIEA (0.020 ml, 0.114 mmol) in CH₂Cl₂ (3 mL) was added 2-(4-fluorophenyl)acetyl chloride (9.36 0.068 mmol) at rt. After 1 h, concentrated and purified by prep-HPLC to afford (S)-2-(tert-butoxy)-2-(4-(4,4-dimethylpiperidin-1-yl)-5-(4-(2-(4-fluorophenyl)acetamido)phenyl)-2,6-dimethylpyridin-3-yl)acetic acid (0.0209 g, 0.036 mmol, 63.8% yield) as white solid. ¹H NMR (500 MHz, METHANOL-d₄) δ 7.86 (dd, J=8.4, 2.2 Hz, 1H), 7.71 (dd, J=8.2, 2.2 Hz, 1H), 7.43-7.39 (m, 2H), 7.37 (dd, J=8.4, 2.0 Hz, 1H), 7.15 (dd, J=8.4, 2.0 Hz, 1H), 7.12-7.07 (m, 2H), 5.53 (s, 1H), 3.74 (s, 2H), 2.76 (br. s., 2H), 2.70 (s, 3H), 2.30 (s, 3H), 1.38 (br. s., 4H), 1.21 (s, 9H), 0.85 (s, 6H). 2H of piperidine are missing. LCMS (M+H)=576.4.

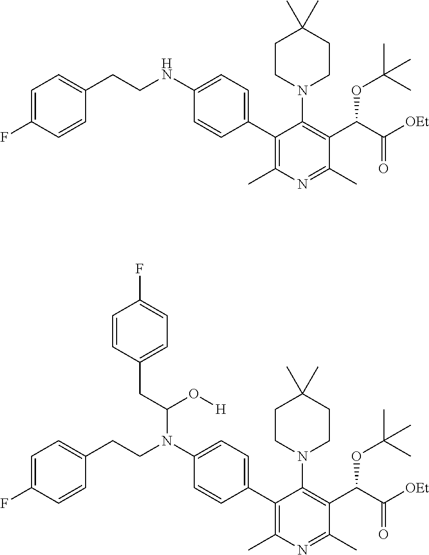 [SMILES: CCOC(=O)[C@@H](OC(C)(C)C)c1c(C)nc(C)c(-c2ccc(NCCc3ccc(F)cc3)cc2)c1N1CCC(C)(C)CC1 ; CCOC(=O)[C@@H](OC(C)(C)C)c1c(C)nc(C)c(-c2ccc(N(CCc3ccc(F)cc3)C(O)Cc3ccc(F)cc3)cc2)c1N1CCC(C)(C)CC1]

(S)-Ethyl 2-(tert-butoxy)-2-(4-(4,4-dimethylpiperidin-1-yl)-5-(4-((4-fluorophenethyl)amino)phenyl)-2,6-dimethylpyridin-3-yl)acetate: To a stirred solution of (S)-ethyl 2-(5-(4-aminophenyl)-4-(4,4-dimethylpiperidin-1-yl)-2,6-dimethylpyridin-3-yl)-2-(tert-butoxy)acetate (0.233 g, 0.498 mmol) and 2-(4-fluorophenyl)acetaldehyde (0.069 g, 0.498 mmol) in MeOH (5 mL) was added at once NaCNBH4 (0.063 g, 0.996 mmol) and ZnCl2 (0.068 g, 0.498 mmol) at rt. After 24 h, diluted with ether (50 mL), washed with sat Na2CO3 (10 mL), water (2×5 mL), brine (5 mL), dried (MgSO₄), filtered, concentrated and purified by prep-HPLC to afford (S)-ethyl 2-(tert-butoxy)-2-(4-(4,4-dimethylpiperidin-1-yl)-5-(4-((4-fluorophenethyl)amino)phenyl)-2,6-dimethylpyridin-3-yl)acetate as white solid and contaminated with an impurity with M+H=728.7. ¹H NMR (500 MHz, CDCl₃) δ 7.23-7.18 (m, 2H), 7.07-7.01 (m, 3H), 6.98 (d, J=8.4 Hz, 1H), 6.69 (t, J=7.6 Hz, 2H), 6.10 (s, 1H), 4.30-4.22 (m, 1H), 4.21-4.13 (m, 1H), 3.75 (br. s., 1H), 3.46 (t, J=6.6 Hz, 2H), 3.18 (d, J=11.3 Hz, 1H), 2.95 (t, J=6.8 Hz, 2H), 2.92-2.86 (m, 1H), 2.60 (s, 3H), 2.29 (d, J=11.5 Hz, 1H), 2.23 (s, 3H), 2.22-2.15 (m, 1H), 1.57 (t, J=12.6 Hz, 1H), 1.43-1.35 (m, 1H), 1.26 (t, J=7.1 Hz, 3H), 1.21 (s, 10H), 1.10 (d, J=13.4 Hz, 1H), 0.91 (s, 3H), 0.70 (s, 3H). LCMS (M+H)=590.6.

### EXAMPLE 73 AND 74

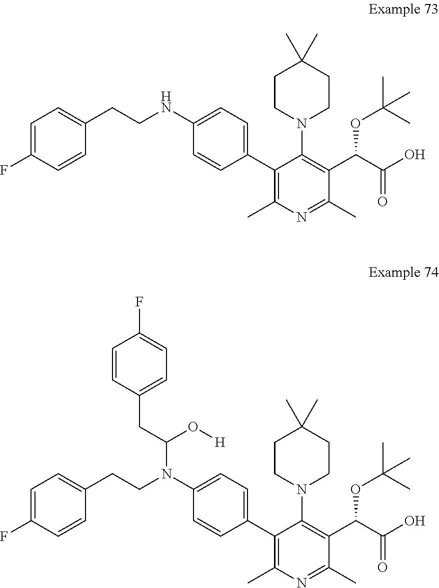 [SMILES: Cc1nc(C)c([C@H](OC(C)(C)C)C(=O)O)c(N2CCC(C)(C)CC2)c1-c1ccc(NCCc2ccc(F)cc2)cc1 ; Cc1nc(C)c([C@H](OC(C)(C)C)C(=O)O)c(N2CCC(C)(C)CC2)c1-c1ccc(N(CCc2ccc(F)cc2)C(O)Cc2ccc(F)cc2)cc1]

A solution of (S)-ethyl 2-(tert-butoxy)-2-(4-(4,4-dimethylpiperidin-1-yl)-5-(4-((4-fluorophenethyl)amino)phenyl)-2,6-dimethylpyridin-3-yl)acetate (0.09 g, 0.153 mmol) and 1M NaOH (1.526 ml, 1.526 mmol) in EtOH (3 mL) was refluxed for 3 h. Then, cooled and purified by prep-HPLC to afford Example 73 and Example 74.

### EXAMPLE 73

(S)-2-(tert-Butoxy)-2-(4-(4,4-dimethylpiperidin-1-yl)-5-(4-((4-fluorophenethyl)amino)phenyl)-2,6-dimethylpyridin-3-yl)acetic acid (0.0574 g, 0.102 mmol, 67.0% yield) as white solid. ¹H NMR (500 MHz, METHANOL-d₄) δ 7.31-7.26 (m, 2H), 7.12-7.08 (m, 1H), 7.06-7.00 (m, 2H), 6.95-6.91 (m, 1H), 6.82-6.76 (m, 2H), 5.50 (s, 1H), 3.44-3.40 (m, 2H), 2.93 (t, J=7.3 Hz, 2H), 2.83-2.70 (m, 2H), 2.69 (s, 3H), 2.34 (s, 3H), 1.46-1.34 (m, 4H), 1.20 (s, 9H), 0.88 (s, 6H). 2H of piperidine, NH and CO2H are missing. LCMS (M+H)=562.6.

### EXAMPLE 74

(2S)-2-(tert-Butoxy)-2-(4-(4,4-dimethylpiperidin-1-yl)-5-(4-((4-fluorophenethyl)(2-(4-fluorophenyl)-1-hydroxyethyl)amino)phenyl)-2,6-dimethylpyridin-3-yl)acetic acid (0.0184 g, 0.026 mmol, 17.23% yield) as white solid, structure tentatively assigned based on HNMR. ¹H NMR (500 MHz, METHANOL-d₄) δ 7.41-7.35 (m, 1.7H), 7.31-7.26 (m, 0.3H), 7.21-7.12 (m, 2H), 7.11-7.02 (m, 3H), 7.01-6.94 (m, 2H), 6.90-6.85 (m, 1H), 6.76-6.68 (m, 2H), 5.50 (s, 1H), 4.29-4.23 (m, 0.9H), 4.09-4.02 (m, 0.1H), 3.96-3.90 (m, 0.1H), 3.70 (dt, J=13.4, 6.6 Hz, 0.9H), 3.53-3.39 (m, 1H), 3.10-3.02 (m, 1H), 2.79-2.63 (m, 3H), 2.68 (s, 3H), 2.57-2.49 (m, 1H), 2.32 (d, J=1.9 Hz, 3H), 1.46-1.34 (m, 4H), 1.20 (s, 9H), 0.88 (s, 3H), 0.87 (s, 3H). LCMS (M+H)=700.7. This compound was formed from an impurity present in the starting material.

### EXAMPLE 75

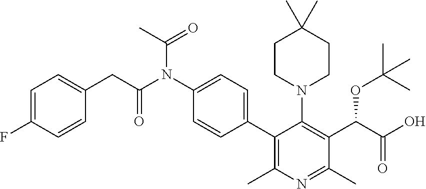 [SMILES: CC(=O)N(C(=O)Cc1ccc(F)cc1)c1ccc(-c2c(C)nc(C)c([C@H](OC(C)(C)C)C(=O)O)c2N2CCC(C)(C)CC2)cc1]

(S)-2-(tert-Butoxy)-2-(4-(4,4-dimethylpiperidin-1-yl)-5-(4-(N-(4-fluorophenethyl)acetamido)phenyl)-2,6-dimethylpyridin-3-yl)acetic acid: To a stirred solution of (S)-2-(tert-butoxy)-2-(4-(4,4-dimethylpiperidin-1-yl)-5-(4-((4-fluorophenethyl)amino)phenyl)-2,6-dimethylpyridin-3-yl)acetic acid (0.01 g, 0.018 mmol) and DIEA (0.016 ml, 0.089 mmol) in CH2Cl2 (0.5 mL) was added Ac2O (8.40 μl, 0.089 mmol) at rt. After 4 h, the reaction was quenched with water/acetonitrile, concentrated and purified by prep-HPLC to afford (S)-2-(tert-butoxy)-2-(4-(4,4-dimethylpiperidin-1-yl)-5-(4-(N-(4-fluorophenethyl)acetamido)-phenyl)-2,6-dimethylpyridin-3-yl)acetic acid (0.0081 g, 0.013 mmol, 75% yield) as solid. ¹H NMR (500 MHz, DMSO-d₆) δ 7.42 (d, J=8.1 Hz, 1H), 7.38 (d, J=8.1 Hz, 1H), 7.33 (d, J=8.1 Hz, 1H), 7.23 (d, J=8.1 Hz, 1H), 7.21-7.16 (m, 2H), 7.12-7.06 (m, 2H), 5.84 (br. s., 1H), 3.91 (t, J=7.2 Hz, 2H), 3.28 (d, J=10.8 Hz, 1H), 2.84-2.75 (m, 3H), 2.46 (s, 3H), 2.24 (d, J=9.5 Hz, 1H), 2.10 (s, 3H), 1.90-1.85 (m, 1H), 1.80 (br. s., 3H), 1.49 (br. s., 1H), 1.35-1.27 (m, 1H), 1.18 (d, J=12.8 Hz, 1H), 1.14 (s, 9H), 0.99 (d, J=12.8 Hz, 1H), 0.84 (br. s., 3H), 0.56 (s, 3H). LCMS (M+H)=604.6.

### EXAMPLE 76

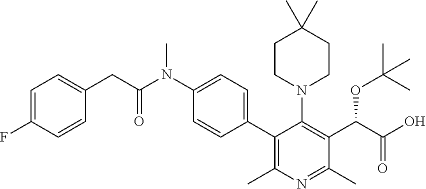 [SMILES: Cc1nc(C)c([C@H](OC(C)(C)C)C(=O)O)c(N2CCC(C)(C)CC2)c1-c1ccc(N(C)C(=O)Cc2ccc(F)cc2)cc1]

(S)-2-(tert-Butoxy)-2-(4-(4,4-dimethylpiperidin-1-yl)-5-(4-((4-fluorophenethyl)(methyl)amino)phenyl)-2,6-dimethylpyridin-3-yl)acetic acid: A 10 mL RB flask was charged with (S)-ethyl 2-(tert-butoxy)-2-(4-(4,4-dimethylpiperidin-1-yl)-5-(4-((4-fluorophenethyl)amino)phenyl)-2,6-dimethylpyridin-3-yl)acetate (0.0247 g, 0.042 mmol), paraformaldehyde (5.03 mg, 0.168 mmol), NaCNBH4 (5.26 mg, 0.084 mmol) and ZnCl2 (5.71 mg, 0.042 mmol). To this was added MeOH (2 mL) and stirred at rt for 6.5 h. LCMS at this point showed completion of N-methylation reaction and presence of (S)-ethyl 2-(tert-butoxy)-2-(4-(4,4-dimethyl-piperidin-1-yl)-5-(4-((4-fluorophenethyl)(methyl)amino)phenyl)-2,6-dimethylpyridin-3-yl)acetate (LCMS M(+H)=). To this was added NaOH (0.419 ml, 0.419 mmol) and refluxed for 16 h, cooled, filtered to remove white solids and purified by prep-HPLC to afford (S)-2-(tert-butoxy)-2-(4-(4,4-dimethylpiperidin-1-yl)-5-(4-((4-fluorophenethyl)(methyl)amino)phenyl)-2,6-dimethylpyridin-3-yl)acetic acid (0.0141 g, 0.024 mmol, 58.5% yield) as solid. ¹H NMR (500 MHz, METHANOL-d₄) δ 7.27-7.21 (m, 2H), 7.17 (dd, J=8.4, 2.1 Hz, 1H), 7.04-6.98 (m, 3H), 6.91-6.84 (m, 2H), 5.51 (s, 1H), 3.77-3.60 (m, 2H), 3.36-3.30 (m, 2H), 2.93 (s, 3H), 2.93-2.89 (m, 2H), 2.83-2.71 (m, 2H), 2.70 (s, 3H), 2.34 (s, 3H), 1.46-1.34 (m, 4H), 1.21 (s, 9H), 0.87 (s, 6H). LCMS (M+H)=576.7.

### EXAMPLE 77

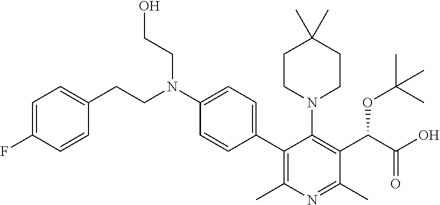 [SMILES: Cc1nc(C)c([C@H](OC(C)(C)C)C(=O)O)c(N2CCC(C)(C)CC2)c1-c1ccc(N(CCO)CCc2ccc(F)cc2)cc1]

(S)-2-(tert-Butoxy)-2-(4-(4,4-dimethylpiperidin-1-yl)-5-(4-((4-fluorophenethyl)(2-hydroxyethyl)amino)phenyl)-2,6-dimethylpyridin-3-yl)acetic acid: A 10 mL RB flask was charged with (S)-ethyl 2-(tert-butoxy)-2-(4-(4,4-dimethylpiperidin-1-yl)-5-(4-((4-fluorophenethyl)amino)phenyl)-2,6-dimethylpyridin-3-yl)acetate (0.0222 g, 0.038 mmol), 1,4-dioxane-2,5-diol (9.04 mg, 0.075 mmol), NaCNBH4 (4.73 mg, 0.075 mmol) and ZnCl2 (5.13 mg, 0.038 mmol). To this was added MeOH (2 mL) and stirred at rt for 10 h. LCMS showed presence of desired N-alkylated product (S)-ethyl 2-(tert-butoxy)-2-(4-(4,4-dimethylpiperidin-1-yl)-5-(4-((4-fluorophenethyl)(2-hydroxyethyl)amino)phenyl)-2,6-dimethylpyridin-3-yl)acetate; LCMS (M+H)=634.7). Then, NaOH (0.376 ml, 0.376 mmol) was added and refluxed for 6 h, cooled, filtered to remove brown solids and purified by prep-HPLC to afford (S)-2-(tert-butoxy)-2-(4-(4,4-dimethylpiperidin-1-yl)-5-(4-((4-fluorophenethyl)(2-hydroxyethyl)amino)phenyl)-2,6-dimethylpyridin-3-yl)acetic acid (0.0183 g, 0.030 mmol, 80% yield) as solid. ¹H NMR (500 MHz, METHANOL-d₄) δ 7.28-7.23 (m, 2H), 7.20-7.16 (m, 1H), 7.05-7.00 (m, 3H), 6.95-6.90 (m, 2H), 5.52 (s, 1H), 3.80-3.72 (m, 1H), 3.71-3.63 (m, 3H), 3.50-3.43 (m, 1H), 3.42-3.30 (m, 3H), 2.94 (t, J=7.3 Hz, 2H), 2.86-2.72 (m, 2H), 2.70 (s, 3H), 2.36 (s, 3H), 1.44-1.34 (m, 4H), 1.21 (s, 9H), 0.88 (s, 6H). LCMS (M+H)=606.7.

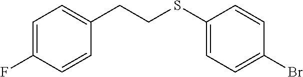 [SMILES: Fc1ccc(CCSc2ccc(Br)cc2)cc1]

(4-Bromophenyl)(4-fluorophenethyl)sulfane: To a stirred ice-cold solution of 4-bromobenzenethiol (1.891 g, 10 mmol), 2-(4-fluorophenyl)ethanol (1.752 g, 12.50 mmol) and Ph3P (3.15 g, 12.00 mmol) in THF (20 mL) was added dropwise40% DEAD/toluene (5.47 ml, 12.00 mmol) over min. After 1 h, cold bath was removed and stirred at rt for 15 h. Then, the reaction mixture was concentrated, the resulting residue triturated with hexanes, filtered and the filter cake washed with 10% ether/hexanes (2-lit). The filterate was concentrated and purified by flash chromatography (silicagel column 3″×11″) using 4-lit hexanes and 2-lit 2% EtOAc/Hex to afford (4-bromophenyl)(4-fluorophenethyl)sulfane (1.2618 g, 4.05 mmol, 40.5% yield) as colorless liquid and contaminated with about 7% Ph₃P. ¹H NMR (500 MHz, CDCl₃) δ 7.46-7.41 (m, 2H), 7.24-7.21 (m, 2H), 7.19-7.13 (m, 2H), 7.04-6.98 (m, 2H), 3.17-3.12 (m, 2H), 2.93-2.88 (m, 2H).

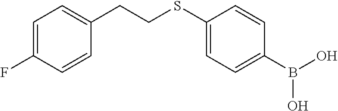 [SMILES: OB(O)c1ccc(SCCc2ccc(F)cc2)cc1]

(4-((4-Fluorophenethyl)thio)phenyl)boronic acid: To a stirred solution of (4-bromophenyl)(4-fluorophenethyl)sulfane (1.25 g, 4.02 mmol) in THF (25 mL) was added dropwise 2M n-BuLi/cyclohexane (2.51 ml, 5.02 mmol) at −78 C. After 1 h, triisopropyl borate (1.119 ml, 4.82 mmol) was added to the yellow reaction mixture over 5 min and stirred for 2 h at −78° C. Then, the reaction was quenched by careful addition of 3M HCl (3.5 mL), cold bath replaced with water bath, stirred for 1 h, diluted with ether (100 mL), aq layer separated and org layer washed with water (2×20 mL). The combined aq layers extracted with ether (50 mL) and combined ether layers washed with brine (20 mL), dried (MgSO₄), filtered and concentrated to give (4-((4-fluorophenethyl)thio)phenyl)boronic acid (1.23 g, 4.45 mmol, 111% yield) as viscous oil which turned to white solid under vaccum. This material was used as is in the next step. ¹H NMR (500 MHz, CDCl₃) δ 8.14 (d, J=8.2 Hz, 1H), 7.72-7.65 (m, 1H), 7.43 (d, J=8.4 Hz, 1H), 7.37-7.33 (m, 1H), 7.25-7.16 (m, 2H), 7.07-6.98 (m, 2H), 3.30-3.14 (m, 2H), 3.04-2.89 (m, 2H).

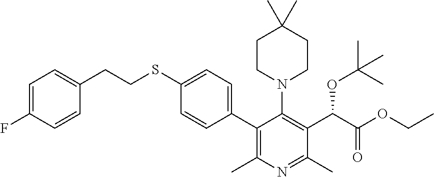 [SMILES: CCOC(=O)[C@@H](OC(C)(C)C)c1c(C)nc(C)c(-c2ccc(SCCc3ccc(F)cc3)cc2)c1N1CCC(C)(C)CC1]

(S)-Ethyl 2-(tert-butoxy)-2-(4-(4,4-dimethylpiperidin-1-yl)-5-(4-((4-fluorophenethyl)thio)phenyl)-2,6-dimethylpyridin-3-yl)acetate: A mixture of (S)-ethyl 2-(5-bromo-4-(4,4-dimethylpiperidin-1-yl)-2,6-dimethylpyridin-3-yl)-2-(tert-butoxy)acetate (0.228 g, 0.5 mmol), (4-((4-fluorophenethyl)thio)phenyl)boronic acid (0.207 g, 0.750 mmol) and 2M Na2CO3 (0.625 ml, 1.250 mmol) in DMF (10 mL) was degassed for 10 min. Then, Pd(Ph3P)4 (0.040 g, 0.035 mmol) was added, degassed for 5 min and placed in a pre-heated oil bath at 100° C. After 3 h at 110° C., the reaction mixture cooled, filtered and purified by prep-HPLC to afford (S)-ethyl 2-(tert-butoxy)-2-(4-(4,4-dimethylpiperidin-1-yl)-5-(4-((4-fluorophenethyl)thio)phenyl)-2,6-dimethylpyridin-3-yl)acetate (0.166 g, 0.274 mmol, 54.7% yield) as foam. ¹H NMR (500 MHz, CDCl₃) δ 7.45 (ddd, J=13.7, 7.9, 1.7 Hz, 2H), 7.23 (dd, J=8.0, 1.7 Hz, 1H), 7.20-7.16 (m, 2H), 7.13 (dd, J=8.0, 1.8 Hz, 1H), 7.04-6.99 (m, 2H), 6.06 (s, 1H), 4.27 (dq, J=10.8, 7.1 Hz, 1H), 4.18 (dq, J=10.9, 7.1 Hz, 1H), 3.24-3.17 (m, 3H), 2.97-2.92 (m, 2H), 2.87 (t, J=11.9 Hz, 1H), 2.61 (s, 3H), 2.32-2.26 (m, 1H), 2.21 (s, 3H), 2.06 (t, J=11.0 Hz, 1H), 1.56 (td, J=12.5, 3.9 Hz, 1H), 1.41-1.34 (m, 1H), 1.27 (t, J=7.2 Hz, 3H), 1.21 (s, 9H), 1.20-1.17 (m, 1H), 1.07 (d, J=13.2 Hz, 1H), 0.89 (s, 3H), 0.63 (s, 3H). LCMS (M+H)=607.7.

### EXAMPLE 78

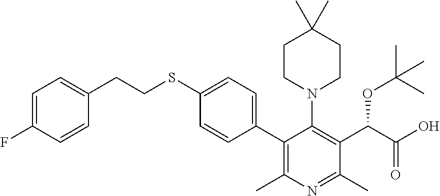 [SMILES: Cc1nc(C)c([C@H](OC(C)(C)C)C(=O)O)c(N2CCC(C)(C)CC2)c1-c1ccc(SCCc2ccc(F)cc2)cc1]

(S)-2-(tert-Butoxy)-2-(4-(4,4-dimethylpiperidin-1-yl)-5-(4-((4-fluorophenethyl)thio)phenyl)-2,6-dimethylpyridin-3-yl)acetic acid: A mixture of (S)-ethyl 2-(tert-butoxy)-2-(4-(4,4-dimethylpiperidin-1-yl)-5-(4-((4-fluorophenethyl)thio)phenyl)-2,6-dimethylpyridin-3-yl)acetate (0.155 g, 0.255 mmol) and 1M NaOH (0.766 ml, 0.766 mmol) in EtOH (5 mL) was refluxed for5 h. Then cooled, nuetralized with 1M HCl (0.8 mL), concentrated and taken in EtOAc (50 mL), washed with water (5 mL), brine (5 mL), dried (MgSO4), filtered and concentrated to give (S)-2-(tert-butoxy)-2-(4-(4,4-dimethylpiperidin-1-yl)-5-(4-((4-fluorophenethyl)thio)phenyl)-2,6-dimethylpyridin-3-yl)acetic acid (0.142 g, 0.245 mmol, 96% yield) as white solid. ¹H NMR (500 MHz, METHANOL-d₄) δ 7.57 (dd, J=8.0, 1.9 Hz, 1H), 7.52 (dd, J=8.0, 1.9 Hz, 1H), 7.37 (dd, J=8.0, 1.9 Hz, 1H), 7.28-7.22 (m, 2H), 7.16 (dd, J=8.0, 1.9 Hz, 1H), 7.06-6.99 (m, 2H), 5.55 (s, 1H), 3.36-3.32 (m, 2H), 3.31-3.24 (m, 2H), 2.98-2.91 (m, 2H), 2.85-2.72 (m, 2H), 2.70 (s, 3H), 2.32 (s, 3H), 1.45-1.31 (m, 4H), 1.21 (s, 9H), 0.83 (s, 6H). LCMS (M+H)=579.6.

### EXAMPLE 79

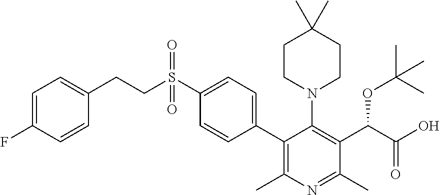 [SMILES: Cc1nc(C)c([C@H](OC(C)(C)C)C(=O)O)c(N2CCC(C)(C)CC2)c1-c1ccc(S(=O)(=O)CCc2ccc(F)cc2)cc1]

(S)-2-(tert-Butoxy)-2-(4-(4,4-dimethylpiperidin-1-yl)-5-(4-((4-fluorophenethyl)sulfonyl)phenyl)-2,6-dimethylpyridin-3-yl)acetic acid: To a solution of (S)-2-(tert-butoxy)-2-(4-(4,4-dimethylpiperidin-1-yl)-5-(4-((4-fluorophenethyl)thio)phenyl)-2,6-dimethylpyridin-3-yl)acetic acid (0.0425 g, 0.073 mmol) in MeOH (2 mL) and water (2 mL) was added oxone (0.135 g, 0.220 mmol) and stirred for 1 h at rt. Then, diluted with water (10 mL), extracted with CH2Cl2 (2×10 mL), dried (MgSO4), filtered, concentrated and purified by prep-HPLC to give (S)-2-(tert-butoxy)-2-(4-(4,4-dimethylpiperidin-1-yl)-5-(4-((4-fluorophenethyl)sulfonyl)phenyl)-2,6-dimethylpyridin-3-yl)acetic acid (0.0298 g, 0.049 mmol, 66.4% yield) as white solid. ¹H NMR (500 MHz, CDCl₃) δ 8.08 (dd, J=8.0, 1.8 Hz, 1H), 8.03 (dd, 1.9 Hz, 1H), 7.57 (dd, J=8.0, 1.4 Hz, 1H), 7.44 (dd, J=8.0, 1.5 Hz, 1H), 7.16-7.10 (m, 2H), 7.03-6.97 (m, 2H), 6.06 (br. s., 1H), 3.55 (d, J=9.3 Hz, 1H), 3.44-3.39 (m, 2H), 3.13-3.05 (m, 2H), 2.88 (t, J=12.0 Hz, 1H), 2.61 (s, 3H), 2.27 (d, J=10.4 Hz, 1H), 2.18 (s, 3H), 1.92-1.87 (m, 1H), 1.56-1.49 (m, 2H), 1.30-1.27 (m, 1H), 1.26 (s, 9H), 1.09-1.04 (m, 1H), 0.89 (br. s., 3H), 0.58 (br. s., 3H). LCMS (M+H)=611.7.

### EXAMPLE 80

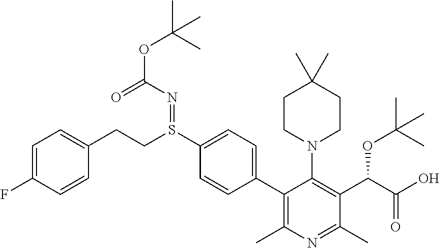 [SMILES: Cc1nc(C)c([C@H](OC(C)(C)C)C(=O)O)c(N2CCC(C)(C)CC2)c1-c1ccc(/S(CCc2ccc(F)cc2)=N/C(=O)OC(C)(C)C)cc1]

(2S)-2-(tert-Butoxy)-2-(5-(4-((E)-N-(tert-butoxycarbonyl)-S-(4-fluorophenethyl)sulfinimidoyl)phenyl)-4-(4,4-dimethylpiperidin-1-yl)-2,6-dimethylpyridin-3-yl)acetic acid: A solution of (S)-2-(tert-butoxy)-2-(4-(4,4-dimethylpiperidin-1-yl)-5-(4-((4-fluorophenethyl)thio)phenyl)-2,6-dimethylpyridin-3-yl)acetic acid (0.022 g, 0.038 mmol) and tert-butyl carbonazidate (5.44 mg, 0.038 mmol) in anhydrous CH₂Cl₂ (1 mL) was degassed for 5 min by bubbling N2 through the reaction mixture. Then, FeCl2 (4.82 mg, 0.038 mmol) was added and rigorously stirred for 23 h at rt. The solvent was removed and the brown residue was dissolved in MeOH and purified by prep-HPLC to afford (2S)-2-(tert-butoxy)-2-(5-(4-((E)-N-(tert-butoxycarbonyl)-S-(4-fluorophenethyl)sulfinimidoyl)phenyl)-4-(4,4-dimethyl-piperidin-1-yl)-2,6-dimethylpyridin-3-yl)acetic acid (0.0175 g, 0.025 mmol, 66.3% yield) as white solid and 91% purity. ¹H NMR (500 MHz, METHANOL-d₄) δ 8.02-7.98 (m, 1H), 7.96 (dd, J=8.2, 1.9 Hz, 1H), 7.74 (dd, J=8.2, 1.6 Hz, 1H), 7.49 (dd, J=8.0, 1.4 Hz, 1H), 7.33-7.27 (m, 2H), 7.10-7.04 (m, 2H), 5.61 (s, 1H), 3.51-3.45 (m, 2H), 3.14-2.93 (m, 3H), 2.80-2.70 (m, 2H), 2.69 (s, 3H), 2.27 (d, J=0.6 Hz, 3H), 1.54-1.51 (m, 2H), 1.47 (s, 4.5H), 1.47 (s, 4.5H), 1.38-1.30 (m, 2H), 1.21 (s, 9H), 0.78 (br. s., 6H). Two protons of piperidine are burried under CD3OD peak. LCMS (M+H)=694.7.

### EXAMPLE 81

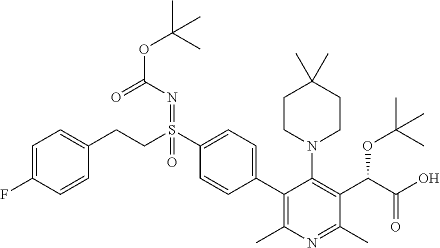 [SMILES: Cc1nc(C)c([C@H](OC(C)(C)C)C(=O)O)c(N2CCC(C)(C)CC2)c1-c1ccc(S(=O)(CCc2ccc(F)cc2)=NC(=O)OC(C)(C)C)cc1]

(2S)-2-(tert-Butoxy)-2-(5-(4-(N-(tert-butoxycarbonyl)-2-(4-fluorophenyl)ethylsulfonimidoyl)phenyl)-4-(4,4-dimethylpiperidin-1-yl)-2,6-dimethylpyridin-3-yl)acetic acid: To a stirred solution of (2S)-2-(tert-butoxy)-2-(5-(4-((E)-N-(tert-butoxycarbonyl)-S-(4-fluorophenethyl)sulfinimidoyl)phenyl)-4-(4,4-dimethylpiperidin-1-yl)-2,6-dimethylpyridin-3-yl)acetic acid (0.011 g, 0.016 mmol) in 1:1 MeOH/H2O (1 mL) was added oxone (0.019 g, 0.032 mmol) at once at rt. After19 h, the reaction mixture was filtered and purified by prep-HPLC to afford (2S)-2-(tert-butoxy)-2-(5-(4-(N-(tert-butoxycarbonyl)-2-(4-fluorophenyl)ethyl-sulfonimidoyl)phenyl)-4-(4,4-dimethylpiperidin-1-yl)-2,6-dimethylpyridin-3-yl)acetic acid (0.0005 g, 0.704 μmol, 4.44% yield) as white solid. LCMS (M+H)=710.7.

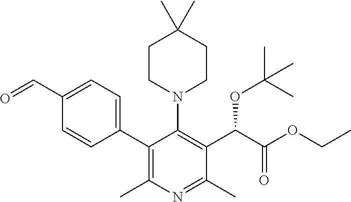 [SMILES: CCOC(=O)[C@@H](OC(C)(C)C)c1c(C)nc(C)c(-c2ccc(C=O)cc2)c1N1CCC(C)(C)CC1]

(S)-Ethyl 2-(tert-butoxy)-2-(4-(4,4-dimethylpiperidin-1-yl)-5-(4-formylphenyl)-2,6-dimethylpyridin-3-yl)acetate: A mixture of (S)-ethyl 2-(5-bromo-4-(4,4-dimethylpiperidin-1-yl)-2,6-dimethylpyridin-3-yl)-2-(tert-butoxy)acetate (0.505 g, 1.109 mmol), (4-formylphenyl)boronic acid (0.333 g, 2.218 mmol) and 2M Na₂CO₃ (1.663 ml, 3.33 mmol) in DMF (10 mL) was degassed for 10 min. Then, Pd(Ph₃P)₄ (0.064 g, 0.055 mmol) was added, degassed for 5 min and placed in a pre-heated oilbath at 110° C. After 2 h, cooled, diluted with ether (50 mL), washed with water (4×10 mL), brine (10 mL), dried (MgSO₄), filtered, concentrated and purified by flash chromatography using 20, 30 and 40% EtOAc/Hex to afford (S)-ethyl 2-(tert-butoxy)-2-(4-(4,4-dimethylpiperidin-1-yl)-5-(4-formylphenyl)-2,6-dimethylpyridin-3-yl)acetate (0.426 g, 0.886 mmol, 80% yield) as off-white solid. ¹H NMR (500 MHz, CDCl₃) δ 10.13 (s, 1H), 8.00 (dt, J=1.4, 8.6 Hz, 2H), 7.49-7.53 (m, 1H), 7.38 (dd, J=1.3, 7.6 Hz, 1H), 6.03 (s, 1H), 4.24-4.31 (m, 1H), 4.16-4.24 (m, 1H), 3.26 (d, J=12.0 Hz, 1H), 2.85 (t, J=12.1 Hz, 1H), 2.63 (s, 3H), 2.26-2.33 (m, 1H), 2.19 (s, 3H), 1.94 (t, J=11.4 Hz, 1H), 1.56 (dt, J=3.6, 12.9 Hz, 1H), 1.32-1.42 (m, 1H), 1.28 (t, J=7.1 Hz, 3H), 1.21 (s, 9H), 1.02-1.08 (m, 1H), 0.90 (br. s., 3H), 0.60 (s, 3H). LCMS (M+H)=481.3.

### EXAMPLE 82 AND 83

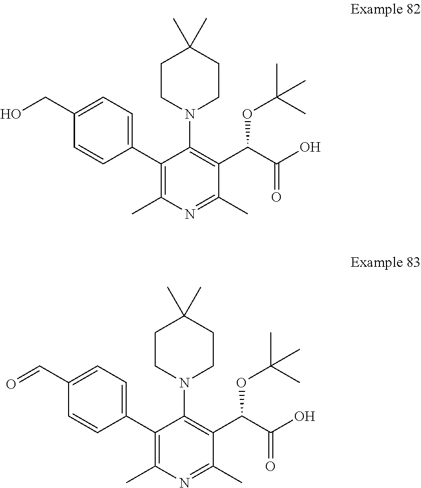 [SMILES: Cc1nc(C)c([C@H](OC(C)(C)C)C(=O)O)c(N2CCC(C)(C)CC2)c1-c1ccc(CO)cc1 ; Cc1nc(C)c([C@H](OC(C)(C)C)C(=O)O)c(N2CCC(C)(C)CC2)c1-c1ccc(C=O)cc1]

(S)-2-(tert-Butoxy)-2-(4-(4,4-dimethylpiperidin-1-yl)-5-(4-(hydroxymethyl)phenyl)-2,6-dimethylpyridin-3-yl)acetic acid and: (S)-2-(tert-butoxy)-2-(4-(4,4-dimethylpiperidin-1-yl)-5-(4-formylphenyl)-2,6-dimethylpyridin-3-yl)acetic acid: A mixture of (S)-ethyl 2-(tert-butoxy)-2-(4-(4,4-dimethylpiperidin-1-yl)-5-(4-formylphenyl)-2,6-dimethylpyridin-3-yl)acetate (0.1 g, 0.208 mmol) and LiOH (0.025 g, 1.040 mmol) in 9:1 EtOH/H₂O was refluxed for 3 h. Then, cooled and purified twice by prep-HPLC to afford Example 82 and Example 83.

### EXAMPLE 82

(S)-2-(tert-Butoxy)-2-(4-(4,4-dimethylpiperidin-1-yl)-5-(4-(hydroxymethyl)phenyl)-2,6-dimethylpyridin-3-yl)acetic acid.NH₄OAc (0.0432 g, 0.081 mmol, 39.1% yield) as white solid. ¹H NMR (500 MHz, CDCl₃) δ 7.47 (d, J=8.2 Hz, 2H), 7.25-7.28 (m, 1H), 7.12-7.16 (m, 1H), 5.85 (br. s., 1H), 4.81 (s, 2H), 3.55 (br. s., 1H), 2.90 (t, J=8.3 Hz, 1H), 2.72 (s, 3H), 2.24 (s, 3H), 1.59-1.67 (m, 1H), 1.49-1.57 (m, 1H), 1.26-1.35 (m, 2H), 1.24 (s, 9H), 0.76 (br. s., 6H). 2H of piperidine and OH hydrogen were not resolved. LCMS (M+H)=455.3.

### EXAMPLE 83

(S)-2-(tert-Butoxy)-2-(4-(4,4-dimethylpiperidin-1-yl)-5-(4-formylphenyl)-2,6-dimethylpyridin-3-yl)acetic acid (0.0205 g, 0.045 mmol, 21.77% yield) as. ¹H NMR (500 MHz, CDCl₃) δ 10.13 (s, 1H), 7.98-8.03 (m, 2H), 7.49-7.53 (m, 1H), 7.37-7.41 (m, 1H), 6.08 (br. s., 1H), 3.49-3.58 (m, 1H), 2.87-2.97 (m, 1H), 2.61 (s, 3H), 2.23-2.30 (m, 1H), 2.19 (s, 3H), 1.89-1.99 (m, 1H), 1.47-1.62 (m, 2H), 1.32-1.40 (m, 1H), 1.26 (s, 9H), 1.04-1.11 (m, 1H), 0.89 (br. s., 4H), 0.60 (br. s., 4H). LCMS (M+H)=453.3.

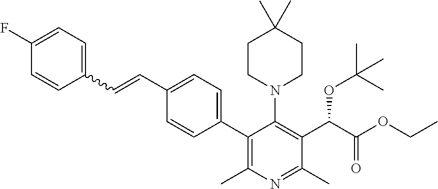 [SMILES: CCOC(=O)[C@@H](OC(C)(C)C)c1c(C)nc(C)c(-c2ccc(C=Cc3ccc(F)cc3)cc2)c1N1CCC(C)(C)CC1]

(S)-Ethyl 2-(tert-butoxy)-2-(4-(4,4-dimethylpiperidin-1-yl)-5-(4-(4-fluorostyryl)phenyl)-2,6-dimethylpyridin-3-yl)acetate: To a stirred white slurry of (4-fluorobenzyl)triphenylphosphonium, chloride salt (0.169 g, 0.416 mmol) in THF (5 mL) was added dropwise 2M BuLi/hex (0.208 ml, 0.416 mmol) at 0° C. After10 min, cold bath was removed and the orange reaction mixture was stirred additional 30 min at rt. Then, solid (S)-ethyl 2-(tert-butoxy)-2-(4-(4,4-dimethylpiperidin-1-yl)-5-(4-formylphenyl)-2,6-dimethylpyridin-3-yl)acetate (0.1 g, 0.208 mmol) was added at once and stirred for 1 h. Then, concentrated and purified by flash chromatography using 10 and 20% EtOAc/Hex to afford (S)-ethyl 2-(tert-butoxy)-2-(4-(4,4-dimethylpiperidin-1-yl)-5-(4-(4-fluorostyryl)phenyl)-2,6-dimethylpyridin-3-yl)acetate (0.097 g, 0.169 mmol, 81% yield) as white solid. ¹H NMR indicates that this is a 2:1 mixture of isomers. ¹H NMR (500 MHz, CDCl₃) δ 7.58-7.61 (m, 1H), 7.52-7.56 (m, 1H), 7.36 (dd, J=1.7, 7.9 Hz, 1H), 7.26-7.30 (m, 3H), 7.14-7.19 (m, 2H), 7.08-7.13 (m, 2H), 7.06 (dd, J=1.7, 7.9 Hz, 1H), 6.90-6.95 (m, 2H), 6.68 (d, J=12.3 Hz, 1H), 6.62 (d, J=12.3 Hz, 1H), 6.08 (s, 1.5H), 4.14-4.31 (m, 3H), 3.17-3.23 (m, 1.5H), 2.83-2.94 (m, 1.5H), 2.62 (s, 1.5H), 2.61 (s, 3H), 2.28-2.36 (m, 1.5H), 2.23 (s, 1.5H), 2.20 (s, 3H), 2.06-2.18 (m, 1.5H), 1.33-1.62 (m, 6H), 1.25-1.29 (m, 4.5H), 1.22 (s, 4.5H), 1.21 (s, 9H), 0.94 (s, 3H), 0.90 (br.s., 1.5H), 0.73 (s, 3H), 0.65 (br. s., 1.5H). LCMS (M+H)=573.5.

### EXAMPLE 84

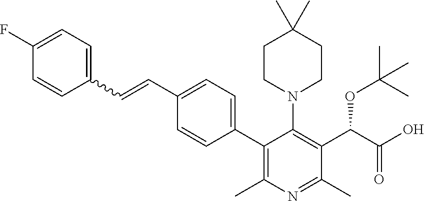 [SMILES: Cc1nc(C)c([C@H](OC(C)(C)C)C(=O)O)c(N2CCC(C)(C)CC2)c1-c1ccc(C=Cc2ccc(F)cc2)cc1]

(S)-2-(tert-Butoxy)-2-(4-(4,4-dimethylpiperidin-1-yl)-5-(4-(4-fluorostyryl)phenyl)-2,6-dimethylpyridin-3-yl)acetic acid: A mixture of (S)-ethyl 2-(tert-butoxy)-2-(4-(4,4-dimethylpiperidin-1-yl)-5-(4-(4-fluorostyryl)phenyl)-2,6-dimethylpyridin-3-yl)acetate (0.091 g, 0.159 mmol) and LiOH (0.027 g, 1.112 mmol) in 9:1 EtOH/H₂O (3 mL) was refluxed for 4 h. Then, cooled and purified by prep-HPLC to afford (S)-2-(tert-butoxy)-2-(4-(4,4-dimethylpiperidin-1-yl)-5-(4-(4-fluorostyryl)phenyl)-2,6-dimethylpyridin-3-yl)acetic acid (0.079 g, 0.145 mmol, 91% yield) as white solid and 3:1 mixture of geometric isomers. LCMS (M+H)=545.5.

### EXAMPLE 85

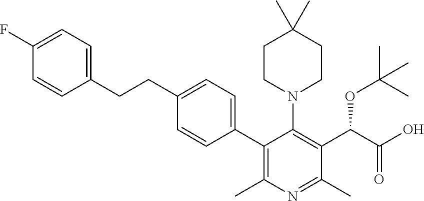 [SMILES: Cc1nc(C)c([C@H](OC(C)(C)C)C(=O)O)c(N2CCC(C)(C)CC2)c1-c1ccc(CCc2ccc(F)cc2)cc1]

(S)-2-(tert-Butoxy)-2-(4-(4,4-dimethylpiperidin-1-yl)-5-(4-(4-fluorophenethyl)-phenyl)-2,6-dimethylpyridin-3-yl)acetic acid: A mixture of (S)-2-(tert-butoxy)-2-(4-(4,4-dimethylpiperidin-1-yl)-5-(4-(4-fluorostyryl)phenyl)-2,6-dimethylpyridin-3-yl)acetic acid (0.047 g, 0.086 mmol) and 10% Pd/C (9.18 mg, 8.63 μmol) in 1:1 MeOH/EtOAc was evaculed and stirred under balloon H2 atmosphere for 2 h. Then, filtered through a plug of celite and concentrated to provide (S)-2-(tert-butoxy)-2-(4-(4,4-dimethylpiperidin-1-yl)-5-(4-(4-fluorophenethyl)phenyl)-2,6-dimethylpyridin-3-yl)acetic acid (0.042 g, 0.077 mmol, 89% yield) as white solid. ¹H NMR (500 MHz, CDCl₃) δ 7.25 (d, J=7.7 Hz, 1H), 7.10-7.17 (m, 4H), 7.06 (d, J=7.9 Hz, 1H), 6.94-7.00 (m, 2H), 5.87 (br. s., 1H), 3.25-4.01 (m, 2H), 2.90-3.03 (m, 4H), 2.72 (s, 3H), 2.24 (s, 3H), 1.27-1.38 (m, 4H), 1.24 (s, 9H), 0.77 (br.s., 9H). 2H of piperadine are missing. LCMS (M+H)=547.5.

### EXAMPLE 86

### (S)-2-(tert-Butoxy)-2-(4-(4,4-dimethylpiperidin-1-yl)-5-(4-(1-(4-fluorobenzyl)-1H-tetrazol-5-yl)phenyl)-2,6-dimethylpyridin-3-yl)acetic acid and/or (S)-2-(tert-Butoxy)-2-(4-(4,4-dimethylpiperidin-1-yl)-5-(4-(2-(4-fluorobenzyl)-2H-tetrazol-5-yl)phenyl)-2,6-dimethylpyridin-3-yl)acetic acid

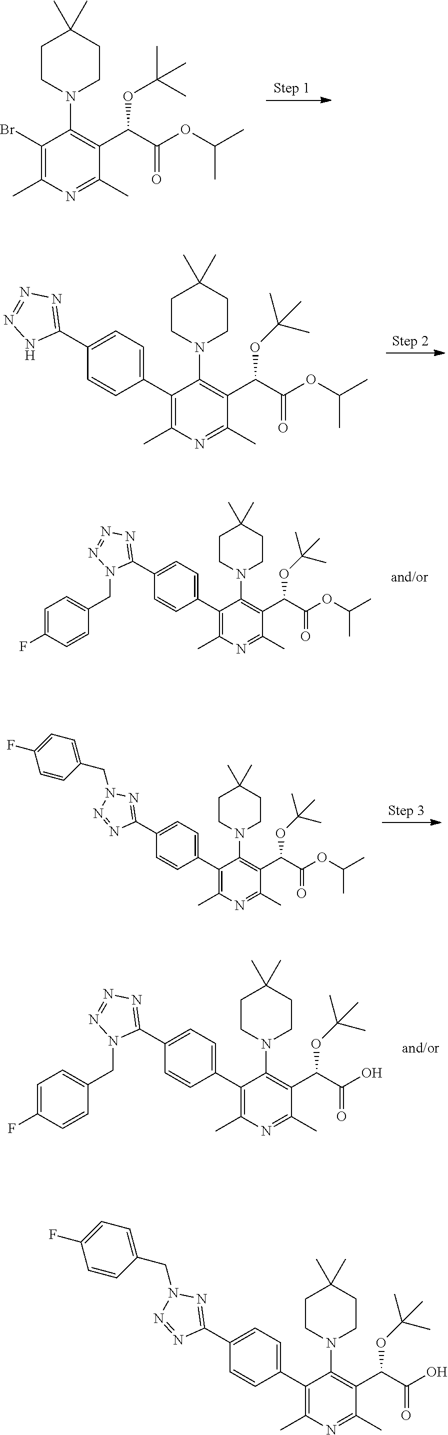 [SMILES: Cc1nc(C)c([C@H](OC(C)(C)C)C(=O)OC(C)C)c(N2CCC(C)(C)CC2)c1Br ; Cc1nc(C)c([C@H](OC(C)(C)C)C(=O)OC(C)C)c(N2CCC(C)(C)CC2)c1-c1ccc(-c2nnn[nH]2)cc1 ; Cc1nc(C)c([C@H](OC(C)(C)C)C(=O)OC(C)C)c(N2CCC(C)(C)CC2)c1-c1ccc(-c2nnnn2Cc2ccc(F)cc2)cc1 ; Cc1nc(C)c([C@H](OC(C)(C)C)C(=O)OC(C)C)c(N2CCC(C)(C)CC2)c1-c1ccc(-c2nnn(Cc3ccc(F)cc3)n2)cc1 ; Cc1nc(C)c([C@H](OC(C)(C)C)C(=O)O)c(N2CCC(C)(C)CC2)c1-c1ccc(-c2nnnn2Cc2ccc(F)cc2)cc1 ; Cc1nc(C)c([C@H](OC(C)(C)C)C(=O)O)c(N2CCC(C)(C)CC2)c1-c1ccc(-c2nnn(Cc3ccc(F)cc3)n2)cc1]

Step 1: To a mixture of (S)-isopropyl 2-(5-bromo-4-(4,4-dimethylpiperidin-1-yl)-2,6-dimethylpyridin-3-yl)-2-(tert-butoxy)acetate (200 mg), (4-(2H-tetrazol-5-yl)phenyl)boronic acid (121 mg) and Cs₂CO₃ (278 mg) in 1,4-dioxane (2 mL) and water (0.4 mL) was added Pd(PPh₃)₄ (49.2 mg). The mixture was flushed with nitrogen and then heated at 85° C. for 6 hours. The mixture was diluted with water (10 mL) and then extracted with EtOAc (2×20 mL). The organic layers were combined, washed with brine and concentrated under vaccum to give a residue which was purified by preparative HPLC to give (S)-isopropyl 2-(5-(4-(1H-tetrazol-5-yl)phenyl)-4-(4,4-dimethylpiperidin-1-yl)-2,6-dimethylpyridin-3-yl)-2-(tert-butoxy)acetate. LCMS (M+H): 535.4.

Step 2: To a solution of (S)-isopropyl 2-(5-(4-(1H-tetrazol-5-yl)phenyl)-4-(4,4-dimethylpiperidin-1-yl)-2,6-dimethylpyridin-3-yl)-2-(tert-butoxy)acetate (50 mg) in DMF (2 mL) was added K₂CO₃ (25.8 mg) and 1-(bromomethyl)-4-fluorobenzene (35.4 mg). The mixture was stirred at room temperature for 4 hours. The mixture was diluted with EtOAc (10 mL), washed with water and brine. The organic layer was dried over MgSO₄ and concentrated under vaccum to give a residue was purified by preparative HPLC to give (S)-isopropyl 2-(tert-butoxy)-2-(4-(4,4-dimethylpiperidin-1-yl)-5-(4-(1-(4-fluorobenzyl)-1H-tetrazol-5-yl)phenyl)-2,6-dimethylpyridin-3-yl)acetate and/or (S)-isopropyl 2-(tert-butoxy)-2-(4-(4,4-dimethylpiperidin-1-yl)-5-(4-(2-(4-fluorobenzyl)-2H-tetrazol-5-yl)phenyl)-2,6-dimethylpyridin-3-yl)acetate. LCMS (M+H): 643.4.

Step 3: To a solution of the product obtained in the step 2 (31 mg) in MeOH (1 mL) and THF (1 mL) was added NaOH (0.482 mL, 1N). The mixture was stirred at 80° C. for 2 hours, before KOH (50 mg) and 1 mL of EtOH were added. The reaction was further stirred at 80° C. for 4 hours, before being acidified by 1N HCl to pH˜4. Solvents were removed under vacuum to give a residue which was purified by preparative HPLC to afford (S)-2-(tert-butoxy)-2-(4-(4,4-dimethylpiperidin-1-yl)-5-(4-(1-(4-fluorobenzyl)-1H-tetrazol-5-yl)phenyl)-2,6-dimethylpyridin-3-yl)acetic acid and/or (S)-2-(tert-butoxy)-2-(4-(4,4-dimethylpiperidin-1-yl)-5-(4-(2-(4-fluorobenzyl)-2H-tetrazol-5-yl)phenyl)-2,6-dimethylpyridin-3-yl)acetic acid. LCMS (M+H): 601.3.

### EXAMPLE 87

### (S)-2-(tert-Butoxy)-2-(4-(4,4-dimethylpiperidin-1-yl)-5-(3-(1-(4-fluorobenzyl)-1H-tetrazol-5-yl)phenyl)-2,6-dimethylpyridin-3-yl)acetic acid and/or (S)-2-(tert-Butoxy)-2-(4-(4,4-dimethylpiperidin-1-yl)-5-(3-(2-(4-fluorobenzyl)-2H-tetrazol-5-yl)phenyl)-2,6-dimethylpyridin-3-yl)acetic acid

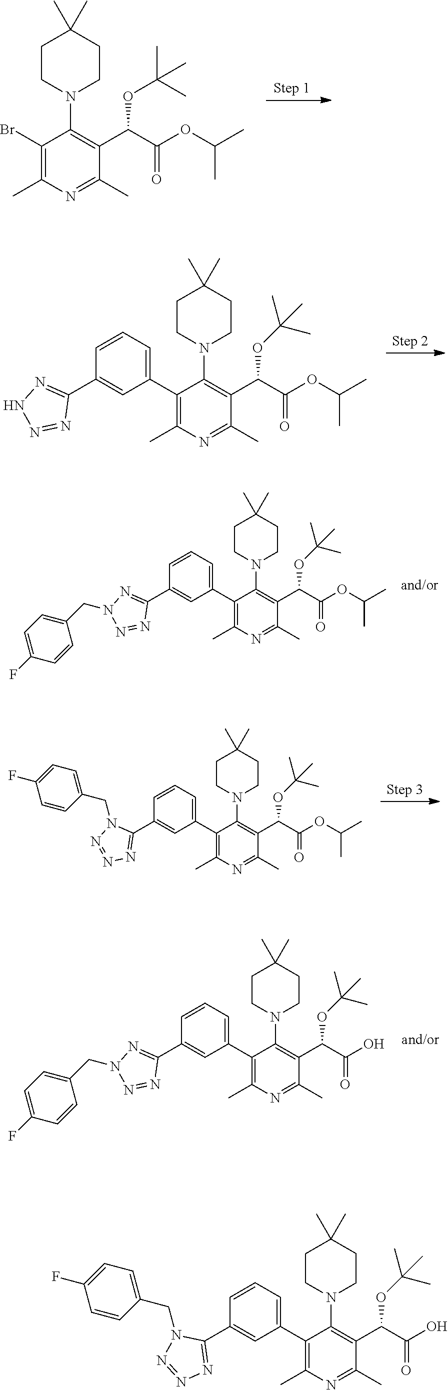 [SMILES: Cc1nc(C)c([C@H](OC(C)(C)C)C(=O)OC(C)C)c(N2CCC(C)(C)CC2)c1Br ; Cc1nc(C)c([C@H](OC(C)(C)C)C(=O)OC(C)C)c(N2CCC(C)(C)CC2)c1-c1cccc(-c2nn[nH]n2)c1 ; Cc1nc(C)c([C@H](OC(C)(C)C)C(=O)OC(C)C)c(N2CCC(C)(C)CC2)c1-c1cccc(-c2nnn(Cc3ccc(F)cc3)n2)c1 ; Cc1nc(C)c([C@H](OC(C)(C)C)C(=O)OC(C)C)c(N2CCC(C)(C)CC2)c1-c1cccc(-c2nnnn2Cc2ccc(F)cc2)c1 ; Cc1nc(C)c([C@H](OC(C)(C)C)C(=O)O)c(N2CCC(C)(C)CC2)c1-c1cccc(-c2nnn(Cc3ccc(F)cc3)n2)c1 ; Cc1nc(C)c([C@H](OC(C)(C)C)C(=O)O)c(N2CCC(C)(C)CC2)c1-c1cccc(-c2nnnn2Cc2ccc(F)cc2)c1]

Step 1: To a mixture of (S)-isopropyl 2-(5-bromo-4-(4,4-dimethylpiperidin-1-yl)-2,6-dimethylpyridin-3-yl)-2-(tert-butoxy)acetate (200 mg), (4-(2H-tetrazol-5-yl)phenyl)boronic acid (121 mg) and Cs₂CO₃ (278 mg) in 1,4-dioxane (2 mL) and water (0.4 mL) was added Pd(PPh₃)₄ (49.2 mg). The mixture was flushed with nitrogen and then heated at 85° C. for 6 hours. The mixture was diluted with water (10 mL) and then extracted with EtOAc (2×20 mL). The organic layers were combined, washed with brine and concentrated under vaccum to give a residue which was purified by preparative HPLC to give (S)-isopropyl 2-(5-(3-(2H-tetrazol-5-yl)phenyl)-4-(4,4-dimethylpiperidin-1-yl)-2,6-dimethylpyridin-3-yl)-2-(tert-butoxy)acetate. LCMS (M+H): 535.4.

Step 2: To a solution of (S)-isopropyl 2-(5-(4-(1H-tetrazol-5-yl)phenyl)-4-(4,4-dimethylpiperidin-1-yl)-2,6-dimethylpyridin-3-yl)-2-(tert-butoxy)acetate (25 mg) in DMF (2 mL) was added K₂CO₃ (12.92 mg) and 1-(bromomethyl)-4-fluorobenzene (17.68 mg). The mixture was stirred at room temperature for 4 hours. The mixture was diluted with EtOAc (10 mL), washed with water and brine. The organic layer was dried over MgSO₄ and concentrated under vaccum to give a residue was purified by preparative HPLC to give (S)-isopropyl 2-(tert-butoxy)-2-(4-(4,4-dimethylpiperidin-1-yl)-5-(3-(1-(4-fluorobenzyl)-1H-tetrazol-5-yl)phenyl)-2,6-dimethylpyridin-3-yl)acetate and/or (S)-isopropyl 2-(tert-butoxy)-2-(4-(4,4-dimethylpiperidin-1-yl)-5-(3-(2-(4-fluorobenzyl)-2H-tetrazol-5-yl)phenyl)-2,6-dimethylpyridin-3-yl)acetate. LCMS (M+H): 643.4.

Step 3: To a solution of the product obtained in the step 2 (24 mg) in MeOH (1 mL) and THF (1 mL) was added NaOH (0.373 mL, 1N). The mixture was stirred at 80° C. for 2 hours, before KOH (50 mg) and 1 mL of EtOH were added. The reaction was further stirred at 80° C. for 4 hours, before being acidified by 1N HCl to pH˜4. Solvents were removed under vacuum to give a residue which was purified by preparative HPLC to afford (S)-2-(tert-butoxy)-2-(4-(4,4-dimethylpiperidin-1-yl)-5-(3-(1-(4-fluorobenzyl)-1H-tetrazol-5-yl)phenyl)-2,6-dimethylpyridin-3-yl)acetic acid and/or (S)-2-(tert-butoxy)-2-(4-(4,4-dimethylpiperidin-1-yl)-5-(3-(2-(4-fluorobenzyl)-2H-tetrazol-5-yl)phenyl)-2,6-dimethylpyridin-3-yl)acetic acid. LCMS (M+H):. 601.3.

### EXAMPLE 88

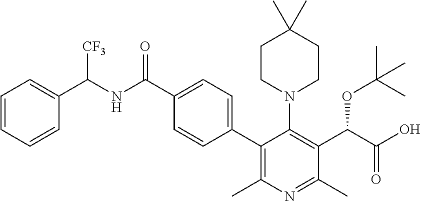 [SMILES: Cc1nc(C)c([C@H](OC(C)(C)C)C(=O)O)c(N2CCC(C)(C)CC2)c1-c1ccc(C(=O)NC(c2ccccc2)C(F)(F)F)cc1]

(2S)-2-(tert-Butoxy)-2-(4-(4,4-dimethylpiperidin-1-yl)-2,6-dimethyl-5-(4-((2,2,2-trifluoro-1-phenylethyl)carbamoyl)phenyl)pyridin-3-yl)acetic acid: The PyBOP (13.33 mg, 0.026 mmol) was added to a solution of (S)-4-(5-(tert-butoxy(carboxy)methyl)-4-(4,4-dimethylpiperidin-1-yl)-2,6-dimethylpyridin-3-yl)benzoic acid (20 mg, 0.043 mmol), 2,2,2-trifluoro-1-phenylethanamine, HCl (5.42 mg, 0.026 mmol), and Hunig's base (0.026 mL, 0.149 mmol) in DMF (1 mL) and stirred for 4 h at 24° C. The reaction was concentrated to 1/3 volume and an additional equivalent of reagents was added, and the temperature was raised to 55° C. and the reaction was stirred for 48 h. The reaction mixture was diluted with EtOAc, washed with water, and brine. The crude product was purified by prep HPLC (XBridge C18, 19×200 mm, 5-μm particles; Mobile Phase A: 5:95 acetonitrile:water with 10-mM ammonium acetate; Mobile Phase B: 95:5 acetonitrile:water with 10-mM ammonium acetate; Gradient: 40-85% B over 20 minutes, then a 5-minute hold at 100% B; Flow: 20 mL/min. There was obtained (2S)-2-(tert-butoxy)-2-(4-(4,4-dimethylpiperidin-1-yl)-2,6-dimethyl-5-(4-((2,2,2-trifluoro-1-phenylethyl)carbamoyl)phenyl)pyridin-3-yl)acetic acid 3 mg (11%). ¹H NMR (500 MHz, DMSO) δ 9.64-9.60 (m, 1H), 8.09-8.03 (m, 2H), 7.74 (d, J=7.0 Hz, 2H), 7.53 (d, J=8.0 Hz, 1H), 7.47-7.42 (m, 3H), 7.32-7.30 (m, 1H), 6.13-6.06 (m, 1H), 5.83 (s, 1H), 3.33-3.30 (m, 1H), 2.81 (t, J=13 Hz, 1H), 2.46 (s, 3H), 2.23 (br. s, 1H), 2.05 (s, 3H), 1.90-1.85 (m, 1H), 1.53-1.46 (m, 1H), 1.32-1.26 (m, 1H), 1.19-1.17 (m, 1H), 1.14 (s, 9H), 1.02-0.99 (m, 1H), 0.84 (s, 3H), 0.56 (s, 3H). UPLC (M+H)=626.4.

### Biological Methods

Inhibition of HIV replication: A recombinant NL-RLuc proviral clone was constructed in which a section of the nef gene from NL4-3 was replaced with the Renilla Luciferase gene. This virus is fully infectious and can undergo multiple cycles of replication in cell culture. In addition, the luciferous reporter provides a simple and easy method for quantitating the extent of virus growth and consequently, the antiviral activity of test compounds. The plasmid pNLRLuc contains the proviral NL-Rluc DNA cloned into pUC18 at the PvuII site. The NL-RLuc virus was prepared by transfection of 293T cells with the plasmid pNLRLuc. Transfections were performed using the LipofectAMINE PLUS kit from Invitrogen (Carlsbad, Calif.) according to the manufacturer and the virus generated was titered in MT-2 cells. For susceptibility analyses, the titrated virus was used to infect MT-2 cells in the presence of compound, and after 5 days of incubation, cells were processed and quantitated for virus growth by the amount of expressed luciferase. Assay media was RPMI 1640 supplemented with 10% heat inactivated fetal bovine serum (FBS), 100 units/ml penicillin G/100 units/ml streptomycin, 10 mM HEPES buffer pH 7.55 and 2 mM L-glutamine. The results from at least 2 experiments were used to calculate the EC₅₀ values. Luciferase was quantitated using the Dual Luciferase kit from Promega (Madison, Wis.). Susceptibility of viruses to compounds was determined by incubation in the presence of serial dilutions of the compound. The 50% effective concentration (EC₅₀) was calculated by using the exponential form of the median effect equation where (Fa)=1/[1+(ED₅₀/drug conc.)^{m}] (Johnson V A, Byington R T. Infectivity Assay. In Techniques in HIV Research. ed. Aldovini A, Walker B D. 71-76. New York: Stockton Press.1990). Results are shown in Table 1. Activity equal to A refers to a compound having an EC₅₀≤100 nM, while B and C denote compounds having an EC₅₀ between 100 nM and 1 uM (B) or >1 uM (C).

TABLE 1

| Example | Activity | EC₅₀ μM |
|---|---|---|
| 1 | A | 0.007 |
| 2 | A | |
| 3 | A | |
| 4 | A | |
| 5 | A | |
| 6 | A | |
| 7 | A | |
| 8 | A | 0.031 |
| 9 | A | |
| 10 | A | |
| 11 | B | 0.135 |
| 12 | B | |
| 13 | A | |
| 14 | A | |
| 15 | A | |
| 16 | A | |
| 17 | A | 0.053 |
| 18 | A | |
| 19 | A | |
| 20 | A | 0.161 |
| 21 | A | |
| 22 | A | |
| 23 | A | |
| 24 | A | |
| 25 | B | |
| 26 | B | 0.373 |
| 27 | A | |
| 28 | B | |
| 29 | A | |
| 30 | A | |
| 31 | A | |
| 32 | B | |
| 33 | A | |
| 34 | A | |
| 35 | B | |
| 36 | A | |
| 37 | C | 1.152 |
| 38 | A | |
| 39 | A | |
| 40 | A | |
| 41 | A | |
| 42 | A | |
| 43 | A | |
| 44 | A | |
| 45 | B | |
| 46 | C | |
| 47 | C | |
| 48 | B | 0.109 |
| 49 | B | |
| 50 | A | |
| 51 | A | |
| 52 | A | |
| 53 | B | |
| 54 | A | |
| 55 | A | 0.053 |
| 56 | A | |
| 57 | C | 2.543 |
| 58 | A | |
| 59 | C | |
| 60 | A | |
| 61 | B | |
| 62 | C | |
| 63 | B | |
| 64 | B | |
| 65 | A | |
| 66 | A | |
| 67 | A | |
| 68 | nd | nd |
| 69 | A | 0.004 |
| 70 | B | |
| 71 | A | |
| 72 | A | |
| 73 | A | |
| 74 | A | |
| 75 | A | |
| 76 | A | |
| 77 | A | |
| 78 | A | 0.016 |
| 79 | A | |
| 80 | A | |
| 81 | A | |
| 82 | A | |
| 83 | A | |
| 84 | B | 0.126 |
| 85 | A | |
| 86 | A | |
| 87 | A | |
| 88 | A | |

nd = not determined

It will be evident to one skilled in the art that the present disclosure is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

1. A compound of Formula I or a pharmaceutically acceptable salt thereof 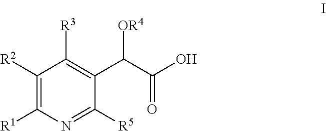
wherein:
R¹ is selected from hydrogen or alkyl;
R² is selected from phenyl, pyridinyl, or pyrimidinyl, and is substituted with 0-1 substituent selected from R⁶, R⁷, R⁸, and R⁹, and also substituted with 0-3 substituents selected from cyano, halo, alkyl, cyanoalkyl, haloalkyl, hydroxyalkyl, alkoxyalkyl, cyanoalkenyl, cycloalkyl, cyanocycloalkyl, hydroxyalkyl, alkoxy, haloalkoxy, formyl, carboxy, and CH₃CONHNHCO—;
R³ is selected from azetidinyl, pyrrolidinyl, piperidinyl, piperazinyl, morpholinyl, homopiperidinyl, homopiperazinyl, or homomorpholinyl, and is substituted with 0-3 substituents selected from cyano, halo, alkyl, haloalkyl, cyanoalkyl, cycloalkyl, alkenyl, alkoxy, haloalkoxy, phenyl, or benzyl;
R⁴ is selected from alkyl or haloalkyl;
R⁵ is alkyl;
R⁶ is selected from CONR¹⁰R¹¹ or (CONR¹⁰R¹¹)alkyl;
R⁷ is selected from Ar², (Ar²)alkyl, (Ar²)hydroxyalkyl , (Ar²)alkenyl, or (Ar²)alkylcarbonyl;
R⁸ is selected from alkylthio, (Ar¹)alkylthio, alkylsulfonyl, (Ar¹)alkylsulfonyl, ((Ar¹)alkyl)(alkoxycarbonylN═)S, ((Ar¹)alkyl)(alkoxycarbonylN═)(O)S, or SONR¹²R¹³;
R⁹ is NR¹⁴R¹⁵;
R¹⁰ is selected from hydrogen, alkyl, cycloalkyl, (Ar¹)alkyl, (Ar¹)haloalkyl, ((Ar¹)CO)alkyl, ((Ar¹)CH₂CO)alkyl;
R¹¹ is selected from hydrogen or alkyl;
or NR¹⁰R¹¹ taken together is selected from azetidinyl, pyrrolidinyl, piperidinyl, piperazinyl, morpholinyl, homopiperidinyl, homopiperazinyl, or homomorpholinyl;
R¹² is selected from hydrogen, alkyl, or cycloalkyl;
R¹³ is selected from hydrogen, alkyl, or cycloalkyl;
or NR¹²R¹³ taken together is selected from azetidinyl, pyrrolidinyl, piperidinyl, piperazinyl, morpholinyl, homopiperidinyl, homopiperazinyl, or homomorpholinyl;
R¹⁴ is selected from hydrogen, alkyl, (Ar¹)alkyl, (Ar¹)hydroxyalkyl, (Ar¹)alkylcarbonyl, or benzyloxycarbonyl;
R¹⁵ is selected from hydrogen, alkyl, hydroxyalkyl, (Ar¹)alkyl, or alkylcarbonyl;
Ar¹ is selected from phenyl or pyridinyl and is substituted with 0-3 substituents selected from halo, alkyl, haloalkyl, alkoxy, haloalkoxy, carboxy, and alkoxycarbonyl; and
Ar² is selected from phenyl or naphthyl and is substituted with 0-3 substituents selected from halo, alkyl, haloalkyl, alkoxy, haloalkoxy, phenyl, and pyridinyl; or Ar² is selected from oxazolyl, thiazolyl, imidazolyl, or tetrazolyl, and is substituted with 0-2 substituents selected from halo, (Ar¹), and (Ar¹)alkyl.

2. A compound or salt of claim 1 wherein R² is selected from phenyl, pyridinyl, or pyrimidinyl, and is substituted with 1 R⁶ substituent and also substituted with 0-3 substituents selected from cyano, halo, alkyl, cyanoalkyl, haloalkyl, hydroxyalkyl, alkoxyalkyl, cyanoalkenyl, cycloalkyl, cyanocycloalkyl, hydroxyalkyl, alkoxy, haloalkoxy, formyl, carboxy, and CH₃CONHNHCO—.

3. A compound or salt of claim 1 wherein R² is selected from phenyl, pyridinyl, or pyrimidinyl, and is substituted with 1 R⁷ substituent and also substituted with 0-3 substituents selected from cyano, halo, alkyl, cyanoalkyl, haloalkyl, hydroxyalkyl, alkoxyalkyl, cyanoalkenyl, cycloalkyl, cyanocycloalkyl, hydroxyalkyl, alkoxy, haloalkoxy, formyl, carboxy, and CH₃CONHNHCO—.

4. A compound or salt of claim 1 wherein R² is selected from phenyl, pyridinyl, or pyrimidinyl, and is substituted with 1 R⁸ substituent and also substituted with 0-3 substituents selected from cyano, halo, alkyl, cyanoalkyl, haloalkyl, hydroxyalkyl, alkoxyalkyl, cyanoalkenyl, cycloalkyl, cyanocycloalkyl, hydroxyalkyl, alkoxy, haloalkoxy, formyl, carboxy, and CH₃CONHNHCO—.

5. A compound or salt of claim 1 wherein R² is selected from phenyl, pyridinyl, or pyrimidinyl, and is substituted with 1 R⁹ substituent and also substituted with 0-3 substituents selected from cyano, halo, alkyl, cyanoalkyl, haloalkyl, hydroxyalkyl, alkoxyalkyl, cyanoalkenyl, cycloalkyl, cyanocycloalkyl, hydroxyalkyl, alkoxy, haloalkoxy, formyl, carboxy, and CH₃CONHNHCO—.

6. A compound or salt of claim 1 wherein R³ is piperidinyl, gem-disubstituted in the 4-position with 2 substituents selected from cyano, halo, alkyl, haloalkyl, cycloalkyl, halocycloalkyl, alkenyl, alkoxy, haloalkoxy, CON(R⁶)(R⁷), phenyl, benzyl, or (alkyl)oxadiazolyl.

7. A compound or salt of claim 1 wherein R¹⁰ is selected from hydrogen, alkyl, cycloalkyl, (Ar¹)alkyl, (Ar¹)haloalkyl, ((Ar¹)CO)alkyl, ((Ar¹)CH₂CO)alkyl; and R¹¹ is selected from hydrogen or alkyl.

8. A compound or salt of claim 1 wherein NR¹⁰R¹¹ taken together is selected from azetidinyl, pyrrolidinyl, piperidinyl, piperazinyl, morpholinyl, homopiperidinyl, homopiperazinyl, or homomorpholinyl.

9. A compound or salt of claim 1 wherein R¹² and R¹³ are selected from hydrogen, alkyl, or cycloalkyl.

10. A compound or salt of claim 1 wherein NR¹²R¹³ taken together is selected from azetidinyl, pyrrolidinyl, piperidinyl, piperazinyl, morpholinyl, homopiperidinyl, homopiperazinyl, or homomorpholinyl.

11. A compound or salt of claim 1 wherein Ar² is selected from phenyl or naphthyl and is substituted with 0-3 substituents selected from halo, alkyl, haloalkyl, alkoxy, haloalkoxy, phenyl, and pyridinyl.

12. A compound or salt of claim 1 wherein Ar² is selected from oxazolyl, thiazolyl, imidazolyl, or tetrazolyl, and is substituted with 0-2 substituents selected from halo, (Ar¹), and (Ar¹)alkyl.

13. A composition useful for treating HIV infection comprising a compound or salt of claim 1 and a pharmaceutically acceptable carrier.

14. The composition of claim 13 further comprising at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors.

15. The composition of claim 14 wherein the other agent is dolutegravir.

16. A method for treating HIV infection comprising administering a compound of claim 1, or a pharmaceutically acceptable salt thereof, to a patient in need thereof.

17. The method of claim 16 further comprising administering at least one other agent used for treatment of AIDS or HIV infection selected from nucleoside HIV reverse transcriptase inhibitors, non-nucleoside HIV reverse transcriptase inhibitors, HIV protease inhibitors, HIV fusion inhibitors, HIV attachment inhibitors, CCR5 inhibitors, CXCR4 inhibitors, HIV budding or maturation inhibitors, and HIV integrase inhibitors.

18. The method of claim 17 wherein the other agent is dolutegravir.

19. The method of claim 17 wherein the other agent is administered to the patient prior to, simultaneously with, or subsequently to the compound of claim 1.
